SEQUENCE LISTING (93 sequences)

SEQ ID NO: 1 - DNA, 19 bp, synthetic construct
  source 1..19: mol_type = other DNA; note = TetO Promoter operator of a first sequence
tccctatcagtgatagaga

SEQ ID NO: 2 - DNA, 19 bp, synthetic construct
  source 1..19: mol_type = other DNA; note = tetO-4C5G Promoter operator of a second sequence and/or array operator of a second sequence
tccccgtcagtgacggaga

SEQ ID NO: 3 - DNA, 615 bp, synthetic construct
  source 1..615: mol_type = other DNA; note = rtTA
atgtcaagattggacaaaagcaaggttattaacggtgccttggaactgttgaatggtgtaggtatagaaggcttaacgaccagaaaattagcacaaaagttaggtgtagagcagccaacattatattggcacgtcaaaaacaagagagcattgctagacgcattgccaatcgaaatgttagatcgtcatcacactcatttttgcccattagagggtgagagttggcaagactttttgcgtaacaatgctaaatcctttagatgcgccttgttgagccatcgtgatggtgccaaggttcatctaggtacaaggccaacagaaaaacagtatgagactttggagaatcagctagcctttttatgccaacagggtttttcattggagaatgccttatacgctttaagtgcagttggtcacttcactttaggttgcgtactagaagaacaagagcaccaggttgctaaagaagagagagaaacacctacaacagattcaatgcctccattgctaagacaagctatcgaactgtttgatagacagggtgcagaacctgcattcttgttcggtttagagttgattatttgcggcttggaaaagcagctaaaatgcgaatct

SEQ ID NO: 4 - DNA, 615 bp, unidentified
  source 1..615: mol_type = other DNA; note = TetR
atgtcaagattggataagtccaaggttatcaactctgctttggagttgttgaacgaagttggtattgaaggtttgaccactagaaagttggctcaaaaattgggtgttgctcaaaaaaccttgtactggcacgttaagaacaaaagagctttgttggacgctttggccattgaaatgttggatagacatcatacccatttctgtccattggaaggtgaatcatggcaagatttcttgagaaacaacgccaagtctttcagatgtgctttgttgtctcatagagatggtgctaaagttcacttgggtactagaccaactgaaaagcaatacgaaactttggaaaaccagttggctttcttgtgtcaacaaggtttctctttagaaaacgccttgtatgctttgtctgctgttggtcattttaccttgggttgtgttttggaagatcaagaacatcaagtcgccaaagaagaaagggaaactccaactactgattctatgccaccattattgagacaagccattgagttgtttgatcatcaaggtgctgaaccagcttttttgtttggtttggaattgatcatctgcggtttggagaaacaattgaagtgtgaatct

SEQ ID NO: 5 - DNA, 1059 bp, synthetic construct
  source 1..1059: mol_type = other DNA; note = VP domain
aaattaggtggttctggtggttccccaaagaagaaaagaaaggttggtagagctgaagcttctggttcaggtagagcagatgctttggatgatttcgatttggacatgttgggttctgatgcattggacgatttcgacttagatatgttaggtagtgacgccttggatgacttcgatctggatatgctaggttccgatgccttagacgactttgatttagacatgctgattaactcaagatcctctggttctcctaagaagaagagaaaagtcggttctcaatacttgccagataccgatgatagacacagaatcgaagaaaagaggaagagaacttacgaaaccttcaagtccatcatgaagaagtctccattttctggtccaactgatccaagaccaccaccaagaagaattgctgttccttcaagatcctctgcctctgttccaaaaccagctccacaaccttatccttttacatcttctttgtccaccatcaactacgatgaatttccaactatggttttcccatccggtcaaatttctcaagcttctgctttggctccagctcctccacaagttttgccacaagctccagcaccagctccagctccagcaatggtttctgctttagctcaagccccagctcctgttccagttttagctccaggtccacctcaagctgttgctccaccagcaccaaaacctactcaagctggtgaaggtactttgtctgaagctttgttgcagttgcaattcgatgatgaagatttgggtgctttgttgggtaattctactgatccagctgtttttaccgatttggcttctgttgataactccgaatttcagcagttgttgaatcaaggtattccagttgctcctcatacaactgaacctatgttgatggaatatccagaagcaattactagattagttactggtgctcaaagacctccagatcctgcacctgctcctttgggtgctccaggtttaccaaatggtctattgtctggtgacgaagatttttcctctattgccgatatggatttctccgctttgttg

SEQ ID NO: 6 - DNA, 342 bp, unidentified
  source 1..342: mol_type = other DNA; note = Gal4
gctaatttcaatcagtctggtaatattgccgactcctctttgtctttcactttcactaattcttccaacggtccaaacttgatcactacccaaactaattctcaagctttgtctcagccaattgcctcttctaatgttcacgataacttcatgaacaacgaaatcaccgcctccaaaattgatgatggtaacaattctaagccattgtctccaggttggactgatcaaactgcttataatgctttcggtattaccaccggtatgtttaacactactactatggatgacgtctacaactacttgttcgatgatgaagatacaccaccaaatccaaagaaagaa

SEQ ID NO: 7 - DNA, 123 bp, unidentified
  source 1..123: mol_type = other DNA; note = Mxi1
atggaacgtgtgagaatgattaatgtgcaaaggctgttagaagccgcagagtttttagaaagaagagaaagagaatgcgaacacgggtatgccagttctttccctagcatgccctctcccaga

SEQ ID NO: 8 - DNA, 120 bp, unidentified
  source 1..120: mol_type = other DNA; note = Mxi1
gaaagggttagaatgattaacgttcaaaggttgttggaagctgccgaatttttggaaagacgtgaaagggaatgtgaacatggttacgcttcatcttttccatctatgccatctccaaga

SEQ ID NO: 9 - DNA, 24 bp, synthetic construct
  source 1..24: mol_type = other DNA; note = NLS
ccaccaaagaaaaaacgtaaggtc

SEQ ID NO: 10 - DNA, 24 bp, synthetic construct
  source 1..24: mol_type = other DNA; note = NLS
ccaccaaaaaagaaaagaaaggtt

SEQ ID NO: 11 - DNA, 24 bp, synthetic construct
  source 1..24: mol_type = other DNA; note = NLS
gacccaaagaaaaaaagaaaagtt

SEQ ID NO: 12 - DNA, 24 bp, synthetic construct
  source 1..24: mol_type = other DNA; note = NLS
gacccaaagaagaagagaaaggtt

SEQ ID NO: 13 - DNA, 24 bp, synthetic construct
  source 1..24: mol_type = other DNA; note = NLS
cctcctaagaaaaagagaaaagtt

SEQ ID NO: 14 - DNA, 24 bp, synthetic construct
  source 1..24: mol_type = other DNA; note = NLS
ccaccaaagaagaaaagaaaggtt

SEQ ID NO: 15 - DNA, 21 bp, synthetic construct
  source 1..21: mol_type = other DNA; note = NLS
ccaaaaaagaagagaaaggtc

SEQ ID NO: 16 - DNA, 1014 bp, synthetic construct
  source 1..1014: mol_type = other DNA; note = rtTA-Gal4
atgtcaagattggacaaaagcaaggttattaacggtgccttggaactgttgaatggtgtaggtatagaaggcttaacgaccagaaaattagcacaaaagttaggtgtagagcagccaacattatattggcacgtcaaaaacaagagagcattgctagacgcattgccaatcgaaatgttagatcgtcatcacactcatttttgcccattagagggtgagagttggcaagactttttgcgtaacaatgctaaatcctttagatgcgccttgttgagccatcgtgatggtgccaaggttcatctaggtacaaggccaacagaaaaacagtatgagactttggagaatcagctagcctttttatgccaacagggtttttcattggagaatgccttatacgctttaagtgcagttggtcacttcactttaggttgcgtactagaagaacaagagcaccaggttgctaaagaagagagagaaacacctacaacagattcaatgcctccattgctaagacaagctatcgaactgtttgatagacagggtgcagaacctgcattcttgttcggtttagagttgattatttgcggcttggaaaagcagctaaaatgcgaatctggtagtggttcacctcctaagaaaaagagaaaagttggttccggtagcgctaatttcaatcagtctggtaatattgccgactcctctttgtctttcactttcactaattcttccaacggtccaaacttgatcactacccaaactaattctcaagctttgtctcagccaattgcctcttctaatgttcacgataacttcatgaacaacgaaatcaccgcctccaaaattgatgatggtaacaattctaagccattgtctccaggttggactgatcaaactgcttataatgctttcggtattaccaccggtatgtttaacactactactatggatgacgtctacaactacttgttcgatgatgaagatacaccaccaaatccaaagaaagaaggatcctaa

SEQ ID NO: 17 - DNA, 792 bp, synthetic construct
  source 1..792: mol_type = other DNA; note = TetR-Mxi1
atgtcaagattggataagtccaaggttatcaactctgctttggagttgttgaacgaagttggtattgaaggtttgaccactagaaagttggctcaaaaattgggtgttgctcaaaaaaccttgtactggcacgttaagaacaaaagagctttgttggacgctttggccattgaaatgttggatagacatcatacccatttctgtccattggaaggtgaatcatggcaagatttcttgagaaacaacgccaagtctttcagatgtgctttgttgtctcatagagatggtgctaaagttcacttgggtactagaccaactgaaaagcaatacgaaactttggaaaaccagttggctttcttgtgtcaacaaggtttctctttagaaaacgccttgtatgctttgtctgctgttggtcattttaccttgggttgtgttttggaagatcaagaacatcaagtcgccaaagaagaaagggaaactccaactactgattctatgccaccattattgagacaagccattgagttgtttgatcatcaaggtgctgaaccagcttttttgtttggtttggaattgatcatctgcggtttggagaaacaattgaagtgtgaatctggttctggttctccaccaaaaaagaaaagaaaggttggttcaggttccgaaagggttagaatgattaacgttcaaaggttgttggaagctgccgaatttttggaaagacgtgaaagggaatgtgaacatggttacgcttcatcttttccatctatgccatctccaagaggatcctaa

SEQ ID NO: 18 - DNA, 20 bp, unidentified
  source 1..20: mol_type = other DNA; note = 20nt DNA Csy4 cleavage site
gttcactgccgtataggcag

SEQ ID NO: 19 - DNA, 28 bp, unidentified
  source 1..28: mol_type = other DNA; note = 28nt DNA Csy4 cleavage site
gttcactgccgtataggcagctaagaaa

SEQ ID NO: 20 - RNA, 20 nt, unidentified
  source 1..20: mol_type = other RNA; note = 20nt RNA Csy4 cleavage site
gttcactgccgtataggcag

SEQ ID NO: 21 - RNA, 28 nt, unidentified
  source 1..28: mol_type = other RNA; note = 28nt RNA Csy4 cleavage site
gttcactgccgtataggcagctaagaaa

SEQ ID NO: 22 - DNA, 38 bp, synthetic construct
  source 1..38: mol_type = other DNA
aacaaagcaccagtggtctagtggtagaatagtaccct

SEQ ID NO: 23 - DNA, 75 bp, synthetic construct
  source 1..75: mol_type = other DNA; note = Dr-RNAGly(GCC)
gtgagcattggtggttcagtggtagaattctcgcctgccacgcgggaggcccgggttcgattcccggccaatgca

SEQ ID NO: 24 - DNA, 83 bp, synthetic construct
  source 1..83: mol_type = other DNA; note = Dr-tRNALys(CTT)
gttctcatcagcccggctagctcagtcggtagagcatgagactcttaatctcagggtcgtgggttcgagccccacgtcgggcg

SEQ ID NO: 25 - DNA, 81 bp, synthetic construct
  source 1..81: mol_type = other DNA; note = Dr-tRNAAsn(GTT)
gctatctgtctctgtggcgcaatcggttagcgcgttcggctgttaaccgaaaggttggtggttcgagcccacccagggacg

SEQ ID NO: 26 - DNA, 81 bp, synthetic construct
  source 1..81: mol_type = other DNA; note = Dr-tRNAMet(CAT)
gcctgaaggtttccgtagtgtagtggttatcacgttcgcctcatacgcgaaaggtccccagttcgaaactgggcggaaaca

SEQ ID NO: 27 - DNA, 79 bp, synthetic construct
  source 1..79: mol_type = other DNA; note = Dr-tRNAGln(CTG)
gacttgaggttccatggtgtaatggttagcactctggactctgaatccagcgatccgagttcaaatctcggtgggacca

SEQ ID NO: 28 - DNA, 89 bp, synthetic construct
  source 1..89: mol_type = other DNA; note = Dr-tRNASer(GCT)
ggaaaatgacgaggtggccgagtggttaaggcgatggactgctaatccattgtgctttgcacgcatgggttcgaatcccatcctcgtcg

SEQ ID NO: 29 - DNA, 79 bp, synthetic construct
  source 1..79: mol_type = other DNA; note = Dr-tRNAThr(AGT)
gcagcggcgccgtggcttagttggttaaagcgcctgtctagtaaacaggagatcctgggttcgaatcccagcggtgcct

SEQ ID NO: 30 - DNA, 76 bp, synthetic construct
  source 1..76: mol_type = other DNA; note = Dr-tRNAHis(GTG)
gctcgccgtgatcgtacagtggttagtactctgcgttgtggccgcagcaaccccggttcgaatccgggtcacggca

SEQ ID NO: 31 - DNA, 87 bp, synthetic construct
  source 1..87: mol_type = other DNA; note = Dr-tRNALeu(CAG)
gcatgtcaggatggccgagtggtctaaggcgctgcgttcaggtcgcagtctcccctggaggcgtgggttcgaatcccacttctgaca

SEQ ID NO: 32 - DNA, 78 bp, synthetic construct
  source 1..78: mol_type = other DNA; note = Os-tRNAGly(GCC)
gaacaaagcaccagtggtctagtggtagaatagtaccctgccacggtacagacccgggttcgattcccggctggtgca

SEQ ID NO: 33 - DNA, 78 bp, synthetic construct
  source 1..78: mol_type = other DNA; note = Os-tRNAGly(GCC)-scrambled
gaacctcttacacgcgcagatcaactaaatgtacactgcgacggtccgtggctccgagaggggttacagggtacgctg

SEQ ID NO: 34 - DNA, 75 bp, synthetic construct
  source 1..75: mol_type = other DNA; note = Dr-tRNAGly(GCC)-scrambled
gcgctgtggcgtaccgggtacgtactcgcttgactgggttggtactaggcgaaaccagctccgtgggattgcacc

SEQ ID NO: 35 - DNA, 44 bp, unidentified
  source 1..44: mol_type = other DNA; note = Hammerhead ribozyme
gttccccctgatgagtccgtgaggacgaaacgagtaagctcgtc

SEQ ID NO: 36 - DNA, 68 bp, Hepatitis D virus
  source 1..68: mol_type = other DNA; note = Hepatitus delta virus ribozyme
ggccggcatggtcccagcctcctcgctggcgccggctgggcaacatgcttcggcatggcgaatgggac

SEQ ID NO: 37 - DNA, 4323 bp, synthetic construct
  source 1..4323: mol_type = other DNA; note = dCas9-Mxi1
atgtctgatccaaaaaagaagagaaaggtcggttctggttctgacaagaaatactctattggtttggctattggcaccaattctgttggttgggctgttattactgacgagtacaaagttccctccaagaagtttaaggttttgggtaacactgacaggcactccattaagaaaaatttgattggtgccctgttgttcgattctggtgaaactgctgaagctactagattgaaaagaaccgctagaagaagatacaccagaagaaagaacagaatctgctacttgcaagagattttctctaacgaaatggccaaggttgatgattcattcttccacagattggaagagtccttcttggttgaagaggataagaaacacgaaagacatccaatcttcggtaacatcgttgatgaagttgcttaccacgaaaagtacccaactatctaccatttgagaaagaagttggttgactctaccgataaggctgatttgagattgatctatttggctttggcccacatgattaagttcagaggtcatttcttgatcgagggtgatttgaatccagataactccgatgttgacaagttgttcatccaattggtccaaacttacaaccagttgttcgaagaaaatccaatcaatgcctctggtgttgatgctaaggctattttgtctgctagactgtctaagtcaagaaggttggaaaacttgattgctcaattgccaggtgaaaagaagaatggtttgttcggtaatttgatcgccttgtctttgggtttgactccaaatttcaagtccaacttcgatttggctgaagatgctaagttgcaattgtctaaggatacctacgatgatgacttggataatttgttggcccaaatcggtgatcaatacgctgatttgtttttggctgctaagaacttgtccgatgccattttgttgtccgatatcttgagagttaacaccgaaattactaaggctccattgtctgcctctatgatcaaaagatacgatgaacaccaccaagacttgactttgttgaaagctttggtcagacaacagttgccagaaaagtacaaagaaatcttcttcgaccagtccaaaaatggttacgctggttatattgatggtggtgcttctcaagaagagttctacaagttcattaagcccatcttggaaaagatggatggtactgaagaactgttggtcaagttgaacagagaagatttgttgagaaagcagagaaccttcgataacggttctattccacatcaaatccacttgggtgaattgcatgctattttgagaaggcaagaggatttctacccattcttgaaggataacagagaaaagatcgagaagatcctgaccttcagaatcccatattatgttggtccattggccagaggtaattcaagatttgcttggatgactaggaagtccgaagaaactattactccatggaacttcgaagaggttgttgataagggtgcttctgctcaatccttcattgaaagaatgactaacttcgacaagaacctgccaaacgaaaaggttttgccaaaacactctttgctgtacgaatacttcaccgtctacaacgaattgactaaggttaagtacgtcaccgaaggtatgagaaaaccagcttttttgtctggtgaacaaaagaaggctatcgtcgatttgttgttcaagactaacagaaaggttaccgtcaagcaattgaaagaggactacttcaagaaaatcgagtgcttcgactccgttgaaattagtggtgttgaagatagattcaacgcctccttaggtacttaccatgacttattgaagatcatcaaggacaaggatttcttggacaacgaagaaaacgaggatatcttggaggatatcgttttgactttgaccttgttcgaggatagggaaatgatcgaagaaaggttgaaaacctacgctcacttgtttgatgataaggtcatgaagcagttgaagagaagaaggtatactggttggggtagattgtctaggaaattgattaacggtatcagggacaagcaatccggtaagactattttggactttttgaagtctgatggcttcgccaatagaaacttcatgcaattgatccacgatgacagcttgaccttcaaagaagatattcaaaaggcccaagtttccggtcaaggtgattctttacatgaacatattgctaacttggctggttccccagctattaagaaaggtattttacaaaccgttaaggtggtcgacgaattggtaaaagttatgggtagacataagccagagaacatcgttattgaaatggctagagaaaatcagactacccaaaagggtcaaaagaactccagagaaaggatgaagagaattgaagagggtatcaaagaattgggctcccagattttgaaagaacacccagttgaaaacacccaattgcagaacgaaaagctgtacttgtactacttgcagaatggtagagatatgtacgtcgaccaagaattggatatcaacagattgtccgattacgatgtcgatgctatcgttccacagtcttttttgaaggatgactccatcgataacaaggtcttgactagatccgataagaacagaggtaagtctgataacgttccatctgaagaggtcgtcaagaagatgaagaattattggagacagttgctgaacgccaagttgattactcaaagaaagttcgacaatttgaccaaggctgaaagaggtggtttgtctgaattggacaaagcaggtttcattaagaggcaattggttgaaactaggcaaatcaccaaacacgttgcccaaattttggattccagaatgaataccaagtacgacgaaaacgataagttgatcagggaagttaaggtcatcacgttgaagtctaagttggtttccgatttcagaaaggacttccaattctacaaggtcagggaaatcaacaactaccatcatgctcatgatgcttacttgaatgctgttgttggtactgccttgattaagaagtatccaaagttggaatccgagttcgtttacggtgattacaaggtttacgacgtcagaaagatgattgccaagtccgaacaagaaattggtaaggctactgctaagtacttcttctactccaacatcatgaatttcttcaagaccgaaatcactttggccaacggtgaaattagaaaaaggccattgattgagactaacggtgagactggtgaaatagtttgggataagggtagagatttcgctaccgttagaaaagttttgtccatgccacaagttaacatcgtcaaaaagaccgaagttcaaactggtggtttctccaaagaatccatcttgccaaaaagaaactccgacaaattgatcgccagaaagaaagattgggacccaaaaaagtatggtggttttgattctccaaccgttgcttactctgttttggttgttgctaaagttgaaaagggcaagagcaagaagttgaagtcagtcaaagaactattgggcatcacgatcatggaaaggtcatcttttgaaaaaaacccaatcgactttttggaggccaagggttacaaagaagttaagaaggatctgattatcaagctgcccaagtactctttgttcgaattggaaaacggtcgtaagagaatgttggcttctgctggtgaattacaaaagggtaatgaattggctctgccctctaagtacgttaactttctatacttggcctctcactacgaaaagttgaagggttctcctgaagataacgaacaaaagcagttgtttgtcgaacagcataagcactacttggacgaaatcatcgaacagatttctgaattttccaagagggttattttggctgatgccaatttggataaggtcttgtctgcttacaacaagcacagagataagccaattagagaacaagccgaaaacatcattcacctgttcactttgactaatttgggtgctccagctgcttttaagtatttcgataccaccatcgacagaaagagatacacttctacaaaagaggttctggacgctaccttgattcatcaatctattactggcttgtacgaaaccaggatcgacttgtctcaattaggtggtgatggttcaggttctccaccaaagaaaaaacgtaaggtcggatctggttctatggaacgtgtgagaatgattaatgtgcaaaggctgttagaagccgcagagtttttagaaagaagagaaagagaatgcgaacacgggtatgccagttctttccctagcatgccctctcccagaggatcctaa

SEQ ID NO: 38 - DNA, 4839 bp, synthetic construct
  source 1..4839: mol_type = other DNA; note = dCas12a-VP
atgtctgacccaaagaagaagagaaaggttggttctggttccaagttggaaaagtttaccaactgctactctctgtctaagaccttgagattcaaagctattcctgttggtaagacccaagaaaacattgacaacaagaggttgttggttgaggacgaaaaaagagctgaagattacaagggtgtcaagaagttgctggacagatattacttgtccttcatcaacgatgtcctgcactctattaagctgaagaacttgaacaactacatctccctgttcagaaaaaagaccagaaccgagaaagagaacaaagagttagaaaacttggagatcaacctgaggaaagaaattgctaaggctttcaagggtaacgagggttacaaatctttgttcaagaaggacatcatcgaaaccatcttgccagaatttttggatgataaggacgaaattgccttggtcaattctttcaacggtttcactactgctttcactggtttctttgacaacagggaaaacatgttctctgaagaagctaagtctacctccattgctttcagatgcattaacgaaaacttgaccaggtacatctccaacatggatattttcgaaaaggttgatgccatcttcgataagcacgaagtccaagaaatcaaagagaagattttgaactccgattacgacgtcgaagattttttcgaaggtgagttcttcaacttcgttttgactcaagaaggtatcgacgtttacaacgctattattggtggtttcgttaccgaatctggtgaaaagattaagggtttgaacgagtacatcaacctgtacaatcaaaagaccaagcagaagttgccaaagtttaagcccttgtacaaacaagtcttgtccgacagagaatccttgtctttttatggtgaaggttacacctccgatgaagaagttttggaagttttcagaaacaccctgaacaagaactccgaaatcttctcatccattaagaagttggagaagctgtttaagaacttcgacgaatattcctccgctggtatctttgtcaaaaatggtccagctatttccaccatctccaaggatatttttggtgaatggaacgtcatcagagataagtggaatgctgaatacgatgacatccacttgaaaaagaaggctgttgttaccgaaaagtacgaggatgatagacgtaagtccttcaagaagattggctctttctctctggaacaattgcaagaatatgctgatgccgatttgtccgttgtcgaaaaattgaaagaaatcatcatccagaaggtcgacgaaatctacaaagtttacggttcctctgaaaagttgttcgatgctgatttcgttttggagaagtcgttgaagaaaaacgatgctgttgttgccatcatgaaggatttgttggattccgttaagagcttcgagaactacattaaggcttttttcggtgagggcaaagaaactaacagagatgaatcattctacggcgattttgttttggcctacgatattttgttgaaggttgatcatatctacgacgccatcagaaactacgttactcaaaaaccatactccaaagacaagttcaagctgtactttcaaaacccacaattcatgggtggttgggacaaagacaaagaaacagattacagagccaccatcttgagatatggttctaagtactacttggccatcatggataagaaatacgctaagtgcttgcaaaagatcgataaggatgatgtcaacggtaactacgagaagatcaactacaaattattgcccggtccaaacaagatgttgcctaaggttttcttcagcaaaaagtggatggcttactacaacccatccgaagatattcaaaagatttacaagaacggcaccttcaaaaagggtgatatgttcaacttgaacgactgccataagctgatcgatttcttcaaggattccatctcaagatacccaaagtggtctaatgcttacgatttcaacttctctgaaaccgagaagtacaaagatattgccggtttttacagggaagtcgaagaacaaggttacaaggtttcattcgaatccgcctctaaaaaagaagttgacaagttggttgaagagggcaagttgtacatgttccagatttataacaaggacttctccgataagtctcatggtactccaaacttgcataccatgtacttcaagttgttgttcgacgaaaacaatcacggtcagattagattgtctggtggtgctgagttgtttatgagaagggcttctttgaagaaagaagagttggttgttcatccagccaattctccaattgctaacaagaatccagacaacccaaaaaagactaccaccttgtcttacgatgtttacaaggataagagattctccgaggaccaatacgaattgcatattccaattgccattaacaagtgccccaagaacattttcaagatcaacaccgaagttagggtgttgttgaagcacgatgataacccatacgttattggtattgctaggggtgaaagaaacttgttgtacatcgttgttgttgatggtaagggtaacatcgtggaacaatactcattgaacgagattatcaacaacttcaacggcatcagaatcaagaccgattaccattctctgcttgacaagaaagaaaaagaaagattcgaagccagacagaactggacctctattgaaaacatcaaagagttgaaggccggttacatttcacaagttgttcataagatttgcgagctggttgaaaagtatgatgcagttattgctttggaggacttgaactctggtttcaagaactccagagttaaggtcgaaaagcaggtctaccaaaagttcgaaaagatgttgatcgacaagctgaactacatggtcgacaaaaagtctaatccatgtgctacaggtggtgctttgaaaggttatcaaattacgaacaagttcgagagcttcaagtctatgtctactcagaatggtttcatcttctacattccagcttggttgacctctaagattgatccatctactggtttcgtcaacctgctgaaaactaagtacacttctattgccgactccaagaagttcatctcttcattcgatagaatcatgtacgttccagaagaggacttattcgaatttgctttggattacaagaacttctcaagaactgatgctgactacatcaagaagtggaagttgtactcttacggtaacagaatcaggatcttcagaaaccctaagaagaacaacgttttcgattgggaagaagtctgtttgacctctgcttacaaagaactgtttaacaagtacggcatcaactaccaacaaggtgatattagagctttgttgtgcgaacaatctgataaggccttctactcatcttttatggccttgatgtcactgatgttgcagatgagaaattccattactggtagaaccgatgtcgactttttgatctctccagttaagaactctgacggtattttctacgactcaagaaactatgaagctcaagaaaacgccattttgccaaaaaacgctgatgctaatggtgcttacaacattgctagaaaagttttgtgggctatcggccaattcaaaaaagcagaagatgagaagctggacaaggttaagattgccatctcaaacaaagaatggttggaatacgctcagacttctgtcaaacatggttctggttctccaccaaagaagaaaagaaaggttggttccggttctaaattaggtggttctggtggttccccaaagaagaaaagaaaggttggtagagctgaagcttctggttcaggtagagcagatgctttggatgatttcgatttggacatgttgggttctgatgcattggacgatttcgacttagatatgttaggtagtgacgccttggatgacttcgatctggatatgctaggttccgatgccttagacgactttgatttagacatgctgattaactcaagatcctctggttctcctaagaagaagagaaaagtcggttctcaatacttgccagataccgatgatagacacagaatcgaagaaaagaggaagagaacttacgaaaccttcaagtccatcatgaagaagtctccattttctggtccaactgatccaagaccaccaccaagaagaattgctgttccttcaagatcctctgcctctgttccaaaaccagctccacaaccttatccttttacatcttctttgtccaccatcaactacgatgaatttccaactatggttttcccatccggtcaaatttctcaagcttctgctttggctccagctcctccacaagttttgccacaagctccagcaccagctccagctccagcaatggtttctgctttagctcaagccccagctcctgttccagttttagctccaggtccacctcaagctgttgctccaccagcaccaaaacctactcaagctggtgaaggtactttgtctgaagctttgttgcagttgcaattcgatgatgaagatttgggtgctttgttgggtaattctactgatccagctgtttttaccgatttggcttctgttgataactccgaatttcagcagttgttgaatcaaggtattccagttgctcctcatacaactgaacctatgttgatggaatatccagaagcaattactagattagttactggtgctcaaagacctccagatcctgcacctgctcctttgggtgctccaggtttaccaaatggtctattgtctggtgacgaagatttttcctctattgccgatatggatttctccgctttgttgtgaggatcctaa

SEQ ID NO: 39 - DNA, 609 bp, synthetic construct
  source 1..609: mol_type = other DNA; note = Csy4
atgtctgacccaaagaaaaaaagaaaagttggttcaggttctgaccactacttggatattagattaagaccagatccagaatttccaccagctcaattgatgtctgttttgttcggtaaattgcaccaagctttggttgctcaaggtggtgatagaattggtgtttcatttccagatttggacgaatccagatcaagattgggtgaaagattgagaatccatgcttccgctgatgatttgagagctttgttggctagaccatggttggaaggtttgagagatcacttgcaatttggtgaaccagctgttgttccacatccaactccatatagacaagtttcaagagttcaagccaagtctaacccagaaagattgcgtagaagattgatgagaaggcacgatttgtctgaagaagaagccagaaaaagaatcccagatactgttgctagagctttggatttgccattcgttactttgagatcacaatctaccggtcaacatttcaggttgttcattagacatggtccattgcaagttactgctgaagaaggtggttttacttgttacggtttgtctaaaggtggattcgttccatggtttggatcctaa

SEQ ID NO: 40 - DNA, 22907 bp, synthetic construct
  source 1..22907: mol_type = other DNA; note = pWS4033 CRISPRai vector
gcggccgccaatgaagactatctaattgtatcgagatcacttttcgtgatccgctaatcagcgacggtcacattaggtttgccaagtcagggtatgaaccatacgatcagttttcgtgaacctggtacgtatattgtggcgtttgtgtatattttcattctttgacaacaatcaataccaacctcaaataggaaaagtaataagtttggcgttacaccccaaaagacgccaaacggatcgaacttactcaatagcaattagcgagacaaaacctacgttaagacctgtaaccgatttatcaaagcactctgcggttctttcttgggaatattacctggacattttgtgccctcaagaaacgaggctctacgagcctgttggagcccctcagacattagccgccacgaatcaaactttttacgcgattcggcccaaatcagtttctcacagatcattcgtagagtgaaaaagcacatcgattatttgatacccctttgggttaattactgttgaggtctttgccgtaagttccatcgctgtccctctgaactggccgataattgcagacgaacggtgttgttatccgatacaaccggatatttttcttttaatgagtctaaaccgtgatagcttcaggttaatacaatcaaaaaaagctcaaatattcttttaatgccgcgttcacagattccaattgaatacaactaggtagttcattatatgaagcctttgctactatttttcactatagtctgccttcaccttaatgcagacatccacatattttaatcactttaaaataaaaaggaagatatattagaagctatgatccaatctgtaagccagattaaaattcacgaactcttctttcatttgaattgaatgctttgagttggggtagattatcgcaaattactcatcacatttattgactacgaacttgctgatgtcctttttttatttatatttttcttcagtgaagcgattttttttttacacagaccaagacggaaaaaagtagctaaggaagaaaacaaaatcatgaaaaaaatgtgaagtgatcatgcacatcgcatcaacttaaacattggcttagagatatatagagttagagtttacggcaacctttaagcaccaataccttttggcatagtctaaagacctggttcttaattttaaacaaatttaactaaagatttccctatcaaagaagtaacgagttgacagattttctcaaaataaatcgatactgcatttctaggcatatccagcgagatctatgtctgacccaaagaagaagagaaaggttggttctggttccaagttggaaaagtttaccaactgctactctctgtctaagaccttgagattcaaagctattcctgttggtaagacccaagaaaacattgacaacaagaggttgttggttgaggacgaaaaaagagctgaagattacaagggtgtcaagaagttgctggacagatattacttgtccttcatcaacgatgtcctgcactctattaagctgaagaacttgaacaactacatctccctgttcagaaaaaagaccagaaccgagaaagagaacaaagagttagaaaacttggagatcaacctgaggaaagaaattgctaaggctttcaagggtaacgagggttacaaatctttgttcaagaaggacatcatcgaaaccatcttgccagaatttttggatgataaggacgaaattgccttggtcaattctttcaacggtttcactactgctttcactggtttctttgacaacagggaaaacatgttctctgaagaagctaagtctacctccattgctttcagatgcattaacgaaaacttgaccaggtacatctccaacatggatattttcgaaaaggttgatgccatcttcgataagcacgaagtccaagaaatcaaagagaagattttgaactccgattacgacgtcgaagattttttcgaaggtgagttcttcaacttcgttttgactcaagaaggtatcgacgtttacaacgctattattggtggtttcgttaccgaatctggtgaaaagattaagggtttgaacgagtacatcaacctgtacaatcaaaagaccaagcagaagttgccaaagtttaagcccttgtacaaacaagtcttgtccgacagagaatccttgtctttttatggtgaaggttacacctccgatgaagaagttttggaagttttcagaaacaccctgaacaagaactccgaaatcttctcatccattaagaagttggagaagctgtttaagaacttcgacgaatattcctccgctggtatctttgtcaaaaatggtccagctatttccaccatctccaaggatatttttggtgaatggaacgtcatcagagataagtggaatgctgaatacgatgacatccacttgaaaaagaaggctgttgttaccgaaaagtacgaggatgatagacgtaagtccttcaagaagattggctctttctctctggaacaattgcaagaatatgctgatgccgatttgtccgttgtcgaaaaattgaaagaaatcatcatccagaaggtcgacgaaatctacaaagtttacggttcctctgaaaagttgttcgatgctgatttcgttttggagaagtcgttgaagaaaaacgatgctgttgttgccatcatgaaggatttgttggattccgttaagagcttcgagaactacattaaggcttttttcggtgagggcaaagaaactaacagagatgaatcattctacggcgattttgttttggcctacgatattttgttgaaggttgatcatatctacgacgccatcagaaactacgttactcaaaaaccatactccaaagacaagttcaagctgtactttcaaaacccacaattcatgggtggttgggacaaagacaaagaaacagattacagagccaccatcttgagatatggttctaagtactacttggccatcatggataagaaatacgctaagtgcttgcaaaagatcgataaggatgatgtcaacggtaactacgagaagatcaactacaaattattgcccggtccaaacaagatgttgcctaaggttttcttcagcaaaaagtggatggcttactacaacccatccgaagatattcaaaagatttacaagaacggcaccttcaaaaagggtgatatgttcaacttgaacgactgccataagctgatcgatttcttcaaggattccatctcaagatacccaaagtggtctaatgcttacgatttcaacttctctgaaaccgagaagtacaaagatattgccggtttttacagggaagtcgaagaacaaggttacaaggtttcattcgaatccgcctctaaaaaagaagttgacaagttggttgaagagggcaagttgtacatgttccagatttataacaaggacttctccgataagtctcatggtactccaaacttgcataccatgtacttcaagttgttgttcgacgaaaacaatcacggtcagattagattgtctggtggtgctgagttgtttatgagaagggcttctttgaagaaagaagagttggttgttcatccagccaattctccaattgctaacaagaatccagacaacccaaaaaagactaccaccttgtcttacgatgtttacaaggataagagattctccgaggaccaatacgaattgcatattccaattgccattaacaagtgccccaagaacattttcaagatcaacaccgaagttagggtgttgttgaagcacgatgataacccatacgttattggtattgctaggggtgaaagaaacttgttgtacatcgttgttgttgatggtaagggtaacatcgtggaacaatactcattgaacgagattatcaacaacttcaacggcatcagaatcaagaccgattaccattctctgcttgacaagaaagaaaaagaaagattcgaagccagacagaactggacctctattgaaaacatcaaagagttgaaggccggttacatttcacaagttgttcataagatttgcgagctggttgaaaagtatgatgcagttattgctttggaggacttgaactctggtttcaagaactccagagttaaggtcgaaaagcaggtctaccaaaagttcgaaaagatgttgatcgacaagctgaactacatggtcgacaaaaagtctaatccatgtgctacaggtggtgctttgaaaggttatcaaattacgaacaagttcgagagcttcaagtctatgtctactcagaatggtttcatcttctacattccagcttggttgacctctaagattgatccatctactggtttcgtcaacctgctgaaaactaagtacacttctattgccgactccaagaagttcatctcttcattcgatagaatcatgtacgttccagaagaggacttattcgaatttgctttggattacaagaacttctcaagaactgatgctgactacatcaagaagtggaagttgtactcttacggtaacagaatcaggatcttcagaaaccctaagaagaacaacgttttcgattgggaagaagtctgtttgacctctgcttacaaagaactgtttaacaagtacggcatcaactaccaacaaggtgatattagagctttgttgtgcgaacaatctgataaggccttctactcatcttttatggccttgatgtcactgatgttgcagatgagaaattccattactggtagaaccgatgtcgactttttgatctctccagttaagaactctgacggtattttctacgactcaagaaactatgaagctcaagaaaacgccattttgccaaaaaacgctgatgctaatggtgcttacaacattgctagaaaagttttgtgggctatcggccaattcaaaaaagcagaagatgagaagctggacaaggttaagattgccatctcaaacaaagaatggttggaatacgctcagacttctgtcaaacatggttctggttctccaccaaagaagaaaagaaaggttggttccggttctaaattaggtggttctggtggttccccaaagaagaaaagaaaggttggtagagctgaagcttctggttcaggtagagcagatgctttggatgatttcgatttggacatgttgggttctgatgcattggacgatttcgacttagatatgttaggtagtgacgccttggatgacttcgatctggatatgctaggttccgatgccttagacgactttgatttagacatgctgattaactcaagatcctctggttctcctaagaagaagagaaaagtcggttctcaatacttgccagataccgatgatagacacagaatcgaagaaaagaggaagagaacttacgaaaccttcaagtccatcatgaagaagtctccattttctggtccaactgatccaagaccaccaccaagaagaattgctgttccttcaagatcctctgcctctgttccaaaaccagctccacaaccttatccttttacatcttctttgtccaccatcaactacgatgaatttccaactatggttttcccatccggtcaaatttctcaagcttctgctttggctccagctcctccacaagttttgccacaagctccagcaccagctccagctccagcaatggtttctgctttagctcaagccccagctcctgttccagttttagctccaggtccacctcaagctgttgctccaccagcaccaaaacctactcaagctggtgaaggtactttgtctgaagctttgttgcagttgcaattcgatgatgaagatttgggtgctttgttgggtaattctactgatccagctgtttttaccgatttggcttctgttgataactccgaatttcagcagttgttgaatcaaggtattccagttgctcctcatacaactgaacctatgttgatggaatatccagaagcaattactagattagttactggtgctcaaagacctccagatcctgcacctgctcctttgggtgctccaggtttaccaaatggtctattgtctggtgacgaagatttttcctctattgccgatatggatttctccgctttgttgtgaggatcctaactcgagagtgcttttaactaagaattattagtcttttctgcttattttttcatcatagtttagaacactttatattaacgaatagtttatgaatctatttaggtttaaaaattgatacagttttataagttactttttcaaagactcgtgctgtctattgcataatgcactggaaggggaaaaaaaaggtgcacacgcgtggctttttcttgaatttgcagtttgaaaaatgctgccaaaccagatgtcaacacagctacaacgtgacccaacatcagatgacccaaggtccacctcttattaaaggacgtttgatccttcgacaccatggctctgttgaacttttatctgagagaggaaaaaaaggaaggaaaaaaaagaagaaacttcctttatttatttgtcttaaccacaacacacaatgcaataagatgcaatataatatcaaagccaatatcttatgttgctgatcctgagaaggaatatatacaatttatgtagtaaaataccttttcttctgcgagttgcaagaaatagaaaagactccgattgcgcatcgccagaataaaatttcacaaccacactttttggctgaactttttattacctgattaaacagagagagaaaaggtagaggtcaaaattttttaagcaaaactaaaaaagatgcaaaatcacgtgctgaaaatctaacataagggttaagattagagttttataggacttgttttgtaatatttcaaatacgagctaaccctactgatttcaattaggtctaatttagggttgagctgcactgaaatttcggaaattttgggttattttaaatgagacagaagaactacagagatacgttcttcagactttaaagcttatctccacaaagaattggtcaagaaatcatcctagaaaaacacgtttgctcactcgatcttaatcacatagagtgctggaacgggaagaaagatctatgtctgatccaaaaaagaagagaaaggtcggttctggttctgacaagaaatactctattggtttggctattggcaccaattctgttggttgggctgttattactgacgagtacaaagttccctccaagaagtttaaggttttgggtaacactgacaggcactccattaagaaaaatttgattggtgccctgttgttcgattctggtgaaactgctgaagctactagattgaaaagaaccgctagaagaagatacaccagaagaaagaacagaatctgctacttgcaagagattttctctaacgaaatggccaaggttgatgattcattcttccacagattggaagagtccttcttggttgaagaggataagaaacacgaaagacatccaatcttcggtaacatcgttgatgaagttgcttaccacgaaaagtacccaactatctaccatttgagaaagaagttggttgactctaccgataaggctgatttgagattgatctatttggctttggcccacatgattaagttcagaggtcatttcttgatcgagggtgatttgaatccagataactccgatgttgacaagttgttcatccaattggtccaaacttacaaccagttgttcgaagaaaatccaatcaatgcctctggtgttgatgctaaggctattttgtctgctagactgtctaagtcaagaaggttggaaaacttgattgctcaattgccaggtgaaaagaagaatggtttgttcggtaatttgatcgccttgtctttgggtttgactccaaatttcaagtccaacttcgatttggctgaagatgctaagttgcaattgtctaaggatacctacgatgatgacttggataatttgttggcccaaatcggtgatcaatacgctgatttgtttttggctgctaagaacttgtccgatgccattttgttgtccgatatcttgagagttaacaccgaaattactaaggctccattgtctgcctctatgatcaaaagatacgatgaacaccaccaagacttgactttgttgaaagctttggtcagacaacagttgccagaaaagtacaaagaaatcttcttcgaccagtccaaaaatggttacgctggttatattgatggtggtgcttctcaagaagagttctacaagttcattaagcccatcttggaaaagatggatggtactgaagaactgttggtcaagttgaacagagaagatttgttgagaaagcagagaaccttcgataacggttctattccacatcaaatccacttgggtgaattgcatgctattttgagaaggcaagaggatttctacccattcttgaaggataacagagaaaagatcgagaagatcctgaccttcagaatcccatattatgttggtccattggccagaggtaattcaagatttgcttggatgactaggaagtccgaagaaactattactccatggaacttcgaagaggttgttgataagggtgcttctgctcaatccttcattgaaagaatgactaacttcgacaagaacctgccaaacgaaaaggttttgccaaaacactctttgctgtacgaatacttcaccgtctacaacgaattgactaaggttaagtacgtcaccgaaggtatgagaaaaccagcttttttgtctggtgaacaaaagaaggctatcgtcgatttgttgttcaagactaacagaaaggttaccgtcaagcaattgaaagaggactacttcaagaaaatcgagtgcttcgactccgttgaaattagtggtgttgaagatagattcaacgcctccttaggtacttaccatgacttattgaagatcatcaaggacaaggatttcttggacaacgaagaaaacgaggatatcttggaggatatcgttttgactttgaccttgttcgaggatagggaaatgatcgaagaaaggttgaaaacctacgctcacttgtttgatgataaggtcatgaagcagttgaagagaagaaggtatactggttggggtagattgtctaggaaattgattaacggtatcagggacaagcaatccggtaagactattttggactttttgaagtctgatggcttcgccaatagaaacttcatgcaattgatccacgatgacagcttgaccttcaaagaagatattcaaaaggcccaagtttccggtcaaggtgattctttacatgaacatattgctaacttggctggttccccagctattaagaaaggtattttacaaaccgttaaggtggtcgacgaattggtaaaagttatgggtagacataagccagagaacatcgttattgaaatggctagagaaaatcagactacccaaaagggtcaaaagaactccagagaaaggatgaagagaattgaagagggtatcaaagaattgggctcccagattttgaaagaacacccagttgaaaacacccaattgcagaacgaaaagctgtacttgtactacttgcagaatggtagagatatgtacgtcgaccaagaattggatatcaacagattgtccgattacgatgtcgatgctatcgttccacagtcttttttgaaggatgactccatcgataacaaggtcttgactagatccgataagaacagaggtaagtctgataacgttccatctgaagaggtcgtcaagaagatgaagaattattggagacagttgctgaacgccaagttgattactcaaagaaagttcgacaatttgaccaaggctgaaagaggtggtttgtctgaattggacaaagcaggtttcattaagaggcaattggttgaaactaggcaaatcaccaaacacgttgcccaaattttggattccagaatgaataccaagtacgacgaaaacgataagttgatcagggaagttaaggtcatcacgttgaagtctaagttggtttccgatttcagaaaggacttccaattctacaaggtcagggaaatcaacaactaccatcatgctcatgatgcttacttgaatgctgttgttggtactgccttgattaagaagtatccaaagttggaatccgagttcgtttacggtgattacaaggtttacgacgtcagaaagatgattgccaagtccgaacaagaaattggtaaggctactgctaagtacttcttctactccaacatcatgaatttcttcaagaccgaaatcactttggccaacggtgaaattagaaaaaggccattgattgagactaacggtgagactggtgaaatagtttgggataagggtagagatttcgctaccgttagaaaagttttgtccatgccacaagttaacatcgtcaaaaagaccgaagttcaaactggtggtttctccaaagaatccatcttgccaaaaagaaactccgacaaattgatcgccagaaagaaagattgggacccaaaaaagtatggtggttttgattctccaaccgttgcttactctgttttggttgttgctaaagttgaaaagggcaagagcaagaagttgaagtcagtcaaagaactattgggcatcacgatcatggaaaggtcatcttttgaaaaaaacccaatcgactttttggaggccaagggttacaaagaagttaagaaggatctgattatcaagctgcccaagtactctttgttcgaattggaaaacggtcgtaagagaatgttggcttctgctggtgaattacaaaagggtaatgaattggctctgccctctaagtacgttaactttctatacttggcctctcactacgaaaagttgaagggttctcctgaagataacgaacaaaagcagttgtttgtcgaacagcataagcactacttggacgaaatcatcgaacagatttctgaattttccaagagggttattttggctgatgccaatttggataaggtcttgtctgcttacaacaagcacagagataagccaattagagaacaagccgaaaacatcattcacctgttcactttgactaatttgggtgctccagctgcttttaagtatttcgataccaccatcgacagaaagagatacacttctacaaaagaggttctggacgctaccttgattcatcaatctattactggcttgtacgaaaccaggatcgacttgtctcaattaggtggtgatggttcaggttctccaccaaagaaaaaacgtaaggtcggatctggttctatggaacgtgtgagaatgattaatgtgcaaaggctgttagaagccgcagagtttttagaaagaagagaaagagaatgcgaacacgggtatgccagttctttccctagcatgccctctcccagaggatcctaactcgagattgaattgaattgaaatcgatagatcaatttttttcttttctctttccccatcctttacgctaaaataatagtttattttattttttgaatattttttatttatatacgtatatatagactattatttatcttttaatgattattaagatttttattaaaaaaaaattcgctcctcttttaatgcctttatgcagtttttttttcccattcgatatttctatgtgctggatgcacacactggcttaagatgacaacgtatatattaaatttgctcttgttctgtactttcctaattcttatgtaaaaagacaagaatttatgatactatttaataacaaaaaactacctaagaaaagcatcatgcagtcgaaattgaaatcgaaaagtaaaactttaacggaacatgtttgaaattctaagaaagcatacatcttcatcccttatatatagagttatgtttgatattagtagtcatgttgtaatctctggcctaagtatacgtaacgaaaatggtagcacgtcgcgtttatggcccccaggttaatgtgttctctgaaattcgcatcactttgagaaataatgggaacaccttacgcgtgagctgtgcccaccgcttcgcctaataaagcggtgttctcaaaatttctccccgttttcaggatcacgagcgccatctagttctggtaaaatcgcgcttacaagaacaaagaaaagaaacatcgcgtaatgcaacagtgagacacttgccgtcatatataaggttttggatcagtaaccgttatttgagcataacacaggtttttaaatatattattatatatcatggtatatgtgtaaaatttttttgctgactggttttgtttatttatttagctttttaaaaattttactttcttcttgttaattttttctgattgctctatactcaaaccaacaacaacttactctacaactaagatctatgtctgacccaaagaaaaaaagaaaagttggttcaggttctgaccactacttggatattagattaagaccagatccagaatttccaccagctcaattgatgtctgttttgttcggtaaattgcaccaagctttggttgctcaaggtggtgatagaattggtgtttcatttccagatttggacgaatccagatcaagattgggtgaaagattgagaatccatgcttccgctgatgatttgagagctttgttggctagaccatggttggaaggtttgagagatcacttgcaatttggtgaaccagctgttgttccacatccaactccatatagacaagtttcaagagttcaagccaagtctaacccagaaagattgcgtagaagattgatgagaaggcacgatttgtctgaagaagaagccagaaaaagaatcccagatactgttgctagagctttggatttgccattcgttactttgagatcacaatctaccggtcaacatttcaggttgttcattagacatggtccattgcaagttactgctgaagaaggtggttttacttgttacggtttgtctaaaggtggattcgttccatggtttggatcctaactcgaggcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttcgctggttctgtatgggcacagacaacctaaacgccttgtgaaattgcaaatatggtgatttgaaacgtttcctagtgcagcaggatcacagataacgtgtaaagggcttagcagttgataatcctctctagttaagacctaaacaaaatgctgtcactaaccgtagtattaaatgacacactttggtgactttcgttaatggggatgtggtagtggccattgccaataaacaaaaagaacagggaaagaagtagaaagtgatataagtttgcttgccacttttcgtttttcacgaaaaaaacaggcgaaaaaaaatgctagacaagtacccggctgaatcacacctcgttaacagtgactttcggtgacagatacccgattgggcacccggctggtaagttatgatagaaagccaacgctgtactattggcttagctatggcaatattttgattatcagctagttttattaacgttataattagtgtaaccagtttttcatctatttcatttatttcatttatttactttaattgcagatccccctaacgcgtttaaagcttttattcactagcttatgtattttttataggaaacgcgacgcgtaacatcgcgcaaatgaaggttttgatgtattataatgaggtattcttccttatatacatcgatgaaaagcgttgacagcatacattggaaagaaataggaaacggacaccggaagaaaaaatagatctatgtcaagattggacaaaagcaaggttattaacggtgccttggaactgttgaatggtgtaggtatagaaggcttaacgaccagaaaattagcacaaaagttaggtgtagagcagccaacattatattggcacgtcaaaaacaagagagcattgctagacgcattgccaatcgaaatgttagatcgtcatcacactcatttttgcccattagagggtgagagttggcaagactttttgcgtaacaatgctaaatcctttagatgcgccttgttgagccatcgtgatggtgccaaggttcatctaggtacaaggccaacagaaaaacagtatgagactttggagaatcagctagcctttttatgccaacagggtttttcattggagaatgccttatacgctttaagtgcagttggtcacttcactttaggttgcgtactagaagaacaagagcaccaggttgctaaagaagagagagaaacacctacaacagattcaatgcctccattgctaagacaagctatcgaactgtttgatagacagggtgcagaacctgcattcttgttcggtttagagttgattatttgcggcttggaaaagcagctaaaatgcgaatctggtagtggttcacctcctaagaaaaagagaaaagttggttccggtagcgctaatttcaatcagtctggtaatattgccgactcctctttgtctttcactttcactaattcttccaacggtccaaacttgatcactacccaaactaattctcaagctttgtctcagccaattgcctcttctaatgttcacgataacttcatgaacaacgaaatcaccgcctccaaaattgatgatggtaacaattctaagccattgtctccaggttggactgatcaaactgcttataatgctttcggtattaccaccggtatgtttaacactactactatggatgacgtctacaactacttgttcgatgatgaagatacaccaccaaatccaaagaaagaaggatcctaactcgaggccaattggtgcggcaattgataataacgaaaatgtcttttaatgatctgggtataatgaggaattttccgaacgtttttactttatatatatatatacatgtaacatatattctatacgctatagagaaaggaaatttttcaattaaaaaaaaaatagagaaagagtttcacttcttgattatcgctaacactaatggttgaagtactgctactttaattttatgctgggtagactacccatgagtcacaatgaacgttcgtgctttgtgataaagtgtttcacgtcatccgacatgacttcgtagttatggactgaactgtgtggtgaggttccatgatttcttaggtccagcagatacatgtctcttcccaatttcttgttaaggttacggccaatgcttcggttgttgagcttgttaccgaataagccgtgaagtatgataataggtggtcttggcttcccttcatccccagtttttactgcatctctcttgattatgtcatatgaaaggtccagtgggacttgcttttgttgcagcacctttgctaatgaatgaaaggcacatagtgactgcttaaaaatgcaggaacttaaattattccgaatggtattttgtctcacatatattgtcccatactgtgccaagatcccggctttacccagtatcatcattgtaccgttaccaattctcctcgtatatcacggttagtttttaaacctcggggtgacgtttactattggcgtactaatatattcttattttcttttcttttttgttggcagtttcaagcaacacatgtactggataaccaacccccgcacgctcttggaaaaaattgagaaggcatcggacacttgctgatgagtatttcgaaaaattccatgaagatgaggccaagattgtttggaagagattgaaaagaagaagaagaaaaaaagataaaagcaaatcaaaagatctatgtcaagattggataagtccaaggttatcaactctgctttggagttgttgaacgaagttggtattgaaggtttgaccactagaaagttggctcaaaaattgggtgttgctcaaaaaaccttgtactggcacgttaagaacaaaagagctttgttggacgctttggccattgaaatgttggatagacatcatacccatttctgtccattggaaggtgaatcatggcaagatttcttgagaaacaacgccaagtctttcagatgtgctttgttgtctcatagagatggtgctaaagttcacttgggtactagaccaactgaaaagcaatacgaaactttggaaaaccagttggctttcttgtgtcaacaaggtttctctttagaaaacgccttgtatgctttgtctgctgttggtcattttaccttgggttgtgttttggaagatcaagaacatcaagtcgccaaagaagaaagggaaactccaactactgattctatgccaccattattgagacaagccattgagttgtttgatcatcaaggtgctgaaccagcttttttgtttggtttggaattgatcatctgcggtttggagaaacaattgaagtgtgaatctggttctggttctccaccaaaaaagaaaagaaaggttggttcaggttccgaaagggttagaatgattaacgttcaaaggttgttggaagctgccgaatttttggaaagacgtgaaagggaatgtgaacatggttacgcttcatcttttccatctatgccatctccaagaggatcctaactcgagagcttttgattaagccttctagtccaaaaaacacgtttttttgtcatttatttcattttcttagaatagtttagtttattcattttatagtcacgaatgttttatgattctatatagggttgcaaacaagcatttttcattttatgttaaaacaatttcaggtttaccttttattctgcttgtggtgacgcgtgtatccgcccgctcttttggtcacccatgtatgctgaagtgaataaagctccacacagtcgaacgggtagtccatcgttgtaggatactccctatcagtgatagagaaatctatgcggcatccctatcagtgatagagatcggtaacggagtccctatcagtgatagagaagtggtgttcagttccctatcagtgatagagaagtttgacactatccctatcagtgatagagagaacagcaatgactccctatcagtgatagagatcaaatggcgtatccctatcagtgatagagataattaacttgtaatattctaatcaattgatataaaaatccccgtcagtgacggagattttcttttttttttatttttccccgtcagtgacggagaatatcaagctacaaaaagaattcgcatctagattcttgagacggttcactgccgtataggcagtgagaccgaaagtgaaacgtgatttcatgcgtcattttgaacattttgtaaatcttatttaataatgtgtgcggcaattcacatttaatttatgaatgttttcttaacatcgcggcaactcaagaaacggcaggttcggatcttagctactagagaaagaggagaaatactagatgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggttccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaggacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaatgaccaggcatcaaataaaacgaaaggctcagtcgaaagactgggcctttcgttttatctgttgtttgtcggtgaacgctctctactagagtcacactggctcaccttcgggtgggcctttctgcgtttataggtctcatccccgtcagtgacggagatcgtctctaccaactagtgcactgcagtacaataaagcaatcttgatgaggataatgatttttttttgaatatacataaatactaccgtttttctgctagattttgtgatgacgtaaataagtacatattactttttaagccaagacaagattaagcattaactttacccttttctttctaagtttcaatattagttatcactgtttaaaagttatggcgagaacgtcggcggttaaaatatattaccctgaacgagcatacatcgaggagaacttctagtatatctacatacctaatattattgccttattaaaaatggaatcccaacaattacatcaaaatccacattctcttcaaaatcaattgtcctgtacttccttgttcatgtgtgttcaaaaacgttatatttataggataattatactctatttctcaacaagtaattggttgtttggccgagcggtctaaggcgcctgattcaagaaatatcttgaccgcagttaactgtgggaatactcaggtatcgtaagatgcaagagttcgaatctcttagcaaccattatttttttcctcaacataacgagaacacacaggggcgctatcgcacagaatcaaattcgatgactggaaattttttgttaatttcagaggtcgcctgacgcatatacctttttcaactgaaaaattgggagaaaaaggaaaggtgagagcgccggaaccggcttttcatatagaatagagaagcgttcatgactaaatgcttgcatcacaatacttgaagttgacaatattatttaaggacctattgttttttccaataggtggttagcaatcgtcttactttctaacttttcttaccttttacatttcagcaatatatatatatatatttcaaggatataccattctaatgtctgcccctaagaagatcgtcgttttgccaggtgaccacgttggtcaagaaatcacagccgaagccattaaggttcttaaagctatttctgatgttcgttccaatgtcaagttcgatttcgaaaatcatttaattggtggtgctgctatcgatgctacaggtgttccacttccagatgaggcgctggaagcctccaagaaggctgatgccgttttgttaggtgctgtgggtggtcctaaatggggaaccggtagtgttagacctgaacaaggtttactaaaaatccgtaaagaacttcaattgtacgccaacttaagaccatgtaactttgcatccgactctcttttagacttatctccaatcaagccacaatttgctaaaggtactgacttcgttgttgtcagagaattagtgggaggtatttactttggtaagagaaaggaggacgatggtgatggtgtcgcttgggatagtgaacaatacaccgttccagaagtgcaaagaatcacaagaatggccgctttcatggccctacaacatgagccaccattgcctatttggtccttggataaagctaatgttttggcctcttcaagattatggagaaaaactgtggaggaaaccatcaagaacgaatttcctacattgaaggttcaacatcaattgattgattctgccgccatgatcctagttaagaacccaacccacctaaatggtattataatcaccagcaacatgtttggtgatatcatctccgatgaagcctccgttatcccaggttccttgggtttgttgccatctgcgtccttggcctctttgccagacaagaacaccgcatttggtttgtacgaaccatgccacggttctgctccagatttgccaaagaataaggtcaaccctatcgccactatcttgtctgctgcaatgatgttgaaattgtcattgaacttgcctgaagaaggtaaggccattgaagatgcagttaaaaaggttttggatgcaggtatcagaactggtgatttaggtggttccaacagtaccaccgaagtcggtgatgctgtcgccgaagaagttaagaaaatccttgcttaacctggaggacccttctctttagactattctactcttatgcacgtaaaaaattctaggaaatatgtattaactaggagtaaaataaccggctagtggcattcatatagccgtctgtttacatctacatcacacatttcgagtgtatatctcgcaacgttggcgttaaataggcaggagttatcgtgttgcatctgcggctttaaattgatgtatctcatcgcaggcacgggcagtacagtgccctgagcgtagggaaaaatgaaaaaaaggatgtaacttttaacataattccagcacgcagcgattgggtataatgaagattgttaagttcaacaacatgtgaaattcttagctccgcaacttgcttgaattatgagctctaagattcaagaagtaaataggtcatataatggaatgatagtagtacttgataaaggaaccatgtgatcttacgttgatatgaaaataattctctcacagaagtcagtttgtatgactaacatagagagtaattgcttttcggataaaatcgcacctaacaaagattaccttttgtaaagcctccagaacagctatgaatgttcgttattctacgcactttctttcatacatcttgatccgccttttttgatgtaggttttaacgcggcaaaacatattaatttttctgatttggaaaaataaagagttagttctgcctagtcttcccgagcggccgcgtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctggagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgt

SEQ ID NO: 41 - DNA, 1978 bp, synthetic construct
  source 1..1978: mol_type = other DNA; note = exemplary nucleic acid construct according to the invention
ggtagtccatcgttgtaggatactccctatcagtgatagagaaatctatgcggcatccctatcagtgatagagatcggtaacggagtccctatcagtgatagagaagtggtgttcagttccctatcagtgatagagaagtttgacactatccctatcagtgatagagagaacagcaatgactccctatcagtgatagagatcaaatggcgtatccctatcagtgatagagataattaacttgtaatattctaatcaattgatataaaaatccccgtcagtgacggagattttcttttttttttatttttccccgtcagtgacggagaatatcaagctacaaaaagaattcgcatctagattctgttcactgccgtataggcagctttgtattccaacttaccggttttagagctagaaatagcaagttaaaataaggctagtccgttatcaacttgaaaaagtggcaccgagtcggtgcttttgttcactgccgtataggcagtgttgacgttctcaacatgagttttagagctagaaatagcaagttaaaataaggctagtccgttatcaacttgaaaaagtggcaccgagtcggtgcttttgttcactgccgtataggcaggtgcctgctgataagtactggttttagagctagaaatagcaagttaaaataaggctagtccgttatcaacttgaaaaagtggcaccgagtcggtgcttttgttcactgccgtataggcagtccccgtcagtgacggagatgccgttcactgccgtataggcagtgtgagacaacagacgcacagttttagagctagaaatagcaagttaaaataaggctagtccgttatcaacttgaaaaagtggcaccgagtcggtgcttttgttcactgccgtataggcagccgcgtccagatctgcagacgttttagagctagaaatagcaagttaaaataaggctagtccgttatcaacttgaaaaagtggcaccgagtcggtgcttttgttcactgccgtataggcagcagctgccttgagctcctcggttttagagctagaaatagcaagttaaaataaggctagtccgttatcaacttgaaaaagtggcaccgagtcggtgcttttgttcactgccgtataggcagtccccgtcagtgacggagactaagttcactgccgtataggcagcaacaagctatgaatatgaggttttagagctagaaatagcaagttaaaataaggctagtccgttatcaacttgaaaaagtggcaccgagtcggtgcttttgttcactgccgtataggcaggaacgtgctattgagaaggagttttagagctagaaatagcaagttaaaataaggctagtccgttatcaacttgaaaaagtggcaccgagtcggtgcttttgttcactgccgtataggcagctgaataagacatgttaggggttttagagctagaaatagcaagttaaaataaggctagtccgttatcaacttgaaaaagtggcaccgagtcggtgcttttgttcactgccgtataggcagtccccgtcagtgacggagaatgagttcactgccgtataggcagaatttctactaagtgtagatgctatgacaccttttctttcgttcactgccgtataggcagaatttctactaagtgtagatgcaccaatattgagtcacatgttcactgccgtataggcagtccccgtcagtgacggagaaccaactagtgcactgcagtacaataaagcaatcttgatgaggataatgatttttttttgaatatacataaatactaccgtttttctgctagattttgtgatgacgtaaataagtacatattactttttaagccaagacaagattaagcattaactttacccttttctttctaagtttcaatattagttatcactgtttaaaagttatggcgagaacgtcggcggttaaaatatattaccctgaacg

SEQ ID NO: 42 - DNA, 1690 bp, synthetic construct
  source 1..1690: mol_type = other DNA; note = exemplary inducible array
ggtagtccatcgttgtaggatactccctatcagtgatagagaaatctatgcggcatccctatcagtgatagagatcggtaacggagtccctatcagtgatagagaagtggtgttcagttccctatcagtgatagagaagtttgacactatccctatcagtgatagagagaacagcaatgactccctatcagtgatagagatcaaatggcgtatccctatcagtgatagagataattaacttgtaatattctaatcaattgatataaaaatccccgtcagtgacggagattttcttttttttttatttttccccgtcagtgacggagaatatcaagctacaaaaagaattcgcatctagattcttgagacggttcactgccgtataggcagtgagaccgaaagtgaaacgtgatttcatgcgtcattttgaacattttgtaaatcttatttaataatgtgtgcggcaattcacatttaatttatgaatgttttcttaacatcgcggcaactcaagaaacggcaggttcggatcttagctactagagaaagaggagaaatactagatgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggttccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaggacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaatgaccaggcatcaaataaaacgaaaggctcagtcgaaagactgggcctttcgttttatctgttgtttgtcggtgaacgctctctactagagtcacactggctcaccttcgggtgggcctttctgcgtttataggtctcatccccgtcagtgacggagatcgtctctaccaactagtgcactgcagtacaataaagcaatcttgatgaggataatgatttttttttgaatatacataaatactaccgtttttctgctagattttgtgatgacgtaaataagtacatattactttttaagccaagacaagattaagcattaactttacccttttctttctaagtttcaatattagttatcactgtttaaaagttatggcgagaacgtcggcggttaaaatatattaccctgaacg

SEQ ID NO: 43 - DNA, 224 bp, unidentified
  source 1..224: mol_type = other DNA; note = ScTDH1 Terminator
ataaagcaatcttgatgaggataatgatttttttttgaatatacataaatactaccgtttttctgctagattttgtgatgacgtaaataagtacatattactttttaagccaagacaagattaagcattaactttacccttttctttctaagtttcaatattagttatcactgtttaaaagttatggcgagaacgtcggcggttaaaatatattaccctgaacg

SEQ ID NO: 44 - DNA, 217 bp, unidentified
  source 1..217: mol_type = other DNA; note = AgLEU2 Terminator
cctggaggacccttctctttagactattctactcttatgcacgtaaaaaattctaggaaatatgtattaactaggagtaaaataaccggctagtggcattcatatagccgtctgtttacatctacatcacacatttcgagtgtatatctcgcaacgttggcgttaaataggcaggagttatcgtgttgcatctgcggctttaaattgatgtatctca

SEQ ID NO: 45 - DNA, 17 bp, unidentified
  source 1..17: mol_type = other DNA; note = Gal4
cggrnnrcynyncnccg

SEQ ID NO: 46 - DNA, 20 bp, synthetic construct
  source 1..20: mol_type = other DNA
tataaatgtaataagaagtt

SEQ ID NO: 47 - DNA, 20 bp, synthetic construct
  source 1..20: mol_type = other DNA
acaccgttcgaggtcaagcc

SEQ ID NO: 48 - DNA, 20 bp, synthetic construct
  source 1..20: mol_type = other DNA
gctgtttgagctgactaaca

SEQ ID NO: 49 - DNA, 20 bp, synthetic construct
  source 1..20: mol_type = other DNA
ttattacgaatttgcccaca

SEQ ID NO: 50 - DNA, 20 bp, synthetic construct
  source 1..20: mol_type = other DNA
cattgatctcctcttgggaa

SEQ ID NO: 51 - DNA, 20 bp, synthetic construct
  source 1..20: mol_type = other DNA
agttaaagcgatatagaagc

SEQ ID NO: 52 - DNA, 20 bp, synthetic construct
  source 1..20: mol_type = other DNA
aaaagagggtgggcgcgccg

SEQ ID NO: 53 - DNA, 20 bp, synthetic construct
  source 1..20: mol_type = other DNA
tcggctgccttattcccgtg

SEQ ID NO: 54 - DNA, 20 bp, synthetic construct
  source 1..20: mol_type = other DNA
ttaacttaaatatcaatggg

SEQ ID NO: 55 - DNA, 20 bp, synthetic construct
  source 1..20: mol_type = other DNA
acgaagaaaaagaaacgagg

SEQ ID NO: 56 - DNA, 20 bp, synthetic construct
  source 1..20: mol_type = other DNA
ggtaattaacgacaccctag

SEQ ID NO: 57 - DNA, 20 bp, synthetic construct
  source 1..20: mol_type = other DNA
tgaagtggtacggcgatgcg

SEQ ID NO: 58 - DNA, 20 bp, synthetic construct
  source 1..20: mol_type = other DNA
gtagaaacattttgaagcta

SEQ ID NO: 59 - DNA, 20 bp, synthetic construct
  source 1..20: mol_type = other DNA
gggtgtgatgtaaggattcg

SEQ ID NO: 60 - DNA, 20 bp, synthetic construct
  source 1..20: mol_type = other DNA
acatataatacatatcacat

SEQ ID NO: 61 - DNA, 20 bp, synthetic construct
  source 1..20: mol_type = other DNA
tattatcagccaaagttggg

SEQ ID NO: 62 - DNA, 20 bp, synthetic construct
  source 1..20: mol_type = other DNA
atcatatagaaaaaatatct

SEQ ID NO: 63 - DNA, 20 bp, synthetic construct
  source 1..20: mol_type = other DNA
tttgatggataaattggttg

SEQ ID NO: 64 - DNA, 20 bp, synthetic construct
  source 1..20: mol_type = other DNA
gcaaatgcccgcgaatacgg

SEQ ID NO: 65 - DNA, 20 bp, synthetic construct
  source 1..20: mol_type = other DNA
ccattatggggagaagcgct

SEQ ID NO: 66 - DNA, 20 bp, synthetic construct
  source 1..20: mol_type = other DNA
aacgcggtttccaaattcgg

SEQ ID NO: 67 - DNA, 20 bp, synthetic construct
  source 1..20: mol_type = other DNA
ttgtgagaacgataatgtat

SEQ ID NO: 68 - DNA, 20 bp, synthetic construct
  source 1..20: mol_type = other DNA
tattacggccaggatcgcaa

SEQ ID NO: 69 - DNA, 20 bp, synthetic construct
  source 1..20: mol_type = other DNA
accaaaaagaaaaatcgccc

SEQ ID NO: 70 - DNA, 20 bp, synthetic construct
  source 1..20: mol_type = other DNA
gaacacttattctgggaaga

SEQ ID NO: 71 - DNA, 20 bp, synthetic construct
  source 1..20: mol_type = other DNA
caagggtatggtacggtgct

SEQ ID NO: 72 - DNA, 20 bp, synthetic construct
  source 1..20: mol_type = other DNA
ttgatatttaagttaataaa

SEQ ID NO: 73 - DNA, 20 bp, synthetic construct
  source 1..20: mol_type = other DNA
caagggtatggtacggtgct

SEQ ID NO: 74 - DNA, 20 bp, synthetic construct
  source 1..20: mol_type = other DNA
tcagcagcaacaacacgcta

SEQ ID NO: 75 - DNA, 20 bp, synthetic construct
  source 1..20: mol_type = other DNA
cccaaacggttgcccgttgc

SEQ ID NO: 76 - DNA, 20 bp, synthetic construct
  source 1..20: mol_type = other DNA
ttaacttaaatatcaatggg

SEQ ID NO: 77 - DNA, 20 bp, synthetic construct
  source 1..20: mol_type = other DNA
acgaagaaaaagaaacgagg

SEQ ID NO: 78 - DNA, 20 bp, synthetic construct
  source 1..20: mol_type = other DNA
ggtaattaacgacaccctag

SEQ ID NO: 79 - DNA, 20 bp, synthetic construct
  source 1..20: mol_type = other DNA
tgagcgaataacctgttgtc

SEQ ID NO: 80 - DNA, 20 bp, synthetic construct
  source 1..20: mol_type = other DNA
gaacacttattctgggaaga

SEQ ID NO: 81 - DNA, 20 bp, synthetic construct
  source 1..20: mol_type = other DNA
ctttgtattccaacttaccg

SEQ ID NO: 82 - DNA, 20 bp, synthetic construct
  source 1..20: mol_type = other DNA
tgttgacgttctcaacatga

SEQ ID NO: 83 - DNA, 20 bp, synthetic construct
  source 1..20: mol_type = other DNA
gtgcctgctgataagtactg

SEQ ID NO: 84 - DNA, 20 bp, synthetic construct
  source 1..20: mol_type = other DNA
tgtgagacaacagacgcaca

SEQ ID NO: 85 - DNA, 20 bp, synthetic construct
  source 1..20: mol_type = other DNA
ccgcgtccagatctgcagac

SEQ ID NO: 86 - DNA, 20 bp, synthetic construct
  source 1..20: mol_type = other DNA
cagctgccttgagctcctcg

SEQ ID NO: 87 - DNA, 20 bp, synthetic construct
  source 1..20: mol_type = other DNA
caacaagctatgaatatgag

SEQ ID NO: 88 - DNA, 20 bp, synthetic construct
  source 1..20: mol_type = other DNA
gaacgtgctattgagaagga

SEQ ID NO: 89 - DNA, 20 bp, synthetic construct
  source 1..20: mol_type = other DNA
ctgaataagacatgttaggg

SEQ ID NO: 90 - DNA, 20 bp, synthetic construct
  source 1..20: mol_type = other DNA
gctatgacaccttttctttc

SEQ ID NO: 91 - DNA, 20 bp, synthetic construct
  source 1..20: mol_type = other DNA
gcaccaatattgagtcacat

SEQ ID NO: 92 - DNA, 20 bp, synthetic construct
  source 1..20: mol_type = other DNA
tgagcgaataacctgttgtc

SEQ ID NO: 93 - DNA, 20 bp, synthetic construct
  source 1..20: mol_type = other DNA
gaacacttattctgggaaga

(19) United States
(12) Patent Application Publication
(10) Pub. No.: US 2025/0136975 A1
(43) Pub. Date: May 1, 2025

(54) COMPOSITIONS

(71) Applicant: Imperial College Innovations Ltd, London (GB)

(72) Inventors: Rodrigo Ledesma-Amaro, London (GB); William Shaw, London (GB); Lucie Studena, London (GB); Thomas Marc Ellis, London (GB)

(21) Appl. No.: 18/835,557

(22) PCT Filed: Feb. 2, 2023
(86) PCT No.: PCT/GB2023/050228

§ 371 Date: Aug. 2, 2024

(30) Foreign Application Priority Data
Feb. 3, 2022 (GB) 2201404.7

(51) Int. Cl. C12N 15/11; C12N 9/22; C12N 15/79

(52) U.S. Cl. CPC C12N 15/111; C12N 9/22; C12N 15/79; C12N 2310/20; C12N 2830/006

(57) ABSTRACT

The invention provides a nucleic acid construct that is useful in directing RNA mediated gene regulation or RNA mediated gene editing. The invention further provides cells comprising the nucleic acid construct, and methods of using the same.

### FIELD OF THE INVENTION

The present invention is in the field of gene regulation, and in particular metabolic engineering.

### BACKGROUND

CRISPR gene activation (CRISPRa) and inhibition (CRISPRI) have become powerful synthetic tools for modulating endogenous gene expression^{1,2}. The simultaneous activation and inhibition (CRISPRai) of target genes now allows us to fully explore transcriptional landscapes and modify cellular behaviour³. This is especially important in metabolic engineering where metabolic fluxes must be redirected towards a desired product which is usually achieved by upregulating desired reactions and downregulating competing pathways.

In the simplest form, CRISPRai systems are achieved using a single catalytically inactive Cas protein, usually dCas9, linked to a transcriptional activator by a direct fusion or via a modified gRNA containing a protein binding aptamer⁴⁻⁹. By targeting the Cas protein upstream of the 5′UTR, expression of that gene can be increased. The Cas protein can then be targeted to the 5′UTR or coding region to block transcription initiation or elongation, thus reducing expression. However, while this approach leads to effective gene activation, gene inhibition is less successful without a transcriptional repression domain, and efficient inhibition may not be possible if optimally positioned PAM sites do not exist^{6,10.}

A more effective approach for achieving CRISPRai is to employ both transcriptional activation and repression domains. This can be realised using orthogonal Cas proteins, such as dCas9 and dCas12a, with one protein fused to an activator and the other to a repressor^{11,12}. These fusion proteins are then targeted to the chosen genes using their cognate gRNAs. Alternatively, a single Cas protein can be used, and instead, modified gRNAs with two orthogonal protein binding aptamers can be used to specifically recruit an activator or repressor to the target genes¹³⁻¹⁷.

Although these later approaches lead to more effective CRISPRai, the use of mixed identity gRNAs introduces more complexity, often requiring cumbersome cloning methods. Consequently, multiplexing capacities tend to be low and, for example, in the industrially relevant yeast S. cerevisiae, a maximum of 4 gRNAs have been expressed simultaneously for CRISPRai in an attempt to increase beta-carotene production¹⁷. This constrain in the number of perturbations that can be made at any one time, limits our cellular engineering ambitions, since most of the time, desired behaviours are achieved by altering the expression of a large number of targets³. Powerful assembly methods have recently been developed to allow the straightforward and rapid assembly of polycistronic arrays for expressing up to 12 Cas9 gRNAs in yeast and Cas12a gRNAs in mammalian cells, for highly multiplexed CRISPR applications¹⁸⁻²⁰. However, such methods have yet to be applied to the combined expression of activation and repression gRNAs.

An additional desirable feature for any transcriptional regulation method is inducibility. It is known that prolonged transcriptional perturbation of genes can impose a fitness cost, leading to genetic instability and phenotypic loss^{21,22}. Furthermore, the regulation of essential genes can be difficult or impossible to modulate continuously or without impacting cell growth²³. To date, inducibility of large gRNA arrays for multiplexed CRISPR regulation has only been demonstrated for CRISPRa or CRISPRi, separately¹⁸.

The present invention addresses the disadvantages and challenges of the known methods and constructs and provides a nucleic acid construct that allows tight control of components in the CRISPRai system. The present invention provides the first system to combine simultaneous activation and repression, large multiplexing capacity, and inducibility.

### SUMMARY OF THE INVENTION

To overcome the challenges discussed above, the inventors have developed a nucleic acid construct that allows near leak-free and leak-free, inducible expression of a gene-regulating and/or gene-editing array module containing at least a first nucleic acid region that encodes at least one nucleic acid that is capable of directing RNA mediated gene regulation or RNA mediated gene editing (for example a gRNA from orthogonal CRISPR/Cas systems) to increase RNA mediated gene regulation or RNA mediated gene editing (for example CRISPRai) multiplexing capacity and target gene flexibility.

To achieve strong inducibility, the inventors have created a technology to silence expression within the gene-regulating and/or gene-editing array module in the absence of the inducer, since it was surprisingly found by the inventors that, for example, long gRNA arrays for CRISPRai can express themselves even without promoter. The inventors have provided a highly tuned and easy-to-use RNA mediated gene regulation or RNA mediated gene editing toolkit in industrially relevant microorganisms, including Saccharomyces cerevisiae, establishing the first system to combine simultaneous activation and repression, large multiplexing capacity, and inducibility.

### DETAILED DESCRIPTION OF THE INVENTION

The invention is as set out in the claims.

A first aspect of the invention provides a nucleic acid construct comprising:
a) a promoter module wherein the promoter module comprises at least one promoter operator of a first sequence; and
b) a gene-regulating and/or gene-editing array module, that comprises at least one array sub-module, wherein each array sub-module comprises at least a first nucleic acid region that encodes a nucleic acid that is capable of directing RNA mediated gene regulation or RNA mediated gene editing,
   wherein the gene-regulating or gene-editing array module is capable of being transcribed into a single polycistronic nucleic acid transcript from a single promoter,
   and wherein between each nucleic acid region that encodes a nucleic acid that is capable of directing RNA mediated gene regulation or RNA mediated gene editing is a sequence that when in RNA form is an RNA cleavage site; and
   wherein each array sub-module comprises at least one array operator of a second sequence
and wherein the gene-regulating and/or gene-editing array module is operably linked to promoter module.

The invention also provides a nucleic acid construct comprising:
a) a promoter module wherein the promoter module comprises at least one promoter operator of a first sequence; and
b) a gene-regulating and/or gene-editing array module, that comprises at least one array sub-module, wherein each array sub-module comprises at least a first nucleic acid region that encodes a nucleic acid that is capable of directing RNA mediated gene regulation or RNA mediated gene editing and a second nucleic acid region that encodes a nucleic acid that is capable of directing RNA mediated gene regulation or RNA mediated gene editing,
wherein the gene-regulating and/or gene-editing array module is capable of being transcribed into a single polycistronic nucleic acid transcript from a single promoter,
and wherein between each nucleic acid region that encodes a nucleic acid that is capable of directing RNA mediated gene regulation or RNA mediated gene editing is a sequence that when in RNA form is an RNA cleavage site; and
wherein each array sub-module comprises at least one array operator of a second sequence
and wherein the gene-regulating and/or gene-editing array module is operably linked to promoter module.

The invention also provides a nucleic acid construct comprising:
a) a promoter module wherein the promoter module comprises at least one promoter operator of a first sequence; and
b) a gene-regulating and/or gene-editing array module, that comprises at least two array sub-modules, wherein each array sub-module comprises at least a first nucleic acid region that encodes a nucleic acid that is capable of directing RNA mediated gene regulation or RNA mediated gene editing and a second nucleic acid region that encodes a nucleic acid that is capable of directing RNA mediated gene regulation or RNA mediated gene editing,
   wherein the gene-regulating and/or gene-editing array module is capable of being transcribed into a single polycistronic nucleic acid transcript from a single promoter,
   and wherein between each nucleic acid region that encodes a nucleic acid that is capable of directing RNA mediated gene regulation or RNA mediated gene editing is a sequence that when in RNA form is an RNA cleavage site; and
   wherein each array sub-module comprises at least one array operator of a second sequence
and wherein the gene-regulating and/or gene-editing array module is operably linked to promoter module.

As provided herein, the promoter module of the nucleic acid construct comprises at least one promoter operator of a first sequence. It will be appreciated that the promoter module of the nucleic acid construct also comprises at least one promoter and is capable of acting as a promoter—i.e. capable of driving transcription from a downstream sequence in the presence of the appropriate polymerase and associated factors. Suitable promoters are known to the person skilled in the art; and are further disclosed herein.

In some instances, it is advantageous to include additional regulatory elements in the nucleic acid of the present invention. Additional regulatory elements may be included in any component of the nucleic acid of the present invention, including but not limited to: the promoter module; the gene-regulating and/or gene-editing module; the array sub-module; the nucleic acid that is capable of directing RNA mediated gene regulation or RNA mediated gene editing; the single polycistronic nucleic acid transcript, or any combination thereof.

As used herein, a “regulatory element” is any nucleic acid or nucleotide sequence that is capable of regulating the expression and/or transcription of a gene. A “regulatory element” may be selected from the group comprising or consisting of: a promoter; an activator; an operator; an enhancer; a response element; a silencer; a terminator; or any combination thereof.

Accordingly, in some embodiments, the promotor module of the nucleic acid construct further comprises at least one promoter operator of a second sequence.

In some embodiments, the nucleic acid construct comprises a terminator sequence. In some embodiments, the terminator sequence is directly downstream (3′) of the gene-regulating and/or gene-editing array module. As will be understood by the person skilled in the art, terminator sequences are capable of terminating transcription of a nucleic acid. In some embodiments, the terminator sequence is an ScTDH1 terminator (SEQ ID NO: 43) or an AgLEU2 terminator (SEQ ID NO: 44).

In some embodiments, the sequence of the at least one promoter operator of a first sequence and the at least one array operator of a second sequence are different. In yet a further embodiment, the sequence of the at least one promoter operator of a first sequence and the at least one promoter operator of a second sequence are different. In a yet another embodiment, the sequence of the at least one promoter operator of a first sequence and the at least one array operator of a second sequence are the same.

Of course, the person skilled in the art will be aware that it is possible to omit a) a promoter module wherein the promoter module comprises at least one promoter operator of a first sequence. For example, the nucleic acid construct of the invention, comprising the gene regulating and/or gene editing array module, may be designed and supplied by one entity with the intention that the module is cloned into for example a vector that comprises its own promoter. Accordingly, in some embodiments, the nucleic acid construct does not comprise a) a promoter module wherein the promoter module comprises at least one promoter operator of a first sequence.

Accordingly in some instances the invention provides:

A nucleic acid construct comprising:
a gene-regulating and/or gene-editing array module, that comprises at least one array sub-module, wherein each array sub-module comprises at least a first nucleic acid region that encodes a nucleic acid that is capable of directing RNA mediated gene regulation or RNA mediated gene editing,
wherein the gene-regulating and/or gene-editing array module is capable of being transcribed into a single polycistronic nucleic acid transcript from a single promoter,
and wherein between each nucleic acid region that encodes a nucleic acid that is capable of directing RNA mediated gene regulation or RNA mediated gene editing is a sequence that when in RNA form is an RNA cleavage site; and
wherein each array sub-module comprises at least one array operator of a second sequence
and wherein the gene-regulating and/or gene-editing array module is operably linked to promoter module.

Preferences for features of this embodiment, wherein the nucleic acid construct does not comprise a promoter, are as described elsewhere for other embodiments.

Regulation of expression from the promoter module may be tuned by “tuning” or altering the number of operator sequences in the promoter module to achieve a desired level of expression. The level of expression from a “tuned” promoter module may be increased or decreased compared to the level of expression from a promoter module that is not “tuned”. Accordingly, in some embodiments, the promoter module comprises at least 2, at least 3, at least 4, at least 5, at least 6, at least 7, at least 8, at least 9, at least 10 or more promoter operators of a first sequence; optionally 1, 2, 3, 4, 5, 6, 7, 8, 9, or 10 promoter operators of a first sequence. In some embodiments, the promoter module comprises at least 2, at least 3, at least 4, at least 5, at least 6, at least 7, at least 8, at least 9, at least 10 or more promoter operators of a second sequence; optionally 1, 2, 3, 4, 5, 6, 7, 8, 9, or 10 promoter operators of a second sequence. It will be appreciated that the promoter module may comprise other additional operator sequences, for example a third or a fourth operator sequence.

Regulation of the array sub-module may “tuned” by altering the number of operator sequences comprised in each array submodule. Accordingly, in some embodiments, each array sub-module comprises at least 2, at least 3, at least 4, at least 5, at least 6, at least 7, at least 8, at least 9, or at least 10 array operators of a second sequence; optionally 1, 2, 3, 4, 5, 6, 7, 8, 9, or 10 array operators of a second sequence. In some embodiments, however, each array sub-module comprises a single array operator of a second sequence.

It will be appreciated that any number of array sub-molecules may be included in the gene-regulating and/or gene-editing array module as disclosed herein. Accordingly, in some embodiments the gene-regulating and/or gene-editing array module comprises:
between 2 and 100 array sub-modules, optionally between 5 and 95, 10 and 90, 15 and 85, 20 and 80, 25 and 75, 30 and 70, 35 and 65, 40 and 60, 45 and 55 array sub-modules; and/or
at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 77, at least 76, at least 77, at least 78, at least 79, at least 80, at least 81, at least 82, at least 83, at least 84, at least 88, at least 86, at least 87, at least 88, at least 89, at least 90, at least 91, at least 92, at least 93, at least 94, at least 99, at least 96, at least 97, at least 98, at least 99, or more array sub-modules; optionally comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 66, 66, 67, 68, 69, 70, 71, 72, 73, 74, 77, 76, 77, 78, 79, 80, 81, 82, 83, 84, 88, 86, 87, 88, 89, 90, 91, 92, 93, 94, 99, 96, 97, 98, 99, or 100 array sub-modules.

It will be appreciated that any number of nucleic acid regions that each encode a nucleic acid that is capable of directing RNA mediated gene regulation or RNA mediated gene editing may be included in each array sub-module of the gene-regulating and/or gene-editing array module as disclosed herein. Accordingly, in some embodiments, at least one array sub-module comprises:
between 2 and 100 nucleic acid regions that each encode a nucleic acid that is capable of directing RNA mediated gene regulation or RNA mediated gene editing;
optionally between 5 and 95, 10 and 90, 15 and 85, 20 and 80, 25 and 75, 30 and 70, 35 and 65, 40 and 60, 45 and 55 nucleic acid regions that each encode a nucleic acid that is capable of directing RNA mediated gene regulation or RNA mediated gene editing; and/or
at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 77, at least 76, at least 77, at least 78, at least 79, at least 80, at least 81, at least 82, at least 83, at least 84, at least 88, at least 86, at least 87, at least 88, at least 89, at least 90, at least 91, at least 92, at least 93, at least 94, at least 99, at least 96, at least 97, at least 98, at least 99, or more nucleic acid regions that each encode a nucleic acid that is capable of directing RNA mediated gene regulation or RNA mediated gene editing; optionally comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 66, 66, 67, 68, 69, 70, 71, 72, 73, 74, 77, 76, 77, 78, 79, 80, 81, 82, 83, 84, 88, 86, 87, 88, 89, 90, 91, 92, 93, 94, 99, 96, 97, 98, 99, or 100 nucleic acid regions that each encode a nucleic acid that is capable of directing RNA mediated gene regulation or RNA mediated gene editing.

In some embodiments, within each array sub-module either the array operator is located upstream (5′) of the at least a first nucleic acid region that encodes a nucleic acid that is capable of directing RNA mediated gene regulation or RNA mediated gene editing; or the array operator is located downstream (3′) of the at least a first nucleic acid region that encodes a nucleic acid that is capable of directing RNA mediated gene regulation or RNA mediated gene editing, so as to regulate transcription of the sub-module; or to regulate transcription of the at least a first nucleic acid region that encodes a nucleic acid that is capable of directing RNA mediated gene regulation or RNA mediated gene editing.

In some embodiments, the at least one array sub-module of the nucleic acid construct provided herein comprises:
a) at least a first and a second nucleic acid region that encodes a nucleic acid that is capable of directing RNA mediated gene regulation or RNA mediated gene editing;
b) at least a first, a second and a third nucleic acid region that encodes a nucleic acid that is capable of directing RNA mediated gene regulation or RNA mediated gene editing;
c) at least a first, a second, a third and a fourth nucleic acid region that encodes a nucleic acid that is capable of directing RNA mediated gene regulation or RNA mediated gene editing;
d) at least a first, a second, a third, a fourth and a fifth nucleic acid region that encodes a nucleic acid that is capable of directing RNA mediated gene regulation or RNA mediated gene editing;
e) at least a first, a second, a third, a fourth, a fifth and a sixth nucleic acid region that encodes a nucleic acid that is capable of directing RNA mediated gene regulation or RNA mediated gene editing;
f) at least a first, a second, a third, a fourth, a fifth, a sixth and a seventh nucleic acid region that encodes a nucleic acid that is capable of directing RNA mediated gene regulation or RNA mediated gene editing;
g) at least a first, a second, a third, a fourth, a fifth, a sixth, a seventh and an eighth nucleic acid region that encodes a nucleic acid that is capable of directing RNA mediated gene regulation or RNA mediated gene editing;
h) at least a first, a second, a third, a fourth, a fifth, a sixth, a seventh, an eighth and a ninth nucleic acid region that encodes a nucleic acid that is capable of directing RNA mediated gene regulation or RNA mediated gene editing;
i) at least a first, a second, a third, a fourth, a fifth, a sixth, a seventh, an eighth, a ninth and a tenth nucleic acid region that encodes a nucleic acid that is capable of directing RNA mediated gene regulation or RNA mediated gene editing;
j) between 2 and 100 nucleic acid regions that each encode a nucleic acid that is capable of directing RNA mediated gene regulation or RNA mediated gene editing; optionally between 5 and 95, 10 and 90, 15 and 85, 20 and 80, 25 and 75, 30 and 70, 35 and 65, 40 and 60, 45 and 55 nucleic acid regions that each encode a nucleic acid that is capable of directing RNA mediated gene regulation or RNA mediated gene editing; and/or
k) at least 2, at least 3, at least 4, at least 5, at least 6, at least 7, at least 8, at 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 77, at least 76, at least 77, at least 78, at least 79, at least 80, at least 81, at least 82, at least 83, at least 84, at least 88, at least 86, at least 87, at least 88, at least 89, at least 90, at least 91, at least 92, at least 93, at least 94, at least 99, at least 96, at least 97, at least 98, at least 99, or more nucleic acid regions that each encode a nucleic acid that is capable of directing RNA mediated gene regulation or RNA mediated gene editing; optionally comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 66, 66, 67, 68, 69, 70, 71, 72, 73, 74, 77, 76, 77, 78, 79, 80, 81, 82, 83, 84, 88, 86, 87, 88, 89, 90, 91, 92, 93, 94, 99, 96, 97, 98, 99, or 100 nucleic acid regions that each encode a nucleic acid that is capable of directing RNA mediated gene regulation or RNA mediated gene editing.

In any of the embodiments of the invention, including the immediately foregoing embodiments a)-k), the (e.g., first, second, third, fourth, fifth, sixth, seventh, eighth, ninth and tenth) nucleic acid region that encodes a nucleic acid that is capable of directing RNA mediated gene regulation or RNA mediated gene editing are ordered as they are arranged in the array submodule in a 5′ to 3′ direction.

In some embodiments of the invention, including the embodiments of any of the immediately foregoing embodiments a)-k):
the array operator is located upstream (5′) of the first nucleic acid region that encodes a nucleic acid that is capable of directing RNA mediated gene regulation or RNA mediated gene editing within the array sub-module; or
downstream (3′) of the last nucleic acid region that encodes a nucleic acid that is capable of directing RNA mediated gene regulation or RNA mediated gene editing within the array sub-module,
   so as to regulate transcription of the sub-module; or to regulate transcription of the at least a first nucleic acid region that encodes a nucleic acid that is capable of directing RNA mediated gene regulation or RNA mediated gene editing.

In some embodiments, the nucleic acid construct comprises at least a first array sub-module and a second array sub-module that each comprises in 5′ to 3′ direction:
a) at least a first and a second nucleic acid nucleic acid region that encodes a nucleic acid that is capable of directing RNA mediated gene regulation or RNA mediated gene editing;
b) at least a first, a second and a third nucleic acid nucleic acid region that encodes a nucleic acid that is capable of directing RNA mediated gene regulation or RNA mediated gene editing;
c) at least a first, a second, a third and a fourth nucleic acid region that encodes a nucleic acid that is capable of directing RNA mediated gene regulation or RNA mediated gene editing;
d) at least a first, a second, a third, a fourth and a fifth nucleic acid region that encodes a nucleic acid that is capable of directing RNA mediated gene regulation or RNA mediated gene editing;
e) at least a first, a second, a third, a fourth, a fifth and a sixth nucleic acid region that encodes a nucleic acid that is capable of directing RNA mediated gene regulation or RNA mediated gene editing;
f) at least a first, a second, a third, a fourth, a fifth, a sixth and a seventh nucleic acid region that encodes a nucleic acid that is capable of directing RNA mediated gene regulation or RNA mediated gene editing;
g) at least a first, a second, a third, a fourth, a fifth, a sixth, a seventh and an eighth nucleic acid region that encodes a nucleic acid that is capable of directing RNA mediated gene regulation or RNA mediated gene editing;
h) at least a first, a second, a third, a fourth, a fifth, a sixth, a seventh, an eighth and a ninth nucleic acid region that encodes a nucleic acid that is capable of directing RNA mediated gene regulation or RNA mediated gene editing;
i) at least a first, a second, a third, a fourth, a fifth, a sixth, a seventh, an eighth, a ninth and a tenth nucleic acid region that encodes a nucleic acid that is capable of directing RNA mediated gene regulation or RNA mediated gene editing;
j) between 2 and 100 nucleic acid regions that each encode a nucleic acid that is capable of directing RNA mediated gene regulation or RNA mediated gene editing; optionally between 5 and 95, 10 and 90, 15 and 85, 20 and 80, 25 and 75, 30 and 70, 35 and 65, 40 and 60, 45 and 55 nucleic acid regions that each encode a nucleic acid that is capable of directing RNA mediated gene regulation or RNA mediated gene editing; and/or
k) at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 77, at least 76, at least 77, at least 78, at least 79, at least 80, at least 81, at least 82, at least 83, at least 84, at least 88, at least 86, at least 87, at least 88, at least 89, at least 90, at least 91, at least 92, at least 93, at least 94, at least 99, at least 96, at least 97, at least 98, at least 99, or more nucleic acid regions that each encode a nucleic acid that is capable of directing RNA mediated gene regulation or RNA mediated gene editing; optionally comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 66, 66, 67, 68, 69, 70, 71, 72, 73, 74, 77, 76, 77, 78, 79, 80, 81, 82, 83, 84, 88, 86, 87, 88, 89, 90, 91, 92, 93, 94, 99, 96, 97, 98, 99, or 100 nucleic acid regions that each encode a nucleic acid that is capable of directing RNA mediated gene regulation or RNA mediated gene editing.

In some embodiments of the invention, including the embodiments of any of the immediately foregoing embodiments a)-k), the (e.g., first, second, third, fourth, fifth, sixth, seventh, eighth, ninth and tenth) nucleic acid region that encodes a nucleic acid that is capable of directing RNA mediated gene regulation or RNA mediated gene editing are ordered as they are arranged in the array submodule in a 5′ to 3′ direction.

In any embodiment of the invention, including the immediately foregoing embodiments a)-k):
the array operator is located upstream (5′) of the first nucleic acid sequence region that encodes a nucleic acid that is capable of directing RNA mediated gene regulation or RNA mediated gene editing within each array sub-module, so as to regulate transcription of each sub-module; and
the first array sub-module is located upstream (5′) to the second array sub-module so that the array operator of the second array sub-module is positioned 3′ to the final nucleic acid region that encodes a nucleic acid that is capable of directing RNA mediated gene regulation or RNA mediated gene editing of the first array sub-module;
or
the array operator is located upstream (3′) of the last nucleic acid region that encodes a nucleic acid that is capable of directing RNA mediated gene regulation or RNA mediated gene editing within each array sub-module, so as to regulate transcription of each sub-module; and
the first array sub-module is located upstream (5′) to the second array sub-module so that the array operator of the first array sub-module is positioned 5′ to the first nucleic acid region that encodes a nucleic acid that is capable of directing RNA mediated gene regulation or RNA mediated gene editing of the second array sub-module,
   so as to regulate transcription of the sub-module; or to regulate transcription of the at least a first nucleic acid region that encodes a nucleic acid that is capable of directing RNA mediated gene regulation or RNA mediated gene editing.

In some embodiments, each array sub-module comprises 6 nucleic acid regions that each encode a nucleic acid that is capable of directing RNA mediated gene regulation or RNA mediated gene editing and one array operator of a second sequence.

In some embodiments, the promoter operator of a first sequence is capable of binding to a first activator protein and/or a first repressor protein.

In some embodiments, the promoter operator of a first sequence is capable binding to a first activator protein in the presence of an inducing agent.

In some embodiments, the promoter operator of a first sequence is capable of binding to a first activator protein in the absence of an inducing agent.

In some embodiments, the promoter operator of a first sequence is capable of binding to a first repressor protein in the absence of an inducing agent.

In some embodiments, the promoter operator of a first sequence is capable of binding to a first repressor protein in the presence of an inducing agent.

In some instances, the promoter module is an inducible promoter module. The skilled person will readily understand the term “inducible promoter”. An inducible promoter is essentially a promoter that is silent, or close to silent in the absence of some inducing agent, meaning that in the absence of the inducing agent the promoter is not transcriptionally active. The skilled person will however appreciate that some inducible promoters are “leaky” and a low level of transcription may occur in the absence of the inducing agent.

Some inducible promoters require the binding of an activator protein to an operator sequence in the promoter to become active.

Some inducible promoters comprise operator sequences which are binding sites for active repressor proteins—the binding of which is alleviated by addition of the inducing agent.

Some inducible promoters comprising binding sites both for an activator protein and for a repressor protein.

All such embodiments of inducible promoter are contemplated here and are considered to be suitable as a promoter module of the present invention.

In some embodiments, the promoter operator of a first sequence is capable of binding to a first activator protein in the presence of an inducing agent and wherein said promoter operator of a first sequence is capable of binding to a first repressor protein in the absence of same said inducing agent. In this instance the skilled person will appreciate that the presence of the inducing agent causes the promoter to become transcriptionally active.

In some embodiments, the promoter operator of a first sequence is capable of binding to a first repressor protein in the absence of an inducing agent. In this instance the skilled person will appreciate that the absence of the inducing agent causes the promoter to become transcriptionally inactive.

In some embodiments, the promoter operator of a first sequence is incapable of binding to a first repressor protein in the presence of an inducing agent. In this instance the skilled person will appreciate that the presence of the inducing agent causes the promoter to become transcriptionally active.

The present inventors surprisingly found that, despite a lack of internal promoter sequences within an array of gRNA sequences, transcription can be initiated internally from within the array, meaning that simply silencing or repression of the promoter operably linked to the array is not sufficient to produce appropriately silenced transcription—i.e., even though the operably linked promoter may be repressed, internally initiated transcription from the array would present as a “leaky” system. There are many instances where a total or substantially low transcription level from an array of for example gRNA sequences or other sequences that encodes a nucleic acid that is capable of directing RNA mediated gene regulation or RNA mediated gene editing is required.

The inventors have surprisingly found that including operator sequences within an array of for example gRNA sequences or other sequences that encodes a nucleic acid that is capable of directing RNA mediated gene regulation or RNA mediated gene editing addresses this problem and results in no, or substantially no, transcription of any of the nucleic acid sequences in the array.

Accordingly, each array sub-module comprises at least one array operator of a second sequence.

In some embodiments it is considered to be important that the promoter operator of a first sequence that is present in the promoter module is not found also within the array sub-modules. For example, in some embodiments the repressor/activator system that controls transcription of the entire gene-regulating and/or gene-editing array is a different repressor system that controls repression of transcription from internal sites within the gene-regulating and/or gene-editing array. Accordingly, in some embodiments each array sub-module comprises at least one operator of a sequence that is not the sequence of the promoter operator of a first sequence that is present in the promoter module. In line with this then, each array sub-module may have a different array operator to each other array sub-module, but wherein each of the operators within each array sub-module has a sequence that is not the sequence of the first operator present in the promoter module.

Accordingly throughout the disclosure herein, discussion of “wherein each array sub-module comprises at least one array operator of a second sequence” can be taken to mean “wherein each array sub-module comprises at least one array operator of a sequence that is not the sequence of the first operator sequence present in the promoter module”. For example, the invention provides:
a nucleic acid construct comprising:
   a) a promoter module wherein the promoter module comprises at least one promoter operator of a first sequence; and
   b) a gene-regulating and/or gene-editing array module, that comprises at least one array sub-module, wherein each array sub-module comprises at least a first nucleic acid region that encodes a nucleic acid that is capable of directing RNA mediated gene regulation or RNA mediated gene editing,
      wherein the gene-regulating and/or gene-editing array module is capable of being transcribed into a single polycistronic nucleic acid transcript from a single promoter,
      and wherein between each nucleic acid region that encodes a nucleic acid that is capable of directing RNA mediated gene regulation or RNA mediated gene editing is a sequence that when in RNA form is an RNA cleavage site; and
      wherein each array sub-module comprises at least one array operator of a of a sequence that is not the sequence of the first operator sequence present in the promoter module
   and wherein the gene-regulating and/or gene-editing array module is operably linked to promoter module.

In some embodiments, the array operator of a second sequence is capable of binding to a second repressor protein.

In some embodiments, the array operator of a second sequence is capable of binding to a second repressor protein in the absence of an inducing agent.

In some embodiments, the array operator of a second sequence is capable of binding to a second repressor protein in the presence of a repressing agent.

In some embodiments, the array operator of a second sequence is incapable of binding to a protein in the presence of an inducing agent. In some embodiments, the array operator of a second sequence is incapable of binding to a protein that is an activator protein and that can bind to the first operator sequence in the promoter module. In the presence of an inducing agent optionally incapable of binding to the first activator protein, optionally is incapable of binding to the first activator protein in the presence of an inducing agent.

Where the promoter module comprises a promoter operator of a second sequence, in some embodiments the promoter operator of a second sequence is capable of binding to a second repressor protein. In some embodiments, the promoter operator of a second sequence is capable of binding to a second repressor protein in the absence of an inducing agent. In some embodiments, the promoter operator of a second sequence is incapable of binding to a protein in the presence of an inducing agent, optionally incapable of binding to the first activator protein, optionally is incapable of binding to the first activator protein in the presence of an inducing agent.

The term “inducing agent” will be well-known to the person skilled in the art. As used herein, the term “inducing agent” refers to a chemical entity, polypeptide, or nucleic acid which is capable of controlling transcription or expression from a promoter. The inducing agent may act directly on the promoter or may act on the promoter via an intermediate molecule, for example an activator protein or a repressor protein. Exemplary discussion of transcriptional control or control of expression from a promoter by inducing agents (“inducers”) may be found in Krebs et al., 2011, Lewin's Genes X, 10^{th} Ed., Jones and Bartlett Publishers, LLC, Sudbury MA; and Alberts et al., 2008, Molecular Biology of the Cell, 5^{th} Ed., Garland Science.

Accordingly, in some embodiments, the inducing agent is selected from the group comprising or consisting of: tetracycline (Tc); anhydrotetracycline (aTc); and Doxycycline. In some embodiments, the inducer molecule is anhydrotetracycline (aTc).

As will be appreciated by the person skilled in the art, the promoter operator of a first sequence, the array operator of a second sequence, and the promoter operator of a second sequence may bind the same or different repressor proteins. Accordingly, in some embodiments, the first repressor protein and the second repressor protein are the same repressor protein. In some embodiments, the first repressor protein and the second repressor protein are different repressor proteins. In some embodiments, the array operator of a second sequence is not capable of binding to an activator protein.

In some embodiments:
the promoter module is capable of initiating transcription of the gene-regulating and/or gene-editing array module in the presence of an inducing agent; and
the promoter module is not capable of initiating transcription of the gene-regulating and/or gene-editing array module in the absence of said inducing agent.

In some embodiments:
the promoter module is capable of initiating transcription of the gene-regulating and/or gene-editing array module in the absence of the first repressor protein and/or the second repressor protein; and/or
the promoter module is not capable of initiating transcription of the gene-regulating and/or gene-editing array module in the presence of the first repressor protein and/or the second repressor protein.

In some embodiments:
the promoter module is capable of initiating transcription of the gene-regulating and/or gene-editing array module when the first activator protein is present and the first repressor protein and/or the second repressor protein is absent; and/or
the promoter module is not capable of initiating transcription of the gene-regulating and/or gene-editing array module when the first activator protein is absent and the first and/or second repressor protein is present.

In some embodiments, in the absence of an inducing agent the array operator(s) present in each array sub-module (for example, the array operator of a second sequence) are occupied by a repressor protein. In some embodiments, in the absence of an inducing agent, the promoter operator(s) present in the promoter module (for example, the promoter operator of a first sequence or the promoter operator of a second sequence) are occupied by a repressor protein.

In some embodiments, in the absence of an inducing agent the promoter operator(s) present in the promoter module (for example, the promoter operator of a first) is not occupied by an activator protein.

In some embodiments, in the presence of an inducing agent the array operator(s) present in each array sub-module (for example, the array operator of a second sequence) is not occupied by a repressor protein. In some embodiments, in the presence of an inducing agent, the promoter operator(s) present in the promoter module (for example, the promoter operator of a first sequence or the promoter operator of a second sequence) is not occupied by a repressor protein.

In some embodiments, in the presence of an inducing agent the promoter operator(s) present in the promoter module (for example, the promoter operator of a first) are occupied by an activator protein.

Any operator sequence that it suitable for inclusion in the nucleic acid construct as provided herein is contemplated by the present disclosure.

Accordingly, in some embodiments, the promoter operator of a first sequence is a TetO operator. In some embodiments, the promoter operator of a first sequence is a TetO operator that has a sequence that has at least 80%, or optionally at least 85%, at least 90%, at least 91%, at least 92%, at least 93%, at least 94%, at least 95%, at least 96%, at least 97%, at least 98%, at least 99%, or 100% sequence identity to SEQ ID NO: 1; or wherein the promoter operator of a first sequence has a sequence that is SEQ ID NO: 1.

In some embodiments, the array operator of a second sequence is a mutTetO operator sequence; optionally wherein the array operator of a second sequence has is at least 80%, at least 85%, at least 90%, at least 91%, at least 92%, at least 93%, at least 94%, at least 95%, at least 96%, at least 97%, at least 98%, at least 99%, or more sequence identity to SEQ ID NO: 2 or wherein the array operator of a second sequence has a sequence that is SEQ ID NO: 2.

In some embodiments, the promoter operator of a second sequence is a mutTetO operator sequence; optionally wherein the promoter operator of a second sequence has at least 80%, at least 85%, at least 90%, at least 91%, at least 92%, at least 93%, at least 94%, at least 95%, at least 96%, at least 97%, at least 98%, at least 99%, or more sequence identity to SEQ ID NO: 2 or wherein the promoter operator of a second sequence has a sequence that is SEQ ID NO: 2.

Any of the activator proteins or repressor proteins as disclosed herein or a fragment thereof may comprise be fused to another protein or a fragment thereof, i.e., the activator protein or repressor protein may be a fusion protein. Any of the activator proteins or repressor proteins as disclosed herein may comprise a polypeptide or fragment thereof fused to another protein or a fragment thereof, i.e., the activator protein or repressor protein may be a fusion protein. The activator protein or repressor protein may comprise—or polypeptide comprised by the activator protein or repressor protein may be—a nucleic acid binding (for example, DNA binding or RNA binding) activator protein, repressor protein, or polypeptide. Nucleic acid binding (for example, DNA binding or RNA binding) activator protein, repressor protein, or polypeptides are well known to the person skilled in the art, and include, for example transcription factors; operator proteins; sigma factors; mediator proteins; activator proteins; enhancer proteins; repressor proteins; or any combination thereof.

The activator protein and/or the repressor protein may comprise one polypeptide or a fragment thereof fused to an activation domain or a repressor domain or fragment thereof, wherein the activator domain and/or repressor domain is a transcription factor or a fragment thereof. Transcription factors are well-known to the person skilled in the art and are discussed, for example, in Krebs et al., 2011, Lewin's Genes X, 10^{th} Ed., Jones and Bartlett Publishers, LLC, Sudbury MA; and Alberts et al., 2008, Molecular Biology of the Cell, 5^{th} Ed., Garland Science. Exemplary activation domains include VP, VP16, VP64, GAL4 and B42. Exemplary repressor domains include KRAB-like effectors (optionally Mxi1), RD1152, RD11, RD5, and/or RD2.

Accordingly, in some embodiments, the first activator protein is selected from the group comprising or consisting of: rtTA-VP and rtTA-Gal4, optionally wherein the first activator protein is rtTA-Gal4. In some embodiments, the first repressor protein is TetR-Mxi1. In some embodiments, the second repressor protein is mutTetR-Mxi1. In some embodiments, the first activator protein is rtTA-VP or rtTA-Gal4, optionally rtTA-Gal4; the first repressor protein is TetR-Mxi1; and the second repressor protein is mutTetR-Mxi1.

rtTA is the reverse tetracycline-controlled transactivator protein, which is a fusion of the TetR protein and the herpes simplex virus VP16 activator protein. In some embodiments, the coding sequence of rtTA has at least 80%, at least 85%, at least 90%, at least 91%, at least 92%, at least 93%, at least 94%, at least 95%, at least 96%, at least 97%, at least 98%, at least 99%, or more sequence identity to SEQ ID NO: 3. In some embodiments, the coding sequence of rtTA has a sequence that is SEQ ID NO: 3.

TetR (e.g., UniProtKB P04483) is the repressor of the tetracycline resistance element; Its N-terminal region forms a helix-turn-helix structure and binds DNA. Binding of tetracycline to TetR reduces the repressor affinity for the tetracycline resistance gene (tetA) promoter operator sites. In some embodiments, the coding sequence of TetR has at least 80%, at least 85%, at least 90%, at least 91%, at least 92%, at least 93%, at least 94%, at least 95%, at least 96%, at least 97%, at least 98%, at least 99%, or more sequence identity to SEQ ID NO: 4. In some embodiments, the coding sequence of TetR has a sequence that is SEQ ID NO: 4.

VP or VP domain is a transcriptional activation domain composed of a fusion of the VP64 transcriptional activator protein and transcription factor p65, disclosed as VP64-p65 by Chavez et al., 2015, “Highly efficient Cas9-mediated transcriptional programming”, Nature Methods, 12:326-328. In some embodiments, the coding sequence of VP has at least 80%, at least 85%, at least 90%, at least 91%, at least 92%, at least 93%, at least 94%, at least 95%, at least 96%, at least 97%, at least 98%, at least 99%, or more sequence identity to SEQ ID NO: 5. In some embodiments, the coding sequence of VP has a sequence that is SEQ ID NO: 5.

Gal4 (UniProtKB-P04386) is a positive regulator for the gene expression of the galactose-induced genes such as GAL1, GAL2, GAL7, GAL10, and MEL1 which code for the enzymes used to convert galactose to glucose. It recognizes a 17 base pair sequence in (consensus: 5′-CGGRNNRCYNYNCNCCG-3′) the upstream activating sequence (UAS-G) of these genes. In some embodiments, the coding sequence of Gal4 has at least 80%, at least 85%, at least 90%, at least 91%, at least 92%, at least 93%, at least 94%, at least 95%, at least 96%, at least 97%, at least 98%, at least 99%, or more sequence identity to SEQ ID NO: 6. In some embodiments, the coding sequence of Gal4 has a sequence that is SEQ ID NO: 6.

Mxi1 (UniProtKB-P50539) is a transcriptional repressor. Mxi1 binds with MAX to form a sequence-specific DNA-binding protein complex which recognizes the core sequence 5′-CAC [GA] TG-3′. MXI1 thus antagonizes MYC transcriptional activity by competing for MAX. In some embodiments, the coding sequence of Mxi1 has at least 80%, at least 85%, at least 90%, at least 91%, at least 92%, at least 93%, at least 94%, at least 95%, at least 96%, at least 97%, at least 98%, at least 99%, or more sequence identity to SEQ ID NO: 7 or SEQ ID NO: 8. In some embodiments, the coding sequence of Mxi1 has a sequence that is SEQ ID NO: 7 or SEQ ID NO: 8.

In some embodiments, any of the first activator protein, the first repressor protein, and/or the second repressor protein further comprise a nuclear localisation signal (NLS), for example the NLS encoded by SEQ ID NO: 9, SEQ ID NO: 10, SEQ ID NO: 11, SEQ ID NO: 12, SEQ ID NO: 13, SEQ ID NO: 14, and/or SEQ ID NO: 15.

In some embodiments, the coding sequence of rtTA-Gal4 has at least 80%, at least 85%, at least 90%, at least 91%, at least 92%, at least 93%, at least 94%, at least 95%, at least 96%, at least 97%, at least 98%, at least 99%, or more sequence identity to SEQ ID NO: 16. In some embodiments, the coding sequence of rtTA-Gal4 is SEQ ID NO: 16.

In some embodiments, the coding sequence of TetR-Mxi1 has at least 80%, at least 85%, at least 90%, at least 91%, at least 92%, at least 93%, at least 94%, at least 95%, at least 96%, at least 97%, at least 98%, at least 99%, or more sequence identity to SEQ ID NO: 17. In some embodiments, the coding sequence of TetR-Mxi1 is SEQ ID NO: 17.

The skilled person will understand that the term “coding sequence” refers to a nucleic acid sequence, for example a DNA sequence or an RNA sequence, encoding an RNA or an amino acid sequence, e.g., a polypeptide. The person skilled in the art is readily able to determine the amino acid sequence of a polypeptide from the nucleic acid sequence or coding sequence encoding the amino acid sequence of said polypeptide, for example by using the exemplary online nucleotide sequence translation tools available at: https://web.expasy.org/translate/; https://www.ebl.ac.uk/Tools/st/emboss_transeq/; https://www.ebi.ac.uk/Tools/st/emboss_sixpack/; and/or https://www.ncbi.nim.nih.gov/orffinder/.

As disclosed above, the promoter module of the nucleic acid construct comprises at least one promoter. The promoter that drives expression of the gene-regulating and/or gene-editing array module can be any promoter. The skilled person will understand what is meant by the term promoter, and suitable promoters can be obtained from various organisms. Some promoters are species specific whilst other promoters can be used in multiple species.

Promoters are typically classed as either strong or weak depending on their affinity for RNA polymerase. The promoters used to drive expression of the at least two sequences that are transcribed into nucleic acid polymers can be a RNA Pol II promoter or a RNA Pol III promoter. Where the nucleic acid sequence that when in RNA form comprises a cleavage site is a tRNA sequence the promoter should be a RNA Pol III Promoter.

However, preferably the promoter is a RNA Pol II promoter. For example, where the cleavage site is a Csy4 cleavage sequence, a ribozyme sequence or an intron (as disclosed herein below), the promoter is preferably a RNA Pol II promoter.

The promoter, whether RNA Pol II or III, may be a strong promoter. By a “strong promoter” we include the meaning of a promoter that produces RNA molecules at a rate that is significantly faster than the average promoter within the genome of any given organism or in vitro. The strong promoters described herein have been characterised in accordance with Lee et al., 2015, ACS Synth Biol 9:975-986 which is specifically incorporated by reference, particularly the methods relating to analysis of promoter strength under the heading “Characterization of promoters” on page 978-979. The skilled person will understand how to identify a strong promoter. For example, the strength of various promoters that are native to a particular organism can be tested by, for example, analysing the amount of fluorescent protein produced from a gene under the control of each promoter to be tested. It will then be readily apparent to the skilled person which of these promoters are strong and which are not strong. In one embodiment a strong promoter for use in a particular organism is a promoter that produces RNA molecules at a rate that is significantly faster than the average promoter found within the genome of the particular organism. See also Qin et al., 2010, PLOS One, 5 (5): e10611.

Other strong promoters are considered to include the Human elongation factor 1 promoter (EF1A) and the chicken β-Actin promoter coupled with CMV early enhancer (CAGG) promoter.

In one embodiment the promoter is a RNA Pol II promoter. In a further embodiment the promoter is a strong RNA Pol II promoter. In yet a further embodiment the promoter is an inducible RNA Pol II promoter, optionally an inducible strong RNA Pol II promoter.

In one embodiment the Pol II promoter is selected from the group consisting of the TDH3 promoter, TEF1 promoter, PGK1 promoter, pCCW12 promoter, pTEF2 promoter, pHHF1 promoter, pHHF2 promoter, pALD6 promoter, Gal1 promoter, pPGK1 promoter, pHTB2 promoter or the CUP1 promoter. The Gal1 promoter is inducible by galactose and the CUP1 promoter is inducible by copper-sulphate. Tetracycline inducible promoters are also considered to be useful. In a preferred embodiment the promoter is a Pol II promoter and is a TDH3 promoter (See for example Lee et al 2015 ACS Synthetic Biology 4:975-986).

The promoters discussed above are yeast promoters and may not work in some other organisms. However, as described in detail above, the skilled person will be able to identify suitable strong promoters for use in other organisms without undue burden. Indeed, the strength of many promoters have already been characterised as discussed above.

In one embodiment the promoter is a RNA Pol III promoter. In a further embodiment the promoter is a strong RNA Pol III promoter. In yet a further embodiment the promoter is an inducible RNA Pol III promoter, optionally an inducible strong RNA Pol III promoter. In one embodiment the Pol III promoter is selected from the group consisting of the tRNA Phe promoter with a 5′ HDV ribozyme, the U6 promoter or the H1 promoter.

The promoter, for example the strong promoter, for use in the invention may be a naturally occurring promoter or may be a synthetic promoter.

In some specific embodiments, the at least one promoter is:
a) a Pol II promoter, optionally
   wherein the promoter is an inducible promoter
   wherein the Pol II promoter is classed as a strong promoter;
   and/or
   wherein the Pol II promoter is selected from the group consisting of a TDH3 promoter, a TEF1 promoter, a PGK1 promoter, a pCCW12 promoter, a pTEF2 promoter, a pHHF1 promoter, a pHHF2 promoter, a pALD6, promoter, a pGal1 promoter, a pPGK1 promoter, a pHTB2 promoter, a pCUP1 promoter, or a pTet promoter; or
b) a Pol III promoter, optionally
   wherein the Pol III promoter is classed as a strong Pol III promoter;
   wherein the Pol III promoter is an inducible promoter; and/or
   wherein the Pol III promoter is selected from the group consisting of the tRNA Phe promoter with a 5′ HDV ribozyme, the U6 promoter or H1 promoter.

As will be understand that typically it is not the nucleic acid polymer (or portions thereof) of the RNA mediated gene regulating or editing nucleic acid construct or gene-regulating and/or gene-editing array module that performs the RNA mediated gene regulation or editing. Rather, the RNA mediated gene regulating or editing nucleic acid construct comprises sequences that, once transcribed into RNA are then capable of performing the gene regulation or editing. Accordingly, in one embodiment, the gene-regulating or gene-editing array module comprises DNA that is transcribed into RNA that mediates gene regulation or editing; or in one embodiment, the gene-regulating or gene-editing array module comprises DNA that encodes RNA that mediates gene regulation or editing.

The nucleic acid sequences that are transcribed into nucleic acid polymers that each separately direct RNA mediated gene regulation or editing, for example the gene-regulating or gene-editing array module, are suitable for use in any method of RNA mediated gene regulation or editing. For example, in one embodiment the gene-regulating or gene-editing array module is suitable for use in any one or more of CRISPR, sense Suppression/Cosuppression, antisense suppression, double-stranded RNA interference, hairpin RNA interference, intron-containing hairpin RNA interference, siRNA, microRNA, piRNA and snoRNA methods. For example, in one embodiment the nucleic acids that are capable of directing RNA mediated gene regulation or RNA mediated gene editing are gRNA polymers. In another embodiment the nucleic acids that are capable of directing RNA mediated gene regulation or RNA mediated gene editing are siRNA polymers.

Methods of gene regulation or editing such as CRISPR, sense Suppression/Cosuppression, antisense suppression, double-stranded RNA interference, hairpin RNA interference, intron-containing hairpin RNA interference, small interfering (siRNA), microRNA (miRNA), picoRNA (piRNA) and small nucleolar RNA (snoRNA) are well known to the skilled person and the preferences for the components and nucleic acids required to carry out the gene regulation or editing are well known. For example, miRNAs are typically about 20-23 nt in length and are found in plants, animals and certain viruses. miRNAs bind to target RNA molecules and regulate their translation but also appear to have other functions, including cleavage of target mRNAs and destabilization of target mRNAs. miRNAs are typically encoded as a miRNA stem-loop, or pre-processed miRNA. After processing by endogenous cellular machinery, a mature microRNA is released.

Key proteins of the microprocessor are DGCR8, which binds the RNA molecule, and Drosha, an RNase III type enzyme, which cleaves the primary (pri) miRNA transcript into a precursor (pre) miRNA stem-loop molecule of ˜70-80 bases. In the second step, which occurs after its export by exportin-5 to the cytoplasm, the pre-miRNA is cleaved by the RNase III Dicer yielding mature miRNA and its complementary miRNA. The miRNA is then loaded on the RNA-induced silencing complex (RISC), which directs its binding to its target gene.

snoRNAs are typically encoded in the introns of genes. Around 300 have been identified in the human genome. There are three types of snoRNA, the C/D box type, the H/ACA box type, and the composite H/ACA and C/D box type. The different types differ based on secondary structure of the snoRNA.

SIRNA, sometimes known as short interfering RNA or silencing RNA, is a class of double-stranded RNA molecules which are typically 20-25 base pairs in length, similar to miRNA, and operate within the RNA interference (RNAi) pathway. It interferes with the expression of specific genes with complementary nucleotide sequences by degrading mRNA after transcription, preventing translation. The sequence of the siRNA is therefore designed to be complementary to a target RNA molecule, thus impairing translation of said target RNA molecule. Sequences vary greatly, depending on target gene, but siRNAs are typically comprised of a stem-loop structure comprising a 19 bp stem and 9 nt loop with 2-3 U's at the 3′ end. Design guides are readily available to the skilled person, for example at the ThermoFisher website: See: https://www.thermofisher.com/us/en/home/references/ambion-rech-support/rnai-sirna/general-articles/-sima-design-guidelines.html.

As provided herein, the gene-regulating and/or gene-editing array module is capable of being transcribed into a single polycistronic nucleic acid transcript from a single promoter. As will be appreciated, the single polycistronic nucleic acid transcript comprises at least one array sub-module, wherein each array sub-module comprises at least a first nucleic acid region that encodes a nucleic acid that is capable of directing RNA mediated gene regulation or RNA mediated gene editing. Accordingly, the nucleic acid sequences that are transcribed into the single polycistronic nucleic acid transcript may each be gRNA polymers, for example for use in CRISPR interference (CRISPRi), CRISPR activation (CRISPRa), and/or CRISPR activation and interference (CRISPRai). Alternatively, the RNA mediated gene regulating nucleic acid construct may comprise nucleic acid sequences that are transcribed into the single polycistronic nucleic acid transcript comprising nucleic acid sequences that are each capable of directing RNA mediated gene regulation or RNA mediated gene editing which are suitable for use in different methods of RNA mediated gene regulation or editing. For example, the nucleic acid sequences that are each capable of directing RNA mediated gene regulation or RNA mediated gene editing may comprise gRNA sequences and siRNA sequences.

For example, expressing two gRNAs and a microRNA simultaneously from a single transcript and processing this transcript with DROSHA/microRNA machinery can be used to strongly inhibit Hepatitis B virus replication in vivo (see Wang et al., 2017, Theranostics, 7:3090-3105). The skilled person will appreciate that this and other combinations of gene regulating or editing sequences can be incorporated into a single transcript using the methods and components of the present invention.

In preferred embodiments, each nucleic acid that is capable of directing RNA mediated gene regulation or RNA mediated gene editing may be a gRNA, optionally is a gRNA. As disclosed herein, such gRNAs are suitable for use in each nucleic acid that is capable of directing RNA mediated gene regulation or RNA mediated gene editing may be a gRNA, optionally is a gRNA. Accordingly, in these preferred embodiments each nucleic acid that is capable of directing RNA mediated gene regulation or RNA mediated gene editing, when transcribed into RNA form, is independently capable of associating with a polypeptide. Said polypeptide is capable of regulating a gene. Said polypeptide is optionally selected from the group comprising or consisting of: Cas9 or Cas9-like polypeptide; dCas9 or dCas9-like polypeptide; Cas12a; dCas12a; Cas12b; dCas12b; Cas13a; dCas13a; Cas13b; dCas13b; LbCpf1; dLbCpf1; AsCpf1; dAsCpf1; or dFnCpf1; or FnCpf1; or a fusion protein thereof.

By “directing RNA mediated gene regulation or RNA mediate gene editing” we include the meaning of targeting to a particular target gene or locus. For example, the RNA mediated mechanisms discussed herein are targeted to specific nucleic acids by virtue of the RNA sequence of the RNA that mediates the regulation or editing. Accordingly, the sequence of the RNA is important in defining where the regulation or editing will occur.

In some embodiments, the nucleic acid region that encodes a nucleic acid that is capable of directing RNA mediated gene regulation or RNA mediated gene editing disclosed herein is complementary to a target nucleic acid region. In some embodiments, each nucleic acid region that encodes a nucleic acid that is capable of directing RNA mediated gene regulation or RNA mediated gene editing, is complementary to the same target nucleic acid region, optionally wherein the target nucleic acid region is a promoter of a target gene.

In some embodiments, each nucleic acid region that encodes a nucleic acid that is capable of directing RNA mediated gene regulation or RNA mediated gene editing is complementary to the same target nucleic acid region, optionally wherein the target nucleic acid region is a promoter of a target gene, but the sequences of each nucleic acid region that encodes a nucleic acid that is capable of directing RNA mediated gene regulation or RNA mediated gene editing nucleic acid are different.

In some embodiments, within each array sub-module all of the nucleic acid regions that encode a nucleic acid that is capable of directing RNA mediated gene regulation or RNA mediated gene editing are each complementary to the same target nucleic acid region, optionally wherein the target nucleic acid region is a promoter of a target gene, but within each array sub-module the nucleic acid regions that encode a nucleic acid that is capable of directing RNA mediated gene regulation or RNA mediated gene editing are different.

As disclosed herein, the gene-regulating and/or gene-editing array module comprises at least one array sub-molecule; and each array sub-molecule comprises at least a first nucleic acid region that encodes a nucleic acid that is capable of directing RNA mediated gene regulation or RNA mediated gene editing. As disclosed herein, the number of array sub-modules may be 1 or between 2-100 array sub-modules; and the number of nucleic acid regions that encode a nucleic acid that is capable of directing RNA mediated gene regulation or RNA mediated gene editing may be 1 or between 2-100 nucleic acid regions that each encode a nucleic acid that is capable of directing RNA mediated gene regulation or RNA mediated gene editing.

In some embodiments where the gene-regulating and/or gene-editing array module comprises 1 array sub-module, the array sub-module preferably comprises more than 1 nucleic acid region that each encode a nucleic acid that is capable of directing RNA mediated gene regulation or RNA mediated gene editing, for example between 2-100 nucleic acid regions that each encode a nucleic acid that is capable of directing RNA mediated gene regulation or RNA mediated gene editing. In some embodiments where the gene-regulating and/or gene-editing array module comprises more than 1 array sub-module, for example between 2-100 array sub-modules, each array sub-module may preferably comprise 1 nucleic acid region that each encode a nucleic acid that is capable of directing RNA mediated gene regulation or RNA mediated gene editing. In some embodiments where the gene-regulating and/or gene-editing array module comprises more than 1 array sub-module, for example between 2-100 array sub-modules, each array sub-module may preferably comprise more than 1 nucleic acid region that each encode a nucleic acid that is capable of directing RNA mediated gene regulation or RNA mediated gene editing, for example between 2-100 nucleic acid regions that each encode a nucleic acid that is capable of directing RNA mediated gene regulation or RNA mediated gene editing.

In some embodiments, each region that encodes a nucleic acid that is capable of directing RNA mediated gene regulation or RNA mediated gene editing, when transcribed into RNA form, are each independently capable of associating with a regulatory polypeptide, wherein said polypeptide is capable of regulating a gene, optionally wherein said polypeptide is selected from the group comprising or consisting of: Cas9 or Cas9-like polypeptide; dCas9 or dCas9-like polypeptide; Cas12a; dCas12a; Cas12b; dCas12b; Cas13a; dCas13a; Cas13b; dCas13b; LbCpf1; dLbCpf1; AsCpf1; dAsCpf1; or dFnCpf1; or FnCpf1; or a fusion protein thereof. In these embodiments:
a) the region(s) that encode a nucleic acid that is capable of directing RNA mediated gene regulation or RNA mediated gene editing, when transcribed into RNA form, are each independently capable of associating with the same regulatory polypeptide; or
b) the region(s) that encode a nucleic acid that is capable of directing RNA mediated gene regulation or RNA mediated gene editing, when transcribed into RNA form, are each independently capable of associating with one of at least two different regulatory polypeptides.

As disclosed elsewhere herein, between each nucleic acid region that encodes a nucleic acid that is capable of directing RNA mediated gene regulation or RNA mediated gene editing is a sequence that when in RNA form is an RNA cleavage site.

In preferred embodiments the cleavage site is a transcriptionally inert cleavage site i.e. is a site that is not capable of initiating transcription.

In some embodiments, the cleavage site is selected from:
i) an endoribonuclease cleavage site, for example a site-specific RNA endonuclease site, for optionally a Csy4 cleavage sequence or an artificial site-specific RNA endonuclease
ii) a tRNA sequence
iii) a ribozyme sequence
iv) an intron
v) a target sequence for an RNA directed cleavage complex;
vi) a site cleavable by a Cas protein with RNA endonuclease activity, optionally cleavable by Cas12a.

In preferred embodiments, the cleavage site is a Csy4 cleavage site. In some embodiments, the Csy4 cleavage site has a sequence that is at least 80%, at least 85%, at least 90%, at least 91%, at least 92%, at least 93%, at least 94%, at least 95%, at least 96%, at least 97%, at least 98%, at least 99%, or more sequence identity to SEQ ID NO: 18, SEQ ID NO: 19, SEQ ID NO: 20, and/or SEQ ID NO: 21. In some embodiments, the Csy4 cleavage site has a sequence that is SEQ ID NO: 18, SEQ ID NO: 19, SEQ ID NO: 20, or SEQ ID NO: 21.

It will be clear to the skilled person, however, that the requirement for the cleavage sequence is simply that, once transcribed into RNA, it is capable of being specifically cleaved, for example cleaved by an enzyme or polypeptide. There are various ways in which this can be achieved.

For example, site-specific RNA endonucleases exist, for example artificial Site-specific RNA endonucleases, or ASREs, see for example Choudhury et al 2012 Nature Communications 3 Article 1147; and Zhang et al 2013 Molecular Therapy 22 (2) 312-320. The use of such enzymes and the accompanying recognition sequences are encompassed in the present invention.

Another RNA specific endonuclease is Csy4 which is a CRISPR endonuclease that processes RNA. Specifically, Csy4, in native bacterial systems (such as Pseudomonas aeruginosa) processes pre-crRNA transcripts by cleaving a specific, 28 nucleotide long stem-and-loop sequence of RNA. Csy4 specifically cleaves only its cognate pre-crRNA substrate.

Recognition of its cognate pre-crRNA substrate is mediated, in part, by interactions with the following amino acid residues in the Csy4 protein: Q104, A19, U7, G20, C6, F155, R102. See for example Haurwitz et al Science, 2010 Sep. 10; 329 (5997): 1355-8. doi: 10.1126/science.1192272.

The Csy4 cleavage site for use in the invention is considered to be a 20 nucleotide cleavage site, or a 28 nucleotide cleavage site. The Csy4 protein only cleaves the site in RNA, not in DNA. Accordingly, it will be understood that where the nucleic acid construct is DNA, the Csy4 protein does not cleave the DNA vector, but only cleaves the RNA transcript produced from the destination vector, into which the nucleic acid that encodes the Csy4 protein in incorporated. SEQ ID NO: 18-21 provide sequence information for the DNA and RNA Csy4 site sequences. The skilled person will understand that some variation in these sequences may be tolerated and still allow the Csy4 protein to cleave the site.

In other embodiments, the cleavage site is a pre-tRNA sequence. tRNA sequences are cleaved in eukaryotes by RNase P and RNase Z (or RNase E in bacteria), which removes excess 5′ and 3′ sequences. These enzymes recognize the tRNA secondary structure, so must be expressed to cleave ANY desired tRNA sequence. See Shiraki and Kawakami 2018 Scientific Reports 8:13366.

Accordingly, in one embodiment the nucleic acid construct comprises a nucleic acid sequence of SEQ ID NO: 18, SEQ ID NO: 19, SEQ ID NO: 20, and/or SEQ ID NO: 21.

The following shows some exemplary tRNA sequences along with the 5′ leader sequence.

pre-tRNA^{Gly}:
[SEQ ID NO: 22]
5′-AACAAAGCACCAGTGGTCTAGTGGTAGAATAGTACCCT
Dr-RNAGly(GCC)]
[SEQ ID NO: 23]
gtgaGCATTGGTGGTTCAGTGGTAGAATTCTCGCCTGCCACGCGGGAGGCCCGGGTT
CGATTCCCGGCCAATGCA
Dr-tRNALys(CTT)
[SEQ ID NO: 24]
gttctcatcaGCCCGGCTAGCTCAGTCGGTAGAGCATGAGACTCTTAATCTCAGGGTCGTG
GGTTCGAGCCCCACGTCGGGCG
Dr-tRNAAsn(GTT)
[SEQ ID NO: 25]
gctatctGTCTCTGTGGCGCAATCGGTTAGCGCGTTCGGCTGTTAACCGAAAGGTTGGT
GGTTCGAGCCCACCCAGGGACG
Dr-tRNAMet(CAT)
[SEQ ID NO: 26]
gcctgaagGTTTCCGTAGTGTAGTGGTTATCACGTTCGCCTCATACGCGAAAGGTCCCCA
GTTCGAAACTGGGCGGAAACA
Dr-tRNAGln(CTG)
[SEQ ID NO: 27]
gacttgaGGTTCCATGGTGTAATGGTTAGCACTCTGGACTCTGAATCCAGCGATCCGAGT
TCAAATCTCGGTGGGACCA
Dr-tRNASer(GCT)
[SEQ ID NO: 28]
ggaaaatGACGAGGTGGCCGAGTGGTTAAGGCGATGGACTGCTAATCCATTGTGCTTTG
CACGCATGGGTTCGAATCCCATCCTCGTCG
Dr-tRNAThr(AGT)
[SEQ ID NO: 29]
gcagcGGCGCCGTGGCTTAGTTGGTTAAAGCGCCTGTCTAGTAAACAGGAGATCCTGG
GTTCGAATCCCAGCGGTGCCT
Dr-tRNAHis(GTG)
[SEQ ID NO: 30]
gctcGCCGTGATCGTACAGTGGTTAGTACTCTGCGTTGTGGCCGCAGCAACCCCGGTT
CGAATCCGGGTCACGGCA
Dr-tRNALeu(CAG)
[SEQ ID NO: 31]
gcatGTCAGGATGGCCGAGTGGTCTAAGGCGCTGCGTTCAGGTCGCAGTCTCCCCTG
GAGGCGTGGGTTCGAATCCCACTTCTGACA
Os-tRNAGly(GCC)
[SEQ ID NO: 32]
gaacaaaGCACCAGTGGTCTAGTGGTAGAATAGTACCCTGCCACGGTACAGACCCGGG
TTCGATTCCCGGCTGGTGCA
Shiraki and Kawakami 2
Os-tRNAGly(GCC)-scrambled
[SEQ ID NO: 33]
GAACCTCTTACACGCGCAGATCAACTAAATGTACACTGCGACGGTCCGTGGCTCCGA
GAGGGGTTACAGGGTACGCTG
>Dr-tRNAGly(GCC)-scrambled
[SEQ ID NO: 34]
GCGCTGTGGCGTACCGGGTACGTACTCGCTTGACTGGGTTGGTACTAGGCGAAACC
AGCTCCGTGGGATTGCACC

In some embodiments, the cleavage site is not a tRNA sequence.

In some embodiments a tRNA sequence is not considered to be transcriptionally inert, and is considered to be capable of initiating transcription. Accordingly, in some embodiments, the presence of tRNA sequences with the array or sub-array is to be avoided.

The nucleic acid sequence that when in RNA form comprises a cleavage site may also be a ribozyme cleavage site. The skilled person will understand preferences for ribozymes. Exemplary ribozymes and the associated sequences include:

Hammerhead ribozyme (HH)
[SEQ ID NO: 35]
gttccccCTGATGAGTCCGTGAGGACGAAACGAGTAAGCTCGTC
Hepatitis delta virus ribozyme (HDV)
[SEQ ID NO: 36]
GGCCGGCATGGTCCCAGCCTCCTCGCTGGCGCCGGCTGGGCAAC
ATGCTTCGGCATGGCGAATGGGAC

In some embodiments, the cleavage site is not a ribozyme.

As discussed above, the nucleic acid sequence that when in RNA form comprises a cleavage site may also be an intron. Intron sequences are naturally present in some genes. These native genetic promoters have been adapted for use in gRNA multiplexing (e.g., in rice plants, the UBI10p promoter is used; the 5′ UTR of this promoter has a conserved intron). The skilled person will understand what is required to put this embodiment into practice. See for example “Engineering Introns to Express RNA Guides for Cas9- and Cpf1-Mediated Multiplex Genome Editing” by Ding D. et al., 2018, Mol Plant., 11 (4): 542-552. doi: 10.1016/j.molp.2018.02.005. Epub 2018 Feb. 17.

In some embodiments, the cleavage site is not an intron.

As discussed above, the only requirement for the sequence that when in RNA form comprises a cleavage site is that it is cleaved. It will be appreciated that the sequence of this region of the nucleic acid construct can actually be of any sequence, and this sequence can be cleaved by a RNA directed cleavage complex, as siRNA for example an siRNA complexed with Ago2. When using nucleic acid constructs which include such cleavage sites, the appropriate RNA polymers, for example siRNAs, have to be co-expressed. In some embodiments, the nucleic acid construct can be used to produce a nucleic acid construct that comprises sites for, for example RNA directed cleavage, wherein the RNA species or transcript that directs the cleavage is encoded with the same nucleic acid construct. In this way, the nucleic acid construct can essentially be self-processed using self-encoded RNA molecules in combination with co-expressed proteins, for example Ago2.

It will be appreciated that it is advantageous in some applications to differentially control the cleavage of the gene-regulating or gene-editing array module. This may be achieved, for example, by placing different cleavage sites between each nucleic acid region that encodes a nucleic acid that is capable of directing RNA mediated gene regulation or RNA mediated gene editing.

Accordingly, in some embodiments, each cleavage site between each nucleic acid region that encodes a nucleic acid that is capable of directing RNA mediated gene regulation or RNA mediated gene editing is independently selected from:
i) an endoribonuclease cleavage site, for example a site-specific RNA endonuclease site, for optionally a Csy4 cleavage sequence or an artificial site-specific RNA endonuclease
ii) a tRNA sequence
iii) a ribozyme sequence
iv) an intron
v) a target sequence for an RNA directed cleavage complex; or
vi) a site cleavable by a Cas protein with RNA endonuclease activity, optionally cleavable by Cas12a.

In some embodiments, every cleavage site between each nucleic acid region that encodes a nucleic acid that is capable of directing RNA mediated gene regulation or RNA mediated gene editing is:
i) an endoribonuclease cleavage site, for example a site-specific RNA endonuclease site, for optionally a Csy4 cleavage sequence or an artificial site-specific RNA endonuclease
ii) a tRNA sequence
iii) a ribozyme sequence
iv) an intron
v) a target sequence for an RNA directed cleavage complex; or
vi) a site cleavable by a Cas protein with RNA endonuclease activity, optionally cleavable by Cas12a.

As mentioned previously, the type of RNA mediated gene regulation or RNA mediated gene editing that the nucleic acid sequences are capable of directing can be, for example, siRNA or CRISPR. Some of these methods of regulation require additional factors. For example, CRISPR, CRISPRI, CRISPRa, and/or CRISPRai require a regulatory polypeptide that is capable of association with the sgRNA. A commonly used regulatory polypeptide is the Cas9 polypeptide. However, other Cas9-like regulatory polypeptides exist that can also mediate CRISPR-type gene regulation and CRISPR-type gene editing. As will be appreciated, it may be advantageous in some instances to encode the polypeptide on the same nucleic acid construct as the promoter module and/or the gene-regulating and/or gene-editing array module. In some instances, however, it may be preferred to encode the regulatory polypeptide on a different nucleic acid.

Accordingly, in some embodiments, the nucleic acid construct further comprises a regulatory protein module, wherein the regulatory protein module comprises:
a) a first nucleotide region encoding a first regulatory polypeptide; and/or
b) a second nucleotide region encoding a second regulatory polypeptide;
optionally the first regulatory polypeptide and the second regulatory polypeptide are selected from the group comprising or consisting of:
Cas9 or Cas9-like polypeptide; dCas9 or dCas9-like polypeptide; Cas12a; dCas12a; Cas12b; dCas12b; Cas13a; dCas13a; Cas13b; dCas13b; LbCpf1; dLbCpf1; AsCpf1; dAsCpf1; or dFnCpf1; or FnCpf1; or a fusion protein thereof, optionally Cas9-Mxi1, Cas9-like-Mxi1, dCas9-Mxi1, dCas9-like-Mxi1, Cas12a-Mxi1, dCas12a-Mxi1, Cas12b-Mxi1, dCas12b-Mxi1, Cas13a-Mxi1, dCas13a-Mxi1, Cas13b-Mxi1, dCas13b-Mxi1, LbCpf1-Mxi1, dLbCpf1-Mxi1, AsCpf1-Mxi1, dAsCpf1-Mxi1, dFnCpf1-Mxi1, FnCpf1-Mxi1, Cas9-VP, Cas9-like-VP polypeptide, dCas9-VP, dCas9-like-VP polypeptide, Cas12a-VP, dCas12a-VP, Cas12b-VP, dCas12b-VP, Cas13a-VP, dCas13a-VP, Cas13b-VP, dCas13b-VP, LbCpf1-VP, dLbCpf1-VP, AsCpf1-VP, dAsCpf1-VP, dFnCpf1-VP, or FnCpf1-VP.

In order to achieve CRISPR-type control of gene regulation, the regulatory polypeptide may advantageously be fused to an activator domain and/or a repressor domain. For example, the activator protein may be fused to an activation domain or a repressor domain or fragment thereof, wherein the activator domain and/or repressor domain is a transcription factor or a fragment thereof. Transcription factors are well-known to the person skilled in the art and are discussed, for example, in Krebs et al., 2011, Lewin's Genes X, 10^{th} Ed., Jones and Bartlett Publishers, LLC, Sudbury MA; and Alberts et al., 2008, Molecular Biology of the Cell, 5^{th} Ed., Garland Science. Exemplary activation domains include VP, VP16, VP64, GAL4 and B42. Exemplary repressor domains include KRAB-like effectors (optionally Mxi1), RD1152, RD11, RD5, and/or RD2.

Therefore, in some embodiments, the regulatory polypeptide capable of regulating a gene, the first regulatory polypeptide and/or the second regulatory polypeptide is fused to an activator domain and/or a repressor domain; optionally wherein the activator domain is selected from the group comprising or consisting of: VP, VP16, VP64, GAL4 and B42; and/or wherein the repressor domain is selected from the group comprising or consisting of: KRAB-like effectors (optionally Mxi1), RD1152, RD11, RD5, and/or RD2.

In some embodiments, the first regulatory polypeptide and/or the second regulatory polypeptide are each selected from the group comprising or consisting of:
a Cas9-Mxi1 or Cas9-like-Mxi1 polypeptide; a dCas9-Mxi1 or dCas9-like-Mxi1 polypeptide; Cas12a-Mxi1; dCas12a-Mxi1; Cas12b-Mxi1; dCas12b-Mxi1; Cas13a-Mxi1; dCas13a-Mxi1; Cas13b-Mxi1; dCas13b-Mxi1; LbCpf1-Mxi1; dLbCpf1-Mxi1; AsCpf1-Mxi1; dAsCpf1-Mxi1; dFnCpf1-Mxi1; or FnCpf1-Mxi1; and/or
a Cas9-VP or Cas9-like-VP polypeptide; a dCas9-VP or dCas9-like-VP polypeptide; Cas12a-VP; dCas12a-VP; Cas12b-VP; dCas12b-VP; Cas13a-VP; dCas13a-VP; Cas13b-VP; dCas13b-VP; LbCpf1-VP; dLbCpf1-VP; AsCpf1-VP; dAsCpf1-VP; dFnCpf1-VP; or FnCpf1-VP.

In some embodiments:
a) the first regulatory polypeptide is selected from the group comprising or consisting of a Cas9-Mxi1 or Cas9-like-Mxi1 polypeptide; a dCas9-Mxi1 or dCas9-like-Mxi1 polypeptide; Cas12a-Mxi1; dCas12a-Mxi1; Cas12b-Mxi1; dCas12b-Mxi1; Cas13a-Mxi1; dCas13a-Mxi1; Cas13b-Mxi1; dCas13b-Mxi1; LbCpf1-Mxi1; dLbCpf1-Mxi1; AsCpf1-Mxi1; dAsCpf1-Mxi1; dFnCpf1-Mxi1; or FnCpf1-Mxi1, optionally is a dCas9-Mxi1 polypeptide; and/or
b) the second regulatory polypeptide is selected from the group comprising or consisting of a Cas9-VP or Cas9-like-VP polypeptide; a dCas9-VP or dCas9-like-VP polypeptide; Cas12a-VP; dCas12a-VP; Cas12b-VP; dCas12b-VP; Cas13a-VP; dCas13a-VP; Cas13b-VP; dCas13b-VP; LbCpf1-VP; dLbCpf1-VP; AsCpf1-VP; dAsCpf1-VP; dFnCpf1-VP; or FnCpf1-VP, optionally is a dCas12a-VP polypeptide.

In some embodiments:
a) the first regulatory polypeptide is encoded by a sequence that:
   is at least 80%, at least 85%, at least 90%, at least 91%, at least 92%, at least 93%, at least 94%, at least 95%, at least 96%, at least 97%, at least 98%, at least 99%, or more identical to SEQ ID NO: 37; or
   is 80%, 85%, 90%, 91%, 92%, 93%, 94%, 95%, 96%, 99%, 100% identical to SEQ ID NO: 37; or
   is SEQ ID NO: 37; and/or
b) the second regulatory polypeptide is encoded by a sequence that:
   is at least 80%, at least 85%, at least 90%, at least 91%, at least 92%, at least 93%, at least 94%, at least 95%, at least 96%, at least 97%, at least 98%, at least 99%, or more identical to SEQ ID NO: 38; or
   is 80%, 85%, 90%, 91%, 92%, 93%, 94%, 95%, 96%, 99%, 100% identical to SEQ ID NO: 38; or
   is SEQ ID NO: 38.

In some embodiments, the first regulatory polypeptide is capable of activating or increasing expression of a target gene. In some embodiments, the second regulatory polypeptide is capable of interfering with or decreasing the expression of a target gene. In some particular embodiments, to be capable of regulating a gene, the regulatory polypeptide must associate or form a complex with the nucleic acid that is capable of directing RNA mediated gene regulation or RNA mediated gene editing; optionally forms a complex with the nucleic acid that is capable of directing RNA mediated gene regulation or RNA mediated gene editing in RNA form. In some embodiments, to be capable of regulating a gene, the regulatory polypeptide must associate or form a complex with the nucleic acid that is capable of directing RNA mediated gene regulation or RNA mediated gene editing in RNA form, once the single polycistronic transcript has been processed or cleaved by an enzyme or polypeptide that is capable of cleaving the cleavage site.

As will be appreciated, in some instances it is advantageous and/or necessary that the nucleic acid construct encodes a polypeptide that is capable of cleaving the cleavage site present in the array module when in RNA form, for example in instances where an organism does not express a polypeptide that is capable of cleaving the cleavage site present in the array module when in RNA form; or where control over the expression of a polypeptide that is capable of cleaving the cleavage site present in the array module when in RNA form is desired.

Exemplary polypeptides and enzymes capable of cleaving the cleavage site present in the array module when in RNA form are discussed hereinabove.

In some embodiments, the nucleic acid construct comprises a nucleic acid region encoding a polypeptide that is capable of cleaving the cleavage site present in the array module when in RNA form; optionally
wherein the polypeptide that is capable of cleaving the cleavage site when in RNA form is:
i) an endoribonuclease, for example a site-specific RNA endonuclease, for example a Csy4 or an artificial site-specific RNA endonuclease;
ii) a polypeptide capable of cleaving a tRNA sequence
iii) a polypeptide capable of cleaving an intron sequence; or
v) polypeptide capable of cleaving a target sequence for an RNA directed cleavage complex; or
vi) a Cas protein with RNA endonuclease activity, optionally cleavable by Cas12a.

In a preferred embodiment, the polypeptide that is capable of cleaving the cleavage site when in RNA form is Csy4.

In some embodiments, the nucleic acid region encoding a polypeptide that is capable of cleaving the cleavage site present in the array module when in RNA form is encoded by a sequence that is at least 80%, at least 85%, at least 90%, at least 91%, at least 92%, at least 93%, at least 94%, at least 95%, at least 96%, at least 97%, at least 98%, at least 99%, or more identical to SEQ ID NO: 39; or is 80%, 85%, 90%, 91%, 92%, 93%, 94%, 95%, 96%, 99%, 100% identical to SEQ ID NO: 39; or is SEQ ID NO: 39.

Where the nucleic acid construct comprises a first nucleotide region encoding a first regulatory polypeptide; and/or a second nucleotide region encoding a second regulatory polypeptide; and/or a nucleic acid region encoding a polypeptide that is capable of cleaving the cleavage site present in the array module when in RNA form, it is desirable to control expression of said polypeptides from a promoter region. As disclosed herein, the skilled person will understand what is meant by the term promoter, and suitable promoters can be obtained from various organisms. Some promoters are species specific whilst other promoters can be used in multiple species.

Accordingly, in some embodiments:
a) the first nucleotide region encoding a first regulatory polypeptide is operably linked to a promoter region;
b) the second nucleotide region encoding a second regulatory polypeptide is operably linked to a promoter region; and
c) the nucleic acid region encoding a polypeptide that is capable of cleaving the cleavage site present in the array module when in RNA form is operably linked to a promoter region;
optionally where the promoter region of (a), (b) and (c) are different promoters.

In some embodiments, the promoter region of (a), (b) and/or (c) is a weak promoter or a medium-strength promoter; optionally the promoter is selected from the group comprising or consisting of: REV1, PSP2, HTB2, RAD27, or POP6.

Any of the activator and/or repressor proteins as disclosed herein may be encoded by the nucleic acid construct. Accordingly, in some embodiments, the nucleic acid construct further comprises:
i) a nucleic acid sequence encoding a first activator protein;
ii) a nucleic acid sequence encoding a first repressor protein; and/or
iii) a nucleic acid sequence encoding a second repressor protein,
   optionally where the nucleic acid sequence encoding the first activator protein; the nucleic acid sequence encoding the first repressor protein; and/or the nucleic acid sequence encoding the second repressor protein are each independently operably linked to a promoter sequence. In some embodiments, the first repressor protein and the second repressor protein are encoded by the same nucleic acid sequence.

In some embodiments, the nucleic acid sequence encoding a first activator protein has a sequence that is at least 80%, at least 85%, at least 90%, at least 91%, at least 92%, at least 93%, at least 94%, at least 95%, at least 96%, at least 97%, at least 98%, at least 99%, or more sequence identity to SEQ ID NO: 16. In some embodiments, the nucleic acid sequence encoding a first activator protein has a sequence that is SEQ ID NO: 16.

In some embodiments, the nucleic acid sequence encoding a first repressor protein has a sequence that is at least 80%, at least 85%, at least 90%, at least 91%, at least 92%, at least 93%, at least 94%, at least 95%, at least 96%, at least 97%, at least 98%, at least 99%, or more sequence identity to SEQ ID NO: 17. In some embodiments, the nucleic acid sequence encoding a first repressor protein has a sequence that is SEQ ID NO: 17.

In some embodiments, the nucleic acid sequence encoding a second repressor protein has a sequence that is at least 80%, at least 85%, at least 90%, at least 91%, at least 92%, at least 93%, at least 94%, at least 95%, at least 96%, at least 97%, at least 98%, at least 99%, or more sequence identity to SEQ ID NO: 17. In some embodiments, the nucleic acid sequence encoding a second repressor protein has a sequence that is SEQ ID NO: 17.

In some embodiments, the promoter region of (i), (ii) and/or (ili) sequence is:
a weak promoter or a medium-strength promoter; optionally
wherein the promoter is selected from the group comprising or consisting of: REV1, PSP2, HTB2, RAD27, or POP6.

By a “weak promoter” we include the meaning of a promoter that produces RNA molecules at a rate that is significantly slower than the average ‘promoter’ within the genome of any given organism or in vitro.

By a “medium-strength promoter” we include the meaning of a promoter that produces RNA molecules at a rate that is approximately the same as the average ‘promoter’ within the genome of any given organism or in vitro.

The weak promoters and medium-strength promoters described herein have been characterised in accordance with Lee et al. 2015 ACS Synth Biol 9:975-986 which is specifically incorporated by reference. The skilled person will understand how to identify a weak promoter and/or a medium-strength promoter. For example, the strength of various promoters that are native to a particular organism can be tested by, for example, analysing the amount of fluorescent protein produced from a gene under the control of each promoter to be tested. It will then be readily apparent to the skilled person which of these promoters are weak or medium-strength, and which are not weak or medium-strength. In one embodiment a weak promoter for use in a particular organism is a promoter that produces RNA molecules at a rate that is significantly slower than the average promoter found within the genome of the particular organism. In one embodiment a medium-strength promoter for use in a particular organism is a promoter that produces RNA molecules at a rate that is approximately the same as the average promoter found within the genome of the particular organism.

In some embodiments, as exemplified in the Examples:
a) the first activator protein is selected from the group comprising or consisting of: rtTA-VP and rtTA-Gal4; optionally
   wherein the first activator protein is rtTA-Gal4;
b) the first repressor protein is selected from the group comprising or consisting of: TetR-Mxi1; optionally
   wherein the first repressor protein is TetR-Mxi1; and/or
c) the second repressor protein is selected from the group comprising or consisting of: mutTetR-Mxi1; optionally
   wherein the first repressor protein is mutTetR-Mxi1.

In some embodiments, the first regulatory polypeptide and the second regulatory polypeptide are each separately capable of directing RNA mediated gene regulation are capable of:
a) activating a gene; and/or
b) repressing a gene.

The nucleic acid construct provided herein may be composed of any nucleic acid. For example, the nucleic acid construct may be an RNA construct or a DNA construct. In a preferred embodiment, the nucleic acid construct is a DNA construct.

In one embodiment, the nucleic acid construct is a linear construct. It is known that linear strands of DNA transformed into cells, such as E. coli, are transcribed to RNA and can be processed into active gRNA molecules. This is advantageous in some situations, for example in situations where it is desirable to dispose of the gRNA fragments/have the cell break down the gRNAs quickly. Cells naturally dispose of linear DNA fragments if they do not possess homology arms to the genome, and so this is one method by which the skilled person can temporally control CRISPR or other RNA mediated gene regulation or editing applications.

In one embodiment, the nucleic acid construct is a circular nucleic acid construct, for example a circular vector, for example a plasmid. The skilled person is aware of plasmids and methods for their construction. Plasmids may be maintained within a cell, and accordingly are advantageous in situations where it is desirable to maintain gRNA fragments. Plasmids may be maintained episomally or may be integrated into the genome using methods that are known to the skilled person. Such methods typically require additional elements to be included in the plasmid. For example, where it is desired that a plasmid is maintained episomally, the plasmid must typically encode an origin of replication, and may preferably encode a selectable marker. Where it is desired that a plasmid or fragment thereof is integrated into the genome, the plasmid must typically encode recombination sites upstream (5′) and downstream (3′) of the sequence that is to be integrated. Recombination sites are well known to the skilled person, and typically comprise regions of homology to a target locus in a target genome. Integration of the sequence between such recombination sites is mediated by homologous recombination.

Accordingly, in one embodiment, the nucleic acid construct comprises at least one, optionally two regions of homology to a target locus in a target genome, arranged so as to allow homologous recombination to occur between the regions of homology in the nucleic acid construct and the corresponding regions of homology in the target genome so as to result in incorporation of the nucleic acid construct into the target genome.

A second aspect of the invention provides a vector comprising the nucleic acid construct provided herein. Vectors and their components, and conditions for generating, maintaining, and purifying different types of vectors are known to the skilled person. In some embodiments, the vector is selected from the group comprising or consisting of: a plasmid, an artificial chromosome, a bacterial artificial chromosome, a yeast artificial chromosome, a human artificial chromosome, a viral vector, a retroviral vector, a lentiviral vector, an adenoviral vector, an adeno-associated viral vector, a bacteriophage vector, a hybrid viral vector, or any combination thereof.

A third aspect of the invention provides a single polycistronic nucleic acid transcript transcribed from the promoter module of the nucleic acid construct provided herein or the vector provided herein. In a preferred embodiment, the single polycistronic nucleic acid transcript is an RNA transcript.

In one embodiment, the single polycistronic nucleic acid transcript comprises at least 2, optionally at least 3, 4, 5, 6, 7, 8, 9, or at least 10 nucleic acid sequences that are each separately capable of directing RNA mediated gene regulation or RNA mediated gene editing. Between each nucleic acid sequence that directs RNA mediated gene regulation or RNA mediated gene editing is a sequence that is a cleavage site. Preferences for cleavage sites are as described elsewhere herein. The single polycistronic nucleic acid transcript comprises a region transcribed from an operator sequence.

A fourth aspect of the invention provides a cell comprising the nucleic acid construct as provided herien; the vector as provided herein; and/or the single polycistronic nucleic acid transcript as provided herein.

It will be appreciated that the nucleic acid construct, vector, and/or polycistronic nucleic acid transcript comprised by the cell may comprise any one of the modules, elements, or regions as disclosed herein. It will further be understood that the nucleic acid construct, vector, and/or polycistronic nucleic acid transcript may comprise some but not all of the modules, elements, or regions as disclosed herein.

In some embodiments the cell comprises a nucleic acid construct consisting only the promoter module and the gene-regulating module as provided herein. In some embodiments, further modules, elements, or regions as provided herein are encoded by the genome of the cell. For example, the cell may encode in its genome at least one or more modules, elements, or nucleic acid regions selected from the group comprising or consisting of: a promoter operator of a second sequence as disclosed herein; a first nucleotide region encoding a first regulatory polypeptide as disclosed herein, optionally operably linked to a promoter as disclosed herein; a second nucleotide region encoding a second regulatory polypeptide as disclosed herein, optionally operably linked to a promoter as disclosed herein; a polypeptide that is capable of cleaving the cleavage site present in the array module when in RNA form as disclosed herein, optionally operably linked to a promoter as disclosed herein; a nucleic acid sequence encoding a first activator protein as disclosed herein, optionally operably linked to a promoter as disclosed herein; a nucleic acid sequence encoding a first repressor protein as disclosed herein, optionally operably linked to a promoter as disclosed herein; a nucleic acid sequence encoding a second repressor protein as disclosed herein, optionally operably linked to a promoter as disclosed herein; or any combination thereof.

In some embodiments, the nucleic acid construct comprised by the cell comprises the promoter module and the gene-regulating module as provided herein, and further comprises any one or more of the modules, elements, or nucleic acid regions as disclosed herein selected from the group comprising or consisting of: a promoter operator of a second sequence as disclosed herein; a first nucleotide region encoding a first regulatory polypeptide as disclosed herein, optionally operably linked to a promoter as disclosed herein; a second nucleotide region encoding a second regulatory polypeptide as disclosed herein, optionally operably linked to a promoter as disclosed herein; a polypeptide that is capable of cleaving the cleavage site present in the array module when in RNA form as disclosed herein, optionally operably linked to a promoter as disclosed herein; a nucleic acid sequence encoding a first activator protein as disclosed herein, optionally operably linked to a promoter as disclosed herein; a nucleic acid sequence encoding a first repressor protein as disclosed herein, optionally operably linked to a promoter as disclosed herein;

a nucleic acid sequence encoding a second repressor protein as disclosed herein, optionally operably linked to a promoter as disclosed herein; or any combination thereof. In some embodiments, any of the modules, elements, or nucleic acid regions that are not comprised by the nucleic acid construct may be encoded in the genome of the cell.

It will be apparent to the skilled person that the cell may be any cell. The skilled person is well equipped to design the relevant components of the method, for example the nucleic acid construct and the vector so as to allow expression of the transcript in any particular cell type. For example, the skilled person will know to use a promoter that is active in human cells when trying to express the transcript in human cells.

Accordingly, in some embodiments, the cell is a eukaryotic cell, optionally selected from a fungal cell; a plant cell; and an animal cell, optionally wherein the animal cell is a mammalian cell.

In some embodiments, the cell is a fungal cell, optionally is a fungal cell belonging to a genus selected from the group comprising or consisting of: Candida, Hansenula, Komagatella, Pichia (for example Pichia pastoris), Ashbya, Blastobotrys, Cryptococcus, Cutaneotrichosporon, Dekkera, Kluveromyces (for example, Kluveromyces lactis), Rhodosporidium (for example, Rhodosporidium toruloides), Rhodotorula, Lipomyces, Saccharomyces, and Yarrowia (for example Yarrowia lipolytica). In a preferred embodiment, the cell is a Saccharomyces cell; optionally is a Saccharomyces cerevisiae cell.

In some embodiments, the cell is a mammalian cell, optionally is selected from the group comprising or consisting of a HEK239T cell, a CHO cell, a Hela cell, or a T-cell.

In some embodiments, the cell is a prokaryotic cell, optionally is a bacterial cell, optionally is a bacterial cell belonging to a genus selected from the group comprising or consisting of: Escherichia (for example, Escherichia coli), Pseudomonas (for example, Pseudomonas syringae), Vibrio, Bacillus, Clostridium, Lactobacillus, Lactococcus, Streptomyces, and the cyanobacteria Synechocystis (for example Synechocystis PCC 6803m). All that is required to allow the methods to produce a nucleic acid capable of expressing the transcript in bacteria is some minor cloning to ensure that the correct promoters and terminators are used, along with co-expression of the appropriate endoribonuclease, for example Csy4, or appropriate ribozyme, for example.

As discussed above, the nucleic acid may be maintained episomally or may be integrated into a genome. Accordingly, in some embodiments, the nucleic acid construct or the vector:
a) is integrated into one or more chromosomes of the cell; or
b) is maintained episomally.

In some embodiments, the cell comprises polypeptides and/or nucleic acids that are capable of transcribing the single polycistronic nucleic acid transcript from the nucleic acid construct or the vector. Exemplary polypeptides and/or nucleic acids that are capable of transcribing the single polycistronic nucleic acid transcript from the nucleic acid construct or the vector include but are not limited to: RNA Polymerase (“RNA Pol” or “Pol”), optionally RNA Polymerase I, II, III, IV, or V; DNA Polymerase (“DNA Pol” or “Pol”), optionally DNA Polymerase I, II, III, IV, V, β (beta), λ (lambda), σ (sigma), μ (mu), α (alpha), δ (delta), ε (epsilon), η (eta), ι (iota), κ (kappa), Rev1, ι (zeta), γ (gamma), θ (theta), ν (nu), telomerase, reverse transcriptase, T4 DNA polymerase; transcription factors; elongation factors; helicase; gyrase; sigma factors; mediator proteins; activator proteins; enhancer proteins; repressor proteins; ρ (rho) factor; DNA repair proteins; or any combination thereof.

As discussed herein, any of the nucleic acid that is capable of directing RNA mediated gene regulation or RNA mediated gene editing comprised by the gene-regulating and/or gene-editing array module as disclosed herein may be complementary to a target nucleic acid region. Advantageously, the target nucleic acid region is comprised by any of the cells as disclosed herein.

Accordingly, in some embodiments the cell comprises a target nucleic acid region and wherein the at least first nucleic acid region that encodes a nucleic acid that is capable of directing RNA mediated gene regulation or RNA mediated gene editing is complementary to the target nucleic acid region, optionally the target nucleic acid region is a promoter of a target gene. In some embodiments, the cell comprises:
a) between 2 and 100 target nucleic acid regions; optionally between 5 and 95, 10 and 90, 15 and 85, 20 and 80, 25 and 75, 30 and 70, 35 and 65, 40 and 60, 45 and 55 target nucleic acid regions; and/or
b) at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 77, at least 76, at least 77, at least 78, at least 79, at least 80, at least 81, at least 82, at least 83, at least 84, at least 88, at least 86, at least 87, at least 88, at least 89, at least 90, at least 91, at least 92, at least 93, at least 94, at least 99, at least 96, at least 97, at least 98, at least 99, or more target nucleic acid regions; optionally comprises 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 66, 66, 67, 68, 69, 70, 71, 72, 73, 74, 77, 76, 77, 78, 79, 80, 81, 82, 83, 84, 88, 86, 87, 88, 89, 90, 91, 92, 93, 94, 99, 96, 97, 98, 99, or 100 target nucleic acid regions,
   and the nucleic acid construct or vector comprises a gene-regulating and/or gene-editing array module that comprises:
i) between 2 and 100 nucleic acid regions that each encode a nucleic acid that is capable of directing RNA mediated gene regulation or RNA mediated gene editing that is complementary to a target nucleic acid region in the cell; optionally between 5 and 95, 10 and 90, 15 and 85, 20 and 80, 25 and 75, 30 and 70, 35 and 65, 40 and 60, 45 and 55 nucleic acid regions that each encode a nucleic acid that is capable of directing RNA mediated gene regulation or RNA mediated gene editing that is complementary to a target nucleic acid region in the cell;
ii) at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 77, at least 76, at least 77, at least 78, at least 79, at least 80, at least 81, at least 82, at least 83, at least 84, at least 88, at least 86, at least 87, at least 88, at least 89, at least 90, at least 91, at least 92, at least 93, at least 94, at least 99, at least 96, at least 97, at least 98, at least 99, or more target nucleic acid regions; optionally comprises 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 66, 66, 67, 68, 69, 70, 71, 72, 73, 74, 77, 76, 77, 78, 79, 80, 81, 82, 83, 84, 88, 86, 87, 88, 89, 90, 91, 92, 93, 94, 99, 96, 97, 98, 99, or 100 nucleic acid regions that each encode a nucleic acid that is capable of directing RNA mediated gene regulation or RNA mediated gene editing that is complementary to a target nucleic acid region in the cell.

As mentioned previously, some types of RNA mediated gene regulation or RNA mediated gene editing that the nucleic acid sequences are capable of directing can be require additional factors, for example regulatory polypeptides. In some instances, for example where rapid or reliable regulation of a target nucleic acid region, constitutive expression of said regulatory polypeptides may be advantageous. Accordingly, in some embodiments, the cell constitutively expresses:
a) the first and/or second regulatory polypeptide; and/or
b) a polypeptide that is capable of cleaving the nucleic acid construct at the cleavage site when in RNA form.

In some embodiments, however, the cell does not constitutively express:
a) the first and/or second regulatory polypeptide; and/or
b) a polypeptide that is capable of cleaving the nucleic acid construct at the cleavage site when in RNA form.

The present invention has many industrial uses, for example in brewing, large-scale protein production, pharmaceutical production, metabolite production optionally the production of chemicals or fuels, biomass vs, growth or metabolic ‘valves’ (control of metabolic production/growth using inducible promoters to control regulatory RNA expression on time, e.g., after growth phase to separate growth and production, which is useful when producing toxic metabolites). Accordingly, the invention also provides methods and uses of the nucleic acids and methods described herein for use in such purposes.

Specifically, a fifth aspect of the invention provided herein is a method of RNA mediated gene regulation of at least one target gene, the method comprising:
a) contacting the cell as provided herein with an inducer molecule; and
b) maintaining the cell in culture conditions suitable for the expression of the array module.

Appropriate inducer molecules are discussed herein. In some embodiments, the inducer molecule is selected from the group comprising or consisting of: tetracycline (Tc); anhydrotetracycline (aTc); Doxycycline; optionally is anhydrotetracycline (aTc).

As will be appreciated, contacting a cell according to the invention with an inducer molecule according to the invention will alter the expression of the array molecule by the cell. Accordingly, in some embodiments, the array module:
a) is not expressed in the absence of the inducer molecule;
b) is expressed only in the presence of the inducer molecule; and/or
c) has increased expression levels in the presence of the inducer molecule compared to the level of expression of the array module in the absence of the inducer molecule; optionally
   expression of the array module increases by at least 100%, at least 150%, at least 200%, at least 250%, at least 300%, at least 350%, at least 400%, at least 450%, at least 500%, at least 550%, at least 600%, at least 650%, at least 700%, at least 750%, at least 800%, at least 850%, at least 900%, at least 950%, at least 1000%, at least 1500%, at least 2000%, at least 2500%, at least 3000%, at least 3500%, at least 4000%, at least 4500%, at least 5000%, at least 5500%, at least 6000%, at least 6500%, at least 7000%, at least 7500%, at least 8000%, at least 8500%, at least 9000%, at least 10,000%, or more in the presence of the inducer molecule compared to the expression of the array module in the absence of the inducer molecule; optionally
   expression of the array module increases by 100%, 150%, 200%, 250%, 300%, 350%, 400%, 450%, 500%, 550%, 600%, 650%, 700%, 750%, 800%, 850%, 900%, 950%, 1000%, 1500%, 2000%, 2500%, 3000%, 3500%, 4000%, 4500%, 5000%, 5500%, 6000%, 6500%, 7000%, 7500%, 8000%, 8500%, 9000%, or 10,000% in the presence of the inducer molecule compared to the expression of the target gene in the absence of the inducer molecule.

In some embodiments, the method comprises contacting the cell with a nuclease enzyme capable of cleaving the cleavage site when in RNA form. In some embodiments, said contacting is performed by expressing said nuclease enzyme within the cell.

It will be appreciated that, in line with the aspects of the invention, where a cell has been contacted with an inducer molecule, repression of expression of the gene-regulating and/or gene-editing array module by the repressor proteins (optionally the first repressor protein) as disclosed herein is relieved. Furthermore, in the presence of the inducer molecule, the activator proteins (optionally first activator protein) as disclosed herein are capable of binding to the promoter operator (optionally promoter operator of a first sequence), thereby driving transcription of the gene-regulating and/or gene-editing array module into a single polycistronic nucleic acid transcript. In embodiments where type of RNA mediated gene regulation or RNA mediated gene editing requires additional elements, for example regulatory polypeptides that are capable of regulating a gene, the nucleic acid region(s) that encode a nucleic acid that is capable of directing RNA mediated gene regulation or RNA mediated gene editing within the single polycistronic nucleic acid transcript bind to, associate with, or form a complex with a regulatory polypeptide that is capable of regulating a gene.

In embodiments where the cell comprises, expresses, and/or is contacted with a polypeptide or nuclease enzyme that is capable of cleaving the cleavage site present in the array module when in RNA form as disclosed herein, the single polycistronic nucleic acid transcript is processed and/or cleaved by the polypeptide that is capable of cleaving the cleavage site present in the array module when in RNA form to generate a plurality of nucleic acid fragments. It will be appreciated that in embodiments where the cell is not contacted with or does not comprise and/or express a polypeptide or nuclease enzyme that is capable of cleaving the cleavage site present in the array module when in RNA form as disclosed herein, a plurality of nucleic acid fragments may be produced from the single polycistronic nucleic acid transcript by other processes known to the person skilled in the art, for example by endonuclease cleavage; exonuclease degradation; autophagic degradation; and shearing.

In some embodiments, each nucleic acid fragment of the plurality of nucleic acid fragments comprises a sequence that is the identical to or complementary to at least a portion of the gene-regulating and/or gene-editing array module, optionally at least a portion of the nucleic acid region that encodes a nucleic acid that is capable of directing RNA mediated gene regulation or RNA mediated gene editing. In some embodiments, at least one nucleic acid fragment of the plurality of nucleic acid fragments associates with and/or forms a complex with a polypeptide capable of regulating a gene.

In some embodiments, the association of and/or complex of the at least one nucleic acid fragment of the plurality of nucleic acid fragments and the polypeptide capable of regulating a gene, binds to at least a portion of at least one target nucleic acid region, optionally a genomic sequence, that is complementary to at least a portion of the nucleic acid fragment of the plurality of nucleic acid fragments. In some embodiments, the association of and/or complex of the at least one nucleic acid fragment of the plurality of nucleic acid fragments and the polypeptide capable of regulating a gene, binds to at least a portion of at least one target nucleic acid region, optionally a genomic sequence, that is complementary to the nucleic acid fragment of the plurality of nucleic acid fragments. In some embodiments, the target nucleic acid region is a promoter of a target gene. In some embodiments, binding of the association of and/or complex of the at least one nucleic acid fragment of the plurality of nucleic acid fragments and the polypeptide capable of regulating a gene to the target nucleic acid region, optionally a promoter of said target gene, results in a change in expression level of said target gene.

In some embodiments, upon binding of the at least one nucleic acid fragment of the plurality of nucleic acid fragments associated with a polypeptide capable of regulating a target gene, the expression level of said gene:
a) is increased;
b) is decreased; and/or
c) is silenced
compared to the absence of binding of the at least one nucleic acid fragment of the plurality of nucleic acid fragments associated with a polypeptide capable of regulating a gene.

Accordingly, as will be appreciated, in some embodiments contacting a cell according to the invention with an inducer molecule according to the invention will alter the expression of the target gene by the cell. Accordingly, in some embodiments, the target gene:
a) i) is not expressed in the absence of the inducer molecule;
   ii) is expressed only in the presence of the inducer molecule; and/or
   iii) has increased expression levels in the presence of the inducer molecule compared to the level of expression of the target gene in the absence of the inducer molecule; optionally
      expression of the target gene increases by at least 100%, at least 150%, at least 200%, at least 250%, at least 300%, at least 350%, at least 400%, at least 450%, at least 500%, at least 550%, at least 600%, at least 650%, at least 700%, at least 750%, at least 800%, at least 850%, at least 900%, at least 950%, at least 1000%, at least 1500%, at least 2000%, at least 2500%, at least 3000%, at least 3500%, at least 4000%, at least 4500%, at least 5000%, at least 5500%, at least 6000%, at least 6500%, at least 7000%, at least 7500%, at least 8000%, at least 8500%, at least 9000%, at least 10,000%, or more in the presence of the inducer molecule compared to the expression of the target gene in the absence of the inducer molecule; optionally
      expression of the target gene increases by 100%, 150%, 200%, 250%, 300%, 350%, 400%, 450%, 500%, 550%, 600%, 650%, 700%, 750%, 800%, 850%, 900%, 950%, 1000%, 1500%, 2000%, 2500%, 3000%, 3500%, 4000%, 4500%, 5000%, 5500%, 6000%, 6500%, 7000%, 7500%, 8000%, 8500%, 9000%, or 10,000% in the presence of the Inducer molecule compared to the expression of the target gene in the absence of the inducer molecule; or
b) i) is expressed in the absence of the inducer molecule;
   ii) is expressed only in the absence of the inducer molecule; and/or
   iii) has decreased expression levels in the presence of the inducer molecule compared to the level of expression of the target gene in the absence of the inducer molecule; optionally
      expression of the target gene decreases by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% in the presence of the inducer molecule compared to the expression of the target gene in the absence of the inducer molecule; optionally
      expression of the target gene decreases by 10%, 15%, 20%, 25%, 30%, 35%, 40%, 45%, 50%, 55%, 60%, 65%, 70%, 75%, 80%, 85%, 90%, 95%, or 100% in the presence of the inducer molecule compared to the expression of the target gene in the absence of the inducer molecule.

Methods of determining the level of expression of a gene or the level of expression of a nucleic acid from a promoter are known to the person skilled in the art. The level of expression of a gene may determined by detecting the level of the RNA transcript of the gene using, for example, reverse transcriptase PCR (RT-PCR), quantitative RT-PCR (qRT-PCR), fluorescence in situ hybridisation, and/or northern blotting; or may be determined by detecting the level of a protein encoded by the gene, for example by western blotting, immunostaining such as immunofluorescence staining, fluorescence microscopy, and/or quantitative chromatographic and mass spectrometric techniques such as LC/MS. The level of expression of a nucleic acid from a promoter may be determined by detecting the level of the RNA transcript of a nucleic acid that is operably linked to the promoter using the methods discussed above, or by detecting the level of a protein encoded by the nucleic acid using the methods discussed above. The nucleic acid may encode a marker, for example a fluorescent protein such as GFP, mEmerald, EBFP, azurite, mTurquoise, YFP, mOrange, DsRed, mCherry, mScarlet, and HcRed, which may be detected and quantified by fluorescence detection and/or microscopy. The level of expression of a gene or the level of expression of a nucleic acid from a promoter may be normalised to the baseline level of expression of the gene or level of expression of the nucleic acid from a promoter; or normalised to the level of expression of an endogenous gene such as tubulin, actin, RecA and Rho.

In some embodiments, the method comprises RNA mediated gene regulation of between 2 and 100 target genes; optionally between 5 and 95, 10 and 90, 15 and 85, 20 and 80, 25 and 75, 30 and 70, 35 and 65, 40 and 60, 45 and 55 target genes; and/or at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 77, at least 76, at least 77, at least 78, at least 79, at least 80, at least 81, at least 82, at least 83, at least 84, at least 88, at least 86, at least 87, at least 88, at least 89, at least 90, at least 91, at least 92, at least 93, at least 94, at least 99, at least 96, at least 97, at least 98, at least 99, or more target genes; optionally comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 66, 66, 67, 68, 69, 70, 71, 72, 73, 74, 77, 76, 77, 78, 79, 80, 81, 82, 83, 84, 88, 86, 87, 88, 89, 90, 91, 92, 93, 94, 99, 96, 97, 98, 99, or 100 target genes.

In some embodiments, the method comprises RNA mediated gene regulation of between 2 and 10,000 target genes, optionally between 100 and 9900, 200 and 9800, 300 and 9700, 400 and 9600, 500 and 9500, 1000 and 9000, 1500 and 8500, 2000 and 8000, 2500 and 7500, 3000 and 7000, 3500 and 6500, 4000 and 6000, 4500 and 5500 target genes; and/or at least 100, at least 200, at least 300, at least 400, at least 500, at least 600, at least 700, at least 800, at least 900, at least 1000, at least 1100, at least 1200, at least 1300, at least 1400, at least 1500, at least 1600, at least 1700, at least 1800, at least 1900, at least 2000, at least 2100, at least 2200, at least 2300, at least 2400, at least 2500, at least 2600, at least 2700, at least 2800, at least 2900, at least 3000, at least 3100, at least 3200, at least 3300, at least 3400, at least 3500, at least 3600, at least 3700, at least 3800, at least 3900, at least 4000, at least 4100, at least 4200, at least 4300, at least 4400, at least 4500, at least 4600, at least 4700, at least 4800, at least 4900, at least 5000, at least 5100, at least 5200, at least 5300, at least 5400, at least 5500, at least 5600, at least 5700, at least 5800, at least 5900, at least 6000, at least 6100, at least 6200, at least 6300, at least 6400, at least 6500, at least 6600, at least 6700, at least 6800, at least 6900, at least 7000, at least 7100, at least 7200, at least 7300, at least 7400, at least 7700, at least 7600, at least 7700, at least 7800, at least 7900, at least 8000, at least 8100, at least 8200, at least 8300, at least 8400, at least 8800, at least 8600, at least 8700, at least 8800, at least 8900, at least 9000, at least 9100, at least 9200, at least 9300, at least 9400, at least 9900, at least 9600, at least 9700, at least 9800, at least 9900, or more target genes; optionally 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6600, 6600, 6700, 6800, 6900, 7000, 7100, 7200, 7300, 7400, 7700, 7600, 7700, 7800, 7900, 8000, 8100, 8200, 8300, 8400, 8800, 8600, 8700, 8800, 8900, 9000, 9100, 9200, 9300, 9400, 9900, 9600, 9700, 9800, 9900, or 10,000 target genes.

It will be clear to the skilled person that the expression of multiple RNA nucleic acids that can each separately mediate gene regulation has a number of uses, for example in industry. Exemplary industrial uses include brewing, large-scale protein production, pharmaceutical production, metabolite production optionally the production of chemicals or fuels, biomass vs. growth or metabolic ‘valves’ (control of metabolic production/growth using inducible promoters to control regulatory RNA expression on time, e.g., after growth phase to separate growth and production, which is useful when producing toxic metabolites). Accordingly, the invention also provides methods and uses of the nucleic acids and methods described herein for use in such purposes, for example the invention provides the nucleic acid construct of the invention; or the vector of the invention; or the cell according to the invention for use in an industrial process, for example for use in brewing, large-scale protein production, pharmaceutical production, metabolite production optionally the production of chemicals or fuels, biomass vs. growth or metabolic ‘valves’ (control of metabolic production/growth using inducible promoters to control regulatory RNA expression on time, e.g., after growth phase to separate growth and production, which is useful when producing toxic metabolites).

There are numerous applications for nucleic acid constructs that encode RNA mediated gene regulation or editing directing sequences. For example, such a construct has uses both in industrial and medical applications.

One particular application is in the control of metabolism. For example, in one embodiment at least one, or two or more of the nucleic acid sequences that encode an RNA mediated gene regulation or editing directing sequence are directed towards genes that are involved in the control of metabolism. Some such genes from yeast include ADH1, ACC1, GPD1, DGA1, HXK, ICL1, HMG1, ERG9, ERG20, ERG5, PTA, ACK, ACS2, HXT1-7, GAL2, GAPDH, ADH3, FUM1, IDP1, SDH1, SDH3, SER3, SDH2, SER33, and ADR1. Other genes from yeast and other species will be apparent to the skilled person and can be identified in the annotated sequence and organism databases.

In some embodiments, the target gene is selected from the group comprising or consisting of: ADH1, ACC1, GPD1, DGA1, HXK, ICL1, HMG1, ERG9, ERG20, ERG5, PTA, ACK, ACS2, HXT1-7, GAL2, GAPDH, ADH3, FUM1, IDP1, SDH1, SDH3, SER3, SDH2, SER33, and ADR1; or any combination thereof. In some embodiments, the gene-regulating and/or gene-editing array module or a portion thereof; at least one array sub-module or portion thereof; nucleic acid region that encodes a nucleic acid that is capable of directing RNA mediated gene regulation or RNA mediated gene editing or portion thereof; single polycistronic nucleic acid transcript or portion thereof; and/or nucleic acid fragment of the plurality of nucleic acid fragments, or portion thereof, is complementary to a promotor of the target gene, optionally wherein the target gene is selected from the group comprising or consisting of: ADH1, ACC1, GPD1, DGA1, HXK, ICL1, HMG1, ERG9, ERG20, ERG5, PTA, ACK, ACS2, HXT1-7, GAL2, GAPDH, ADH3, FUM1, IDP1, SDH1, SDH3, SER3, SDH2, SER33, and ADR1; or any combination thereof.

Metabolic rewiring of target genes in vivo via transcriptional activation or repression or, optionally, deletion of these target genes can also be achieved using the nucleic acid constructs of the invention. Further uses include metabolic engineering, synthetic biology, biomaterial production, recombinant protein production, etc.

In a sixth aspect, the invention provides a use of the methods provided herein in a process of producing at least one organic molecule.

In some embodiments, the organic molecule is selected from the group comprising or consisting of: a metabolite; a secondary metabolite; a fatty acid; a fat; an oligosaccharide; a polysaccharide; a monosaccharide; a nucleic acid; a polypeptide; or any combination thereof, optionally wherein the metabolite is selected from the group comprising or consisting of flavonoids, terpenoids and polyketides. In some embodiments, the organic molecule is succinic acid.

In some embodiments, use of the methods increases the production of the organic molecule. For example, where the method is used the average (e.g., mean, median, or modal) amount (e.g., weight or volume) of organic molecule produced by a cell or organism, optionally in a given reaction, optionally in a given volume, may be increased compared to the average (e.g., mean, median, or modal) amount (e.g., weight or volume) of organic molecule produced when the method is not used. In some embodiments, where the method is used the average (e.g., mean, median, or modal) amount (e.g., weight or volume) of the organic molecule produced is increased between 2-fold and 100-fold, optionally between 5-fold and 95-fold, 10 and 90-fold, 15-fold and 85-fold, 20-fold and 80-fold, 25-fold and 75-fold, 30-fold and 70-fold, 35-fold and 65-fold, 40-fold and 60-fold, 45-fold and 55-fold; and/or is increased at least 2-fold, at least 3-fold, at least 4-fold, at least 5-fold, at least 6-fold, at least 7-fold, at least 8-fold, at least 9-fold, at least 10-fold, at least 11-fold, at least 12-fold, at least 13-fold, at least 14-fold, at least 15-fold, at least 16-fold, at least 17-fold, at least 18-fold, at least 19-fold, at least 20-fold, at least 21-fold, at least 22-fold, at least 23-fold, at least 24-fold, at least 25-fold, at least 26-fold, at least 27-fold, at least 28-fold, at least 29-fold, at least 30-fold, at least 31-fold, at least 32-fold, at least 33-fold, at least 34-fold, at least 35-fold, at least 36-fold, at least 37-fold, at least 38-fold, at least 39-fold, at least 40-fold, at least 41-fold, at least 42-fold, at least 43-fold, at least 44-fold, at least 45-fold, at least 46-fold, at least 47-fold, at least 48-fold, at least 49-fold, at least 50-fold, at least 51-fold, at least 52-fold, at least 53-fold, at least 54-fold, at least 55-fold, at least 56-fold, at least 57-fold, at least 58-fold, at least 59-fold, at least 60-fold, at least 61-fold, at least 62-fold, at least 63-fold, at least 64-fold, at least 65-fold, at least 66-fold, at least 67-fold, at least 68-fold, at least 69-fold, at least 70-fold, at least 71-fold, at least 72-fold, at least 73-fold, at least 74-fold, at least 77-fold, at least 76-fold, at least 77-fold, at least 78-fold, at least 79-fold, at least 80-fold, at least 81-fold, at least 82-fold, at least 83-fold, at least 84-fold, at least 88-fold, at least 86-fold, at least 87-fold, at least 88-fold, at least 89-fold, at least 90-fold, at least 91-fold, at least 92-fold, at least 93-fold, at least 94-fold, at least 99-fold, at least 96-fold, at least 97-fold, at least 98-fold, at least 99-fold, or more; optionally is increased by 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 26-fold, 27-fold, 28-fold, 29-fold, 30-fold, 31-fold, 32-fold, 33-fold, 34-fold, 35-fold, 36-fold, 37-fold, 38-fold, 39-fold, 40-fold, 41-fold, 42-fold, 43-fold, 44-fold, 45-fold, 46-fold, 47-fold, 48-fold, 49-fold, 50-fold, 51-fold, 52-fold, 53-fold, 54-fold, 55-fold, 56-fold, 57-fold, 58-fold, 59-fold, 60-fold, 61-fold, 62-fold, 63-fold, 64-fold, 66-fold, 66-fold, 67-fold, 68-fold, 69-fold, 70-fold, 71-fold, 72-fold, 73-fold, 74-fold, 77-fold, 76-fold, 77-fold, 78-fold, 79-fold, 80-fold, 81-fold, 82-fold, 83-fold, 84-fold, 88-fold, 86-fold, 87-fold, 88-fold, 89-fold, 90-fold, 91-fold, 92-fold, 93-fold, 94-fold, 99-fold, 96-fold, 97-fold, 98-fold, 99-fold, or 100-fold compared to the average (e.g., mean, median, or modal) amount (e.g., weight or volume) of organic molecule produced when the method is not used.

The nucleic acid constructs of the invention can also be used for the rapid deletion of genes in vivo to engineer strains with the use of fewer numbers of transformations compared to standard methods.

The invention also has applications in genome engineering. For example, multiplexed gRNAs can be used to cleave genomic DNA fragments and move them between organisms for numerous applications in genome synthesis (see Wang et al 2016 Nature 539:59-64).

The invention also has applications in RNA detection with CRISPR-Cas13a/C2c2, for example by multiplexing gRNAs many viruses can be detected/cleaved simultaneously, for example on paper-based diagnostics.

In a seventh aspect, the invention provides a kit comprising the nucleic acid construct provided herein; the vector provided herein; the single polycistronic nucleic acid transcript provided herein; or cell provided herein.

In an eight aspect, the invention provides a nucleic acid construct; vector; single polycistronic nucleic acid transcript; cell; method; use; or kit as described herein.

Preferences for the features described above, including but not limited to, the type of nucleic acid (DNA or RNA; linear or circular), type of gene regulation, size and number/frequency of nucleic acid fragments, promoter modules, gene editing or gene regulating modules, array modules, array sub-modules, operator sequences, activator proteins, repressor proteins, position of primer hybridisation sites, cleavage sites, polypeptides that are capable of regulating a gene, lining primers, cell type, promoters and destination vectors, vectors, and other features, apply equally to all aspects and embodiments described below.

The listing or discussion of an apparently prior-published document in this specification should not necessarily be taken as an acknowledgement that the document is part of the state of the art or is common general knowledge.

It should be apparent that preferences and options for a given aspect, feature or parameter of the invention should, unless the context indicates otherwise, be regarded as having been disclosed in combination with any and all preferences and options for all other aspects, features and parameters of the invention.

It is understood that the examples and embodiments described herein are for illustrative purposes only and that various modifications or changes in light thereof will be suggested to persons skilled in the art and are to be included within the present invention and scope of the appended claims.

### Sequences Disclosed Herein

TetO Promoter operator of a first sequence
>SEQ ID NO: 1
TCCCTATCAGTGATAGAGA
tetO-4C5G Promoter operator of a second sequence and/or array operator of a
second sequence
>SEQ ID NO: 2
TCCCCGTCAGTGACGGAGA
rtTA
>SEQ ID NO: 3
atgTCAAGATTGGACAAAAGCAAGGTTATTAACGGTGCCTTGGAACTGTTGAATGGTGTAGGTATAGAAGGCTTAACGACCAGAAAATTAGCAC
AAAAGTTAGGTGTAGAGCAGCCAACATTATATTGGCACGTCAAAAACAAGAGAGCATTGCTAGACGCATTGCCAATCGAAATGTTAGATCGTCA
TCACACTCATTTTTGCCCATTAGAGGGTGAGAGTTGGCAAGACTTTTTGCGTAACAATGCTAAATCCTTTAGATGCGCCTTGTTGAGCCATCGT
GATGGTGCCAAGGTTCATCTAGGTACAAGGCCAACAGAAAAACAGTATGAGACTTTGGAGAATCAGCTAGCCTTTTTATGCCAACAGGGTTTTT
CATTGGAGAATGCCTTATACGCTTTAAGTGCAGTTGGTCACTTCACTTTAGGTTGCGTACTAGAAGAACAAGAGCACCAGGTTGCTAAAGAAGA
GAGAGAAACACCTACAACAGATTCAATGCCTCCATTGCTAAGACAAGCTATCGAACTGTTTGATAGACAGGGTGCAGAACCTGCATTCTTGTTC
GGTTTAGAGTTGATTATTTGCGGCTTGGAAAAGCAGCTAAAATGCGAATCT
TetR
>SEQ ID NO: 4
atgTCAAGATTGGATAAGTCCAAGGTTATCAACTCTGCTTTGGAGTTGTTGAACGAAGTTGGTATTGAAGGTTTGACCACTAGAAAGTTGGCTC
AAAAATTGGGTGTTGCTCAAAAAACCTTGTACTGGCACGTTAAGAACAAAAGAGCTTTGTTGGACGCTTTGGCCATTGAAATGTTGGATAGACA
TCATACCCATTTCTGTCCATTGGAAGGTGAATCATGGCAAGATTTCTTGAGAAACAACGCCAAGTCTTTCAGATGTGCTTTGTTGTCTCATAGA
GATGGTGCTAAAGTTCACTTGGGTACTAGACCAACTGAAAAGCAATACGAAACTTTGGAAAACCAGTTGGCTTTCTTGTGTCAACAAGGTTTCT
CTTTAGAAAACGCCTTGTATGCTTTGTCTGCTGTTGGTCATTTTACCTTGGGTTGTGTTTTGGAAGATCAAGAACATCAAGTCGCCAAAGAAGA
AAGGGAAACTCCAACTACTGATTCTATGCCACCATTATTGAGACAAGCCATTGAGTTGTTTGATCATCAAGGTGCTGAACCAGCTTTTTTGTTT
GGTTTGGAATTGATCATCTGCGGTTTGGAGAAACAATTGAAGTGTGAATCT
VP domain
>SEQ ID NO: 5
AAATTAGGTGGTTCTGGTGGTTCCCCAAAGAAGAAAAGAAAGGTTGGTAGAGCTGAAGCTTCTGGTTCAGGTAGAGCAGATGCTTTGGATGATT
TCGATTTGGACATGTTGGGTTCTGATGCATTGGACGATTTCGACTTAGATATGTTAGGTAGTGACGCCTTGGATGACTTCGATCTGGATATGCT
AGGTTCCGATGCCTTAGACGACTTTGATTTAGACATGCTGATTAACTCAAGATCCTCTGGTTCTCCTAAGAAGAAGAGAAAAGTCGGTTCTCAA
TACTTGCCAGATACCGATGATAGACACAGAATCGAAGAAAAGAGGAAGAGAACTTACGAAACCTTCAAGTCCATCATGAAGAAGTCTCCATTTT
CTGGTCCAACTGATCCAAGACCACCACCAAGAAGAATTGCTGTTCCTTCAAGATCCTCTGCCTCTGTTCCAAAACCAGCTCCACAACCTTATCC
TTTTACATCTTCTTTGTCCACCATCAACTACGATGAATTTCCAACTATGGTTTTCCCATCCGGTCAAATTTCTCAAGCTTCTGCTTTGGCTCCA
GCTCCTCCACAAGTTTTGCCACAAGCTCCAGCACCAGCTCCAGCTCCAGCAATGGTTTCTGCTTTAGCTCAAGCCCCAGCTCCTGTTCCAGTTT
TAGCTCCAGGTCCACCTCAAGCTGTTGCTCCACCAGCACCAAAACCTACTCAAGCTGGTGAAGGTACTTTGTCTGAAGCTTTGTTGCAGTTGCA
ATTCGATGATGAAGATTTGGGTGCTTTGTTGGGTAATTCTACTGATCCAGCTGTTTTTACCGATTTGGCTTCTGTTGATAACTCCGAATTTCAG
CAGTTGTTGAATCAAGGTATTCCAGTTGCTCCTCATACAACTGAACCTATGTTGATGGAATATCCAGAAGCAATTACTAGATTAGTTACTGGTG
CTCAAAGACCTCCAGATCCTGCACCTGCTCCTTTGGGTGCTCCAGGTTTACCAAATGGTCTATTGTCTGGTGACGAAGATTTTTCCTCTATTGC
CGATATGGATTTCTCCGCTTTGTTG
Gal4
>SEQ ID NO: 6
GCTAATTTCAATCAGTCTGGTAATATTGCCGACTCCTCTTTGTCTTTCACTTTCACTAATTCTTCCAACGGTCCAAACTTGATCACTACCCAAA
CTAATTCTCAAGCTTTGTCTCAGCCAATTGCCTCTTCTAATGTTCACGATAACTTC
ATGAACAACGAAATCACCGCCTCCAAAATTGATGATGGTAACAATTCTAAGCCATTGTCTCCAGGTTGGACTGATCAAACTGCTTATAATGCTT
TCGGTATTACCACCGGTATGTTTAACACTACTACTATGGATGACGTCTACAACTACTTGTTCGATGATGAAGATACACCACCAAATCCAAAGAA
AGAA
Mxi1
>SEQ ID NO: 7
ATGGAACGTGTGAGAATGATTAATGTGCAAAGGCTGTTAGAAGCCGCAGAGTTTTTAGAAAGAAGAGAAAGAGAATGCGAACACGGGTATGCCA
GTTCTTTCCCTAGCATGCCCTCTCCCAGA
Mxi1
>SEQ ID NO: 8
GAAAGGGTTAGAATGATTAACGTTCAAAGGTTGTTGGAAGCTGCCGAATTTTTGGAAAGACGTGAAAGGGAATGTGAACATGGTTACGCTTCAT
CTTTTCCATCTATGCCATCTCCAAGA
NLS
>SEQ ID NO: 9
CCACCAAAGAAAAAACGTAAGGTC
NLS
>SEQ ID NO: 10
CCACCAAAAAAGAAAAGAAAGGTT
NLS
>SEQ ID NO: 11
GACCCAAAGAAAAAAAGAAAAGTT
NLS
>SEQ ID NO: 12
GACCCAAAGAAGAAGAGAAAGGTT
NLS
>SEQ ID NO: 13
CCTCCTAAGAAAAAGAGAAAAGTT
NLS
>SEQ ID NO: 14
CCACCAAAGAAGAAAAGAAAGGTT
NLS
>SEQ ID NO: 15
CCAAAAAAGAAGAGAAAGGTC
rtTA-Gal4
>SEQ ID NO: 16
atgTCAAGATTGGACAAAAGCAAGGTTATTAACGGTGCCTTGGAACTGTTGAATGGTGTAGGTATAGAAGGCTTAACGACCAGAAAATTAGCAC
AAAAGTTAGGTGTAGAGCAGCCAACATTATATTGGCACGTCAAAAACAAGAGAGCATTGCTAGACGCATTGCCAATCGAAATGTTAGATCGTCA
TCACACTCATTTTTGCCCATTAGAGGGTGAGAGTTGGCAAGACTTTTTGCGTAACAATGCTAAATCCTTTAGATGCGCCTTGTTGAGCCATCGT
GATGGTGCCAAGGTTCATCTAGGTACAAGGCCAACAGAAAAACAGTATGAGACTTTGGAGAATCAGCTAGCCTTTTTATGCCAACAGGGTTTTT
CATTGGAGAATGCCTTATACGCTTTAAGTGCAGTTGGTCACTTCACTTTAGGTTGCGTACTAGAAGAACAAGAGCACCAGGTTGCTAAAGAAGA
GAGAGAAACACCTACAACAGATTCAATGCCTCCATTGCTAAGACAAGCTATCGAACTGTTTGATAGACAGGGTGCAGAACCTGCATTCTTGTTC
GGTTTAGAGTTGATTATTTGCGGCTTGGAAAAGCAGCTAAAATGCGAATCTGGTAGTGGTTCACCTCCTAAGAAAAAGAGAAAAGTTGGTTCCG
GTAGCGCTAATTTCAATCAGTCTGGTAATATTGCCGACTCCTCTTTGTCTTTCACTTTCACTAATTCTTCCAACGGTCCAAACTTGATCACTAC
CCAAACTAATTCTCAAGCTTTGTCTCAGCCAATTGCCTCTTCTAATGTTCACGATAACTTCATGAACAACGAAATCACCGCCTCCAAAATTGAT
GATGGTAACAATTCTAAGCCATTGTCTCCAGGTTGGACTGATCAAACTGCTTATAATGCTTTCGGTATTACCACCGGTATGTTTAACACTACTA
CTATGGATGACGTCTACAACTACTTGTTCGATGATGAAGATACACCACCAAATCCAAAGAAAGAAGGATCCtaa
TetR-Mxi1
>SEQ ID NO: 17
atgTCAAGATTGGATAAGTCCAAGGTTATCAACTCTGCTTTGGAGTTGTTGAACGAAGTTGGTATTGAAGGTTTGACCACTAGAAAGTTGGCTC
AAAAATTGGGTGTTGCTCAAAAAACCTTGTACTGGCACGTTAAGAACAAAAGAGCTTTGTTGGACGCTTTGGCCATTGAAATGTTGGATAGACA
TCATACCCATTTCTGTCCATTGGAAGGTGAATCATGGCAAGATTTCTTGAGAAACAACGCCAAGTCTTTCAGATGTGCTTTGTTGTCTCATAGA
GATGGTGCTAAAGTTCACTTGGGTACTAGACCAACTGAAAAGCAATACGAAACTTTGGAAAACCAGTTGGCTTTCTTGTGTCAACAAGGTTTCT
CTTTAGAAAACGCCTTGTATGCTTTGTCTGCTGTTGGTCATTTTACCTTGGGTTGTGTTTTGGAAGATCAAGAACATCAAGTCGCCAAAGAAGA
AAGGGAAACTCCAACTACTGATTCTATGCCACCATTATTGAGACAAGCCATTGAGTTGTTTGATCATCAAGGTGCTGAACCAGCTTTTTTGTTT
GGTTTGGAATTGATCATCTGCGGTTTGGAGAAACAATTGAAGTGTGAATCTGGTTCTGGTTCTCCACCAAAAAAGAAAAGAAAGGTTGGTTCAG
GTTCCGAAAGGGTTAGAATGATTAACGTTCAAAGGTTGTTGGAAGCTGCCGAATTTTTGGAAAGACGTGAAAGGGAATGTGAACATGGTTACGC
TTCATCTTTTCCATCTATGCCATCTCCAAGAGGATCCtaa
20 nt DNA Csy4 cleavage site
>SEQ ID NO: 18
gttcactgccgtataggcag
28 nt DNA Csy4 cleavage site
>SEQ ID NO: 19
GTTCACTGCCGTATAGGCAGCTAAGAAA
20 nt RNA Csy4 cleavage site
>SEQ ID NO: 20
GUUCACUGCCGUAUAGGCAG
28 nt RNA Csy4 cleavage site
>SEQ ID NO: 21
GUUCACUGCCGUAUAGGCAGCUAAGAAA
Dr-RNAGly (GCC)
>SEQ ID NO: 23
gtgaGCATTGGTGGTTCAGTGGTAGAATTCTCGCCTGCCACGCGGGAGGCCCGGGTTCGATTCCCGGCCAATGCA
Dr-tRNALys (CTT)
>SEQ ID NO: 24
gttctcatcaGCCCGGCTAGCTCAGTCGGTAGAGCATGAGACTCTTAATCTCAGGGTCGTGGGTTCGAGCCCCACGTCGGGCG
Dr-tRNAAsn (GTT)
>SEQ ID NO: 25
gctatctGTCTCTGTGGCGCAATCGGTTAGCGCGTTCGGCTGTTAACCGAAAGGTTGGTGGTTCGAGCCCACCCAGGGACG
Dr-tRNAMet (CAT)
>SEQ ID NO: 26
gcctgaagGTTTCCGTAGTGTAGTGGTTATCACGTTCGCCTCATACGCGAAAGGTCCCCAGTTCGAAACTGGGCGGAAACA
Dr-tRNAGln (CTG)
>SEQ ID NO: 27
gacttgaGGTTCCATGGTGTAATGGTTAGCACTCTGGACTCTGAATCCAGCGATCCGAGTTCAAATCTCGGTGGGACCA
Dr-tRNASer (GCT)
>SEQ ID NO: 28
ggaaaatGACGAGGTGGCCGAGTGGTTAAGGCGATGGACTGCTAATCCATTGTGCTTTGCACGCATGGGTTCGAATCCCATCCTCGTCG
Dr-tRNAThr (AGT)
>SEQ ID NO: 29
gcagcGGCGCCGTGGCTTAGTTGGTTAAAGCGCCTGICTAGTAAACAGGAGATCCTGGGTTCGAATCCCAGCGGTGCCT
Dr-tRNAHis (GTG)
>SEQ ID NO: 30
gctcGCCGTGATCGTACAGTGGTTAGTACTCTGCGTTGTGGCCGCAGCAACCCCGGTTCGAATCCGGGTCACGGCA
Dr-tRNALeu (CAG)
>SEQ ID NO: 31
gcatGTCAGGATGGCCGAGTGGTCTAAGGCGCTGCGTTCAGGTCGCAGTCTCCCCTGGAGGCGTGGGTTCGAATCCCACTTCTGACA
Os-tRNAGly (GCC)
>SEQ ID NO: 32
gaacaaaGCACCAGTGGTCTAGIGGTAGAATAGTACCCTGCCACGGTACAGACCCGGGTTCGATTCCCGGCTGGTGCA
Os-tRNAGly (GCC)-scrambled
>SEQ ID NO: 33
GAACCTCTTACACGCGCAGATCAACTAAATGTACACTGCGACGGTCCGTGGCTCCGAGAGGGGTTACAGGGTACGCTG
Dr-tRNAGly (GCC)-scrambled
>SEQ ID NO: 34
GCGCTGTGGCGTACCGGGTACGTACTCGCTTGACTGGGTTGGTACTAGGCGAAACCAGCTCCGTGGGATTGCACC
Hammerhead ribozyme (HH)
>SEQ ID NO: 35
gttccccCTGATGAGTCCGTGAGGACGAAACGAGTAAGCTCGTC
Hepatitis delta virus ribozyme (HDV)
>SEQ ID NO: 36
GGCCGGCATGGTCCCAGCCTCCTCGCTGGCGCCGGCTGGGCAACATGCTTCGGCATGGCGAATGGGAC
dCas9-Mxi1
>SEQ ID NO: 37
atgTCTGATCCAAAAAAGAAGAGAAAGGTCGGTTCTGGTTCTGACAAGAAATACTCTATTGGTTTGGCTATTGGCACCAATTCTGTTGGTTGGG
CTGTTATTACTGACGAGTACAAAGTTCCCTCCAAGAAGTTTAAGGTTTTGGGTAACACTGACAGGCACTCCATTAAGAAAAATTTGATTGGTGC
CCTGTTGTTCGATTCTGGTGAAACTGCTGAAGCTACTAGATTGAAAAGAACCGCTAGAAGAAGATACACCAGAAGAAAGAACAGAATCTGCTAC
TTGCAAGAGATTTTCTCTAACGAAATGGCCAAGGTTGATGATTCATTCTTCCACAGATTGGAAGAGTCCTTCTTGGTTGAAGAGGATAAGAAAC
ACGAAAGACATCCAATCTTCGGTAACATCGTTGATGAAGTTGCTTACCACGAAAAGTACCCAACTATCTACCATTTGAGAAAGAAGTTGGTTGA
CTCTACCGATAAGGCTGATTTGAGATTGATCTATTTGGCTTTGGCCCACATGATTAAGTTCAGAGGTCATTTCTTGATCGAGGGTGATTTGAAT
CCAGATAACTCCGATGTTGACAAGTTGTTCATCCAATTGGTCCAAACTTACAACCAGTTGTTCGAAGAAAATCCAATCAATGCCTCTGGTGTTG
ATGCTAAGGCTATTTTGTCTGCTAGACTGTCTAAGTCAAGAAGGTTGGAAAACTTGATTGCTCAATTGCCAGGTGAAAAGAAGAATGGTTTGTT
CGGTAATTTGATCGCCTTGTCTTTGGGTTTGACTCCAAATTTCAAGTCCAACTTCGATTTGGCTGAAGATGCTAAGTTGCAATTGTCTAAGGAT
ACCTACGATGATGACTTGGATAATTTGTTGGCCCAAATCGGTGATCAATACGCTGATTTGTTTTTGGCTGCTAAGAACTTGTCCGATGCCATTT
TGTTGTCCGATATCTTGAGAGTTAACACCGAAATTACTAAGGCTCCATTGTCTGCCTCTATGATCAAAAGATACGATGAACACCACCAAGACTT
GACTTTGTTGAAAGCTTTGGTCAGACAACAGTTGCCAGAAAAGTACAAAGAAATCTTCTTCGACCAGTCCAAAAATGGTTACGCTGGTTATATT
GATGGTGGTGCTTCTCAAGAAGAGTTCTACAAGTTCATTAAGCCCATCTTGGAAAAGATGGATGGTACTGAAGAACTGTTGGTCAAGTTGAACA
GAGAAGATTTGTTGAGAAAGCAGAGAACCTTCGATAACGGTTCTATTCCACATCAAATCCACTTGGGTGAATTGCATGCTATTTTGAGAAGGCA
AGAGGATTTCTACCCATTCTTGAAGGATAACAGAGAAAAGATCGAGAAGATCCTGACCTTCAGAATCCCATATTATGTTGGTCCATTGGCCAGA
GGTAATTCAAGATTTGCTTGGATGACTAGGAAGTCCGAAGAAACTATTACTCCATGGAACTTCGAAGAGGTTGTTGATAAGGGTGCTTCTGCTC
AATCCTTCATTGAAAGAATGACTAACTTCGACAAGAACCTGCCAAACGAAAAGGTTTTGCCAAAACACTCTTTGCTGTACGAATACTTCACCGT
CTACAACGAATTGACTAAGGTTAAGTACGTCACCGAAGGTATGAGAAAACCAGCTTTTTTGTCTGGTGAACAAAAGAAGGCTATCGTCGATTTG
TTGTTCAAGACTAACAGAAAGGTTACCGTCAAGCAATTGAAAGAGGACTACTTCAAGAAAATCGAGTGCTTCGACTCCGTTGAAATTAGTGGTG
TTGAAGATAGATTCAACGCCTCCTTAGGTACTTACCATGACTTATTGAAGATCATCAAGGACAAGGATTTCTTGGACAACGAAGAAAACGAGGA
TATCTTGGAGGATATCGTTTTGACTTTGACCTTGTTCGAGGATAGGGAAATGATCGAAGAAAGGTTGAAAACCTACGCTCACTTGTTTGATGAT
AAGGTCATGAAGCAGTTGAAGAGAAGAAGGTATACTGGTTGGGGTAGATTGTCTAGGAAATTGATTAACGGTATCAGGGACAAGCAATCCGGTA
AGACTATTTTGGACTTTTTGAAGTCTGATGGCTTCGCCAATAGAAACTTCATGCAATTGATCCACGATGACAGCTTGACCTTCAAAGAAGATAT
TCAAAAGGCCCAAGTTTCCGGTCAAGGTGATTCTTTACATGAACATATTGCTAACTTGGCTGGTTCCCCAGCTATTAAGAAAGGTATTTTACAA
ACCGTTAAGGTGGTCGACGAATTGGTAAAAGTTATGGGTAGACATAAGCCAGAGAACATCGTTATTGAAATGGCTAGAGAAAATCAGACTACCC
AAAAGGGTCAAAAGAACTCCAGAGAAAGGATGAAGAGAATTGAAGAGGGTATCAAAGAATTGGGCTCCCAGATTTTGAAAGAACACCCAGTTGA
AAACACCCAATTGCAGAACGAAAAGCTGTACTTGTACTACTTGCAGAATGGTAGAGATATGTACGTCGACCAAGAATTGGATATCAACAGATTG
TCCGATTACGATGTCGATGCTATCGTTCCACAGTCTTTTTTGAAGGATGACTCCATCGATAACAAGGTCTTGACTAGATCCGATAAGAACAGAG
GTAAGTCTGATAACGTTCCATCTGAAGAGGTCGTCAAGAAGATGAAGAATTATTGGAGACAGTTGCTGAACGCCAAGTTGATTACTCAAAGAAA
GTTCGACAATTTGACCAAGGCTGAAAGAGGTGGTTTGTCTGAATTGGACAAAGCAGGTTTCATTAAGAGGCAATTGGTTGAAACTAGGCAAATC
ACCAAACACGTTGCCCAAATTTTGGATTCCAGAATGAATACCAAGTACGACGAAAACGATAAGTTGATCAGGGAAGTTAAGGTCATCACGTTGA
AGTCTAAGTTGGTTTCCGATTTCAGAAAGGACTTCCAATTCTACAAGGTCAGGGAAATCAACAACTACCATCATGCTCATGATGCTTACTTGAA
TGCTGTTGTTGGTACTGCCTTGATTAAGAAGTATCCAAAGTTGGAATCCGAGTTCGTTTACGGTGATTACAAGGTTTACGACGTCAGAAAGATG
ATTGCCAAGTCCGAACAAGAAATTGGTAAGGCTACTGCTAAGTACTTCTTCTACTCCAACATCATGAATTTCTTCAAGACCGAAATCACTTTGG
CCAACGGTGAAATTAGAAAAAGGCCATTGATTGAGACTAACGGTGAGACTGGTGAAATAGTTTGGGATAAGGGTAGAGATTTCGCTACCGTTAG
AAAAGTTTTGTCCATGCCACAAGTTAACATCGTCAAAAAGACCGAAGTTCAAACTGGTGGTTTCTCCAAAGAATCCATCTTGCCAAAAAGAAAC
TCCGACAAATTGATCGCCAGAAAGAAAGATTGGGACCCAAAAAAGTATGGTGGTTTTGATTCTCCAACCGTTGCTTACTCTGTTTTGGTTGTTG
CTAAAGTTGAAAAGGGCAAGAGCAAGAAGTTGAAGTCAGTCAAAGAACTATTGGGCATCACGATCATGGAAAGGTCATCTTTTGAAAAAAACCC
AATCGACTTTTTGGAGGCCAAGGGTTACAAAGAAGTTAAGAAGGATCTGATTATCAAGCTGCCCAAGTACTCTTTGTTCGAATTGGAAAACGGT
CGTAAGAGAATGTTGGCTTCTGCTGGTGAATTACAAAAGGGTAATGAATTGGCTCTGCCCTCTAAGTACGTTAACTTTCTATACTTGGCCTCTC
ACTACGAAAAGTTGAAGGGTTCTCCTGAAGATAACGAACAAAAGCAGTTGTTTGTCGAACAGCATAAGCACTACTTGGACGAAATCATCGAACA
GATTTCTGAATTTTCCAAGAGGGTTATTTTGGCTGATGCCAATTTGGATAAGGTCTTGTCTGCTTACAACAAGCACAGAGATAAGCCAATTAGA
GAACAAGCCGAAAACATCATTCACCTGTTCACTTTGACTAATTTGGGTGCTCCAGCTGCTTTTAAGTATTTCGATACCACCATCGACAGAAAGA
GATACACTTCTACAAAAGAGGTTCTGGACGCTACCTTGATTCATCAATCTATTACTGGCTTGTACGAAACCAGGATCGACTTGTCTCAATTAGG
TGGTGATGGTTCAGGTTCTCCACCAAAGAAAAAACGTAAGGTCGGATCTGGTTCTATGGAACGTGTGAGAATGATTAATGTGCAAAGGCTGTTA
GAAGCCGCAGAGTTTTTAGAAAGAAGAGAAAGAGAATGCGAACACGGGTATGCCAGTTCTTTCCCTAGCATGCCCTCTCCCAGAGGATCCtaa
dCas12a-VP
>SEQ ID NO: 38
atgTCTGACCCAAAGAAGAAGAGAAAGGTTGGTTCTGGTTCCAAGTTGGAAAAGTTTACCAACTGCTACTCTCTGTCTAAGACCTTGAGATTCA
AAGCTATTCCTGTTGGTAAGACCCAAGAAAACATTGACAACAAGAGGTTGTTGGTTGAGGACGAAAAAAGAGCTGAAGATTACAAGGGTGTCAA
GAAGTTGCTGGACAGATATTACTTGTCCTTCATCAACGATGTCCTGCACTCTATTAAGCTGAAGAACTTGAACAACTACATCTCCCTGTTCAGA
AAAAAGACCAGAACCGAGAAAGAGAACAAAGAGTTAGAAAACTTGGAGATCAACCTGAGGAAAGAAATTGCTAAGGCTTTCAAGGGTAACGAGG
GTTACAAATCTTTGTTCAAGAAGGACATCATCGAAACCATCTTGCCAGAATTtTTGGATGATAAGGACGAAATTGCCTTGGTCAATTCTTTCAA
CGGTTTCACTACTGCTTTCACTGGTTTCTTTGACAACAGGGAAAACATGTTCTCTGAAGAAGCTAAGTCTACCTCCATTGCTTTCAGATGCATT
AACGAAAACTTGACCAGGTACATCTCCAACATGGATATtTTCGAAAAGGTTGATGCCATCTTCGATAAGCACGAAGTCCAAGAAATCAAAGAGA
AGATtTTGAACTCCGATTACGACGTCGAAGATTTTTTCGAAGGTGAGTTCTTCAACTTCGTTTTGACTCAAGAAGGTATCGACGTTTACAACGC
TATTATTGGTGGTTTCGTTACCGAATCTGGTGAAAAGATTAAGGGTTTGAACGAGTACATCAACCTGTACAATCAAAAGACCAAGCAGAAGTTG
CCAAAGTTTAAGCCCTTGTACAAACAAGTCTTGTCCGACAGAGAATCCTTGTCTTTTTATGGTGAAGGTTACACCTCCGATGAAGAAGTTTTGG
AAGTTTTCAGAAACACCCTGAACAAGAACTCCGAAATCTTCTCATCCATTAAGAAGTTGGAGAAGCTGTTTAAGAACTTCGACGAATATTCCTC
CGCTGGTATCTTTGTCAAAAATGGTCCAGCTATTTCCACCATCTCCAAGGATATTTTTGGTGAATGGAACGTCATCAGAGATAAGTGGAATGCT
GAATACGATGACATCCACTTGAAAAAGAAGGCTGTTGTTACCGAAAAGTACGAGGATGATAGACGTAAGTCCTTCAAGAAGATTGGCTCTTTCT
CTCTGGAACAATTGCAAGAATATGCTGATGCCGATTTGTCCGTTGTCGAAAAATTGAAAGAAATCATCATCCAGAAGGTCGACGAAATCTACAA
AGTTTACGGTTCCTCTGAAAAGTTGTTCGATGCTGATTTCGTTTTGGAGAAGTCGTTGAAGAAAAACGATGCTGTTGTTGCCATCATGAAGGAT
TTGTTGGATTCCGTTAAGAGCTTCGAGAACTACATTAAGGCTTTTTTCGGTGAGGGCAAAGAAACTAACAGAGATGAATCATTCTACGGCGATT
TTGTTTTGGCCTACGATATtTTGTTGAAGGTTGATCATATCTACGACGCCATCAGAAACTACGTTACTCAAAAACCATACTCCAAAGACAAGTT
CAAGCTGTACTTTCAAAACCCACAATTCATGGGTGGTTGGGACAAAGACAAAGAAACAGATTACAGAGCCACCATCTTGAGATATGGTTCTAAG
TACTACTTGGCCATCATGGATAAGAAATACGCTAAGTGCTTGCAAAAGATCGATAAGGATGATGTCAACGGTAACTACGAGAAGATCAACTACA
AATTATTGCCCGGTCCAAACAAGATGTTGCCTAAGGTTTTCTTCAGCAAAAAGTGGATGGCTTACTACAACCCATCCGAAGATATTCAAAAGAT
tTACAAGAACGGCACCTTCAAAAAGGGTGATATGTTCAACTTGAACGACTGCCATAAGCTGATCGATTTCTTCAAGGATTCCATCTCaAGATAC
CCAAAGTGGTCTAATGCTTACGATTTCAACTTCTCTGAAACCGAGAAGTACAAAGATATTGCCGGTTTTTACAGGGAAGTCGAAGAACAAGGTT
ACAAGGTTTCATTCGAATCCGCCTCTAAAAAAGAAGTTGACAAGTTGGTTGAAGAGGGCAAGTTGTACATGTTCCAGATtTATAACAAGGACTT
CTCCGATAAGTCTCATGGTACTCCAAACTTGCATACCATGTACTTCAAGTTGTTGTTCGACGAAAACAATCACGGTCAGATTAGATTGTCTGGT
GGTGCTGAGTTGTTTATGAGAAGGGCTTCTTTGAAGAAAGAAGAGTTGGTTGTTCATCCAGCCAATTCTCCAATTGCTAACAAGAATCCAGACA
ACCCAAAAAAGACTACCACCTTGTCTTACGATGTTTACAAGGATAAGAGATTCTCCGAGGACCAATACGAATTGCATATTCCAATTGCCATTAA
CAAGTGCCCCAAGAACATTTTCAAGATCAACACCGAAGTTAGGGTGTTGTTGAAGCACGATGATAACCCATACGTTATTGGTATTGCTAGGGGT
GAAAGAAACTTGTTGTACATCGTTGTTGTTGATGGTAAGGGTAACATCGTGGAACAATACTCATTGAACGAGATTATCAACAACTTCAACGGCA
TCAGAATCAAGACCGATTACCATTCTCTGCTTGACAAGAAAGAAAAAGAAAGATTCGAAGCCAGACAGAACTGGACCTCTATTGAAAACATCAA
AGAGTTGAAGGCCGGTTACATTTCACAAGTTGTTCATAAGATtTGCGAGCTGGTTGAAAAGTATGATGCAGTTATTGCTTTGGAGGACTTGAAC
TCTGGTTTCAAGAACTCCAGAGTTAAGGTCGAAAAGCAGGTCTACCAAAAGTTCGAAAAGATGTTGATCGACAAGCTGAACTACATGGTCGACA
AAAAGTCTAATCCATGTGCTACAGGTGGTGCTTTGAAAGGTTATCAAATTACGAACAAGTTCGAGAGCTTCAAGTCTATGTCTACTCAGAATGG
TTTCATCTTCTACATTCCAGCTTGGTTGACCTCTAAGATTGATCCATCTACTGGTTTCGTCAACCTGCTGAAAACTAAGTACACTTCTATTGCC
GACTCCAAGAAGTTCATCTCTTCATTCGATAGAATCATGTACGTTCCAGAAGAGGACTTATTCGAATTtGCTTTGGATTACAAGAACTTCTCaA
GAACTGATGCTGACTACATCAAGAAGTGGAAGTTGTACTCTTACGGTAACAGAATCAGGATCTTCAGAAACCCTAAGAAGAACAACGTTTTCGA
TTGGGAAGAAGTCTGTTTGACCTCTGCTTACAAAGAACTGTTTAACAAGTACGGCATCAACTACCAACAAGGTGATATTAGAGCTTTGTTGTGC
GAACAATCTGATAAGGCCTTCTACTCATCTTTTATGGCCTTGATGTCACTGATGTTGCAGATGAGAAATTCCATTACTGGTAGAACCGATGTCG
ACTTTTTGATCTCTCCAGTTAAGAACTCTGACGGTATTTTCTACGACTCaAGAAACTATGAAGCTCAAGAAAACGCCATTTTGCCAAAAAACGC
TGATGCTAATGGTGCTTACAACATTGCTAGAAAAGTTTTGTGGGCTATCGGCCAATTCAAAAAAGCAGAAGATGAGAAGCTGGACAAGGTTAAG
ATTGCCATCTCAAACAAAGAATGGTTGGAATACGCTCAGACTTCTGTCAAACATGGTTCTGGTTCTCCACCAAAGAAGAAAAGAAAGGTTGGTT
CCGGTTCTAAATTAGGTGGTTCTGGTGGTTCCCCAAAGAAGAAAAGAAAGGTTGGTAGAGCTGAAGCTTCTGGTTCAGGTAGAGCAGATGCTTT
GGATGATTTCGATTTGGACATGTTGGGTTCTGATGCATTGGACGATTTCGACTTAGATATGTTAGGTAGTGACGCCTTGGATGACTTCGATCTG
GATATGCTAGGTTCCGATGCCTTAGACGACTTTGATTTAGACATGCTGATTAACTCAAGATCCTCTGGTTCTCCTAAGAAGAAGAGAAAAGTCG
GTTCTCAATACTTGCCAGATACCGATGATAGACACAGAATCGAAGAAAAGAGGAAGAGAACTTACGAAACCTTCAAGTCCATCATGAAGAAGTC
TCCATTTTCTGGTCCAACTGATCCAAGACCACCACCAAGAAGAATTGCTGTTCCTTCAAGATCCTCTGCCTCTGTTCCAAAACCAGCTCCACAA
CCTTATCCTTTTACATCTTCTTTGTCCACCATCAACTACGATGAATTTCCAACTATGGTTTTCCCATCCGGTCAAATTTCTCAAGCTTCTGCTT
TGGCTCCAGCTCCTCCACAAGTTTTGCCACAAGCTCCAGCACCAGCTCCAGCTCCAGCAATGGTTTCTGCTTTAGCTCAAGCCCCAGCTCCTGT
TCCAGTTTTAGCTCCAGGTCCACCTCAAGCTGTTGCTCCACCAGCACCAAAACCTACTCAAGCTGGTGAAGGTACTTTGTCTGAAGCTTTGTTG
CAGTTGCAATTCGATGATGAAGATTTGGGTGCTTTGTTGGGTAATTCTACTGATCCAGCTGTTTTTACCGATTTGGCTTCTGTTGATAACTCCG
AATTTCAGCAGTTGTTGAATCAAGGTATTCCAGTTGCTCCTCATACAACTGAACCTATGTTGATGGAATATCCAGAAGCAATTACTAGATTAGT
TACTGGTGCTCAAAGACCTCCAGATCCTGCACCTGCTCCTTTGGGTGCTCCAGGTTTACCAAATGGTCTATTGTCTGGTGACGAAGATTTTTCC
TCTATTGCCGATATGGATTTCTCCGCTTTGTTGTGAGGATCCtaa
Csy4
>SEQ ID NO: 39
atgTCTGACCCAAAGAAAAAAAGAAAAGTTGGTTCAGGTTCTGACCACTACTTGGATATTAGATTAAGACCAGATCCAGAATTtCCACCAGCTC
AATTGATGTCTGTTTTGTTCGGTAAATTGCACCAAGCTTTGGTTGCTCAAGGTGGTGATAGAATTGGTGTTTCATTTCCAGATTTGGACGAATC
CAGATCaAGATTGGGTGAAAGATTGAGAATcCATGCTTCCGCTGATGATTTGAGAGCTTTGTTGGCTAGACCATGGTTGGAAGGTTTGAGAGAT
CACTTGCAATTTGGTGAACCAGCTGTTGTTCCACATCCAACTCCATATAGACAAGTTTCaAGAGTTCAAGCCAAGTCTAACCCAGAAAGATTGC
GTAGAAGATTGATGAGAAGGCACGATTTGTCTGAAGAAGAAGCCAGAAAAAGAATCCCAGATACTGTTGCTAGAGCTTTGGATTTGCCATTCGT
TACTTTGAGATCACAATCTACCGGTCAACATTTCAGGTTGTTCATTAGACATGGTCCATTGCAAGTTACTGCTGAAGAAGGTGGTTTTACTTGT
TACGGTTTGTCTAAAGGTGGATTCGTTCCATGGTTTGGATCCtaa
pWS4033 CRISPRai vector
>SEQ ID NO: 40
gcggccgccaatGAAGACTATCTAATTGTATCGAGATCACTTTTCGTGATCCGCTAATCAGCGACGGTCACATTAGGTTTGCCAAGTCAGGGTA
TGAACCATACGATCAGTTTTCGTGAACCTGGTACGTATATTGTGGCGTTTGTGTATATTTTCATTCTTTGACAACAATCAATACCAACCTCAAA
TAGGAAAAGTAATAAGTTTGGCGTTACACCCCAAAAGACGCCAAACGGATCGAACTTACTCAATAGCAATTAGCGAGACAAAACCTACGTTAAG
ACCTGTAACCGATTTATCAAAGCACTCTGCGGTTCTTTCTTGGGAATATTACCTGGACATTTTGTGCCCTCAAGAAACGAGGCTCTACGAGCCT
GTTGGAGCCCCTCAGACATTAGCCGCCACGAATCAAACTTTTTACGCGATTCGGCCCAAATCAGTTTCTCACAGATCATTCGTAGAGTGAAAAA
GCACATCGATTATTTGATACCCCTTTGGGTTAATTACTGTTGAGGTCTTTGCCGTAAGTTCCATCGCTGTCCCTctgaactggccgataattgc
agacgaacggtgttyttatccgatacaaccggatatttttcttttaatgagtctaaaccgtgatagcttcaggttaatAcaatcaaaaaaagct
caaatattcttttaatgccgcgttcacagattccaattgaatacaactaggtagttcattatatgaagcctttgctactatttttcactatAgt
ctgccttcaccttaatgcagacatccacatattttaatcactttaaaataaaaaggaagatatattagaagctatgatccaatctgtaagccag
attaaaattcAcgaactcttctttcatttgaattgaatgctttgagttggggtagattatcgcaaattactcatcacatttattgactacgaac
ttgctgatgtcctttttttatttAtatttttcttcagtgaagcgattttttttttacacagaccaagacggaaaaaagtagctaaggaagaaaa
caaaatcatgaaaaaaatgtgaagtgatcatgcacaTcgcatcaacttaaacattggcttagagatatatagagttagagtttacggcaacctt
taagcaccaataccttttggcatagtctaaagacctggttcttaattttaaacaaatttaactaaagatttccctatcaaagaagtaacgagtt
gacagattttctcaaaataaatcgatactgcatttctaggcatatccagcgagatctatgTCTGACCCAAAGAAGAAGAGAAAGGTTGGTTCTG
GTTCCAAGTTGGAAAAGTTTACCAACTGCTACTCTCTGTCTAAGACCTTGAGATTCAAAGCTATTCCTGTTGGTAAGACCCAAGAAAACATTGA
CAACAAGAGGTTGTTGGTTGAGGACGAAAAAAGAGCTGAAGATTACAAGGGTGTCAAGAAGTTGCTGGACAGATATTACTTGTCCTTCATCAAC
GATGTCCTGCACTCTATTAAGCTGAAGAACTTGAACAACTACATCTCCCTGTTCAGAAAAAAGACCAGAACCGAGAAAGAGAACAAAGAGTTAG
AAAACTTGGAGATCAACCTGAGGAAAGAAATTGCTAAGGCTTTCAAGGGTAACGAGGGTTACAAATCTTTGTTCAAGAAGGACATCATCGAAAC
CATCTTGCCAGAATTtTTGGATGATAAGGACGAAATTGCCTTGGTCAATTCTTTCAACGGTTTCACTACTGCTTTCACTGGTTTCTTTGACAAC
AGGGAAAACATGTTCTCTGAAGAAGCTAAGTCTACCTCCATTGCTTTCAGATGCATTAACGAAAACTTGACCAGGTACATCTCCAACATGGATA
TtTTCGAAAAGGTTGATGCCATCTTCGATAAGCACGAAGTCCAAGAAATCAAAGAGAAGATtTTGAACTCCGATTACGACGTCGAAGATTTTTT
CGAAGGTGAGTTCTTCAACTTCGTTTTGACTCAAGAAGGTATCGACGTTTACAACGCTATTATTGGTGGTTTCGTTACCGAATCTGGTGAAAAG
ATTAAGGGTTTGAACGAGTACATCAACCTGTACAATCAAAAGACCAAGCAGAAGTTGCCAAAGTTTAAGCCCTTGTACAAACAAGTCTTGTCCG
ACAGAGAATCCTTGTCTTTTTATGGTGAAGGTTACACCTCCGATGAAGAAGTTTTGGAAGTTTTCAGAAACACCCTGAACAAGAACTCCGAAAT
CTTCTCATCCATTAAGAAGTTGGAGAAGCTGTTTAAGAACTTCGACGAATATTCCTCCGCTGGTATCTTTGTCAAAAATGGTCCAGCTATTTCC
ACCATCTCCAAGGATATTTTTGGTGAATGGAACGTCATCAGAGATAAGTGGAATGCTGAATACGATGACATCCACTTGAAAAAGAAGGCTGTTG
TTACCGAAAAGTACGAGGATGATAGACGTAAGTCCTTCAAGAAGATTGGCTCTTTCTCTCTGGAACAATTGCAAGAATATGCTGATGCCGATTT
GTCCGTTGTCGAAAAATTGAAAGAAATCATCATCCAGAAGGTCGACGAAATCTACAAAGTTTACGGTTCCTCTGAAAAGTTGTTCGATGCTGAT
TTCGTTTTGGAGAAGTCGTTGAAGAAAAACGATGCTGTTGTTGCCATCATGAAGGATTTGTTGGATTCCGTTAAGAGCTTCGAGAACTACATTA
AGGCTTTTTTCGGTGAGGGCAAAGAAACTAACAGAGATGAATCATTCTACGGCGATTTTGTTTTGGCCTACGATATtTTGTTGAAGGTTGATCA
TATCTACGACGCCATCAGAAACTACGTTACTCAAAAACCATACTCCAAAGACAAGTTCAAGCTGTACTTTCAAAACCCACAATTCATGGGTGGT
TGGGACAAAGACAAAGAAACAGATTACAGAGCCACCATCTTGAGATATGGTTCTAAGTACTACTTGGCCATCATGGATAAGAAATACGCTAAGT
GCTTGCAAAAGATCGATAAGGATGATGTCAACGGTAACTACGAGAAGATCAACTACAAATTATTGCCCGGTCCAAACAAGATGTTGCCTAAGGT
TTTCTTCAGCAAAAAGTGGATGGCTTACTACAACCCATCCGAAGATATTCAAAAGATtTACAAGAACGGCACCTTCAAAAAGGGTGATATGTTC
AACTTGAACGACTGCCATAAGCTGATCGATTTCTTCAAGGATTCCATCTCaAGATACCCAAAGTGGTCTAATGCTTACGATTTCAACTTCTCTG
AAACCGAGAAGTACAAAGATATTGCCGGTTTTTACAGGGAAGTCGAAGAACAAGGTTACAAGGTTTCATTCGAATCCGCCTCTAAAAAAGAAGT
TGACAAGTTGGTTGAAGAGGGCAAGTTGTACATGTTCCAGATtTATAACAAGGACTTCTCCGATAAGTCTCATGGTACTCCAAACTTGCATACC
ATGTACTTCAAGTTGTTGTTCGACGAAAACAATCACGGTCAGATTAGATTGTCTGGTGGTGCTGAGTTGTTTATGAGAAGGGCTTCTTTGAAGA
AAGAAGAGTTGGTTGTTCATCCAGCCAATTCTCCAATTGCTAACAAGAATCCAGACAACCCAAAAAAGACTACCACCTTGTCTTACGATGTTTA
CAAGGATAAGAGATTCTCCGAGGACCAATACGAATTGCATATTCCAATTGCCATTAACAAGTGCCCCAAGAACATTTTCAAGATCAACACCGAA
GTTAGGGTGTTGTTGAAGCACGATGATAACCCATACGTTATTGGTATTGCTAGGGGTGAAAGAAACTTGTTGTACATCGTTGTTGTTGATGGTA
AGGGTAACATCGTGGAACAATACTCATTGAACGAGATTATCAACAACTTCAACGGCATCAGAATCAAGACCGATTACCATTCTCTGCTTGACAA
GAAAGAAAAAGAAAGATTCGAAGCCAGACAGAACTGGACCTCTATTGAAAACATCAAAGAGTTGAAGGCCGGTTACATTTCACAAGTTGTTCAT
AAGATtTGCGAGCTGGTTGAAAAGTATGATGCAGTTATTGCTTTGGAGGACTTGAACTCTGGTTTCAAGAACTCCAGAGTTAAGGTCGAAAAGC
AGGTCTACCAAAAGTTCGAAAAGATGTTGATCGACAAGCTGAACTACATGGTCGACAAAAAGTCTAATCCATGTGCTACAGGTGGTGCTTTGAA
AGGTTATCAAATTACGAACAAGTTCGAGAGCTTCAAGTCTATGTCTACTCAGAATGGTTTCATCTTCTACATTCCAGCTTGGTTGACCTCTAAG
ATTGATCCATCTACTGGTTTCGTCAACCTGCTGAAAACTAAGTACACTTCTATTGCCGACTCCAAGAAGTTCATCTCTTCATTCGATAGAATCA
TGTACGTTCCAGAAGAGGACTTATTCGAATTtGCTTTGGATTACAAGAACTTCTCaAGAACTGATGCTGACTACATCAAGAAGTGGAAGTTGTA
CTCTTACGGTAACAGAATCAGGATCTTCAGAAACCCTAAGAAGAACAACGTTTTCGATTGGGAAGAAGTCTGTTTGACCTCTGCTTACAAAGAA
CTGTTTAACAAGTACGGCATCAACTACCAACAAGGTGATATTAGAGCTTTGTTGTGCGAACAATCTGATAAGGCCTTCTACTCATCTTTTATGG
CCTTGATGTCACTGATGTTGCAGATGAGAAATTCCATTACTGGTAGAACCGATGTCGACTTTTTGATCTCTCCAGTTAAGAACTCTGACGGTAT
TTTCTACGACTCaAGAAACTATGAAGCTCAAGAAAACGCCATTTTGCCAAAAAACGCTGATGCTAATGGTGCTTACAACATTGCTAGAAAAGTT
TTGTGGGCTATCGGCCAATTCAAAAAAGCAGAAGATGAGAAGCTGGACAAGGTTAAGATTGCCATCTCAAACAAAGAATGGTTGGAATACGCTC
AGACTTCTGTCAAACATGGTTCTGGTTCTCCACCAAAGAAGAAAAGAAAGGTTGGTTCCGGTTCTAAATTAGGTGGTTCTGGTGGTTCCCCAAA
GAAGAAAAGAAAGGTTGGTAGAGCTGAAGCTTCTGGTTCAGGTAGAGCAGATGCTTTGGATGATTTCGATTTGGACATGTTGGGTTCTGATGCA
TTGGACGATTTCGACTTAGATATGTTAGGTAGTGACGCCTTGGATGACTTCGATCTGGATATGCTAGGTTCCGATGCCTTAGACGACTTTGATT
TAGACATGCTGATTAACTCAAGATCCTCTGGTTCTCCTAAGAAGAAGAGAAAAGTCGGTTCTCAATACTTGCCAGATACCGATGATAGACACAG
AATCGAAGAAAAGAGGAAGAGAACTTACGAAACCTTCAAGTCCATCATGAAGAAGTCTCCATTTTCTGGTCCAACTGATCCAAGACCACCACCA
AGAAGAATTGCTGTTCCTTCAAGATCCTCTGCCTCTGTTCCAAAACCAGCTCCACAACCTTATCCTTTTACATCTTCTTTGTCCACCATCAACT
ACGATGAATTTCCAACTATGGTTTTCCCATCCGGTCAAATTTCTCAAGCTTCTGCTTTGGCTCCAGCTCCTCCACAAGTTTTGCCACAAGCTCC
AGCACCAGCTCCAGCTCCAGCAATGGTTTCTGCTTTAGCTCAAGCCCCAGCTCCTGTTCCAGTTTTAGCTCCAGGTCCACCTCAAGCTGTTGCT
CCACCAGCACCAAAACCTACTCAAGCTGGTGAAGGTACTTTGTCTGAAGCTTTGTTGCAGTTGCAATTCGATGATGAAGATTTGGGTGCTTTGT
TGGGTAATTCTACTGATCCAGCTGTTTTTACCGATTTGGCTTCTGTTGATAACTCCGAATTTCAGCAGTTGTTGAATCAAGGTATTCCAGTTGC
TCCTCATACAACTGAACCTATGTTGATGGAATATCCAGAAGCAATTACTAGATTAGTTACTGGTGCTCAAAGACCTCCAGATCCTGCACCTGCT
CCTTTGGGTGCTCCAGGTTTACCAAATGGTCTATTGTCTGGTGACGAAGATTTTTCCTCTATTGCCGATATGGATTTCTCCGCTTTGTTGTGAG
GATCCtaactcgagagtgcttttaactaagaattattagtcttttctgcttattttttcatcatagtttagaacactttatattaacgaatagt
ttatgaatctatttaggtttaaaaattgatacagttttataagttactttttcaaagactcgtgctgtctattgcataatgcactggaagggga
aaaaaaaggtgcacacgcgtggctttttcttgaatttgcagtttgaaaaatgctgccaaaccagatgtcaacacagctacaacgtgacccaaca
tcagatgacccaaggtccacctcttattAaaggacgtttgatccttcgacaccatggctctgttgaacttttatctgagagaggaaaaaaagga
aggaaaaaaaagaagaaacttcctttatttatttgtcttaaccacaacacacaatgcaataagatgcaatataatatcaaagccaatatcttat
gttgctgatcctgagaaggaatatatacaatttatgtagtaaaataccttttcttctgcgagttgcaagaaatagaaaagactccgattgcgca
tcgccagaataaaatttcacaaccacactttttggctgaactttttattacctgattasacagagagagaaaaggtagaggtcaaaatttttta
agcaaaactaaaaaagatgcaaaatcacgtgctgaaaatctaacataagggttaagattagagttttataggacttgttttgtaatatttcaaa
tacgagctaaccctactgatttcaattaggtctaatttagggttgagctgcactgaaatttcggaaattttgggttattttaaatgagacagaa
gaactacagagatacgttcttcagactttaaagcttatctccacaaagaattggtcaagaaatcatcctagaaaaacacgtttgctcactcgat
cttaatcacatagagtgctggaacgggaagaaagatctatgTCTGATCCAAAAAAGAAGAGAAAGGTCGGTTCTGGTTCTGACAAGAAATACTC
TATTGGTTTGGCTATTGGCACCAATTCTGTTGGTTGGGCTGTTATTACTGACGAGTACAAAGTTCCCTCCAAGAAGTTTAAGGTTTTGGGTAAC
ACTGACAGGCACTCCATTAAGAAAAATTTGATTGGTGCCCTGTTGTTCGATTCTGGTGAAACTGCTGAAGCTACTAGATTGAAAAGAACCGCTA
GAAGAAGATACACCAGAAGAAAGAACAGAATCTGCTACTTGCAAGAGATTTTCTCTAACGAAATGGCCAAGGTTGATGATTCATTCTTCCACAG
ATTGGAAGAGTCCTTCTTGGTTGAAGAGGATAAGAAACACGAAAGACATCCAATCTTCGGTAACATCGTTGATGAAGTTGCTTACCACGAAAAG
TACCCAACTATCTACCATTTGAGAAAGAAGTTGGTTGACTCTACCGATAAGGCTGATTTGAGATTGATCTATTTGGCTTTGGCCCACATGATTA
AGTTCAGAGGTCATTTCTTGATCGAGGGTGATTTGAATCCAGATAACTCCGATGTTGACAAGTTGTTCATCCAATTGGTCCAAACTTACAACCA
GTTGTTCGAAGAAAATCCAATCAATGCCTCTGGTGTTGATGCTAAGGCTATTTTGTCTGCTAGACTGTCTAAGTCAAGAAGGTTGGAAAACTTG
ATTGCTCAATTGCCAGGTGAAAAGAAGAATGGTTTGTTCGGTAATTTGATCGCCTTGTCTTTGGGTTTGACTCCAAATTTCAAGTCCAACTTCG
ATTTGGCTGAAGATGCTAAGTTGCAATTGTCTAAGGATACCTACGATGATGACTTGGATAATTTGTTGGCCCAAATCGGTGATCAATACGCTGA
TTTGTTTTTGGCTGCTAAGAACTTGTCCGATGCCATTTTGTTGTCCGATATCTTGAGAGTTAACACCGAAATTACTAAGGCTCCATTGTCTGCC
TCTATGATCAAAAGATACGATGAACACCACCAAGACTTGACTTTGTTGAAAGCTTTGGTCAGACAACAGTTGCCAGAAAAGTACAAAGAAATCT
TCTTCGACCAGTCCAAAAATGGTTACGCTGGTTATATTGATGGTGGTGCTTCTCAAGAAGAGTTCTACAAGTTCATTAAGCCCATCTTGGAAAA
GATGGATGGTACTGAAGAACTGTTGGTCAAGTTGAACAGAGAAGATTTGTTGAGAAAGCAGAGAACCTTCGATAACGGTTCTATTCCACATCAA
ATCCACTTGGGTGAATTGCATGCTATTTTGAGAAGGCAAGAGGATTTCTACCCATTCTTGAAGGATAACAGAGAAAAGATCGAGAAGATCCTGA
CCTTCAGAATCCCATATTATGTTGGTCCATTGGCCAGAGGTAATTCAAGATTTGCTTGGATGACTAGGAAGTCCGAAGAAACTATTACTCCATG
GAACTTCGAAGAGGTTGTTGATAAGGGTGCTTCTGCTCAATCCTTCATTGAAAGAATGACTAACTTCGACAAGAACCTGCCAAACGAAAAGGTT
TTGCCAAAACACTCTTTGCTGTACGAATACTTCACCGTCTACAACGAATTGACTAAGGTTAAGTACGTCACCGAAGGTATGAGAAAACCAGCTT
TTTTGTCTGGTGAACAAAAGAAGGCTATCGTCGATTTGTTGTTCAAGACTAACAGAAAGGTTACCGTCAAGCAATTGAAAGAGGACTACTTCAA
GAAAATCGAGTGCTTCGACTCCGTTGAAATTAGTGGTGTTGAAGATAGATTCAACGCCTCCTTAGGTACTTACCATGACTTATTGAAGATCATC
AAGGACAAGGATTTCTTGGACAACGAAGAAAACGAGGATATCTTGGAGGATATCGTTTTGACTTTGACCTTGTTCGAGGATAGGGAAATGATCG
AAGAAAGGTTGAAAACCTACGCTCACTTGTTTGATGATAAGGTCATGAAGCAGTTGAAGAGAAGAAGGTATACTGGTTGGGGTAGATTGTCTAG
GAAATTGATTAACGGTATCAGGGACAAGCAATCCGGTAAGACTATTTTGGACTTTTTGAAGTCTGATGGCTTCGCCAATAGAAACTTCATGCAA
TTGATCCACGATGACAGCTTGACCTTCAAAGAAGATATTCAAAAGGCCCAAGTTTCCGGTCAAGGTGATTCTTTACATGAACATATTGCTAACT
TGGCTGGTTCCCCAGCTATTAAGAAAGGTATTTTACAAACCGTTAAGGTGGTCGACGAATTGGTAAAAGTTATGGGTAGACATAAGCCAGAGAA
CATCGTTATTGAAATGGCTAGAGAAAATCAGACTACCCAAAAGGGTCAAAAGAACTCCAGAGAAAGGATGAAGAGAATTGAAGAGGGTATCAAA
GAATTGGGCTCCCAGATTTTGAAAGAACACCCAGTTGAAAACACCCAATTGCAGAACGAAAAGCTGTACTTGTACTACTTGCAGAATGGTAGAG
ATATGTACGTCGACCAAGAATTGGATATCAACAGATTGTCCGATTACGATGTCGATGCTATCGTTCCACAGTCTTTTTTGAAGGATGACTCCAT
CGATAACAAGGTCTTGACTAGATCCGATAAGAACAGAGGTAAGTCTGATAACGTTCCATCTGAAGAGGTCGTCAAGAAGATGAAGAATTATTGG
AGACAGTTGCTGAACGCCAAGTTGATTACTCAAAGAAAGTTCGACAATTTGACCAAGGCTGAAAGAGGTGGTTTGTCTGAATTGGACAAAGCAG
GTTTCATTAAGAGGCAATTGGTTGAAACTAGGCAAATCACCAAACACGTTGCCCAAATTTTGGATTCCAGAATGAATACCAAGTACGACGAAAA
CGATAAGTTGATCAGGGAAGTTAAGGTCATCACGTTGAAGTCTAAGTTGGTTTCCGATTTCAGAAAGGACTTCCAATTCTACAAGGTCAGGGAA
ATCAACAACTACCATCATGCTCATGATGCTTACTTGAATGCTGTTGTTGGTACTGCCTTGATTAAGAAGTATCCAAAGTTGGAATCCGAGTTCG
TTTACGGTGATTACAAGGTTTACGACGTCAGAAAGATGATTGCCAAGTCCGAACAAGAAATTGGTAAGGCTACTGCTAAGTACTTCTTCTACTC
CAACATCATGAATTTCTTCAAGACCGAAATCACTTTGGCCAACGGTGAAATTAGAAAAAGGCCATTGATTGAGACTAACGGTGAGACTGGTGAA
ATAGTTTGGGATAAGGGTAGAGATTTCGCTACCGTTAGAAAAGTTTTGTCCATGCCACAAGTTAACATCGTCAAAAAGACCGAAGTTCAAACTG
GTGGTTTCTCCAAAGAATCCATCTTGCCAAAAAGAAACTCCGACAAATTGATCGCCAGAAAGAAAGATTGGGACCCAAAAAAGTATGGTGGTTT
TGATTCTCCAACCGTTGCTTACTCTGTTTTGGTTGTTGCTAAAGTTGAAAAGGGCAAGAGCAAGAAGTTGAAGTCAGTCAAAGAACTATTGGGC
ATCACGATCATGGAAAGGTCATCTTTTGAAAAAAACCCAATCGACTTTTTGGAGGCCAAGGGTTACAAAGAAGTTAAGAAGGATCTGATTATCA
AGCTGCCCAAGTACTCTTTGTTCGAATTGGAAAACGGTCGTAAGAGAATGTTGGCTTCTGCTGGTGAATTACAAAAGGGTAATGAATTGGCTCT
GCCCTCTAAGTACGTTAACTTTCTATACTTGGCCTCTCACTACGAAAAGTTGAAGGGTTCTCCTGAAGATAACGAACAAAAGCAGTTGTTTGTC
GAACAGCATAAGCACTACTTGGACGAAATCATCGAACAGATTTCTGAATTTTCCAAGAGGGTTATTTTGGCTGATGCCAATTTGGATAAGGTCT
TGTCTGCTTACAACAAGCACAGAGATAAGCCAATTAGAGAACAAGCCGAAAACATCATTCACCTGTTCACTTTGACTAATTTGGGTGCTCCAGC
TGCTTTTAAGTATTTCGATACCACCATCGACAGAAAGAGATACACTTCTACAAAAGAGGTTCTGGACGCTACCTTGATTCATCAATCTATTACT
GGCTTGTACGAAACCAGGATCGACTTGTCTCAATTAGGTGGTGATGGTTCAGGTTCTCCACCAAAGAAAAAACGTAAGGTCGGATCTGGTTCTA
TGGAACGTGTGAGAATGATTAATGTGCAAAGGCTGTTAGAAGCCGCAGAGTTTTTAGAAAGAAGAGAAAGAGAATGCGAACACGGGTATGCCAG
TTCTTTCCCTAGCATGCCCTCTCCCAGAGGATCCtaactcgagattgaattgaattgaaatcgatagatcaatttttttcttttctctttcccc
atcctttacgctaaaataatagtttattttattttttgaatattttttatttatatacgtatatatagactattatttatcttttaatgattat
taagatttttattaaaaaaaaattcgctcctcttttaatgcctttatgcagtttttttttcccattcgatatttctatgtgctggatgcacaca
ctggcttaagatgacaacgtatatattaaatttgctcttgttctgtactttcctaattcttatgtaaaaagacaagaatttatgatactattta
ataacaaaaaactacctaagAaaagcatcatgcagtcgaaattgaaatcgaaaagtaaaactttaacggaacatgtttgaaattctaagaaagc
atacatcttcatcccttatatatagagttatgtttgatattagtagtcatgttgtaatctctggcctaagtatacgtaacgaaaatggtagcac
gtcgcgtttatggcccccaggttaatgtgttctctgaaattcgcatcactttgagaaataatgggaacaccttacgcgtgagctgtgcccaccg
cttcgcctaataaagcggtgttctcaaaatttctccccgttttcaggatcacgagcgccatctagttctggtaaaatcgcgcttacaagaacaa
agaaaagaaacatcgcgtaatgcaacagtgagacacttgccgtcatatataaggttttggatcagtaaccgttatttgagcataacacaggttt
ttaaatatattattatatatcatggtatatgtgtaaaatttttttgctgactggttttgtttatttatttagctttttaaaaattttactttct
tcttgttaattttttctgattgctctatactcaaaccaacaacaacttactctacaactaagatctatgTCTGACCCAAAGAAAAAAAGAAAAG
TTGGTTCAGGTTCTGACCACTACTTGGATATTAGATTAAGACCAGATCCAGAATTtCCACCAGCTCAATTGATGTCTGTTTTGTTCGGTAAATT
GCACCAAGCTTTGGTTGCTCAAGGTGGTGATAGAATTGGTGTTTCATTTCCAGATTTGGACGAATCCAGATCaAGATTGGGTGAAAGATTGAGA
ATCCATGCTTCCGCTGATGATTTGAGAGCTTTGTTGGCTAGACCATGGTTGGAAGGTTTGAGAGATCACTTGCAATTTGGTGAACCAGCTGTTG
TTCCACATCCAACTCCATATAGACAAGTTTCaAGAGTTCAAGCCAAGTCTAACCCAGAAAGATTGCGTAGAAGATTGATGAGAAGGCACGATTT
GTCTGAAGAAGAAGCCAGAAAAAGAATCCCAGATACTGTTGCTAGAGCTTTGGATTTGCCATTCGTTACTTTGAGATCACAATCTACCGGTCAA
CATTTCAGGTTGTTCATTAGACATGGTCCATTGCAAGTTACTGCTGAAGAAGGTGGTTTTACTTGTTACGGTTTGTCTAAAGGTGGATTCGTTC
CATGGTTTGGATCCtaactcgaggcgaatttcttatgatttatgatttttattattaaataagttataaaaaaaataagtgtatacaaatttta
aagtgactcttaggttttaaaacgaaaattcttattcttgagtaactctttcctgtaggtcaggttgctttctcaggtatagcatgaggtcgct
cttattgaccacacctctaccggcatgccgagcaaatgcctgcaaatcgctccccatttcgctggttctgtatgggcacagacaacctaaacgc
cttgtgaaattgcaastatggtgatttgaaacgtttcctagtgcagcaggatcacagataacgtytaaagggcttagcagttgataatcctctc
tagttaagacctaaacaaaatgctgtcactaaccgtagtattaaatgacacactttgytgactttccttaatggggatgtggtagtggccattg
ccaataaacaaaaagaacagggaaagaagtagaaagtgatataagtttgcttgccacttttcgtttttcacgaaaaaaacaggcgaaaaaaaat
gctagacaagtacccggctgaatcacacctcgttaacagtgactttcggtgacagatacccgattgggcacccggctggtaagttatgatagaa
agccaacgctgtactattggcttagctatggcaatattttgattatcagctagttttattaacgttataattagtgtaaccagtttttcatcta
tttcatttatttcatttatttactttaattgcagatccccctaacgcgtttaaagcttttattcactagcttatgtattttttataggaaacgc
gacgcgtaacatcgcgcaaatgaaggttttgatgtattataatgaggtattcttccttatatacatcgatgaaaagcgttgacagcatacattg
gaaagaaataggaaacggacaccggaagaaaaaatagatctatgTCAAGATTGGACAAAAGCAAGGTTATTAACGGTGCCTTGGAACTGTTGAA
TGGTGTAGGTATAGAAGGCTTAACGACCAGAAAATTAGCACAAAAGTTAGGTGTAGAGCAGCCAACATTATATTGGCACGTCAAAAACAAGAGA
GCATTGCTAGACGCATTGCCAATCGAAATGTTAGATCGTCATCACACTCATTTTTGCCCATTAGAGGGTGAGAGTTGGCAAGACTTTTTGCGTA
ACAATGCTAAATCCTTTAGATGCGCCTTGTTGAGCCATCGTGATGGTGCCAAGGTTCATCTAGGTACAAGGCCAACAGAAAAACAGTATGAGAC
TTTGGAGAATCAGCTAGCCTTTTTATGCCAACAGGGTTTTTCATTGGAGAATGCCTTATACGCTTTAAGTGCAGTTGGTCACTTCACTTTAGGT
TGCGTACTAGAAGAACAAGAGCACCAGGTTGCTAAAGAAGAGAGAGAAACACCTACAACAGATTCAATGCCTCCATTGCTAAGACAAGCTATCG
AACTGTTTGATAGACAGGGTGCAGAACCTGCATTCTTGTTCGGTTTAGAGTTGATTATTTGCGGCTTGGAAAAGCAGCTAAAATGCGAATCTGG
TAGTGGTTCACCTCCTAAGAAAAAGAGAAAAGTTGGTTCCGGTAGCGCTAATTTCAATCAGTCTGGTAATATTGCCGACTCCTCTTTGTCTTTC
ACTTTCACTAATTCTTCCAACGGTCCAAACTTGATCACTACCCAAACTAATTCTCAAGCTTTGTCTCAGCCAATTGCCTCTTCTAATGTTCACG
ATAACTTCATGAACAACGAAATCACCGCCTCCAAAATTGATGATGGTAACAATTCTAAGCCATTGTCTCCAGGTTGGACTGATCAAACTGCTTA
TAATGCTTTCGGTATTACCACCGGTATGTTTAACACTACTACTATGGATGACGTCTACAACTACTTGTTCGATGATGAAGATACACCACCAAAT
CCAAAGAAAGAAGGATCCtaactcgaggccaattggtgcggcaattgataataacgaaaatgtcttttaatgatctgggtataatgaggaattt
tccgaacgtttttactttatatatatatatacatgtaacatatattctatacgctatagagaaaggaaatttttcaattaaaaaaaaaatagag
aaagagtttcacttcttgattatcgctaacactaatggttgaagtactgctactttaattttatgctgggtagactacccatgagtcacaatga
acgttcgtgctttgtgataaagtgtttcacgtcatccgacatgacttcgtagttatggactgaactgtgtggtgaggttccatgatttcttagg
tccagcagatacatgtctcttcccaatttcttgttaaggttacggccaatgcttcggttgttgagcttgttaccgaataagccgtgaagtatga
taataggtggtcttggcttcccttcatccccagtttttactgcatctctcttgattatgtcatatgaaaggtccagtgggacttgcttttgttg
cagcacctttgctaatgaatgaaaggcacatagtgactgcttaaaaatgcaggaacttaaattattccgaatggtattttgtctcacatatatt
gtcccatactytgccaagatcccggctttacccagtatcatcattgtaccgttaccaattctcctcgtatatcacggttagtttttaaacctcg
gggtgacgtttactattggcgtactaatatattcttattttcttttcttttttgttggcagtttcaagcaacacatgtactggataaccaaccc
ccgcacgctcttggaaaaaattgagaaggcatcggacacttgctgatgagtatttcgaaaaattccatgaagatgaggccaagattgtttggaa
gagattgaaaagaagaagaagaaaaaaagataaaagcaaatcaaaagatctatgTCAAGATTGGATAAGTCCAAGGTTATCAACTCTGCTTTGG
AGTTGTTGAACGAAGTTGGTATTGAAGGTTTGACCACTAGAAAGTTGGCTCAAAAATTGGGTGTTGCTCAAAAAACCTTGTACTGGCACGTTAA
GAACAAAAGAGCTTTGTTGGACGCTTTGGCCATTGAAATGTTGGATAGACATCATACCCATTTCTGTCCATTGGAAGGTGAATCATGGCAAGAT
TTCTTGAGAAACAACGCCAAGTCTTTCAGATGTGCTTTGTTGTCTCATAGAGATGGTGCTAAAGTTCACTTGGGTACTAGACCAACTGAAAAGC
AATACGAAACTTTGGAAAACCAGTTGGCTTTCTTGTGTCAACAAGGTTTCTCTTTAGAAAACGCCTTGTATGCTTTGTCTGCTGTTGGTCATTT
TACCTTGGGTTGTGTTTTGGAAGATCAAGAACATCAAGTCGCCAAAGAAGAAAGGGAAACTCCAACTACTGATTCTATGCCACCATTATTGAGA
CAAGCCATTGAGTTGTTTGATCATCAAGGTGCTGAACCAGCTTTTTTGTTTGGTTTGGAATTGATCATCTGCGGTTTGGAGAAACAATTGAAGT
GTGAATCTGGTTCTGGTTCTCCACCAAAAAAGAAAAGAAAGGTTGGTTCAGGTTCCGAAAGGGTTAGAATGATTAACGTTCAAAGGTTGTTGGA
AGCTGCCGAATTTTTGGAAAGACGTGAAAGGGAATGTGAACATGGTTACGCTTCATCTTTTCCATCTATGCCATCTCCAAGAGGATCCtaactc
gagagcttttgattaagccttctagtccaaaaaacacgtttttttgtcatttatttcattttcttagaatagtttagtttattcattttatagt
cacgaatgttttatgattctatatagggttgcaaacaagcatttttcattttatgttaaaacaatttcaggtttaccttttattctgcttgtgg
tgacgcgtgtatccgcccgctcttttggtcacccatgtatgctgaagtgaataaagctccacacagtcgaacgGGTAGTCCATCGTTGTAGGAT
ACTCCCTATCAGTGATAGAGAAATCTATGCGGCATCCCTATCAGTGATAGAGATCGGTAACGGAGTCCCTATCAGTGATAGAGAAGTGGTGTTC
AGTTCCCTATCAGTGATAGAGAAGTTTGACACTATCCCTATCAGTGATAGAGAGAACAGCAATGACTCCCTATCAGTGATAGAGATCAAATGGC
GTATCCCTATCAGTGATAGAGATAATTAACTTGTAATATTCTAATCAATTGATATAAAAATCCCCGTCAGTGACGGAGATTTTCTTTTTTTTTT
ATTTTTCCCCGTCAGTGACGGAGAATATCAAGCTACAAAAAgaattcgcatctagalTCTtgagacggttcactgccgtataggcagtgagacc
gaaagtgaaacgtgatttcatgcgtcattttgaacattttgtaaatcttatttaataatgtgtgcggcaattcacatttaatttatgaatgttt
tcttaacatcgcggcaactcaagaaacggcaggttcggatcttagctactagagaaagaggagaaatactagatgcgtaaaggcgaagagctgt
tcactggtgtcgtccctattctggtggaactggatggtgatgtcaacggtcataagttttccgtgcgtggcgagggtgaaggtgacgcaactaa
tggtaaactgacgctgaagttcatctgtactactggtaaactgccggttccttggccgactctggtaacgacgctgacttatggtgttcagtgc
tttgctcgttatccggaccatatgaagcagcatgacttcttcaagtccgccatgccggaaggctatgtgcaggaacgcacgatttcctttaagg
atgacggcacgtacaaaacgcgtgcggaagtgaaatttgaaggcgataccctggtaaaccgcattgagctgaaaggcattgactttaaagagga
cggcaatatcctgggccataagctggaatacaattttaacagccacaatgtttacatcaccgccgataaacaaaaaaatggcattaaagcgaat
tttaaaattcgccacaacgtggaggatggcagcgtGcagctggctgatcactaccagcaaaacactccaatcggtgatggtcctgttctgctgc
cagacaatcactatctgagcacgcaaagcgttctgtctaaagatccgaacgagaaacgcgatcatatggttctgctggagttcgtaaccgcagc
gggcatcacgcatggtatggatgaactgtacaaatgaccaggcatcaaataaaacgaaaggctcagtcgaaagactgggcctttcgttttatct
gttgtttgtcggtgaacgctctctactagagtcacactggctcaccttcgggtgggcctttctgcgtttataggtctcaTCCCCGTCAGTGACG
GAGAtcgtctctACCAactagtgcactgcagtacaataaagcaatcttgatgaggataatgatttttttttgaatatacataaatactaccgtt
tttctgctagattttgtgatgacgtaaataagtacatattactttttaagccaagacaagattaagcattaactttacccttttctttctaagt
ttcaatattagttatcactgtttaaaagttatggcgagaacgtcggcggttaaaatatattaccctgaacgagcatacatcgaggagaacttct
agtatatctacatacctaatattattgccttattaaaaatggaatcccaacaattacatcaaaatccacattctcttcaaaatcaattgtcctg
tacttccttgttcatgtgtgttcaaaaacgttatatttataggataattatactctatttctcaacaagtaattggttgtttggccgagcggtc
taaggcgcctgattcaagasatatcttgaccgcagttaactgtgggaatactcaggtatcgtaagatgcaagagttcgaatctcttagcaacca
ttatttttttcctcaacataacgagaacacacaggggcgctatcgcacagaatcasattcgatgactggaaattttttgttaatttcagaggtc
gcctgacgcatatacctttttcaactgaaaaattyggagaaaaaggaaaggtgagagcgccggaaccggctTttcatatagaatagagaagcgt
tcatgactaaatgcttgcatcacaatacttgaagttgacaatattatttaaggacctattgttttttccaataggtggttagcaatcgtcttac
tttctaacttttcttaccttttacatttcagcaatatatatatatatatttcaaggatataccattctaatgtctgcccctaagaagatcgtcg
ttttgccaggtgaccacgttggtcaagaaatcacagccgaagccattaaggttcttaaagctatttctgatgttcgttccaatgtcaagttcga
tttcgaaaatcatttaattggtggtgctgctatcgatgctacaggtgttccacttccagatgaggcgctggaagcctccaagaaggctgatgcc
gttttgttaggtgctgtgggtggtcctaaatggggaaccggtagtgttagacctgaacaaggtttactaaaaatccgtaaagaacttcaattgt
acgccaacttaagaccatgtaactttgcatccgactctcttttagacttatctccaatcaagccacaatttgctaaaggtactgacttcgttgt
tgtcagagaattagtgggaggtatttactttggtaagagaaaggaggacgatggtgatggtgtcgcttgggatagtgaacaatacaccgttcca
gaagtgcaaagaatcacaagaatggccgctttcatggccctacaacatgagccaccattgcctatttygtccttggataaagctaatgttttgg
cctcttcaagattatggagaaaaactgtggaggaaaccatcaagaacgaatttcctacattgaaggttcaacatcaattgaTtgattctgccgc
catgatcctagttaagaacccaacccacctaaatggtattataatcaccagcaacatgtttggtgatatcatctccgatgaagcctccgttatc
ccaggttccttgggtttgttgccatctgcgtccttggcctctttgccagacaagaacaccgcatttggtttgtacgaaccatgccacggttctg
ctccagatttgccaaagaataaggtcaaccctatcgccactatcttgtctgctgcaatgatgttgaaattgtcattgaacttgcctgaagaagg
taaggccattgaagatgcagttaaaaaggttttggatgcaggtatcagaactggtgatttaggtggttccaacagtaccaccgaagtcggtgat
gctgtcgccgaagaagttaagaaaatccttgcttaacctggaggacccttctctttagactattctactcttatgcacgtaaaaaattctagga
aatatgtattaactaggagtaaaataaccggctagtggcattcatatagccgtctgtttacatctacatcacacatttcgagtgtatatctcgc
aacgttggcgttaaataggcaggagtTATCGTGTTGCATCTGCGGCTTTAAATTGATGTATCTCATCGCAGGCACGGGCAGTACAGTGCCCTGA
GCGTAGGGAAAAATGAAAAAAAGGATGTAACTTTTAACATAATTCCAGCACGCAGCGATTGGGTATAATGAAGATTGTTAAGTTCAACAACATG
TGAAATTCTTAGCTCCGCAACTTGCTTGAATTATGAGCTCTAAGATTCAAGAAGTAAATAGGTCATATAATGGAATGATAGTAGTACTTGATAA
AGGAACCATGTGATCTTACGTTGATATGAAAATAATTCTCTCACAGAAGTCAGTTTGTATGACTAACATAGAGAGTAATTGCTTTTCGGATAAA
ATCGCACCTAACAAAGATTACCTTTTGTAAAGCCTCCAGAACAGCTATGAATGTTCGTTATTCTACGCACTTTCTTTCATACATCTTGATCCGC
CTTTTTTGATGTAGGTTTTAACGCGGCAAAACATATTAATTTTTCTGATTTGGAAAAATAAAGAGTTAGTTCTGCCTAGTCTTCCCGAgcggcc
gcgtgttacaaccaattaaccaattctgattagaaaaactcatcgagcatcaaatgaaactgcaatttattcatatcaggattatcaataccat
atttttgaaaaagccgtttctgtaatgaaggagaaaactcaccgaggcagttccataggatggcaagatcctggtatcggtctgcgattccgac
tcgtccaacatcaatacaacctattaatttcccctcgtcaaaaataaggttatcaagtgagaaatcaccatgagtgacgactgaatccggtgag
aatggcaaaagcttatgcatttctttccagacttyttcaacaggccagccattacgctcgtcatcaaaatcactcgcatcaaccaaaccgttat
tcattcgtgattgcgcctgagcgaggcgaaatacgcgatcgctgttaaaaggacaattacaaacaggaatcgaatgcaaccggcgcaggaacac
tgccagcgcatcaacaatattttcacctgaatcaggatattcttctaatacctggaatgctgttttcccggggatcgcagtggtgagtaaccat
gcatcatcaggagtacggataaaatgcttgatggtcggaagaggcataaattccgtcagccagtttagtctgaccatctcatctgtaacatcat
tggcaacgctacctttgccatgtttcagaaacaactctggcgcatcgggcttcccatacaatcgatagattgtcgcacctgattgcccgacatt
atcgcgagcccatttatacccatataaatcagcatccatgttggaatttaatcgcggcctggagcaagacgtttcccgttgaatatggctcata
acaccccttgtattactgtttatgtaagcagacagttttattgttcatgatgatatatttttatcttgtgcaatgtaacatcagagattttgag
acacaacgtggctttcttgaataaatcgaacttttgctgagttgaaggatcagtcatgaccaaaatcccttaacgtgagttttcgttccactga
gcgtcagaccccgtagaaaagatcaaaggatcttcttgagatcctttttttctgcgcgtaatctyctgcttgcaaacaaaaaaaccaccgctac
cagcggtggtttgtttgccggatcaagagctaccaactctttttccgaaggtaactggcttcagcagagcgcagataccaaatactgttcttct
agtgtagccgtagttaggccaccacttcaagaactctgtagcaccgcctacatacctcgctctgctaatcctgttaccagtggctgctgccagt
ggcgataagtcgtgtcttaccgggttggactcaagacgatagttaccggataaggcgcagcggtcgggctgaacggggggttcgtgcacacagc
ccagcttggagcgaacgacctacaccgaactgagatacctacagcgtgagctatgagaaagcgccacgcttcccgaagggagaaaggcggacag
gtatccggtaagcggcagggtcggaacaggagagcgcacgagggagcttccagggggaaacgcctggtatctttatagtcctgtcgggtttcgc
cacctctgacttgagcgtcgatttttgtgatgctcgtcaggggggggagcctatggaaaaacgccagcaacgcggcctttttacggttcctgg
ccttttgctgyccttttgctcacatgttctttcctgcgttatcccctgattctgtggataaccgt
exemplary nucleic acid construct according to the invention
>SEQ ID NO: 41
GGTAGTCCATCGTTGTAGGATACTCCCTATCAGTGATAGAGAAATCTATGCGGCATCCCTATCAGTGATAGAGATCGGTAACGGAGTCCCTATC
AGTGATAGAGAAGTGGTGTTCAGTTCCCTATCAGTGATAGAGAAGTTTGACACTATCCCTATCAGTGATAGAGAGAACAGCAATGACTCCCTAT
CAGTGATAGAGATCAAATGGCGTATCCCTATCAGTGATAGAGATAATTAACTTGTAATATTCTAATCAATTGATATAAAAATCCCCGTCAGTGA
CGGAGATTTTCTTTTTTTTTTATTTTTCCCCGTCAGTGACGGAGAATATCAAGCTACAAAAAGAATTCGCATCTAGATTCTgttcactgccgta
taggcagCTTTGTATTCCAACTTACCGGTTTTAGAGCTAGAAATAGCAAGTTAAAATAAGGCTAGTCCGTTATCAACTTGAAAAAGTGGCACCG
AGTCGGTGCTTTTgttcactgccgtataggcagTGTTGACGTTCTCAACATGAGTTTTAGAGCTAGAAATAGCAAGTTAAAATAAGGCTAGTCC
GTTATCAACTTGAAAAAGTGGCACCGAGTCGGTGCTTTTgttcactgccgtataggcagGTGCCTGCTGATAAGTACTGGTTTTAGAGCTAGAA
ATAGCAAGTTAAAATAAGGCTAGTCCGTTATCAACTTGAAAAAGTGGCACCGAGTCGGTGCTTTTgttcactgccgtataggcagTCCCCGTCA
GTGACGGAGATGCCgttcactgccgtataggcagTGTGAGACAACAGACGCACAGTTTTAGAGCTAGAAATAGCAAGTTAAAATAAGGCTAGTC
CGTTATCAACTTGAAAAAGTGGCACCGAGTCGGTGCTTTTgttcactgccgtataggcagCCGCGTCCAGATCTGCAGACGTTTTAGAGCTAGA
AATAGCAAGTTAAAATAAGGCTAGTCCGTTATCAACTTGAAAAAGTGGCACCGAGTCGGTGCTTTTgttcactgccgtataggcagCAGCTGCC
TTGAGCTCCTCGGTTTTAGAGCTAGAAATAGCAAGTTAAAATAAGGCTAGTCCGTTATCAACTTGAAAAAGTGGCACCGAGTCGGTGCTTTTgt
tcactgccgtataggcagTCCCCGTCAGTGACGGAGACTAAgttcactgccgtataggcagCAACAAGCTATGAATATGAGGTTTTAGAGCTAG
AAATAGCAAGTTAAAATAAGGCTAGTCCGTTATCAACTTGAAAAAGTGGCACCGAGTCGGTGCTTTTgttcactgccgtataggcagGAACGTG
CTATTGAGAAGGAGTTTTAGAGCTAGAAATAGCAAGTTAAAATAAGGCTAGTCCGTTATCAACTTGAAAAAGTGGCACCGAGTCGGTGCTTTTg
ttcactgccgtataggcagCTGAATAAGACATGTTAGGGGTTTTAGAGCTAGAAATAGCAAGTTAAAATAAGGCTAGTCCGTTATCAACTTGAA
AAAGTGGCACCGAGTCGGTGCTTTTgttcactgccgtataggcagTCCCCGTCAGTGACGGAGAATGAgttcactgccgtataggcagAATTTC
TACTAAGTGTAGATGCTATGACACCTTTTCTTTCqttcactgccgtataggcagAATTTCTACTAAGTGTAGATGCACCAATATTGAGTCACAT
gttcactgccgtataggcagTCCCCGTCAGTGACGGAGAACCAACTAGTGCACTGCAGTACAATAAAGCAATCTTGATGAGGATAATGATTTTT
TTTTGAATATACATAAATACTACCGTTTTTCTGCTAGATTTTGTGATGACGTAAATAAGTACATATTACTTTTTAAGCCAAGACAAGATTAAGC
ATTAACTTTACCCTTTTCTTTCTAAGTTTCAATATTAGTTATCACTGTTTAAAAGTTATGGCGAGAACGTCGGCGGTTAAAATATATTACCCTG
AACG
exemplary inducible array
>SEQ ID NO: 42
GGTAGTCCATCGTTGTAGGATACTCCCTATCAGTGATAGAGAAATCTATGCGGCATCCCTATCAGTGATAGAGATCGGTAACGGAGTCCCTATC
AGTGATAGAGAAGTGGTGTTCAGTTCCCTATCAGTGATAGAGAAGTTTGACACTATCCCTATCAGTGATAGAGAGAACAGCAATGACTCCCTAT
CAGTGATAGAGATCAAATGGCGTATCCCTATCAGTGATAGAGATAATTAACTTGTAATATTCTAATCAATTGATATAAAAATCCCCGTCAGTGA
CGGAGATTTTCTTTTTTTTTTATTTTTCCCCGTCAGTGACGGAGAATATCAAGCTACAAAAAgaattcgcatctagaTTCTtgagacggttcac
tgccgtataggcagtgagaccgaaagtgaaacgtgatttcatgcgtcattttgaacattttgtaaatcttatttaataatgtgtgcggcaattc
acatttaatttatgaatgttttcttaacatcgcggcaactcaagaaacggcaggttcggatcttagctactagagaaagaggagaaatactaga
tgcgtaaaggcgaagagctgttcactggtgtcgtccctattctggtggaactggatggtgatgtcaacggtcataagttttccgtgcgtggcga
gggtgaaggtgacgcaactaatggtaaactgacgctgaagttcatctgtactactggtaaactgccggttccttggccgactctggtaacgacg
ctgacttatggtgttcagtgctttgctcgttatccggaccatatgaagcagcatgacttcttcaagtccgccatgccggaaggctatgtgcagg
aacgcacgatttcctttaaggatgacggcacgtacaaaacgcgtgcggaagtgaaatttgaaggcgataccctggtaaaccgcattgagctgaa
aggcattgactttaaagaggacggcaatatcctgggccataagctggaatacaattttaacagccacaatgtttacatcaccgccgataaacaa
aaaaatggcattaaagcgaattttaaaattcgccacaacgtggaggatggcagcgtgcagctggctgatcactaccagcaaaacactccaatcg
gtgatggtcctgttctgctgccagacaatcactatctgagcacgcaaagcgttctgtctaaagatccgaacgagaaacgcgatcatatggttct
gctggagttcgtaaccgcagcgggcatcacgcatggtatggatgaactgtacaaatgaccaggcatcaaataaaacgaaaggctcagtcgaaag
actgggcctttcgttttatctgttgtttgtcggtgaacgctctctactagagtcacactggctcaccttcgggtgggcctttctgcctttatag
gtctcalCCCCGTCAGTGACGGAGAtcgtctctACCAactagtgcactgcagtacaataaagcaatcttgatgaggataatgatttttttttga
atatacataaatactaccgtttttctgctagattttgtgatgacgtaaataagtacatattactttttaagccaagacaagattaagcattaac
tttacccttttctttctaagtttcaatattagttatcactgtttaaaagttatggcgagaacgtcggcggttaaaatatattaccctgaacg
ScTDH1 Terminator
>SEQ ID NO: 43
ATAAAGCAATCTTGATGAGGATAATGATTTTTTTTTGAATATACATAAATACTACCGTTTTTCTGCTAGATTTTGTGATGACGTAAATAAGTAC
ATATTACTTTTTAAGCCAAGACAAGATTAAGCATTAACTTTACCCTTTTCTTTCTAAGTTTCAATATTAGTTATCACTGTTTAAAAGTTATGGC
GAGAACGTCGGCGGTTAAAATATATTACCCTGAACG
AgLEU2 Terminator
>SEQ ID NO: 44
cctggaggacccttctctttagactattctactcttatgcacgtaaaaaattctaggaaatatgtattaactaggagtaaaataaccggctagt
ggcattcatatagccgtctgtttacatctacatcacacatttcgagtgtatatctcgcaacgttggcgttaaataggcaggagtTATCGTGTTG
CATCTGCGGCTTTAAATTGATGTATCTCA

The invention also provides the embodiments set out in the following numbered paragraphs:

1. A nucleic acid construct comprising:
a) a promoter module wherein the promoter module comprises at least one promoter operator of a first sequence; and
b) a gene-regulating and/or gene-editing array module, that comprises at least one array sub-module, wherein each array sub-module comprises at least a first nucleic acid region that encodes a nucleic acid that is capable of directing RNA mediated gene regulation or RNA mediated gene editing,
   wherein the gene-regulating and/or gene-editing array module is capable of being transcribed into a single polycistronic nucleic acid transcript from a single promoter,
   and wherein between each nucleic acid region that encodes a nucleic acid that is capable of directing RNA mediated gene regulation or RNA mediated gene editing is a sequence that when in RNA form is an RNA cleavage site; and
   wherein each array sub-module comprises at least one array operator of a second sequence
and wherein the gene-regulating and/or gene-editing array module is operably linked to promoter module.

2. The nucleic acid construct of paragraph 1, wherein the promotor module further comprises at least one promoter operator of a second sequence.

3. The nucleic acid construct of either of paragraphs 1 or 2, wherein the sequence of the at least one promoter operator of a first sequence and the at least one array operator of a second sequence are different.

4. The nucleic acid construct of paragraphs 2 or 3, wherein:
a) the sequence of the at least one promoter operator of a first sequence and the at least one promoter operator of a second sequence are different; and/or
b) the sequence of the at least one promoter operator of a first sequence and the at least one array operator of a second sequence are the same.

5. The nucleic acid construct according to any of the preceding paragraphs, wherein the promoter module comprises at least 2, at least 3, at least 4, at least 5, at least 6, at least 7, at least 8, at least 9, at least 10 or more promoter operators of a first sequence.

6. The nucleic acid construct according to any of paragraphs 2-5, wherein the promoter module comprises at least 2, at least 3, at least 4, at least 5, at least 6, at least 7, at least 8, at least 9, at least 10 or more promoter operators of a second sequence.

7. The nucleic acid construct according to any of the preceding paragraphs, wherein each array sub-module comprises at least 2, at least 3, at least 4, at least 5, at least 6, at least 7, at least 8, at least 9, or at least 10 array operators of a second sequence.

8. The nucleic acid construct of any of the preceding paragraphs wherein the gene-regulating and/or gene-editing array module comprises:
a) between 2 and 100 array sub-modules; optionally between 5 and 95, 10 and 90, 15 and 85, 20 and 80, 25 and 75, 30 and 70, 35 and 65, 40 and 60, 45 and 55 array sub-modules; and/or
b) at least 2, at least 3, at least 4, at least 5, at least 6, at least 7, at least 8, at 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 77, at least 76, at least 77, at least 78, at least 79, at least 80, at least 81, at least 82, at least 83, at least 84, at least 88, at least 86, at least 87, at least 88, at least 89, at least 90, at least 91, at least 92, at least 93, at least 94, at least 99, at least 96, at least 97, at least 98, at least 99, or more array sub-modules; optionally comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 66, 66, 67, 68, 69, 70, 71, 72, 73, 74, 77, 76, 77, 78, 79, 80, 81, 82, 83, 84, 88, 86, 87, 88, 89, 90, 91, 92, 93, 94, 99, 96, 97, 98, 99, or 100 array sub-modules.

9. The nucleic acid construct of any of the preceding paragraphs, wherein at least one array sub-module comprises:
a) between 2 and 100 nucleic acid regions that each encode a nucleic acid that is capable of directing RNA mediated gene regulation or RNA mediated gene editing; optionally between 5 and 95, 10 and 90, 15 and 85, 20 and 80, 25 and 75, 30 and 70, 35 and 65, 40 and 60, 45 and 55 nucleic acid regions that each encode a nucleic acid that is capable of directing RNA mediated gene regulation or RNA mediated gene editing; and/or
b) at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 77, at least 76, at least 77, at least 78, at least 79, at least 80, at least 81, at least 82, at least 83, at least 84, at least 88, at least 86, at least 87, at least 88, at least 89, at least 90, at least 91, at least 92, at least 93, at least 94, at least 99, at least 96, at least 97, at least 98, at least 99, or more nucleic acid regions that each encode a nucleic acid that is capable of directing RNA mediated gene regulation or RNA mediated gene editing; optionally comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 66, 66, 67, 68, 69, 70, 71, 72, 73, 74, 77, 76, 77, 78, 79, 80, 81, 82, 83, 84, 88, 86, 87, 88, 89, 90, 91, 92, 93, 94, 99, 96, 97, 98, 99, or 100 nucleic acid regions that each encode a nucleic acid that is capable of directing RNA mediated gene regulation or RNA mediated gene editing.

10. The nucleic acid construct according to any of the preceding paragraphs wherein each array sub-module comprises a single array operator of a second sequence.

11. The nucleic acid construct according to any of the preceding paragraphs wherein within each array sub-module:
a) the array operator is located upstream (5′) of the at least a first nucleic acid region that encodes a nucleic acid that is capable of directing RNA mediated gene regulation or RNA mediated gene editing; or
b) the array operator is located downstream (3′) of the at least a first nucleic acid region that encodes a nucleic acid that is capable of directing RNA mediated gene regulation or RNA mediated gene editing.

12. The nucleic acid construct according to any of the preceding paragraphs wherein at least one array sub-module comprises at least:
a) a first and a second nucleic acid region that encodes a nucleic acid that is capable of directing RNA mediated gene regulation or RNA mediated gene editing;
b) a first, a second and a third nucleic acid region that encodes a nucleic acid that is capable of directing RNA mediated gene regulation or RNA mediated gene editing;
c) a first, a second, a third and a fourth nucleic acid region that encodes a nucleic acid that is capable of directing RNA mediated gene regulation or RNA mediated gene editing;
d) a first, a second, a third, a fourth and a fifth nucleic acid region that encodes a nucleic acid that is capable of directing RNA mediated gene regulation or RNA mediated gene editing;
e) a first, a second, a third, a fourth, a fifth and a sixth nucleic acid region that encodes a nucleic acid that is capable of directing RNA mediated gene regulation or RNA mediated gene editing;
f) a first, a second, a third, a fourth, a fifth, a sixth and a seventh nucleic acid region that encodes a nucleic acid that is capable of directing RNA mediated gene regulation or RNA mediated gene editing;
g) a first, a second, a third, a fourth, a fifth, a sixth, a seventh and an eighth nucleic acid region that encodes a nucleic acid that is capable of directing RNA mediated gene regulation or RNA mediated gene editing;
h) a first, a second, a third, a fourth, a fifth, a sixth, a seventh, an eighth and a ninth nucleic acid region that encodes a nucleic acid that is capable of directing RNA mediated gene regulation or RNA mediated gene editing;
i) a first, a second, a third, a fourth, a fifth, a sixth, a seventh, an eighth, a ninth and a tenth nucleic acid region that encodes a nucleic acid that is capable of directing RNA mediated gene regulation or RNA mediated gene editing;
and wherein the array operator is located:
   upstream (5′) of the first nucleic acid region that encodes a nucleic acid that is capable of directing RNA mediated gene regulation or RNA mediated gene editing within the array sub-module; or
   downstream (3′) of the last nucleic acid region that encodes a nucleic acid that is capable of directing RNA mediated gene regulation or RNA mediated gene editing within the array sub-module so as to regulate transcription of sub-module.

13. The nucleic acid construct according to any of the preceding paragraphs wherein the nucleic acid construct comprises at least a first array sub-module and a second array sub-module that each comprises at least:
a) a first and a second nucleic acid nucleic acid region that encodes a nucleic acid that is capable of directing RNA mediated gene regulation or RNA mediated gene editing;
b) a first, a second and a third nucleic acid nucleic acid region that encodes a nucleic acid that is capable of directing RNA mediated gene regulation or RNA mediated gene editing;
c) a first, a second, a third and a fourth nucleic acid region that encodes a nucleic acid that is capable of directing RNA mediated gene regulation or RNA mediated gene editing;
d) a first, a second, a third, a fourth and a fifth nucleic acid region that encodes a nucleic acid that is capable of directing RNA mediated gene regulation or RNA mediated gene editing;
e) a first, a second, a third, a fourth, a fifth and a sixth nucleic acid region that encodes a nucleic acid that is capable of directing RNA mediated gene regulation or RNA mediated gene editing;
f) a first, a second, a third, a fourth, a fifth, a sixth and a seventh nucleic acid region that encodes a nucleic acid that is capable of directing RNA mediated gene regulation or RNA mediated gene editing;
g) a first, a second, a third, a fourth, a fifth, a sixth, a seventh and an eighth nucleic acid region that encodes a nucleic acid that is capable of directing RNA mediated gene regulation or RNA mediated gene editing;
h) a first, a second, a third, a fourth, a fifth, a sixth, a seventh, an eighth and a ninth nucleic acid region that encodes a nucleic acid that is capable of directing RNA mediated gene regulation or RNA mediated gene editing; or
i) a first, a second, a third, a fourth, a fifth, a sixth, a seventh, an eighth, a ninth and a tenth nucleic acid region that encodes a nucleic acid that is capable of directing RNA mediated gene regulation or RNA mediated gene editing;
and:
   wherein the array operator is located upstream (5′) of the first nucleic acid sequence region that encodes a nucleic acid that is capable of directing RNA mediated gene regulation or RNA mediated gene editing within each array sub-module, so as to regulate transcription of each sub-module; and
   wherein the first array sub-module is located upstream (5′) to the second array sub-module so that the array operator of the second array sub-module is positioned 3′ to the final nucleic acid region that encodes a nucleic acid that is capable of directing RNA mediated gene regulation or RNA mediated gene editing of the first array sub-module; or
   wherein the array operator is located upstream (3′) of the last nucleic acid region that encodes a nucleic acid that is capable of directing RNA mediated gene regulation or RNA mediated gene editing within each array sub-module, so as to regulate transcription of each sub-module; and
   wherein the first array sub-module is located upstream (5′) to the second array sub-module so that the array operator of the first array sub-module is positioned 5′ to the first nucleic acid region that encodes a nucleic acid that is capable of directing RNA mediated gene regulation or RNA mediated gene editing of the second array sub-module.

14. The nucleic acid construct of any of the preceding paragraphs, wherein the promoter operator of a first sequence is capable of binding to a first activator protein and/or a first repressor protein.

15. The nucleic acid construct of any of the preceding paragraphs wherein the promoter operator of a first sequence is capable binding to a first activator protein in the presence of an inducing agent, optionally where the inducing agent is selected from the group comprising or consisting of: tetracycline (Tc); anhydrotetracycline (aTc); and Doxycycline; optionally
wherein the inducer molecule is anhydrotetracycline (aTc).

16. The nucleic acid construct of any of the preceding paragraphs wherein the promoter operator of a first sequence is capable of binding to a first repressor protein in the absence of an inducing agent, optionally where the inducing agent is selected from the group comprising or consisting of: tetracycline (Tc); anhydrotetracycline (aTc); and Doxycycline; optionally wherein the inducer molecule is anhydrotetracycline (aTc).

17. The nucleic acid construct of any of the preceding paragraphs wherein the promoter operator of a first sequence is capable of binding to a first activator protein in the presence of an inducing agent and wherein said promoter operator of a first sequence is capable of binding to a first repressor protein in the absence of same said inducing agent,
optionally where the inducing agent is selected from the group comprising or consisting of: tetracycline (Tc); anhydrotetracycline (aTc); and Doxycycline; optionally
wherein the inducer molecule is anhydrotetracycline (aTc).

18. The nucleic acid construct of any of the preceding paragraphs wherein the promoter operator of a first sequence is incapable of binding to a first repressor protein in the presence of an inducing agent, optionally where the inducing agent is selected from the group comprising or consisting of: tetracycline (Tc); anhydrotetracycline (aTc); and Doxycycline; optionally
wherein the inducer molecule is anhydrotetracycline (aTc).

19. The nucleic acid construct according to any of the preceding paragraphs wherein the array operator of a second sequence is capable of binding to a second repressor protein.

20. The nucleic acid construct of any of the preceding paragraphs wherein the array operator of a second sequence is capable of binding to a second repressor protein in the absence of an inducing agent,
optionally where the inducing agent is selected from the group comprising or consisting of: tetracycline (Tc); anhydrotetracycline (aTc); and Doxycycline; optionally
wherein the inducer molecule is anhydrotetracycline (aTc).

21. The nucleic acid construct of any of the preceding paragraphs wherein the array operator of a second sequence is incapable of binding to a protein in the presence of an inducing agent, optionally incapable of binding to the first activator protein, optionally is incapable of binding to the first activator protein in the presence of an inducing agent, optionally where the inducing agent is selected from the group comprising or consisting of: tetracycline (Tc); anhydrotetracycline (aTc); and Doxycycline; optionally
wherein the inducer molecule is anhydrotetracycline (aTc).

22. The nucleic acid of any of claims 2-21, wherein the promoter operator of a second sequence is capable of binding to a second repressor protein.

23. The nucleic acid construct of any of paragraphs 2-22, wherein the promoter operator of a second sequence is capable of binding to a second repressor protein in the absence of an inducing agent,
optionally where the inducing agent is selected from the group comprising or consisting of: tetracycline (Tc); anhydrotetracycline (aTc); and Doxycycline; optionally
wherein the inducer molecule is anhydrotetracycline (aTc).

24. The nucleic acid construct of any of paragraphs 2-23, wherein the promoter operator of a second sequence is incapable of binding to a protein in the presence of an inducing agent, optionally incapable of binding to the first activator protein, optionally is incapable of binding to the first activator protein in the presence of an inducing agent, optionally where the inducing agent is selected from the group comprising or consisting of: tetracycline (Tc); anhydrotetracycline (aTc); and Doxycycline; optionally
wherein the inducer molecule is anhydrotetracycline (aTc).

25. The nucleic acid construct of any of the preceding paragraphs wherein the first repressor protein and the second repressor protein are the same repressor protein.

26. The nucleic acid construct of any of the preceding paragraphs wherein the first repressor protein and the second repressor protein are different repressor proteins.

27. The nucleic acid construct according to any of the preceding paragraphs wherein the array operator of a second sequence is not capable of binding to an activator protein.

28. The nucleic acid construct of any of the preceding paragraphs, wherein the nucleic acid construct is a DNA construct.

29. The nucleic acid construct of any of the preceding paragraphs, wherein:
a) the promoter module is capable of initiating transcription of the gene-regulating and/or gene-editing array module in the presence of an inducing agent; and
b) the promoter module is not capable of initiating transcription of the gene-regulating and/or gene-editing array module in the absence of said inducing agent.

30. The nucleic acid construct of any of the preceding paragraphs wherein:
a) the promoter module is capable of initiating transcription of the gene-regulating and/or gene-editing array module in the absence of the first repressor protein and/or the second repressor protein; and/or
b) the promoter module is not capable of initiating transcription of the gene-regulating and/or gene-editing array module in the presence of the first repressor protein and/or the second repressor protein.

31. The nucleic acid construct of any of the preceding paragraphs wherein:
a) the promoter module is capable of initiating transcription of the gene-regulating and/or gene-editing array module when the first activator protein is present and the first repressor protein and/or the second repressor protein is absent; and/or
b) the promoter module is not capable of initiating transcription of the gene-regulating and/or gene-editing array module when the first activator protein is absent and the first and/or second repressor protein is present.

32. The nucleic acid construct according to any of the preceding paragraphs wherein in the absence of an inducing agent, the array operator(s) present in each array sub-module are occupied by a repressor protein,

33. The nucleic acid construct according to any of the preceding paragraphs, wherein in the absence of an inducing agent, the promoter operator(s) present in the promoter module are occupied by a repressor protein.

34. The nucleic acid construct of any of the preceding paragraphs, wherein the promoter operator of a first sequence is a TetO operator.

35. The nucleic acid construct of any of the preceding paragraphs, wherein the promoter operator of a first sequence is a TetO operator that has a sequence that has at least 80%, or optionally at least 85%, at least 90%, at least 91%, at least 92%, at least 93%, at least 94%, at least 95%, at least 96%, at least 97%, at least 98%, at least 99%, or 100% sequence identity to SEQ ID NO: 1; or wherein the first operator sequence has a sequence that is SEQ ID NO: 1.

36. The nucleic acid construct of any of the preceding paragraphs, wherein the array operator of a second sequence is a mutTetO operator sequence; optionally
wherein the array operator of a second sequence has is at least 80%, at least 85%, at least 90%, at least 91%, at least 92%, at least 93%, at least 94%, at least 95%, at least 96%, at least 97%, at least 98%, at least 99%, or more sequence identity to SEQ ID NO: 1; or
wherein the array operator of a second sequence has a sequence that is SEQ ID NO: 2.

37. The nucleic acid construct of any of paragraphs 2-36, wherein the promoter operator of a second sequence is a mutTetO operator sequence; optionally
wherein the promoter operator of a second sequence has at least 80%, at least 85%, at least 90%, at least 91%, at least 92%, at least 93%, at least 94%, at least 95%, at least 96%, at least 97%, at least 98%, at least 99%, or more sequence identity to SEQ ID NO: 2; or
wherein the promoter operator of a second sequence has a sequence that is SEQ ID NO: 2.

38. The nucleic acid construct of any of the preceding paragraphs, wherein the first activator protein is selected from the group comprising or consisting of: rtTA-VP and rtTA-Gal4.

39. The nucleic acid construct of any of the preceding paragraphs, wherein the first repressor protein is selected from the group comprising or consisting of: TetR-Mxi1.

40. The nucleic acid construct of any of the preceding paragraphs, wherein the second repressor protein is selected from the group comprising or consisting of: mutTetR-Mxi1.

41. The nucleic acid construct of any of the preceding paragraphs, wherein:
a) the first activator protein is rtTA-VP or rtTA-Gal4;
b) the first repressor protein is TetR-Mxi1; and
c) the second repressor protein is mutTetR-Mxi1.

42. The nucleic acid construct of any of the preceding paragraphs, wherein the at least one promoter of the promoter module is:
a) a Pol II promoter, optionally
   wherein the promoter is an inducible promoter
   wherein the Pol II promoter is classed as a strong promoter; and/or wherein the Pol II promoter is selected from the group consisting of a TDH3 promoter, a TEF1 promoter, a PGK1 promoter, a pCCW12 promoter, a pTEF2 promoter, a pHHF1 promoter, a pHHF2 promoter, a pALD6, promoter, a pGal1 promoter, a pPGK1 promoter, a pHTB2 promoter, a pCUP1 promoter, or a pTet promoter; or
b) a Pol III promoter, optionally
   wherein the Pol III promoter is classed as a strong Pol III promoter;
   wherein the Pol III promoter is an inducible promoter; and/or
   wherein the Pol III promoter is selected from the group consisting of the tRNA Phe promoter with a 5′ HDV ribozyme, the U6 promoter or H1 promoter.

44. The nucleic acid construct of any of the preceding paragraphs, wherein each nucleic acid that is capable of directing RNA mediated gene regulation or RNA mediated gene editing, when transcribed into RNA form, is independently capable of associating with a polypeptide, wherein said polypeptide is capable of regulating a gene, optionally wherein said polypeptide is selected from the group comprising or consisting of:
Cas9 or Cas9-like polypeptide; dCas9 or dCas9-like polypeptide; Cas12a; dCas12a; Cas12b; dCas12b; Cas13a; dCas13a; Cas13b; dCas13b; LbCpf1; dLbCpf1; AsCpf1; dAsCpf1; or dFnCpf1; or FnCpf1; or a fusion protein thereof.

45. The nucleic acid construct of any of the preceding paragraphs, wherein the nucleic acid region that encodes a nucleic acid that is capable of directing RNA mediated gene regulation or RNA mediated gene editing is complementary to a target nucleic acid region.

46. The nucleic acid construct of any of the preceding paragraphs, wherein each nucleic acid region that encodes a nucleic acid that is capable of directing RNA mediated gene regulation or RNA mediated gene editing, is complementary to the same target nucleic acid region, optionally wherein the target nucleic acid region is a promoter of a target gene.

47. The nucleic acid construct of any of the preceding paragraphs, wherein each nucleic acid region that encodes a nucleic acid that is capable of directing RNA mediated gene regulation or RNA mediated gene editing is complementary to the same target nucleic acid region, optionally wherein the target nucleic acid region is a promoter of a target gene,
but wherein the sequences of each nucleic acid region that encodes a nucleic acid that is capable of directing RNA mediated gene regulation or RNA mediated gene editing nucleic acid are different.

48. The nucleic acid construct of any of the preceding paragraphs, wherein within each array sub-module all of the nucleic acid regions that encode a nucleic acid that is capable of directing RNA mediated gene regulation or RNA mediated gene editing are each complementary to the same target nucleic acid region, optionally wherein the target nucleic acid region is a promoter of a target gene,
but wherein within each array sub-module the nucleic acid regions that encode a nucleic acid that is capable of directing RNA mediated gene regulation or RNA mediated gene editing are different.

49. The nucleic acid construct of any of the preceding paragraphs wherein:
each region that encodes a nucleic acid that is capable of directing RNA mediated gene regulation or RNA mediated gene editing, when transcribed into RNA form, are each independently capable of associating with a regulatory polypeptide, wherein said polypeptide is capable of regulating a gene, optionally wherein said polypeptide is selected from the group comprising or consisting of:
   Cas9 or Cas9-like polypeptide; dCas9 or dCas9-like polypeptide; Cas12a; dCas12a; Cas12b; dCas12b; Cas13a; dCas13a; Cas13b; dCas13b; LbCpf1; dLbCpf1; AsCpf1; dAsCpf1; or dFnCpf1; or FnCpf1; or a fusion protein thereof,
And wherein:
   a) the region(s) that encode a nucleic acid that is capable of directing RNA mediated gene regulation or RNA mediated gene editing, when transcribed into RNA form, are each independently capable of associating with the same regulatory polypeptide; or
   b) the region(s) that encode a nucleic acid that is capable of directing RNA mediated gene regulation or RNA mediated gene editing, when transcribed into RNA form, are each independently capable of associating with one of at least two different regulatory polypeptides.

50. The nucleic acid construct of any of the preceding paragraphs, wherein the cleavage site is selected from:
i) a transcriptionally inert site
ii) an endoribonuclease cleavage site, for example a site-specific RNA endonuclease site, for optionally a Csy4 cleavage sequence or an artificial site-specific RNA endonuclease
iii) a tRNA sequence
iv) a ribozyme sequence
v) an intron
vi) a target sequence for an RNA directed cleavage complex;
vii) a site cleavable by a Cas protein with RNA endonuclease activity, optionally cleavable by Cas12a.

51. The nucleic acid construct of any of the preceding paragraphs, wherein the cleavage site is a Csy4 cleavage sequence, optionally
Wherein the Csy4 cleavage site has a sequence that is at least 80%, at least 85%, at least 90%, at least 91%, at least 92%, at least 93%, at least 94%, at least 95%, at least 96%, at least 97%, at least 98%, at least 99%, or more sequence identity to SEQ ID NO: 18, SEQ ID NO: 19, SEQ ID NO: 20, and/or SEQ ID NO: 21. In some embodiments, the Csy4 cleavage site has a sequence that is SEQ ID NO: 18, SEQ ID NO: 19, SEQ ID NO: 20, or SEQ ID NO: 21.

52. The nucleic acid construct of any of the preceding paragraphs, wherein the nucleic acid construct further comprises a regulatory protein module, wherein the regulatory protein module comprises:
a) a first nucleotide region encoding a first regulatory polypeptide; and/or
b) a second nucleotide region encoding a second regulatory polypeptide;
optionally wherein the first regulatory polypeptide and the second regulatory polypeptide are selected from the group comprising or consisting of:
Cas9 or Cas9-like polypeptide; dCas9 or dCas9-like polypeptide; Cas12a; dCas12a; Cas12b; dCas12b; Cas13a; dCas13a; Cas13b; dCas13b; LbCpf1; dLbCpf1; AsCpf1; dAsCpf1; or dFnCpf1; or FnCpf1; or a fusion protein thereof, optionally Cas9-Mxi1, Cas9-like-Mxi1, dCas9-Mxi1, dCas9-like-Mxi1, Cas12a-Mxi1, dCas12a-Mxi1, Cas12b-Mxi1, dCas12b-Mxi1, Cas13a-Mxi1, dCas13a-Mxi1, Cas13b-Mxi1, dCas13b-Mxi1, LbCpf1-Mxi1, dLbCpf1-Mxi1, AsCpf1-Mxi1, dAsCpf1-Mxi1, dFnCpf1-Mxi1, FnCpf1-Mxi1, Cas9-VP, Cas9-like-VP polypeptide, dCas9-VP, dCas9-like-VP polypeptide, Cas12a-VP, dCas12a-VP, Cas12b-VP, dCas12b-VP, Cas13a-VP, dCas13a-VP, Cas13b-VP, dCas13b-VP, LbCpf1-VP, dLbCpf1-VP, AsCpf1-VP, dAsCpf1-VP, dFnCpf1-VP, or FnCpf1-VP.

53. The nucleic acid construct of paragraph 52, wherein the regulatory polypeptide capable of regulating a gene, the first regulatory polypeptide and/or the second regulatory polypeptide is fused to an activator domain and/or a repressor domain; optionally
wherein the activator domain is selected from the group comprising or consisting of: VP, VP16, VP64, GAL4 and B42; and/or
wherein the repressor domain is selected from the group comprising or consisting of: KRAB-like effectors (optionally Mxi1), RD1152, RD11, RD5, and/or RD2.

54. The nucleic acid construct of paragraph 52 or 53, wherein the first regulatory polypeptide and/or the second regulatory polypeptide are each selected from the group comprising or consisting of:
a Cas9-Mxi1 or Cas9-like-Mxi1 polypeptide; a dCas9-Mxi1 or dCas9-like-Mxi1 polypeptide; Cas12a-Mxi1; dCas12a-Mxi1; Cas12b-Mxi1; dCas12b-Mxi1; Cas13a-Mxi1; dCas13a-Mxi1; Cas13b-Mxi1; dCas13b-Mxi1; LbCpf1-Mxi1; dLbCpf1-Mxi1; AsCpf1-Mxi1; dAsCpf1-Mxi1; dFnCpf1-Mxi1; or FnCpf1-Mxi1; and/or
a Cas9-VP or Cas9-like-VP polypeptide; a dCas9-VP or dCas9-like-VP polypeptide; Cas12a-VP; dCas12a-VP; Cas12b-VP; dCas12b-VP; Cas13a-VP; dCas13a-VP; Cas13b-VP; dCas13b-VP; LbCpf1-VP; dLbCpf1-VP; AsCpf1-VP; dAsCpf1-VP; dFnCpf1-VP; or FnCpf1-VP.

55. The nucleic acid construct of any of paragraphs 52-54, wherein:
a) the first regulatory polypeptide is selected from the group comprising or consisting of a Cas9-Mxi1 or Cas9-like-Mxi1 polypeptide; a dCas9-Mxi1 or dCas9-like-Mxi1 polypeptide; Cas12a-Mxi1; dCas12a-Mxi1; Cas12b-Mxi1; dCas12b-Mxi1; Cas13a-Mxi1; dCas13a-Mxi1; Cas13b-Mxi1; dCas13b-Mxi1; LbCpf1-Mxi1; dLbCpf1-Mxi1; AsCpf1-Mxi1; dAsCpf1-Mxi1; dFnCpf1-Mxi1; or FnCpf1-Mxi1, optionally is a dCas9-Mxi1 polypeptide; and/or
b) the second regulatory polypeptide is selected from the group comprising or consisting of a Cas9-VP or Cas9-like-VP polypeptide; a dCas9-VP or dCas9-like-VP polypeptide; Cas12a-VP; dCas12a-VP; Cas12b-VP; dCas12b-VP; Cas13a-VP; dCas13a-VP; Cas13b-VP; dCas13b-VP; LbCpf1-VP; dLbCpf1-VP; AsCpf1-VP; dAsCpf1-VP; dFnCpf1-VP; or FnCpf1-VP, optionally is a dCas12a-VP polypeptide.

56. The nucleic acid construct of any of paragraphs 52-55, wherein:
a) the first regulatory polypeptide is encoded by a sequence that:
   is at least 80%, at least 85%, at least 90%, at least 91%, at least 92%, at least 93%, at least 94%, at least 95%, at least 96%, at least 97%, at least 98%, at least 99%, or more identical to SEQ ID NO: 37; or
   is 80%, 85%, 90%, 91%, 92%, 93%, 94%, 95%, 96%, 99%, 100% identical to SEQ ID NO: 37; or
   is SEQ ID NO: 37; and/or
b) the second regulatory polypeptide is encoded by a sequence that:
   is at least 80%, at least 85%, at least 90%, at least 91%, at least 92%, at least 93%, at least 94%, at least 95%, at least 96%, at least 97%, at least 98%, at least 99%, or more identical to SEQ ID NO: 38; or
   is 80%, 85%, 90%, 91%, 92%, 93%, 94%, 95%, 96%, 99%, 100% identical to SEQ ID NO: 38; or
   is SEQ ID NO: 38.

57. The nucleic acid construct of any of the preceding paragraphs, further comprising a nucleic acid region encoding a polypeptide that is capable of cleaving the cleavage site present in the array module when in RNA form; optionally wherein the polypeptide that is capable of cleaving the cleavage site when in RNA form is:
i) an endoribonuclease, for example a site-specific RNA endonuclease, for example a Csy4 or an artificial site-specific RNA endonuclease;
ii) a polypeptide capable of cleaving a tRNA sequence
iii) a polypeptide capable of cleaving an intron sequence; or
v) polypeptide capable of cleaving a target sequence for an RNA directed cleavage complex; or
vi) a Cas protein with RNA endonuclease activity, optionally cleavable by Cas12a.

58. The nucleic acid construct of paragraph 57, wherein the polypeptide that is capable of cleaving the cleavage site when in RNA form is Csy4, optionally
wherein the polypeptide that is capable of cleaving the cleavage site when in RNA form is encoded by a sequence that is at least 80%, at least 85%, at least 90%, at least 91%, at least 92%, at least 93%, at least 94%, at least 95%, at least 96%, at least 97%, at least 98%, at least 99%, or more identical to SEQ ID NO: 39; or
is 80%, 85%, 90%, 91%, 92%, 93%, 94%, 95%, 96%, 99%, 100% identical to SEQ ID NO: 39; or
is SEQ ID NO: 39.

59. The nucleic acid construct of any of paragraphs 52-58, wherein:
a) the first nucleotide region encoding a first regulatory polypeptide is operably linked to a promoter region;
b) the second nucleotide region encoding a second regulatory polypeptide is operably linked to a promoter region; and
c) the nucleic acid region encoding a polypeptide that is capable of cleaving the cleavage site present in the array module when in RNA form is operably linked to a promoter region;
optionally where the promoter region of (a), (b) and (c) are different promoters.

60. The nucleic acid construct of paragraph 59, wherein the promoter region of (a), (b) and/or (c) is:
a weak promoter or a medium-strength promoter; optionally
wherein the promoter is selected from the group comprising or consisting of: REV1, PSP2, HTB2, RAD27, or POP6.

61. The nucleic acid construct of any of the preceding paragraphs, further comprising:
i) a nucleic acid sequence encoding a first activator protein;
ii) a nucleic acid sequence encoding a first repressor protein; and/or
iii) a nucleic acid sequence encoding a second repressor protein,
   optionally wherein the nucleic acid sequence encoding the first activator protein; the nucleic acid sequence encoding the first repressor protein; and/or the nucleic acid sequence encoding the second repressor protein are each independently operably linked to a promoter sequence.

62. The nucleic acid construct of paragraph 61, wherein the promoter region of (i), (ii) and/or (iii) sequence is:
a weak promoter or a medium-strength promoter; optionally
wherein the promoter is selected from the group comprising or consisting of: REV1, PSP2, HTB2, RAD27, or POP6.

63. The nucleic acid construct of any of paragraphs 61 or 62, wherein:
a) the first activator protein is selected from the group comprising or consisting of: rtTA-VP and rtTA-Gal4; optionally
   wherein the first activator protein is rtTA-Gal4;
b) the first repressor protein is selected from the group comprising or consisting of: TetR-Mxi1; optionally
   wherein the first repressor protein is TetR-Mxi1; and/or
c) the second repressor protein is selected from the group comprising or consisting of: mutTetR-Mxi1; optionally
   wherein the first repressor protein is mutTetR-Mxi1.

64. The nucleic acid construct of any of the preceding paragraphs, wherein the first regulatory polypeptide and the second regulatory polypeptide are each separately capable of directing RNA mediated gene regulation are capable of:
a) activating a gene; and/or
b) repressing a gene.

65. The nucleic acid construct according to any of the preceding paragraphs wherein the nucleic acid construct is a circular nucleic acid construct.

66 The nucleic acid construct according to any of the preceding paragraphs wherein the nucleic acid construct is a linear nucleic acid construct.

67. The nucleic acid construct according to any of the preceding paragraphs wherein the nucleic acid construct comprises at least one, optionally two regions of homology to a target locus in a target genome, arranged so as to allow homologous recombination to occur between the regions of homology in the nucleic acid construct and the corresponding regions of homology in the target genome so as to result in incorporation of the nucleic acid construct into the target genome.

68. A vector comprising the nucleic acid construct of any of the preceding paragraphs.

69. The vector according to paragraph 68, wherein the vector is selected from the group comprising or consisting of: a plasmid, an artificial chromosome, a bacterial artificial chromosome, a yeast artificial chromosome, a human artificial chromosome, a viral vector, a retroviral vector, a lentiviral vector, an adenoviral vector, an adeno-associated viral vector, a bacteriophage vector, a hybrid viral vector, or any combination thereof.

70. A single polycistronic nucleic acid transcript transcribed from the promoter module of the nucleic acid construct according to any of paragraphs 1-67 or vector according to any of paragraphs 68 or 69.

71. A single polycistronic nucleic acid transcript comprising at least 2, optionally at least 3, 4, 5, 6, 7, 8, 9, or at least 10 nucleic acid sequences that are each separately capable of directing RNA mediated gene regulation or RNA mediated gene editing, and wherein between each nucleic acid sequence that directs RNA mediated gene regulation or RNA mediated gene editing is a sequence that is a cleavage site, and wherein the single polycistronic nucleic acid transcript comprises a region transcribed from an operator sequence.

72. A cell comprising the nucleic acid construct according to any of paragraphs 1-67; the vector according to either of paragraphs 68 or 69; the single polycistronic nucleic acid transcript according to paragraph 71.

73. The cell according to paragraph 72, wherein the cell is a eukaryotic cell, optionally selected from a fungal cell; a plant cell; and an animal cell, optionally wherein the animal cell is a mammalian cell.

74. The cell according to either of paragraphs 72 or 73, wherein the cell is a fungal cell, optionally is a fungal cell belonging to a genus selected from the group comprising or consisting of: Candida, Hansenula, Komagatella, Pichia, Ashbya, Blastobotrys, Cryptococcus, Cutaneotrichosporon, Dekkera, Kluveromyces, Rhodosporidium, Rhodotorula, Lipomyces, Saccharomyces, and Yarrowia.

75. The cell according to any of paragraphs 72-74, wherein the cell is a Saccharomyces cell; optionally
wherein the cell is a Saccharomyces cerevisiae cell.

76. The cell according to paragraph 72 wherein the cell is a prokaryotic cell, optionally is a bacterial cell, optionally is a bacterial cell belonging to a genus selected from the group comprising or consisting of: Escherichia, Pseudomonas, Vibrio, Bacillus, Clostridium, Lactobacillus, Lactococcus, Streptomyces.

77. The cell according to any of paragraphs 72-76, wherein the nucleic acid construct or the vector:
a) is integrated into one or more chromosomes of the cell; or
b) is maintained episomally.

78. The cell according to any of paragraphs 72-77, wherein the cell comprises polypeptides and/or nucleic acids that are capable of transcribing the single polycistronic nucleic acid transcript from the nucleic acid construct or the vector.

79. The cell according to any of paragraphs 72-78, wherein the cell comprises a target nucleic acid region and wherein the at least first nucleic acid region that encodes a nucleic acid that is capable of directing RNA mediated gene regulation or RNA mediated gene editing is complementary to the target nucleic acid region, optionally wherein the target nucleic acid region is a promoter of a target gene.

80. The cell according to any of paragraphs 72-79 wherein the cell comprises
a) between 2 and 100 target nucleic acid regions; optionally between 5 and 95, 10 and 90, 15 and 85, 20 and 80, 25 and 75, 30 and 70, 35 and 65, 40 and 60, 45 and 55 target nucleic acid regions; and/or
b) at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 77, at least 76, at least 77, at least 78, at least 79, at least 80, at least 81, at least 82, at least 83, at least 84, at least 88, at least 86, at least 87, at least 88, at least 89, at least 90, at least 91, at least 92, at least 93, at least 94, at least 99, at least 96, at least 97, at least 98, at least 99, or more target nucleic acid regions; optionally comprises 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 66, 66, 67, 68, 69, 70, 71, 72, 73, 74, 77, 76, 77, 78, 79, 80, 81, 82, 83, 84, 88, 86, 87, 88, 89, 90, 91, 92, 93, 94, 99, 96, 97, 98, 99, or 100 target nucleic acid regions,
   and wherein the nucleic acid construct or vector comprises a gene-regulating and/or gene-editing array module that comprises
i) between 2 and 100 nucleic acid regions that each encode a nucleic acid that is capable of directing RNA mediated gene regulation or RNA mediated gene editing that is complementary to a target nucleic acid region in the cell; optionally between 5 and 95, 10 and 90, 15 and 85, 20 and 80, 25 and 75, 30 and 70, 35 and 65, 40 and 60, 45 and 55 nucleic acid regions that each encode a nucleic acid that is capable of directing RNA mediated gene regulation or RNA mediated gene editing that is complementary to a target nucleic acid region in the cell;
ii) at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 77, at least 76, at least 77, at least 78, at least 79, at least 80, at least 81, at least 82, at least 83, at least 84, at least 88, at least 86, at least 87, at least 88, at least 89, at least 90, at least 91, at least 92, at least 93, at least 94, at least 99, at least 96, at least 97, at least 98, at least 99, or more target nucleic acid regions; optionally comprises 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 66, 66, 67, 68, 69, 70, 71, 72, 73, 74, 77, 76, 77, 78, 79, 80, 81, 82, 83, 84, 88, 86, 87, 88, 89, 90, 91, 92, 93, 94, 99, 96, 97, 98, 99, or 100 nucleic acid regions that each encode a nucleic acid that is capable of directing RNA mediated gene regulation or RNA mediated gene editing that is complementary to a target nucleic acid region in the cell.

81. The cell of any of paragraphs 72-80, wherein the cell constitutively expresses:
a) the first and/or second regulatory polypeptide; and/or
b) a polypeptide that is capable of cleaving the nucleic acid construct at the cleavage site when in RNA form.

82. A method of RNA mediated gene regulation of at least one target gene, the method comprising:
a) contacting the cell according to any of paragraphs 72-81 with an inducer molecule; and
b) maintaining the cell in culture conditions suitable for the expression of the array module.

83. The method of paragraph 82 wherein the method comprises contacting the cell with a nuclease enzyme capable of cleaving the cleavage site when in RNA form.

84. The method of paragraph 83 wherein said contacting is performed by expressing said nuclease enzyme within the cell.

85. The method according to any of 82-84, wherein the inducer molecule is selected from the group comprising or consisting of: tetracycline (Tc); anhydrotetracycline (aTc); Doxycycline; optionally wherein the inducer molecule is anhydrotetracycline (aTc).

86. The method according to either of paragraphs 82-84 wherein the array module;
a) is not expressed in the absence of the inducer molecule;
b) is expressed only in the presence of the inducer molecule; and/or
c) has increased expression levels in the presence of the inducer molecule compared to the level of expression of the array module in the absence of the inducer molecule; optionally
   wherein expression of the array module increases by at least 100%, at least 150%, at least 200%, at least 250%, at least 300%, at least 350%, at least 400%, at least 450%, at least 500%, at least 550%, at least 600%, at least 650%, at least 700%, at least 750%, at least 800%, at least 850%, at least 900%, at least 950%, at least 1000%, at least 1500%, at least 2000%, at least 2500%, at least 3000%, at least 3500%, at least 4000%, at least 4500%, at least 5000%, at least 5500%, at least 6000%, at least 6500%, at least 7000%, at least 7500%, at least 8000%, at least 8500%, at least 9000%, at least 10,000%, or more in the presence of the inducer molecule compared to the expression of the array module in the absence of the inducer molecule; optionally
   wherein expression of the array module increases by 100%, 150%, 200%, 250%, 300%, 350%, 400%, 450%, 500%, 550%, 600%, 650%, 700%, 750%, 800%, 850%, 900%, 950%, 1000%, 1500%, 2000%, 2500%, 3000%, 3500%, 4000%, 4500%, 5000%, 5500%, 6000%, 6500%, 7000%, 7500%, 8000%, 8500%, 9000%, or 10,000% in the presence of the inducer molecule compared to the expression of the target gene in the absence of the inducer molecule.

87. Use of the method according to any of paragraphs 82-84 in a process of producing at least one organic molecule.

88. The use of paragraph 87, wherein the organic molecule is selected from the group comprising or consisting of: a metabolite; a secondary metabolite; a fatty acid; a fat; an oligosaccharide; a polysaccharide; a monosaccharide; a nucleic acid; a polypeptide; or any combination thereof,
optionally wherein the metabolite is selected from the group comprising or consisting of flavonoids, terpenoids and polyketides.

89. A kit comprising the nucleic acid construct according to any of paragraphs 1-67; the vector according to either of paragraphs 68 or 69; the single polycistronic nucleic acid transcript according to any of paragraphs 70-71; or cell according to any of paragraphs 72-81.

90. A nucleic acid construct; the vector; single polycistronic nucleic acid transcript; cell; method; use; or kit as described herein.

91. A nucleic acid construct comprising:
a gene-regulating and/or gene-editing array module, that comprises at least one array sub-module, wherein each array sub-module comprises at least a first nucleic acid region that encodes a nucleic acid that is capable of directing RNA mediated gene regulation or RNA mediated gene editing,
wherein the gene-regulating and/or gene-editing array module is capable of being transcribed into a single polycistronic nucleic acid transcript from a single promoter,
and wherein between each nucleic acid region that encodes a nucleic acid that is capable of directing RNA mediated gene regulation or RNA mediated gene editing is a sequence that when in RNA form is an RNA cleavage site; and
wherein each array sub-module comprises at least one array operator of a second sequence
   and wherein the gene-regulating and/or gene-editing array module is operably linked to promoter module.

92. A nucleic acid construct comprising:
a) a promoter module wherein the promoter module comprises at least one promoter operator of a first sequence; and
b) a gene-regulating and/or gene-editing array module, that comprises at least one array sub-module, wherein each array sub-module comprises at least a first nucleic acid region that encodes a nucleic acid that is capable of directing RNA mediated gene regulation or RNA mediated gene editing and a second nucleic acid region that encodes a nucleic acid that is capable of directing RNA mediated gene regulation or RNA mediated gene editing,
   wherein the gene-regulating and/or gene-editing array module is capable of being transcribed into a single polycistronic nucleic acid transcript from a single promoter,
   and wherein between each nucleic acid region that encodes a nucleic acid that is capable of directing RNA mediated gene regulation or RNA mediated gene editing is a sequence that when in RNA form is an RNA cleavage site; and
   wherein each array sub-module comprises at least one array operator of a second sequence
and wherein the gene-regulating and/or gene-editing array module is operably linked to promoter module.

93. A nucleic acid construct comprising:
a) a promoter module wherein the promoter module comprises at least one promoter operator of a first sequence; and
b) a gene-regulating and/or gene-editing array module, that comprises at least two array sub-modules, wherein each array sub-module comprises at least a first nucleic acid region that encodes a nucleic acid that is capable of directing RNA mediated gene regulation or RNA mediated gene editing and a second nucleic acid region that encodes a nucleic acid that is capable of directing RNA mediated gene regulation or RNA mediated gene editing,
   wherein the gene-regulating and/or gene-editing array module is capable of being transcribed into a single polycistronic nucleic acid transcript from a single promoter,
   and wherein between each nucleic acid region that encodes a nucleic acid that is capable of directing RNA mediated gene regulation or RNA mediated gene editing is a sequence that when in RNA form is an RNA cleavage site; and
   wherein each array sub-module comprises at least one array operator of a second sequence
and wherein the gene-regulating and/or gene-editing array module is operably linked to promoter module.

### BRIEF DESCRIPTION OF THE DRAWINGS

FIG. 1—Development of inducible gRNA arrays, a) Development of a low-leak, aTc inducible promoter, rtTA-Gal4 targeting 7×TetO sites upstream of a core promoter library driving the expression of mGFPmut2. Addition of 1 μM aTc recruits rtTA-Gal4 to the promoter, upregulating the expression of mGFPmut2. The PHOS minimal core promoter (pPHO5m) exhibited the lowest level of basal activity in the absence of inducer, and was used in the initial inducible gRNA array, Design 1. b) Development of a leak-free, aTc inducible promoter. The aTc-repressible TetR protein fused to the strong transcriptional repressor Mxi1 (TetR-Mxi1) was introduced alongside rtTA-Gal4 to bind TetO sites in the absence of inducer and repress low levels of basal transcription in the off state37, reducing all promoters to undetectable levels of mGFPmut2 fluorescence in the absence of inducer. The low-leak, 7×TetO PHO5m promoter was again chosen to build the second iteration of the inducible gRNA array, Design 2. c) Csy4 processed gRNA arrays driven by the various expression systems. Arrays are composed of 18 gRNAs designed to target the constitutive yeast ALD6, TEF1, and HHF1 promoters driving the expression of mScarlet-I, mGFPmut2, and mTagBFP2, respectively, for repression by dCas9-Mxi1. d) Fluorescence measurements of mScarlet-I, mGFPmut2, and mTagBFP2 in the presence and absence of 1 μM aTc across the various gRNA array expression systems, normalised to a no gRNA and a no fluorescent protein control. Experimental measurements are fluorescence levels per cell determined by flow cytometry and shown as the mean±SD from quadruplicate isolates

FIG. 2—CRISPR protein expression optimisation and inducible CRISPRai toolkit architecture. a+b) Impact of CRISPRai protein expression on gene activation and repression. a) Low (small circle) and medium (large circle) strengths promoter combinations used to drive the expression of dCas12a-VP (dark blue), dCas9-Mxi1 (blue), and Csy4 (light blue). The constitutive TDH3 promoter is used drive the transcription of an array containing an activation (dCas12a-VP) and repression (dCas9-Mxi1) gRNA targeting the RNR2 and TEF1 promoters driving the expression of mRuby2 and Venus, respectively. b) Fluorescence measurements of all mid and low strength promoter-CRISPR protein combinations normalised to a control with no proteins (No CRISPR). Experimental measurements are mRuby2 and Venus fluorescence levels per cell determined by flow cytometry and shown as the mean±SD from triplicate isolates. c+d) Impact of CRISPRai protein expression on growth. c) Low and medium strengths promoter combinations used to drive the expression of the CRISPR proteins. d) Maximum growth rates of all low and medium strength promoter combinations driving the expression of the CRISPR proteins and compared to a control with no proteins (No CRISPR). Results are calculated maximum growth rates in YPD medium as determined from growth curves in a plate reader at OD₆₀₀ and are shown as the mean±SD from quadruplicate isolates. e) Inducible CRISPRai vector architecture and gRNA array assembly. Inducible CRISPRai vector backbone (KanR-ColE1) not shown. f) PCR generation of gRNA fragments for scarless BsaI Golden Gate assembly of gRNA arrays.

FIG. 3—Inducible CRISPRai for metabolic engineering. a) Inducible CRISPRai gRNA array containing three activation gRNAs targeting the RNR2 promoter (red) followed by three repression gRNAs targeting the TEF1 promoter (blue), Expression and Csy4 processing of the gRNA array results in the upregulation of mScarlet-I, through the recruitment of dCas12a-VP to the RNR2 promoter, and down regulation of mTagBFP2 through recruitment of dCas9-Mxi1 to the TEF1 promoter. b) Time course of mScarlet-I and mTagBFP2 fluorescence after 1 μM aTc induction at 0 h. Experimental measurements are mScarlet-I and mTagBFP2 fluorescence levels per cell determined by flow cytometry and shown as individual values from triplicate isolates. c) Overview of succinic acid production and CRISPRai targets. CRISPRa and CRISPRi targets in green and red, respectively. d) CRISPRai construct integrated at HO locus and gRNA arrangement in inducible array. e) Quantification of succinic acid from WT and inducible CRISPRai yeast with untargeted gRNAs (Untargeted) and gRNAs targeting the 11 genes highlighted in A (Targeted). Experimental measurements are succinic acid concentrations from triplicate biological samples as determined by LC-MS.

FIG. 4—Limits of gRNA array silencing from mutTetR in the uninduced state. a) mutTetR silencing of the array with mutTetO sites either side of groups of 1-8 gRNAs targeting the ALD6, TEF1, and HHF1 promoters driving the expression of mScarlet-I, mGFPmut2, mTagBFP2, respectively. Expression of the array followed by Csy4 processing and dCas9-Mxi1 mediated repression of the target promoters. b, Fluorescence measurements of the inducible gRNA systems combinatorially repressing pALD6-mScarlet-I, pTEF1-mGFPmut2, and pHHF1-mTagBFP2, in the presence and absence of 1 UM aTc, normalised to a no gRNA and a no fluorescent protein control. Experimental measurements are mScarlet-I, mGFPmut2, and mTagBFP2 fluorescence levels per cell, as determined by flow cytometry, and shown as the mean±SD from quadruplicate isolates. c, Transformation of 6× Constitutive array (left) vs 6× Design 3 array (right) without inducer. Larger colonies in the Constitutive condition are escape mutants that have deleted various regions of the gRNA array, as confirmed by colony PCR.

FIG. 5—Stability of CRISPRai in batch culture. Histograms of mScarlet-I (Top) and mTagBFP2 (Bottom) fluorescence data from FIG. 2c. Saturated overnight cultures were diluted 1:100 into fresh SD media with 1 μM aTc at 0 h and growth at 30° C. shaking. Cells were sampled at 24 h increments with no further changes to initial conditions. After 24 h, mScarlet-I and mTagBFP2 had reached maximum and minimum fluorescence levels, respectively. Cell fluorescence levels were maintained over the course of 6 days measurements were collected, with cell populations remaining as a single peak, suggesting no observable CRISPRai escape mutants,

FIG. 6—Stability of CRISPRai over 1 week of daily cell passaging. a) Early induction of CRISPRai. Saturated overnight cultures were diluted 1:100 into fresh SD media with 1 μM aTc at 0 h and growth at 30° C. shaking. Cells were sampled and back diluted 1:100 into fresh SD media with 1 μM aTc every 24 hours. Continually active transcription of the gRNA array starting at 0 h resulted in emergence of CRISPRai escape mutants (red box) comprising various deletions of the gRNA array, as confirmed by colony PCR (data not shown). b) Late induction of CRISPRai. Saturated overnight cultures were diluted 1:100 into fresh SD media without aTc and growth at 30° C. shaking. Cells were sampled and back diluted 1:100 into fresh SD media without aTc every 24 hours. At 144 h, cells were back diluted 1:100 into fresh SD media with 1 μM aTc and the final measurement was taken 24 h later. CRISPRai regulation of fluorescence performed as expected after late induction in 6 days of continuous culture, suggesting the gRNA array is stable when uninduced. Leftward drift seen in mScarlet-I fluorescence due to day variance in flow cytometer.

FIG. 7—gRNA array fragment generation and assembly. a) Example PCR of gRNA fragments for of 2×gRNA array containing an activation (dCas12a-VP) and repression (dCas9-Mxi1). Activation gRNAs are designed to include the entire gRNA fragment sequence (Cas12a handle, 20 bp target sequence, Csy4 site, and flanking BsaI cloning sequences) within the primers. The primers are designed to anneal to each other at the Csy4 site and use 5 rounds of PCR extension, without a template, to complete the full dsDNA gRNA fragment. Repression gRNAs are designed to include the 20 bp target sequence, flanking BsaI cloning sequences, Cas9 handle, and Csy4 site. The primers are designed to amplify from the pWS3799 Cas9 gRNA-Csy4 template to create the full dsDNA gRNA fragment after 30 cycles of PCR. The BsaI generated overhangs in the CRISPRai vector and sub-array plasmid are within the last (GCAG) and first (TCCC) 4 bp of the Csy4 and mutTetO sites, respectively. By designing the BsaI-generated overhangs between gRNAs to occur within the flanking sequence of the gRNA fragment, such as the 20 bp targeting sequence, gRNAs within the array can be assembled scarlessly to be precisely flanked by Csy4 sites. This can be designed automatically using the Benchling Golden Gate assembly tool, b) gRNA fragments are purified and included in a BsaI Golden Gate assembly reaction with the CRISPRai vector or sub-array plasmid. Completed reactions are transformed into E. coli and initially screened for the absence of green fluorescence on a blue light box (assembled gRNA arrays replace the E. coli GFP expression cassette dropout in the backbone). Correct array identity is then confirmed by colony PCR and Sanger sequencing across the entire array. c) Assembled gRNA arrays in the CRISPRai vector and sub-array plasmid. Fully assembled arrays in the CRISPRai vector position mutTetO sites either side the gRNA cluster for efficient silencing. Subarrays position a single mutTetO site downstream of the sub-array. Assembly of sub-arrays into the CRISPRai vector results in all sub-arrays flanked by mutTetO sites for efficient silencing (FIG. 8). Plasmid backbones not shown.

FIG. 8—Assembly of sub-arrays and spacers into CRISPRai vector. Example assembly of 16×gRNA array from 3 pre-assembled sub-arrays and a 50 bp spacer into a CRISPRai vector by BsmBI Golden Gate assembly. a) All assemblies from sub-arrays and spacers comprise 4 sub-array/spacers with the appropriate BsmBI overhangs (¼, 2/4, ¾, and 4/4). Sub-arrays and spacers are flanked by pre-defined BsmBI-generated overhangs that organize their position within the array during the BsmBI Golden Gate assembly. b) BsmBI assembly of 3 sub-arrays and spacer into the CRISPRai vector. As with gRNA fragment assembly, completed reactions are transformed into E. coli and screened for the absence of green fluorescence on a blue light. Correct array identity is confirmed by colony PCR or restriction digest using unique restriction enzyme sites either side of the entire array (Left; EcoRI/XbaI, Right; SpeI/PstI). c) Fully assembled arrays position mutTetO sites either side of each sub-array. Efficient silencing is seen up to 6 gRNAs in each sub-array. Five 4 bp BsmBI cloning scars result from sub-array assembly (blue). However, these are positioned outside the Csy4 sites and so are not included in the mature gRNA, keeping the Csy4 processed gRNAs within the array free of additional RNA sequence (excluding the cleaved Csy4 sequence). Spacers are 50 bp of biologically neutral DNA designed by R20DNA designer²³.

FIG. 9—Inducible CRISPRai toolkit plasmids

FIG. 10—exemplary gRNA targets disclosed herein. Repression (dCas9-Mxi1) and activation (dCpf1-VP) targets are in red and green, respectively.

FIG. 11—Annotated sequence of an exemplary vector according to the Invention, pWS4033 (SEQ ID NO: 40) Exemplary vector comprises a superfolder green fluorescent protein (sfGFP) coding sequence in place of the gene-regulating and/or gene-editing array module of the invention, which may be incorporated from SEQ ID NO: 41.

FIG. 12—Plasmid map of an exemplary vector according to the invention, pWS4033

FIG. 13—Annotated sequence of an exemplary nucleic acid construct according to the invention (SEQ ID NO: 41)

FIG. 14—Map of an exemplary nucleic acid construct according to the invention

FIG. 15—Annotated sequence of an exemplary inducible array (SEQ ID NO: 42)

FIG. 16—Map of an exemplary inducible array

FIG. 17—Exemplary nucleic acid construct according to the invention

### EXAMPLES

### Example I—Methods

### Inducible CRISPRai Toolkit

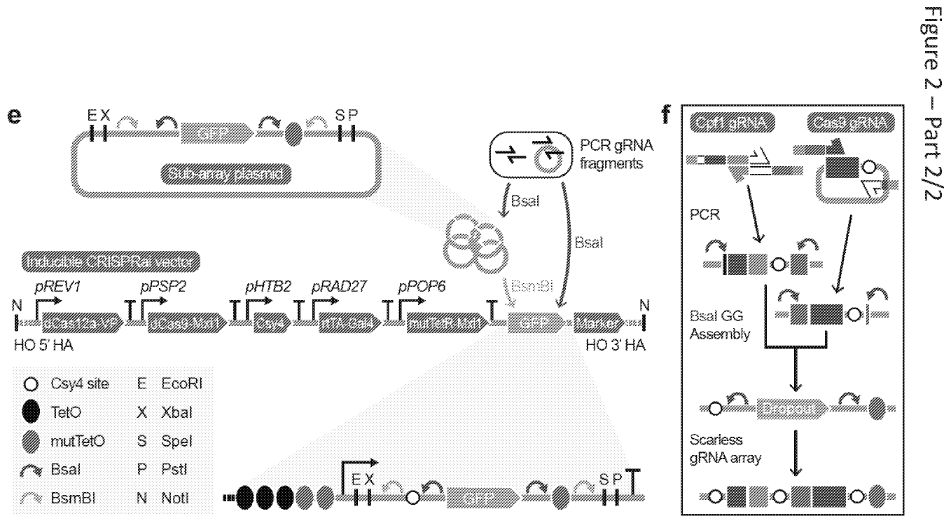

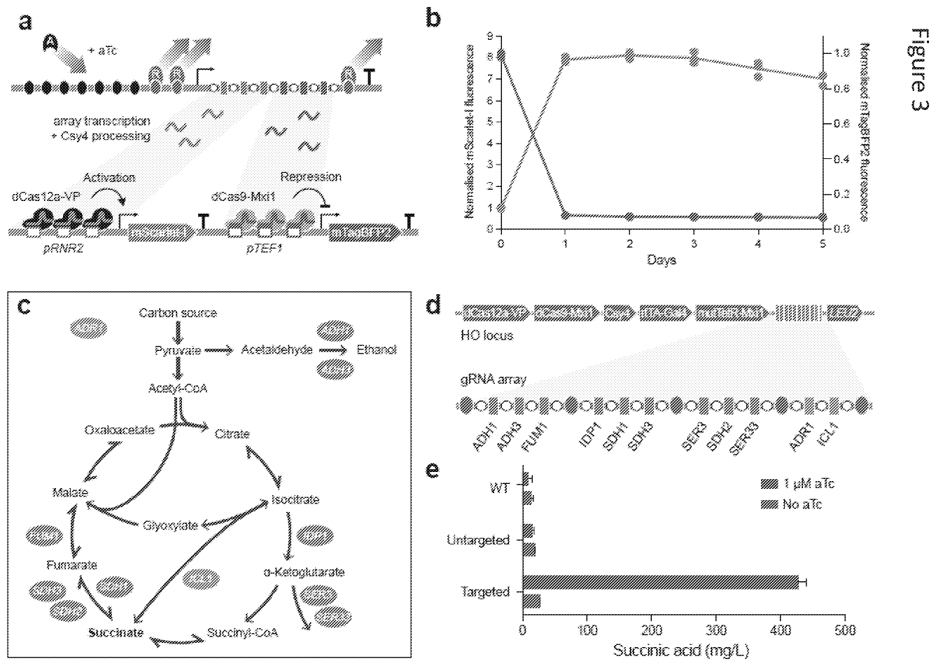

Toolkit overview. The inducible CRISPRai toolkit consists of an all-in-one genomic integration vector containing the full set of proteins required for inducible CRISPRai and a GFP dropout in place of the gRNA array (FIG. 2a). gRNA arrays are cloned into the vector using PCR generated fragments that are assembled directly into the vector for up to 6 gRNAs in a single round of Golden gate assembly (FIG. 7), or up to 24 gRNAs via four intermediate subarray plasmids in two rounds of golden gate assembly (FIG. 8). mutTetO sites are included within the inducible CRISPRai vector and subarray plasmids so that they are distributed throughout the array, and spacers are included in instances where not all 4 subarray vectors are required. The limit of 6 gRNAs per CRISPRai vector or sub-array (24 gRNAs when sub-arrays are added together) is recommended to ensure a tight off state by keeping the distribution of mutTetO sites within the limits of mutTetR silencing.

This also simplifies validation of array identity by Sanger sequencing. The inducible CRISPRai vector has been designed to integrate at the HO locus, which is conserved between common lab strains, and is available with 6 auxotrophic and 4 antibiotic selectable markers (URA3, LEU2, HIS3, TRP1, LYS2, 254 MET17, KanR, NatR, HygR, and ZeoR), and so should be appropriate for most strains and applications. For a full list of plasmids in the inducible CRISPRai toolkit, see FIG. 9.

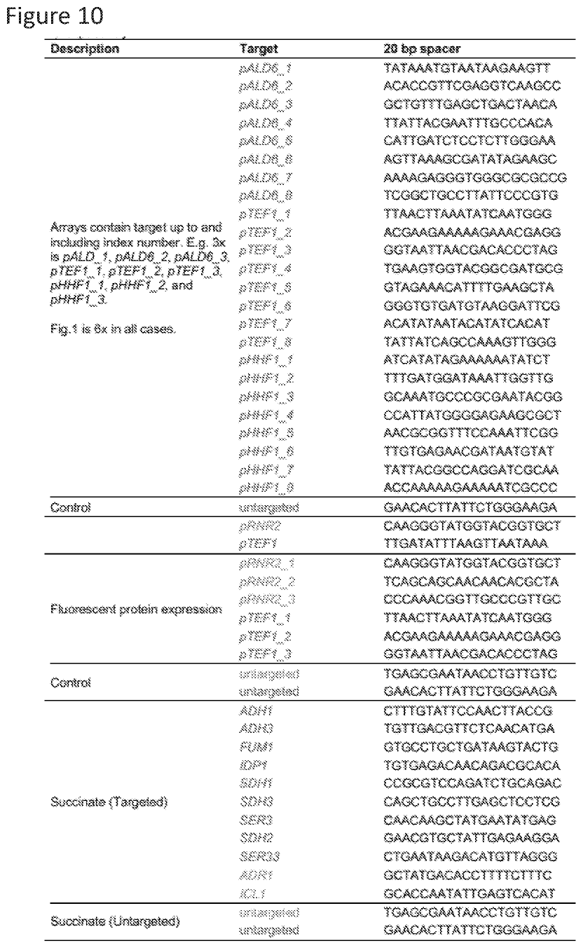

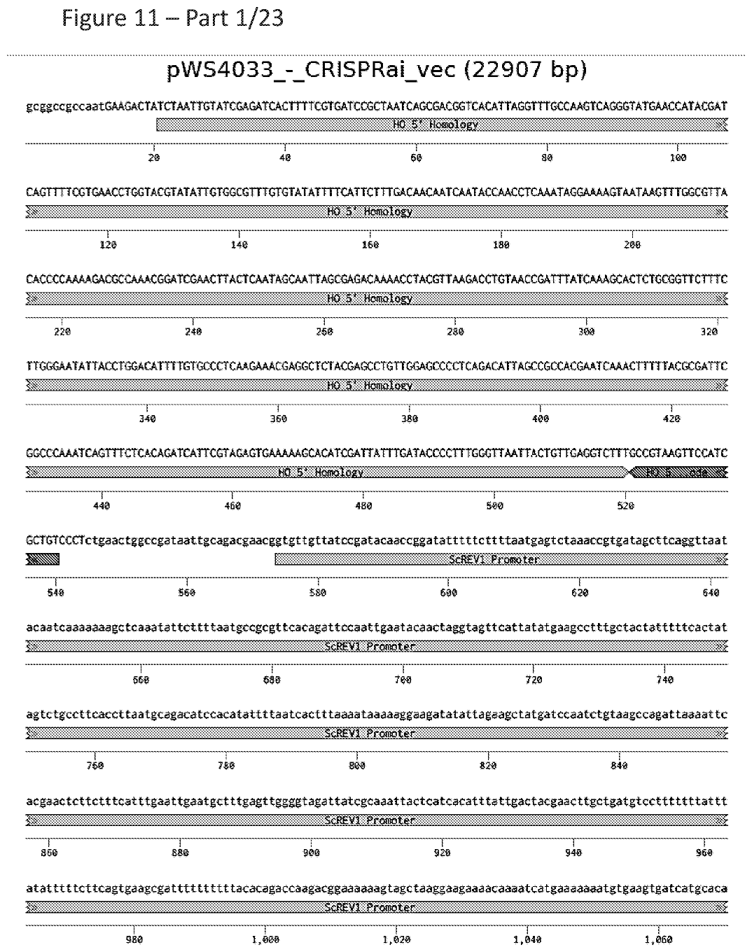

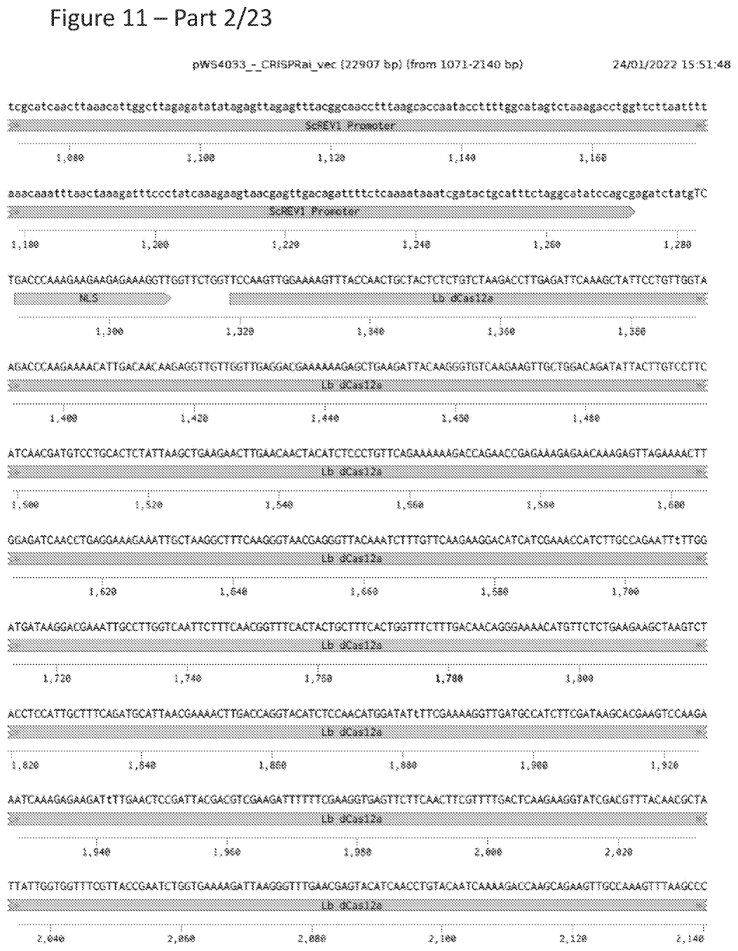

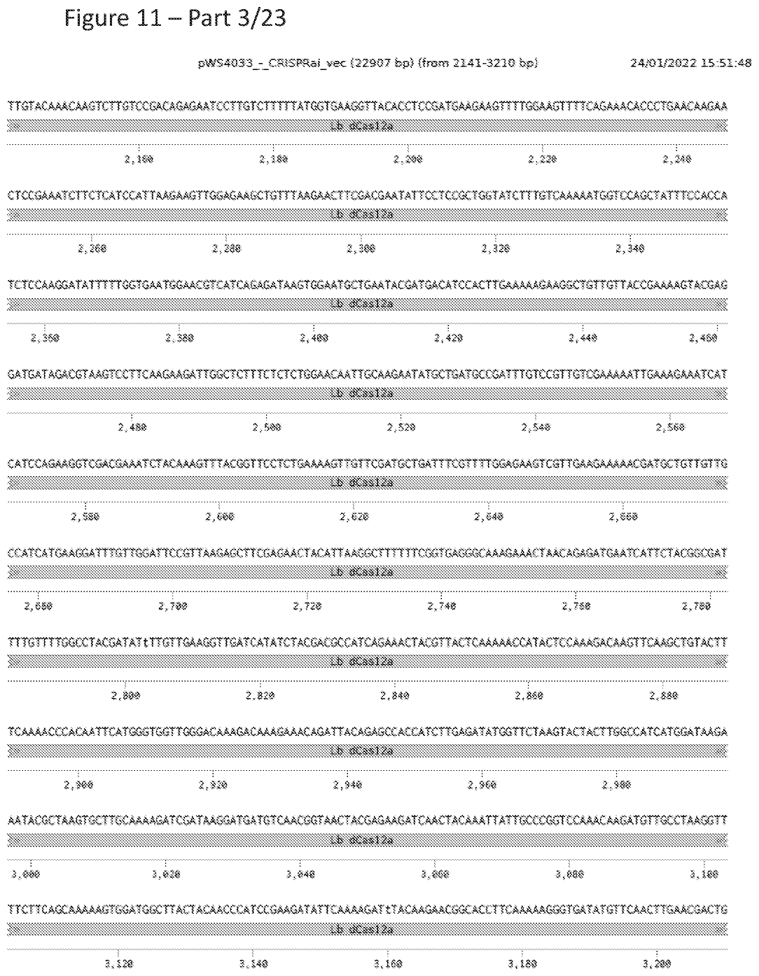

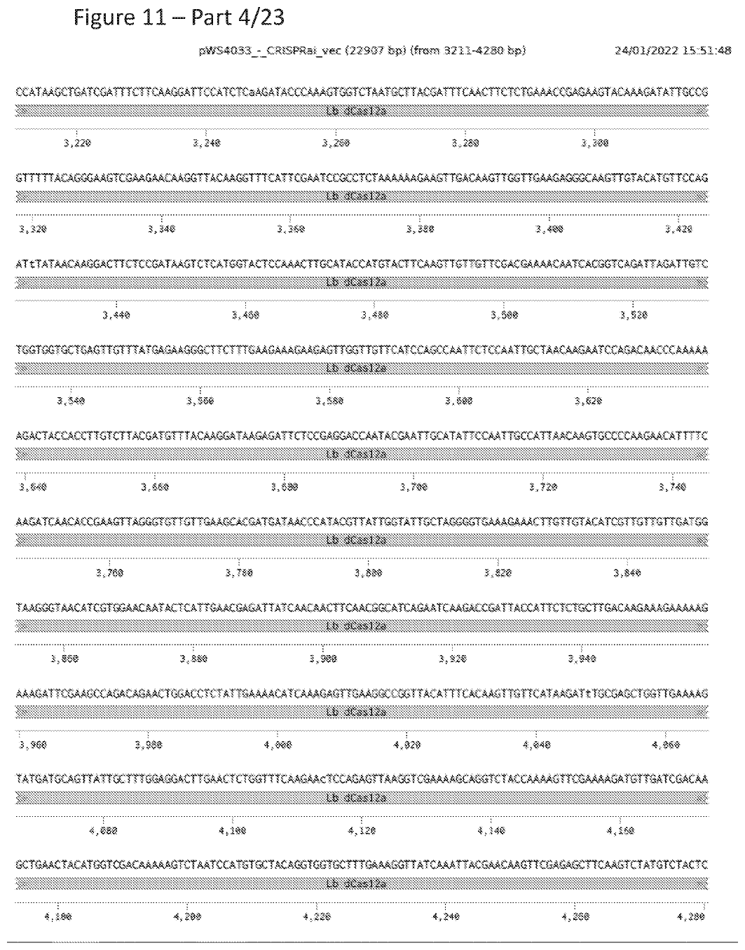

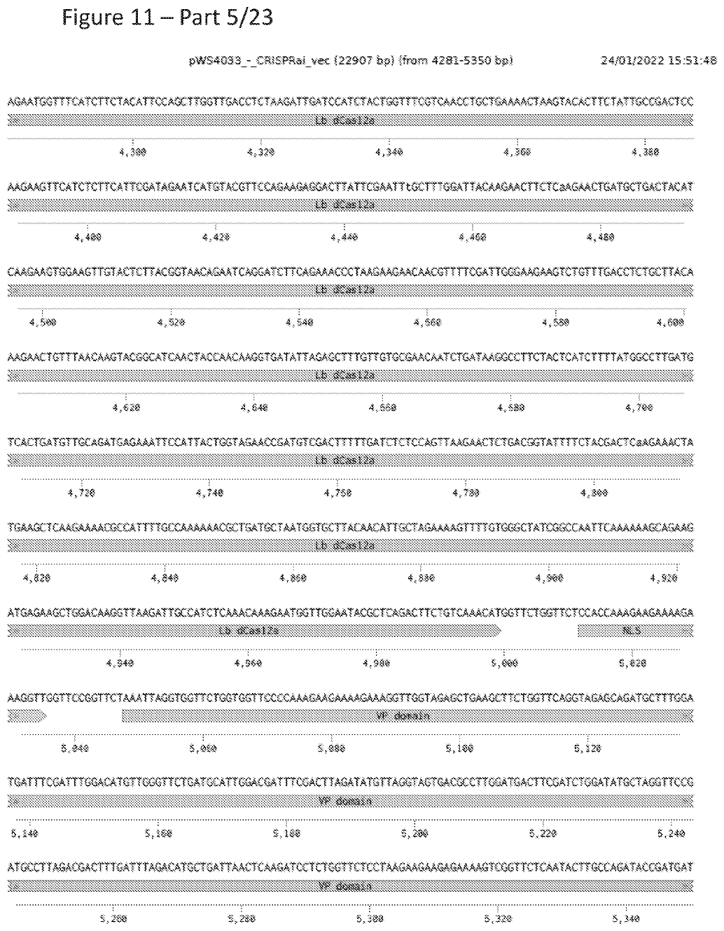

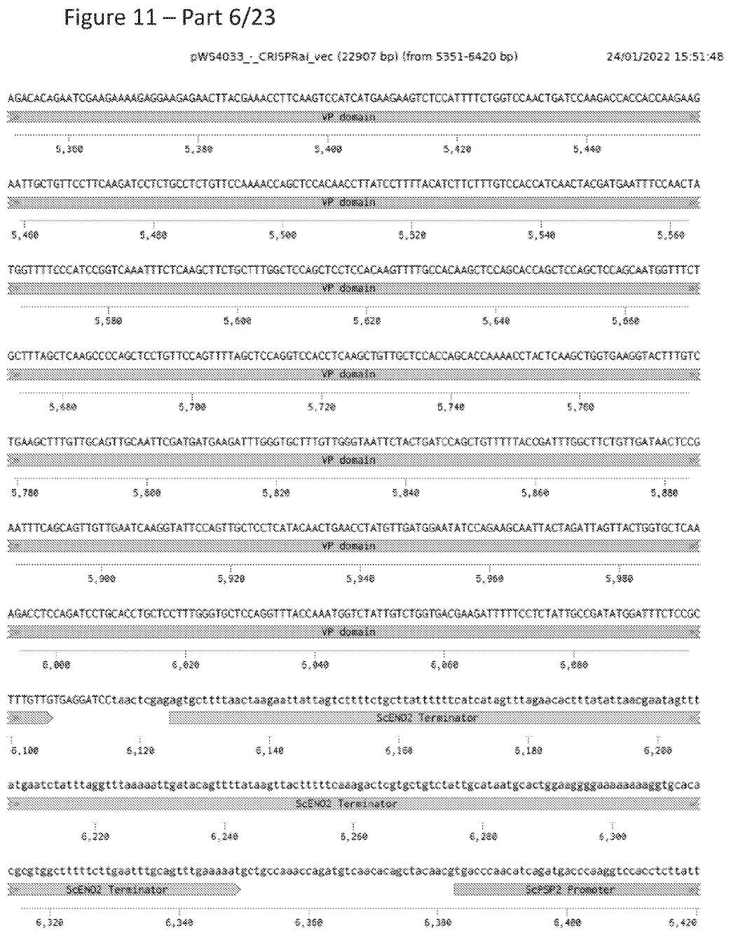

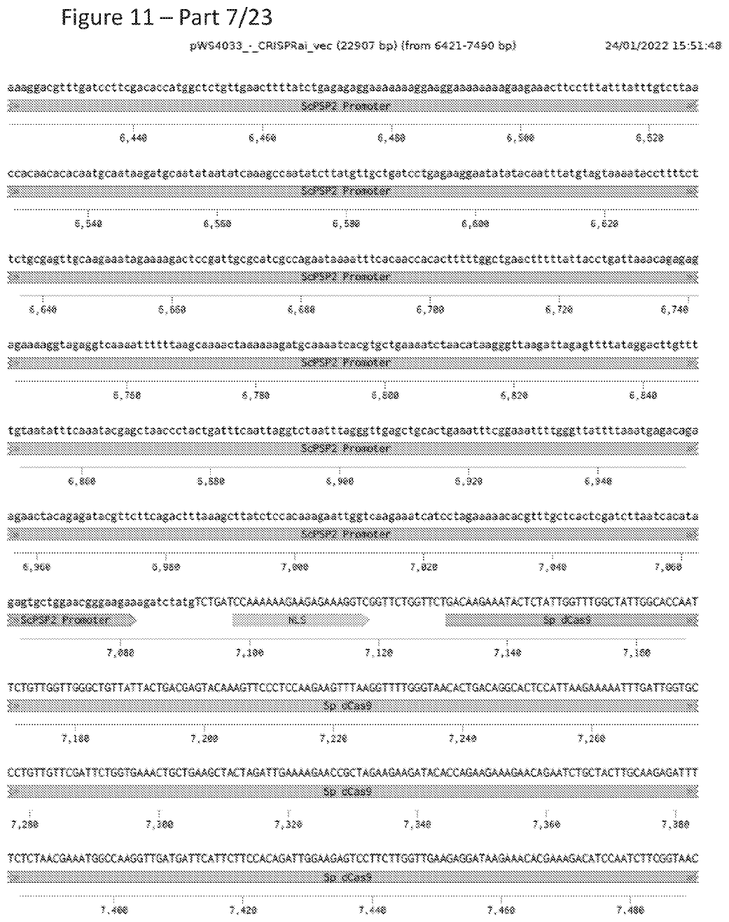

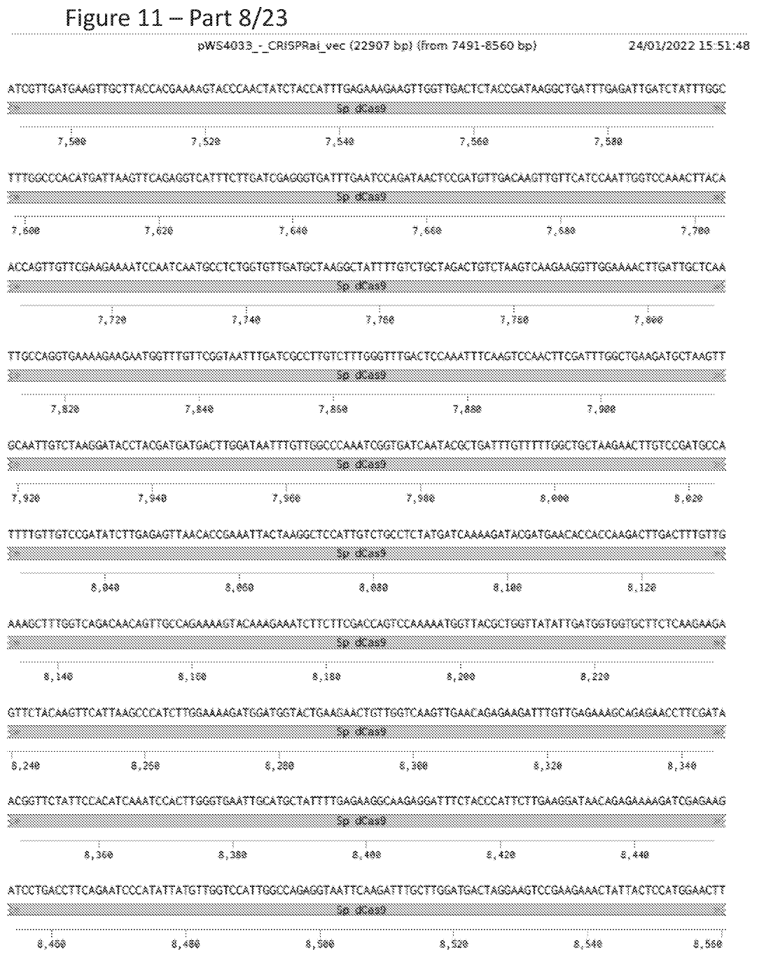

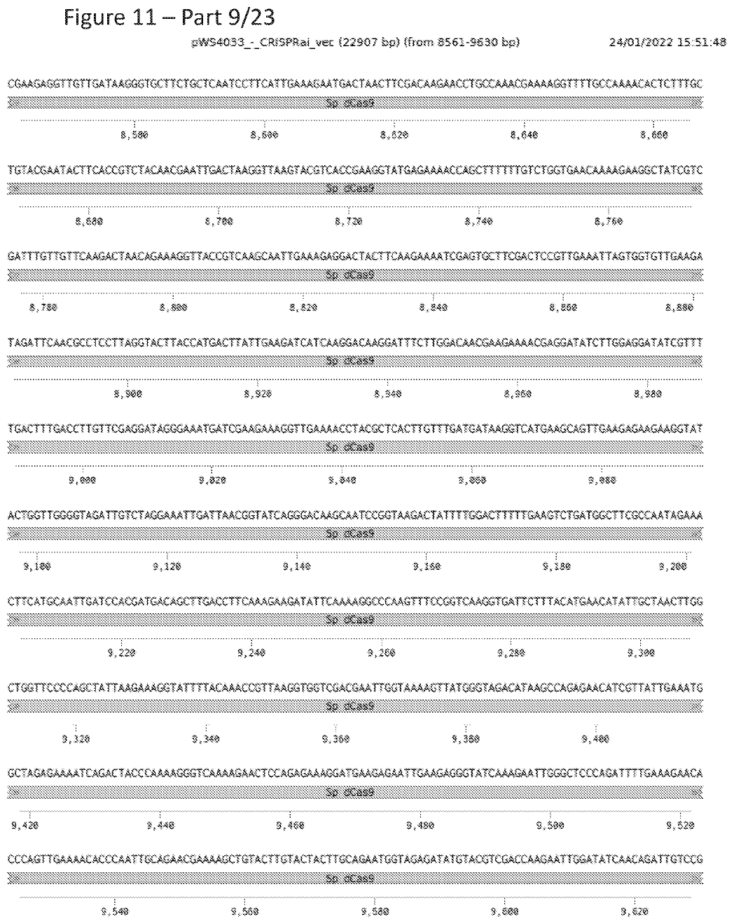

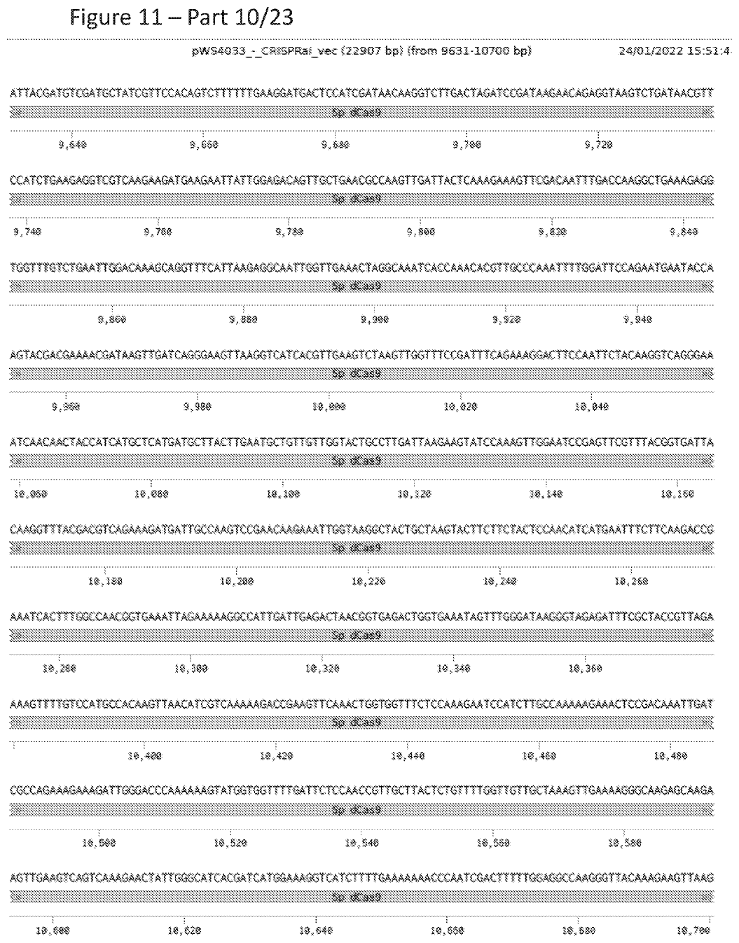

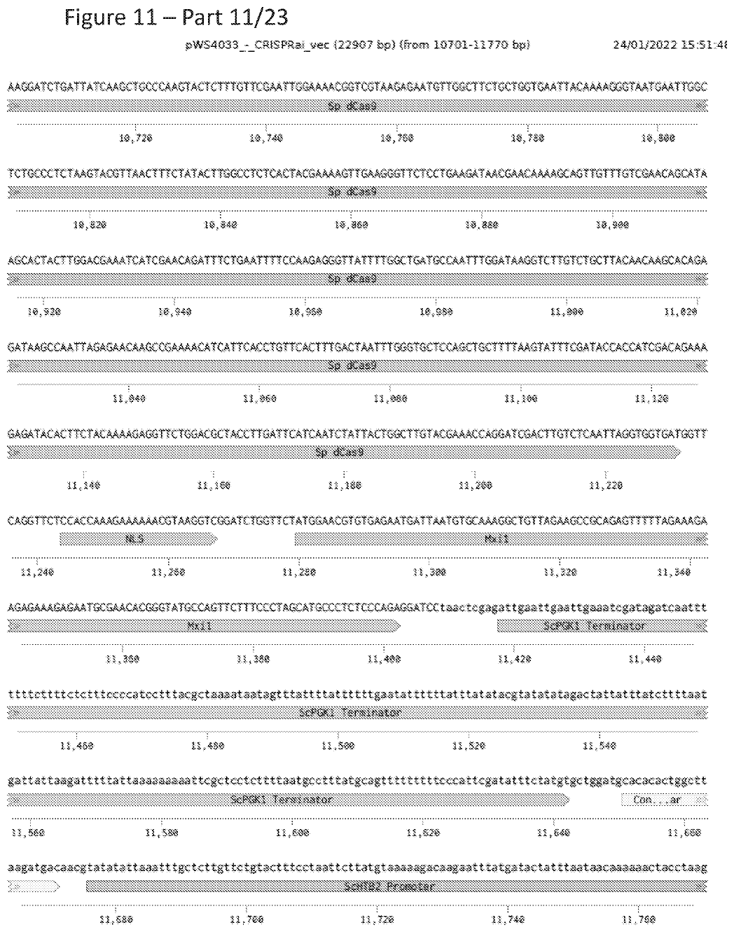

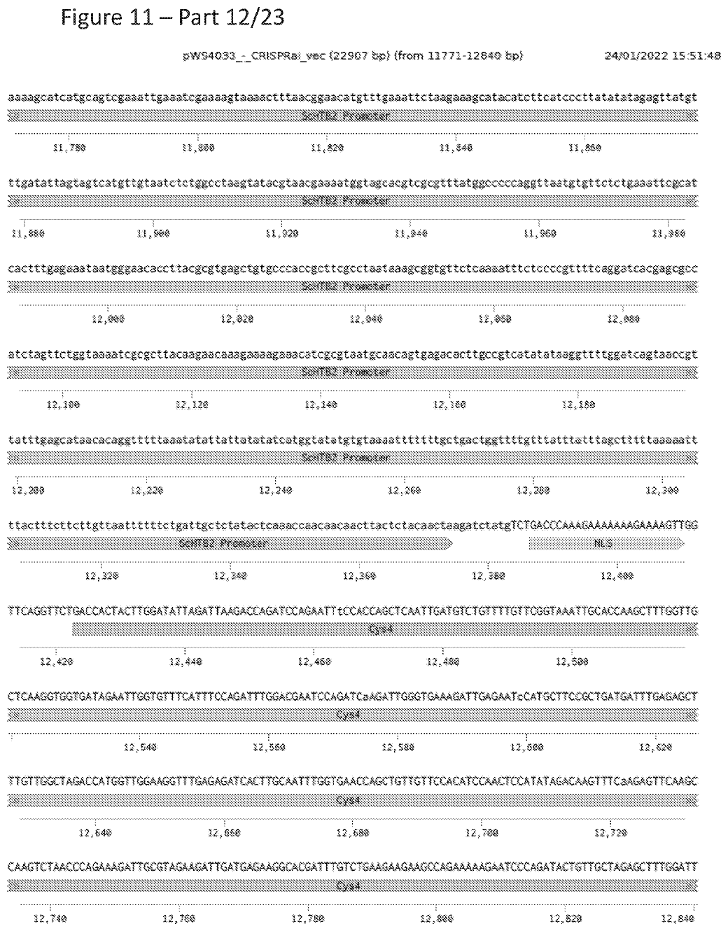

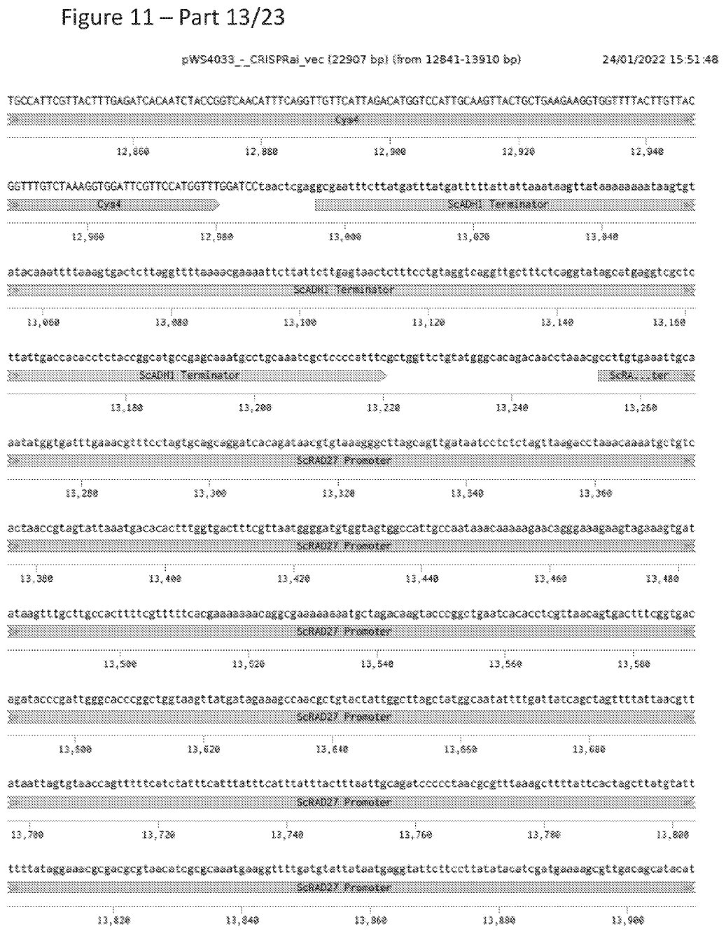

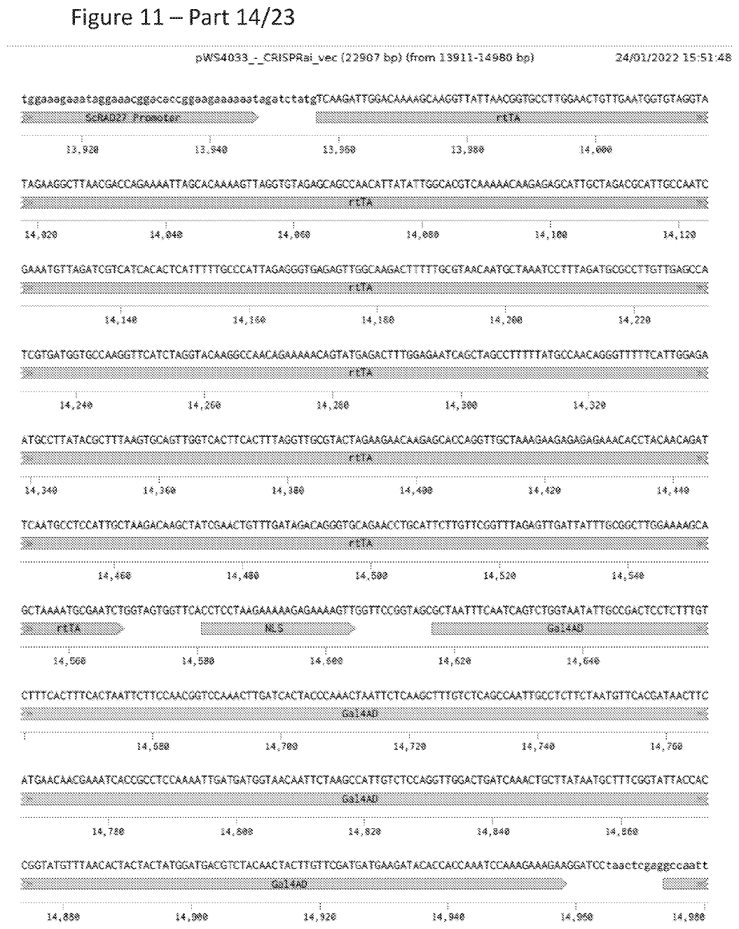

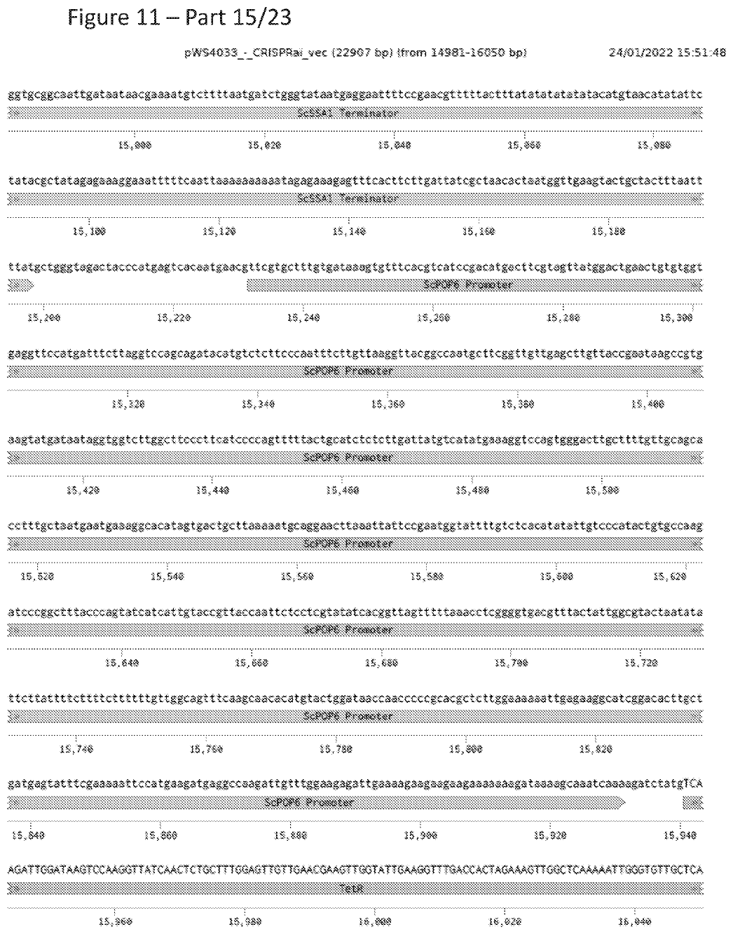

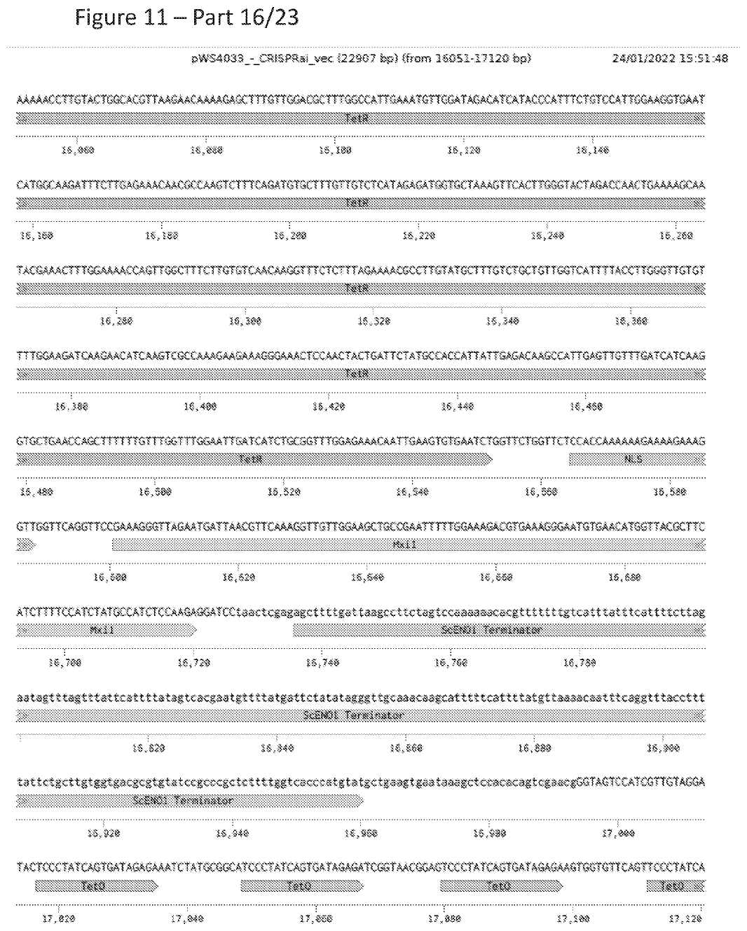

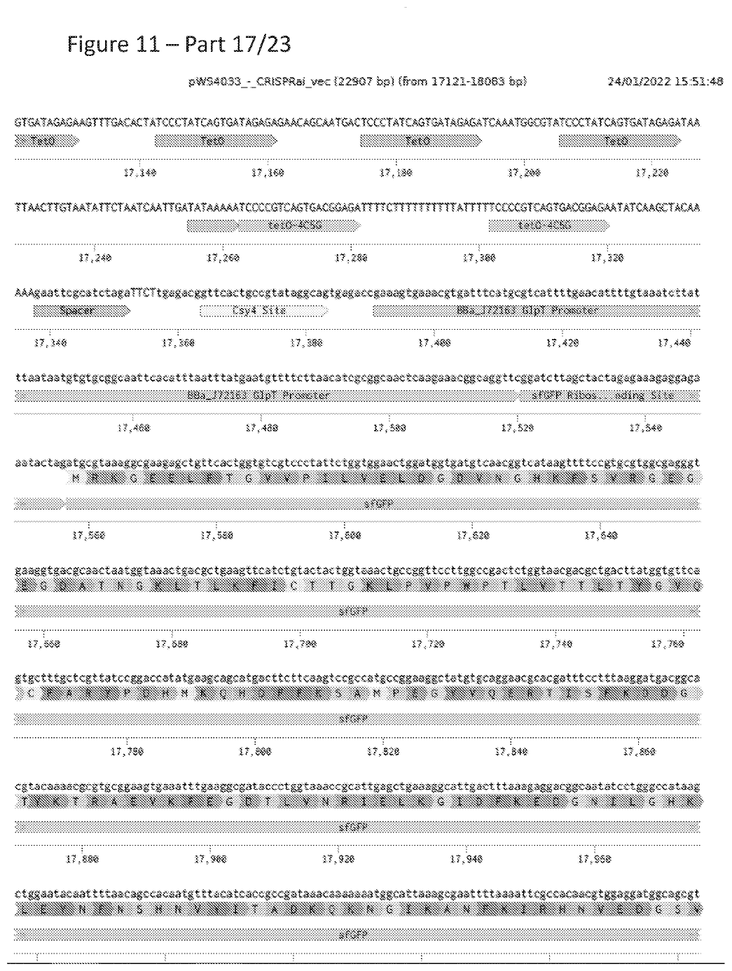

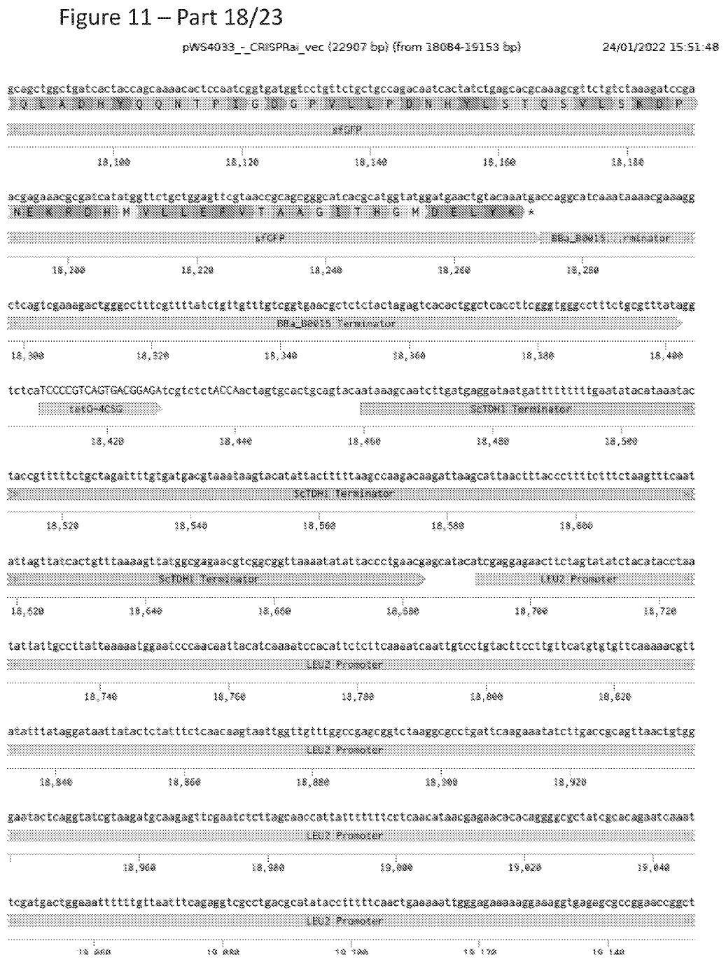

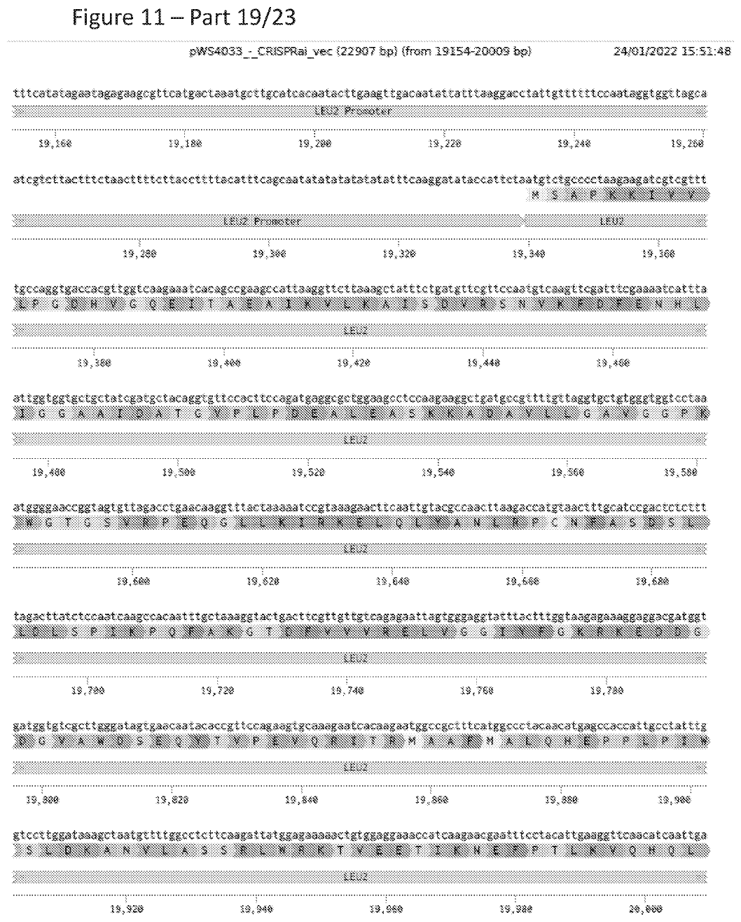

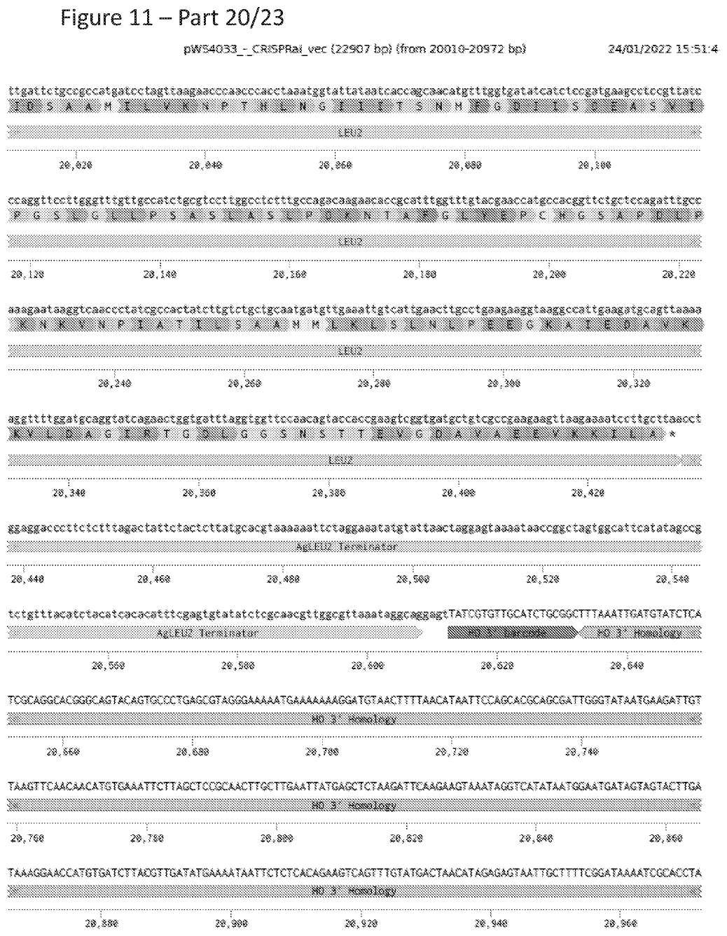

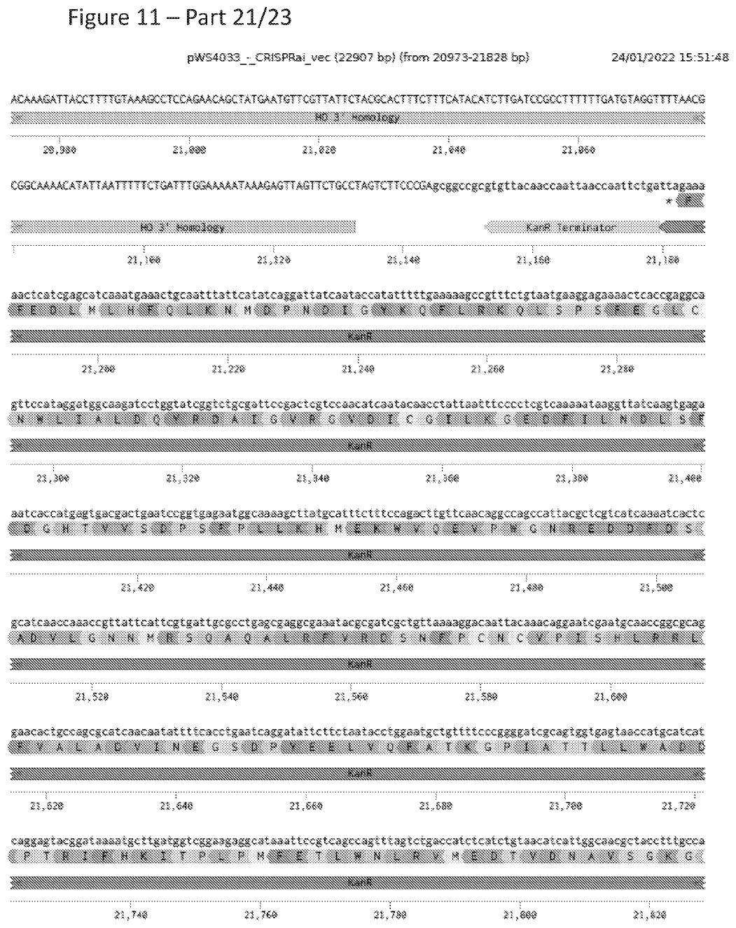

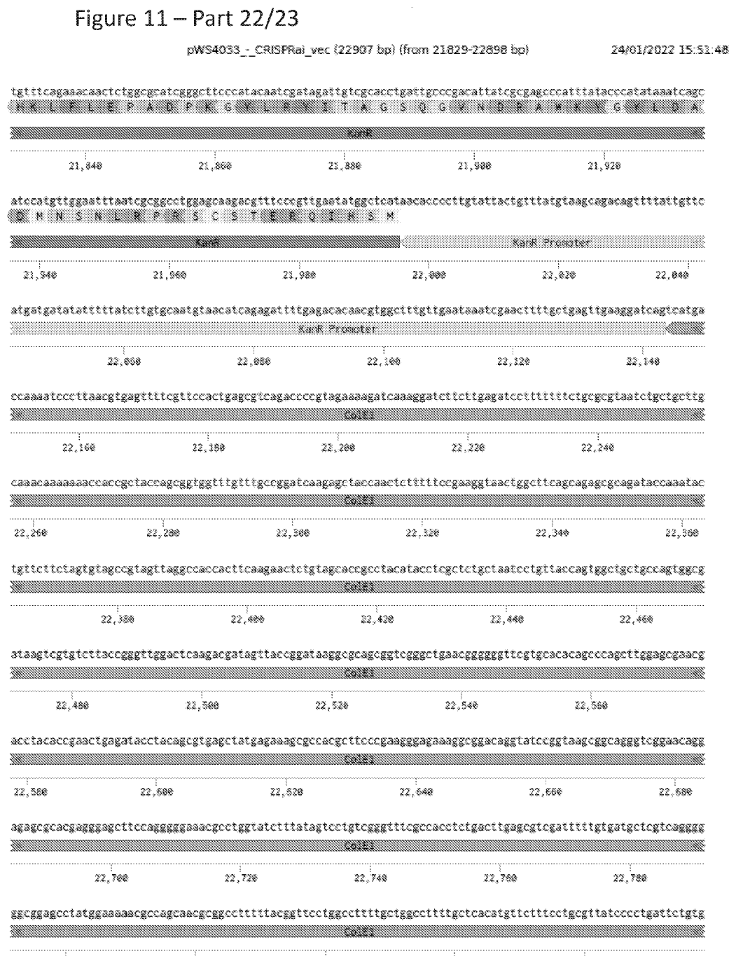

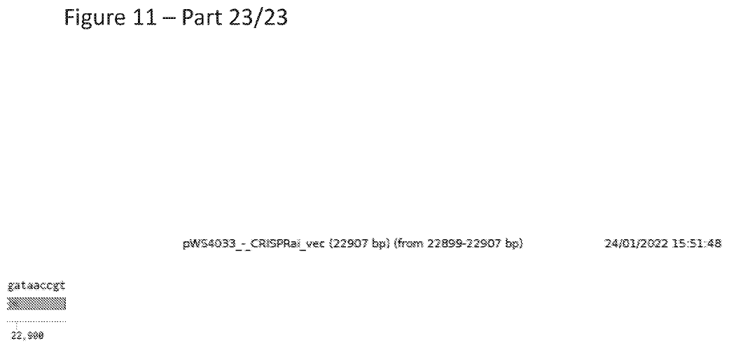

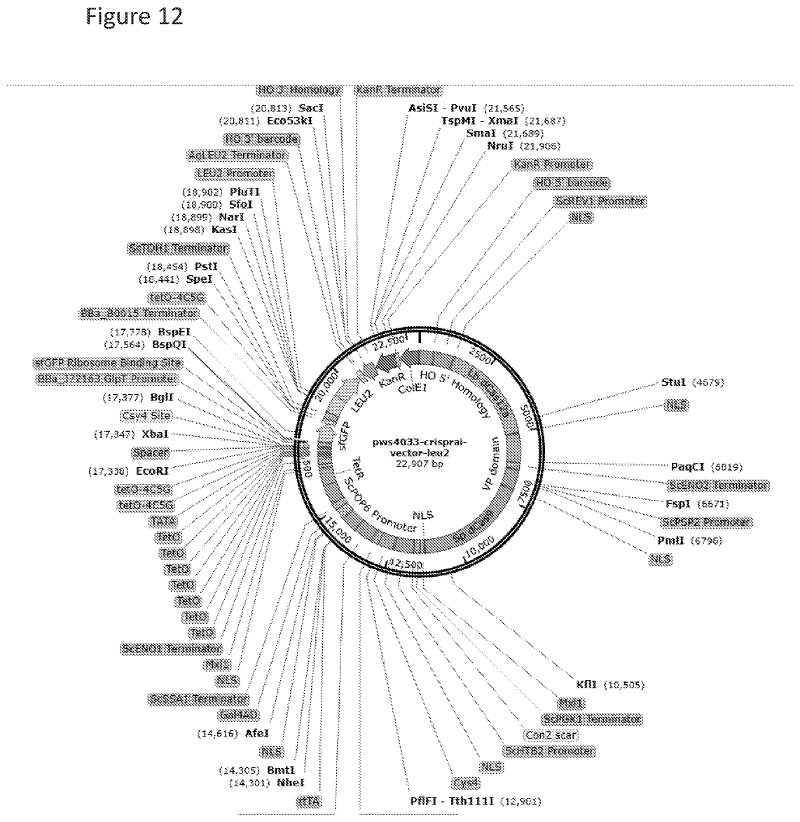

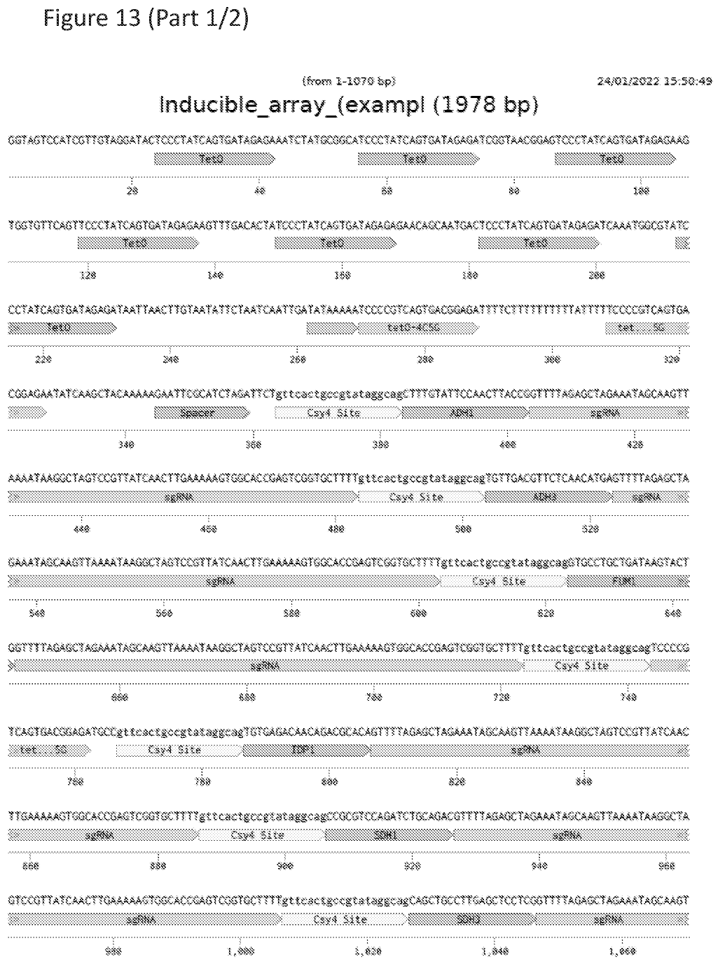

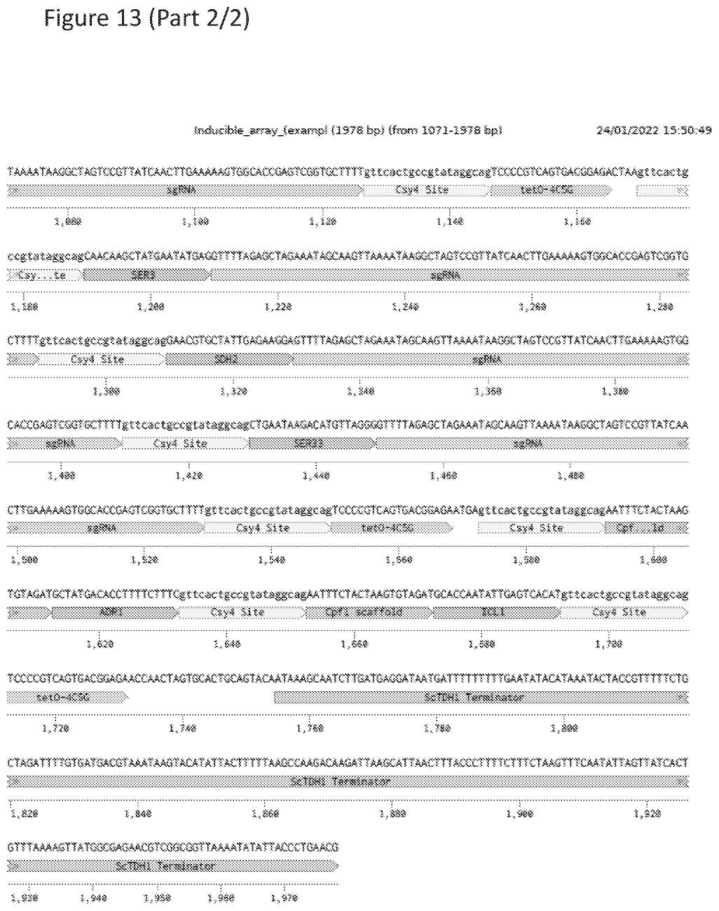

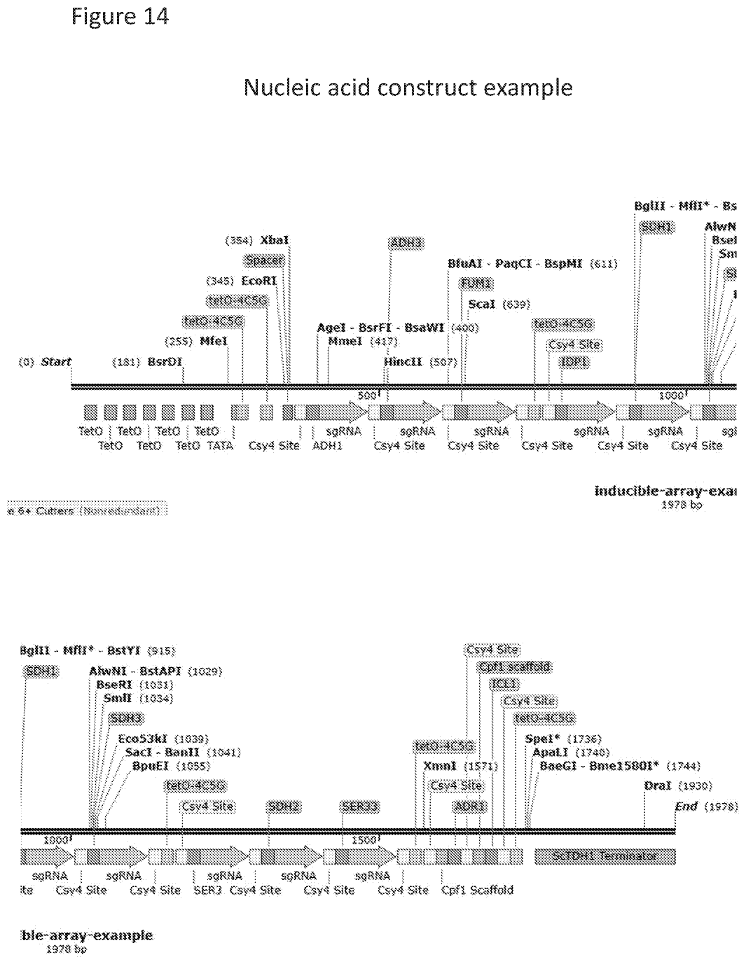

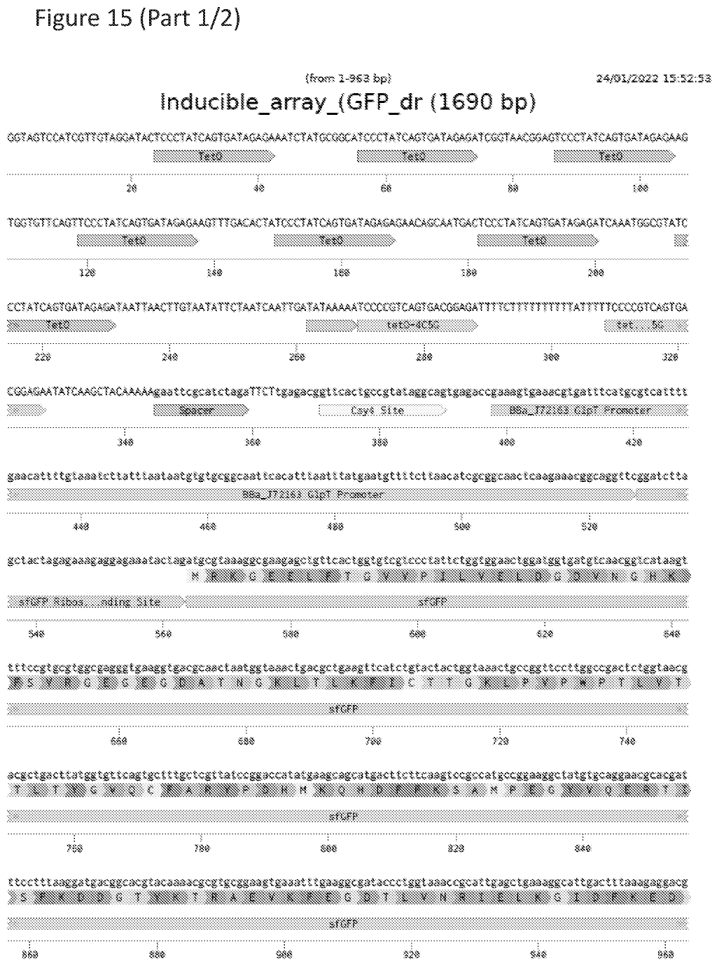

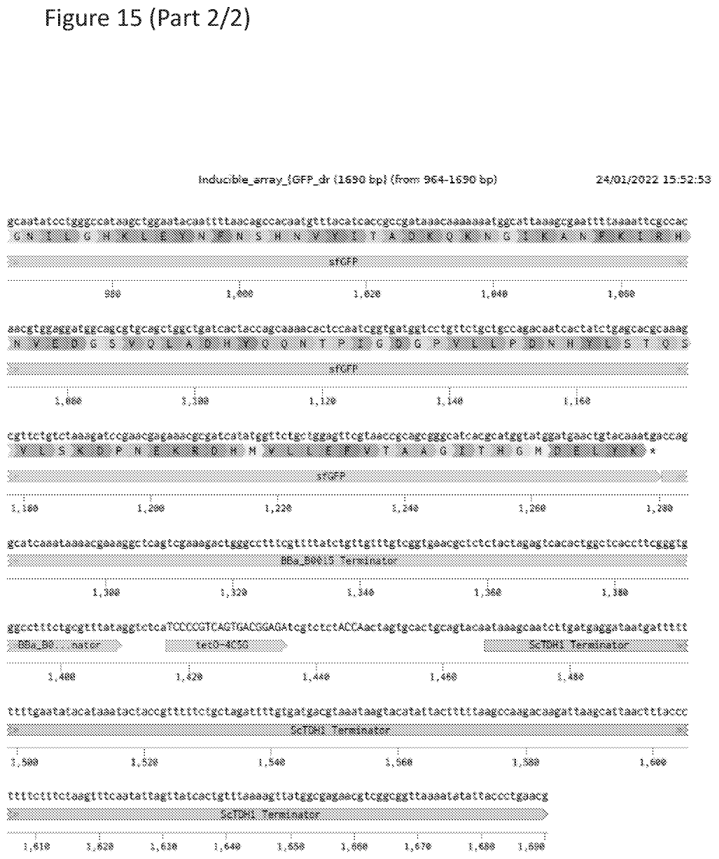

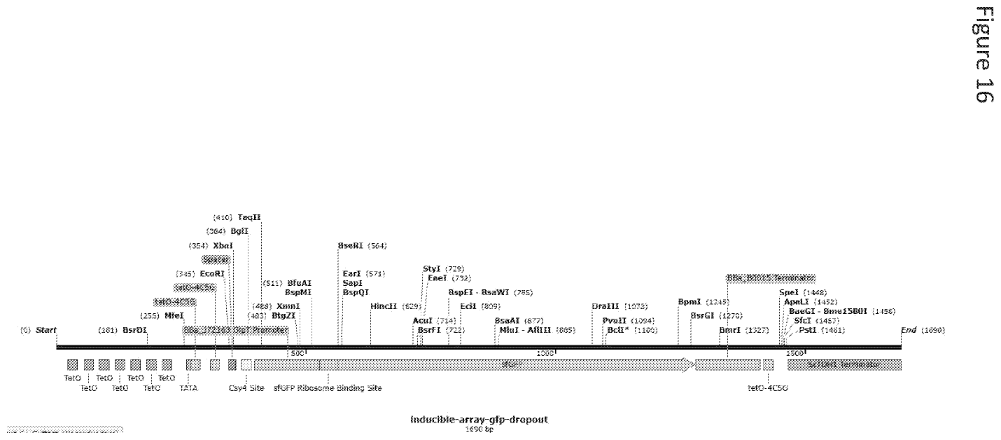

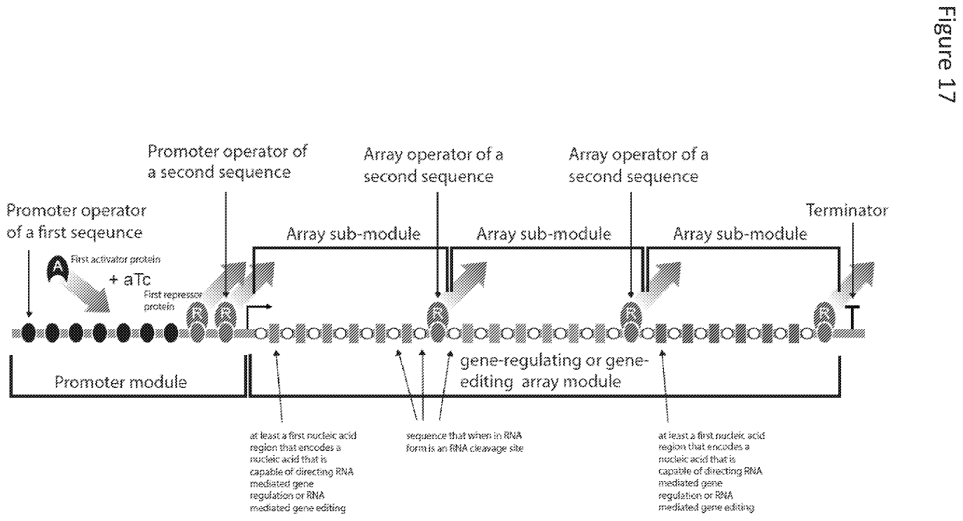

gRNA target design. All gRNAs were designed in Benchling, using the CRISPR Design Tool. For gene activation gRNAs (dCas12a-VP), targets were chosen between −200 and −350 bp relative to the start codon location of the chosen genes. For repression gRNAs (dCas9-Mxi1), targets were chosen between-100 to +150 bp relative to the start codon location of the chosen genes. All gRNAs used in this study are listed in FIG. 10. 20 bp target sequences cannot contain an internal BsaI, BsmBI, or NotI restriction sites, required for downstream cloning and transformation purposes. Additionally avoiding EcoRI, XbaI, SpeI, and PstI is useful for extended cloning of fully assembled arrays using the BioBrick assembly method (see below) and digest verification, although not necessary.

gRNA array design. To generate the gRNA fragments for array assembly, primer pairs were designed to amplify without a template for activation gRNAs and with a template (pWS3799-Cas9 gRNA-Csy4 template) for repression gRNAs (FIG. 7a). Each dsDNA gRNA fragment includes the Cas protein specific gRNA scaffold, 20 bp target sequence, and a Csy4 site at the 3′ end. BsaI generated overhangs within the CRISPRai vector and sub-array plasmids occur within the Csy4 site at the start and mutTetO site at the end of the array, and by designing the BsaI overhangs to occur within adjacent gRNA fragments, gRNA arrays can be made scarlessly. This creates an array of gRNAs each flanked precisely by Csy4 sites (FIG. 7c). Scarless gRNA arrays were designed using the Benchling Golden Gate Assembly Wizard (Benchling.com). There are no constraints on the organization of gRNAs within the array, and activation and repression gRNAs can be designed in any order. Note: Resulting arrays are highly repetitive, particularly around the Cas9 gRNA handle (depending on the number of gRNAs in the final array). Although this was not seen during batch culture, arrays can recombine over multiple cell passages while induced.

Activation (dCas12a-VP) gRNA fragment PCR. Activation gRNA PCRs were setup in 20 μL volume reactions, as follows: 4 μL of 5×Q5 Reaction Buffer (NEB), 0.4 μL of 10 mM dNTPs (NEB), 1 μL of of each primer (100 μM), 0.2 μL of Q5 High-Fidelity DNA Polymerase (NEB), and 13.4 μL ddH₂O. Activation gRNAs were created in 5 cycles of a non-amplifying extension PCR reaction, as follows: 30s at 98° C., (10s at 98° C., 20s at 61° C., 30s at 72° C.)×5 cycles, 30s at 98° C., hold at 4° C.

Repression (dCas9-Mxi1) gRNA fragment PCR. Repression gRNA PCRs were setup in 20 μl volume reactions, as follows: 4 μL of 5×Q5 Reaction Buffer (NEB), 0.4 μL of 10 mM dNTPs (NEB), 1 μL of of each primer (100 μM), 1 μL of pWS3977 plasmid (˜ 10 ng/L), 0.2 μL of Q5 High-Fidelity DNA 286 Polymerase (NEB), and 12.4 μL ddH₂O. Repression gRNAs were generated in a standard, 30-cycle amplifying PCR reaction, as follows: 30s at 98° C., (10s at 98° C., 20s at 57° C., 30s at 72° C.)×30 cycles, 30s at 98° C., hold at 4° C. DpnI digestion of the template DNA is not required following the PCR reaction as subsequent cloning steps use alternative selection markers.

gRNA fragment purification. 4 μL of 6× loading dye (NEB) was added to the 20 μL PCR reaction and run on a 2% agarose until total separation of DNA bands. After gel electrophoresis, gel bands were excised and DNA was extracted using Zymoclean Gel DNA Recovery kit (Zymo Research), following manufacturer instructions. As gRNA fragments are small (˜100 bp for activation gRNAs and ˜150 bp for repression gRNAs), it is important to excise a clean band from the gel, avoiding residual primer sequences which will run close to the desired band. Once purified, gRNA fragment DNA concentration was measured (NanoDrop™ One) and samples were diluted to 100 fmol/μL.

gRNA fragment array assembly. gRNA fragments were assembled into the CRISPRai vector and sub arrays plasmids in a 20 μL BsaI Golden Gate reaction, using the following setup: 1 μL of CRISPRai vector/sub-array plasmid (50 fmol/μL), 1 μL of each gRNA fragment (100 fmol/μL), 2 μL of T4 DNA ligase buffer (NEB), 1 μL of T4 DNA ligase (NEB), 1 μL of BsaI-HF v2 (NEB), and up to 20 μL with ddH₂O. Reaction mixtures were then incubated in a thermocycler using the following program: (37° C. 302 for 5 min, 16° C. for 5 min)×30 cycles, followed by a final digestion step of 55° C. for 10 min, and then heat inactivation at 80° C. for 10 min. Reactions were then transformed into E. coli. GFP negative colonies were screened for the correct array length by colony PCR and then sent for Sanger sequencing to confirm identity.

Sub-array assembly into CRISPRai Vector. Sub-arrays and spacers were assembled into the CRISPRai vectors in a 10 μL BsmBI Golden Gate reaction, using the following setup: 0.5 μL of CRISPRai 308 vector/sub-array plasmid (50 fmol/μL), 1 μL of each sub-array/spacer (50 fmol/μL), 1 μL of T4 DNA ligase buffer (NEB), 0.5 μL of T4 DNA ligase (NEB), 0.5 μL of BsmBI v2 (NEB), and 3.5 μL of ddH₂O. Reaction mixtures were then incubated in a thermocycler using the following program: (42° C. for 2 min, 16° C. for 5 min)×25 cycles, followed by a final digestion step of 55° C. for 10 min, and then heat inactivation at 80° C. for 10 min. Reactions were then transformed into E. coli. GFP negative colonies were screened for the correct array length by colony PCR or restriction digesting using EcoRI/XbaI and SpeI/PstI.

Additional cloning features. To increase flexibility of the toolkit once gRNA arrays have been assembled into the CRISPRai vector, a BioBrick cloning prefix (excluding NotI) is included between the promoter and the start of the gRNA array, and a BioBrick cloning suffix (excluding NotI) is included between the end of the gRNA array and terminator. This allows the user to excise and ligate validated gRNA arrays into different CRISPRai vectors to change the yeast selection marker without recreating the array from scratch. Additionally, gRNA arrays can be concatenated by BioBrick assembly to create combinations of arrays without requiring a redesign.

### Strains and Cultivation Conditions

E. coli DH5a was used for propagating all plasmids and grown at 37° C. in Luria Broth (LB) medium containing the appropriate antibiotics for plasmid selection (ampicillin 100 μg/mL, chloramphenicol 34 μg/mL, or kanamycin 50 μg/mL). S. cerevisiae strain BY4741 (MATa his3Δ1 leu2Δ0 met15Δ0 ura3Δ0) was used for all yeast experiments. For succinic acid experiments, fully complemented yeast strains were created by restoring the missing auxotrophic markers on a single-copy plasmid³⁵. Yeast extract peptone dextrose (YPD) was used for culturing cells in preparation for transformation: 1% (w/v) Bacto Yeast Extract (Merck), 2% (w/V) Bacto Peptone (Merck), 2% glucose (VWR). Fluorescent reporter assay experiments were performed in synthetic complete (SC) medium: 2% (w/v) glucose (VWR), 0.67% (w/v) Yeast Nitrogen Base without amino acids (Sigma), 0.14% (w/v) Yeast Synthetic Drop-out Medium Supplements without histidine, leucine, tryptophan, and uracil (Sigma), 20 mg/L uracil (Sigma), 100 mg/L leucine (Sigma), 20 mg/L histidine (Sigma), and 20 mg/mL tryptophan (Sigma). Succinic acid production experiments were performed in synthetic minimal (SD) medium: 2% (w/v) glucose (VWR), and 0.67% (w/v) Yeast Nitrogen Base without amino acids (Sigma).

### Yeast Transformations

For transformation, 200 ng of the final CRISPRai plasmid was digested by at 37° C. for 1 h NotI in the 338 following setup: 200 ng CRISPRai, 1 μL CutSmart™ Buffer (NEB), 0.2 μL NotI-HF (NEB), up to 10 μL H₂O. Digestions were heat inactivated at 65° C. for 20 minutes before transformation. Chemically competent yeast cells were created following the lithium acetate protocol from Gietz and Schiestl³⁶, as follows: Yeast colonies were grown to saturation overnight in YPD. The following morning the cells were diluted 1:100 in 15 mL of fresh YPD in a 50 ml conical tube and grown for 4-6 h to OD₆₀₀ 0.8-1.0. Cells were pelleted and washed once with 10 mL 0.1 M lithium acetate (LiOAc) (Sigma). Cells were then resuspended in 0.1 M LiOAc to a total volume of 100 μL/transformation. 100 μL of cell suspension was then distributed into 1.5 mL reaction tubes and pelleted. Cells were resuspended in 64 μL of DNA/salmon sperm DNA mixture (10 μL of boiled salmon sperm DNA (Invitrogen)+DNA+ddH₂O), and then mixed with 294 μL of PEG/LiOAc mixture (260 μL 50% (w/v) PEG-3350 (Sigma)+36 μL 1 M LiOAc). The yeast transformation mixture was then heat-shocked at 42° C. for 40 mins, pelleted, resuspended in 200 μL 5 mM CaCl₂ and plated onto the appropriate selection medium.

### Inducible CRISPRai Toolkit Construction

All constructs were created within the Yeast MoClo Toolkit³¹ framework and assembled by Golden Gate assembly. Novel parts were synthesized (IDT) or assembled from PCR generated fragments designed using the Benchling Golden Gate tool. All DNA for Golden Gate reactions was set to equimolar concentrations of 50 fmol/μL prior to experiments. Golden Gate reactions were prepared as follows: 0.25 μL of backbone plasmid, 0.5 μL of each DNA fragment or plasmid, 1 μL T4 DNA ligase buffer (Promega), 0.5 μL T7 DNA Ligase (NEB), 0.5 μL restriction enzyme (BsaI-HF v2/BsmBI v2) (NEB), and H₂O to bring the final volume to 10 μL. Reaction mixtures were then incubated in a thermocycler using the following program: (42° C. for 2 min, 16° C. for 5 min)×25 cycles, followed by a final digestion step of 55° C. for 10 min, and then heat inactivation at 80° C. for 10 min.

### Fluorescent Reporter Assay

All reporter strains were picked into 500 μL of synthetic complete (SC) medium and grown in 2.2 mL 96 362 deep-well plates at 30° C. C in an Infors HT Multitron, shaking at 700 rpm overnight. The next day, saturated strains were diluted 1:100 into fresh media, with and without 1 μM aTc (Alfa Aesar, J66688-MB). For single-point measurements, cultures were incubated for 16 h and cell fluorescence was measured by an Attune™ NXT Flow Cytometer (Thermo Scientific). For batch culture and daily cell 366 passaging assay experiments: daily measurement and culturing as described in the text. Attune™ NXT Flow Cytometer settings: FSC 300 V, SSC 350 V, BL1 500 V, VL2 450 V, YL2 450 V. Fluorescence data was collected from 10,000 cells for each experiment and analysed using FlowJo software. Note: 1 μM (463 ng/μL) aTc was used, rather than the standard 100 ng/μL, to ensure ligand saturation and full release of the mutTetR-Mxi1 protein from the array. 1000× stock solution of aTc (1 mM) was in 100% DMSO. Final concentration of DMSO was 0.1% in all induced conditions.

### Succinic Acid Production, Sampling, and Measurement

All succinic acid production strains were picked into 6 mL of synthetic minimal (SD) medium and grown at 30° C., 250 rpm overnight. The next day, optical density was measured in a spectrophotometer (WPA Biowave II) and cultures were diluted to OD₆₀₀=0.05 in 1 mL SD media, with and without 1 μM aTc (Alfa Aesar, J66688-MB). Cultures were grown in 48-deep-well-plates (Agilent, 201238-100) at 30° C. in an Infors HT Multitron, shaking at 700 rpm. After 2 days, plates were spun down at 4000 rpm, 4° C. for 10 minutes. Then, 300 μL of the supernatant was sampled for each well. The same day, supernatant samples were measured directly by LC-MS alongside a succinic acid standard, as follows: succinic acid was detected and measured by UPLC-MS, using an Agilent 1290 Affinity chromatograph linked to an Agilent 6550 Q-TOF mass spectrometer. Separation was achieved using an Agilent Zorbax Eclipse Plus C18 column (2.1×50 mm, 1.8 μm) and an acetonitrile gradient of 0% for 2 minutes then an increase to 98% over 0.5 minutes at a flow rate of 0.3 ml/min. Mass spectral data was acquired in negative ion mode from m/z 90 to 1000 at the rate of 3 spectra per second throughout the separation. 0.2 μl was injected from both sample wells and standard solutions. Succinic acid concentrations were calculated from a succinic acid standard curve in Microsoft Excel.

### Statistics and Reproducibility

Unless otherwise stated, all data was analysed in Prism (GraphPad). Error bars represent the standard deviation of the mean and samples compared with Student's unpaired t-test where significance is noted. The respective number of replicates are given in the figure legend and all replicates are included.

### Example II—Inducible Expression of Large Polycistronic gRNA Arrays

Inducible CRISPR-based systems can be achieved by controlling the expression or state of the Cas protein or the gRNA via an exogenous stimulus, such as a chemical or light²⁴. For multiplexed CRISPRai, controlling the activity of the system through the inducible expression of a polycistronic gRNA array presents itself as promising approach. In this way, the entire system can be regulated through the expression of a single transcript, irrespective of the number of CRISPR proteins involved¹¹. Instead, protein expression can be tuned to balance CRISPRai performance with fitness. Moreover, induction of the system should not impose a severe burden on the host metabolism, as only transcription of the array (and not translation) is required²⁵. Additionally, by modulating the level of gRNA abundance, rather than the active state of the CRISPR components, alternate Cas proteins and their cognate gRNAs can be used where activatable versions are not yet developed, providing a universal approach 98 that should be applicable to most CRISPR-Cas systems.

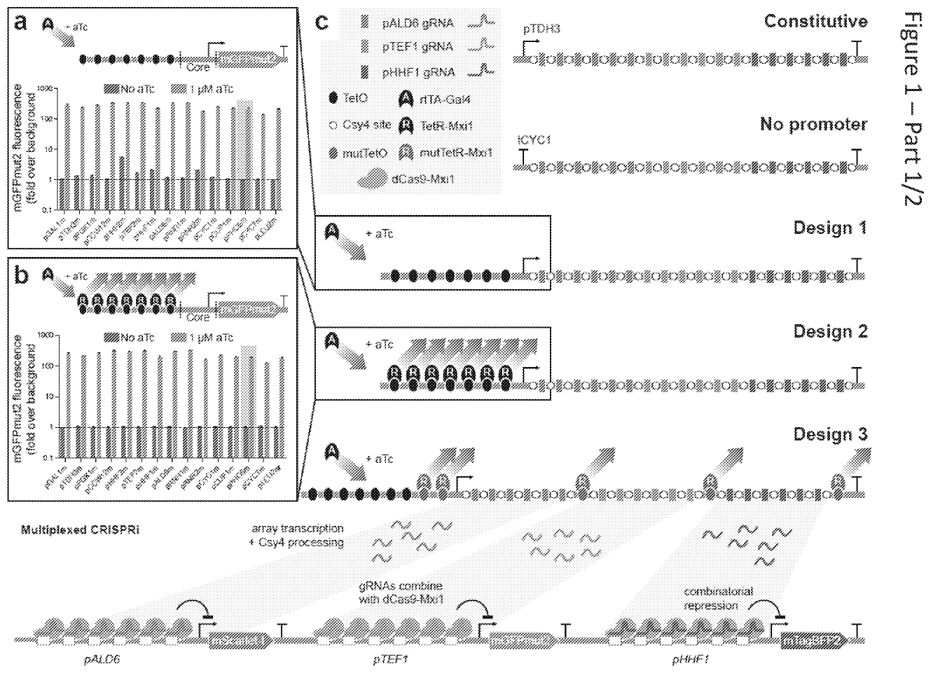

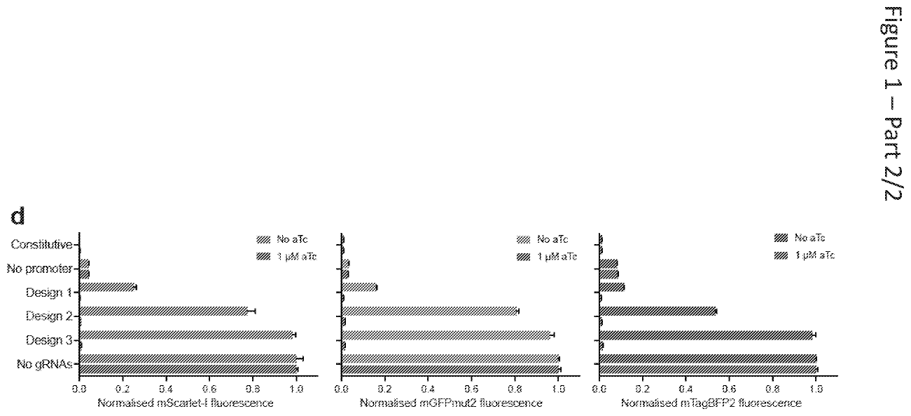

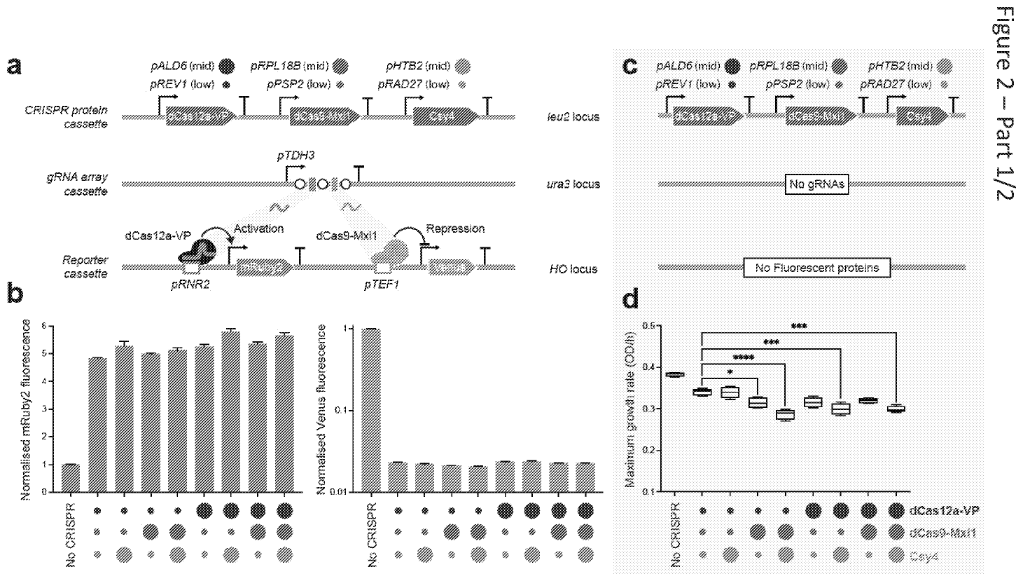

In order to explore possible strategies for creating inducible polycistronic gRNA arrays, we built on our previous work for assembling and expressing multiple gRNAs from a constitutive, Pol II-driven RNA transcript, which are then processed by the Csy4 endonuclease for multiplexed CRISPRi using dCas9-Mxi1¹⁹. Based on previous success of expressing individual gRNAs, we decided to develop inducibility using the Tet expression system^{9,17,24}. However, in the absence of the inducer anhydrotetracycline (aTc), where we desire no repression from CRISPRi, our first two designs which incorporated a low leak and then leak-free promoter reduced respective expression of our fluorescent protein reporters to 106 10% and 54%, therefore showing leakiness in the system (FIG. 1a-d, Design 1+2). This led us to the key discovery that gRNA arrays can transcribe without a promoter (FIG. 1c+d, No promoter). Since gRNA arrays that target promoters are themselves made of 20 bp fragments of those promoters, we reasoned that these short sequences are sufficient to clear nucleosomes, allowing transcriptional machinery to gain access and initiate transcription from within the array. We therefore required a method to repress transcription along the entire length of the array, in a way that would also be scalable to widely varied numbers of gRNAs.

To solve this problem, we used the opposing actions of orthogonal Tet-ON and Tet-OFF systems to drive expression of the array in the presence of aTc and silence the array in the absence of aTc. The Tet-ON system is composed of the reverse TetR protein fused to the Gal4 transcriptional activation domain (rtTA-Gal4)²⁶. This protein binds to Tet operator (TetO) sites upstream of the 5′ UTR in the presence of inducer to drive expression of the gRNA array. The Tet-OFF system uses a mutated version of the TetR protein (E37A P39K) fused to the Mxi1 transcriptional repression domain (mutTetR-Mxi1), and binds to an orthogonal TetO variant sequence (Tet4C5G, mutTetO)²⁷. We specifically target the mutTetR-Mxi1 protein to surround clusters of gRNAs to silence transcription across the entire array in the absence of inducer, without recruiting rtTA-Gal4 to these sites and interfering with array transcription (FIG. 1c, Design 3). We also targeted mutTetR-Mxi1 to the core promoter, using a Tet-repressible promoter adapted from Chen et al²⁸ (substituting the TetO sites for mutTetO sites), to prevent basal transcription.

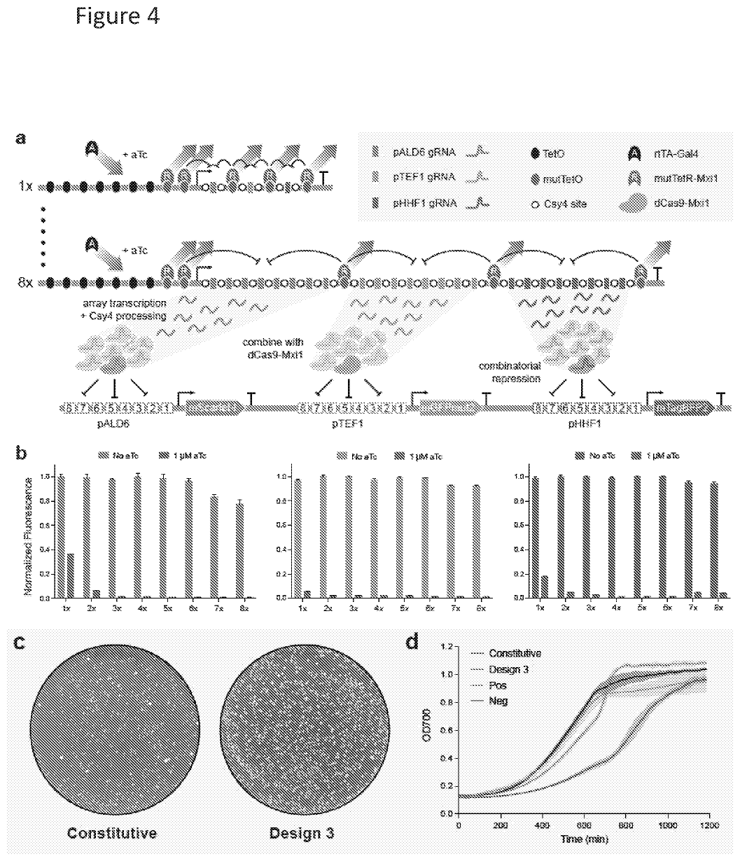

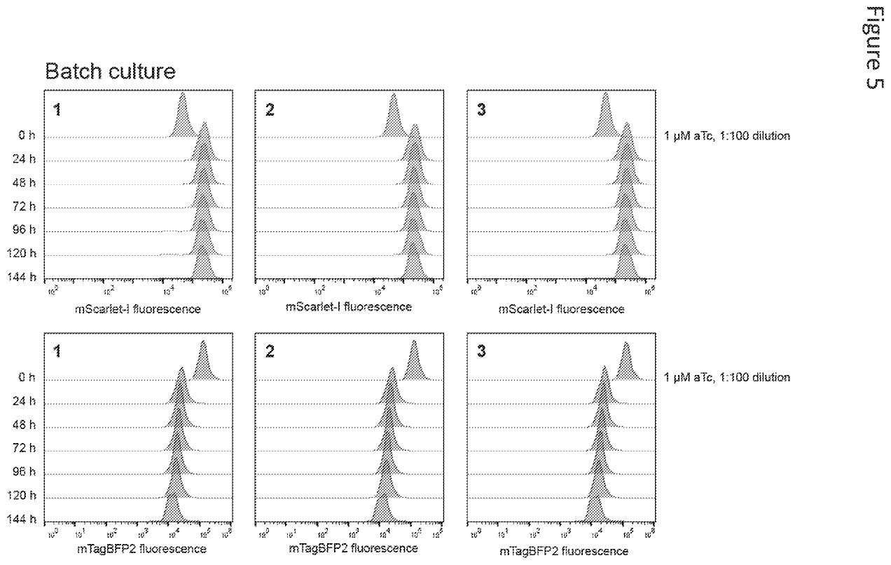

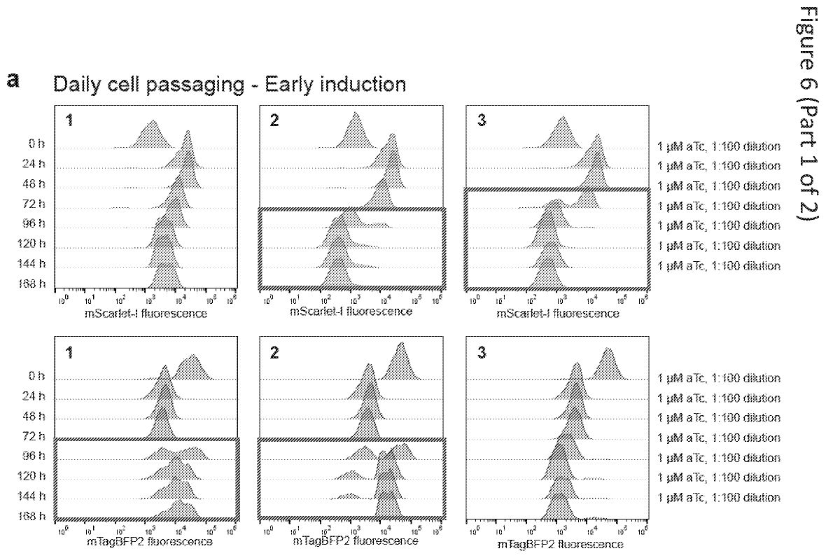

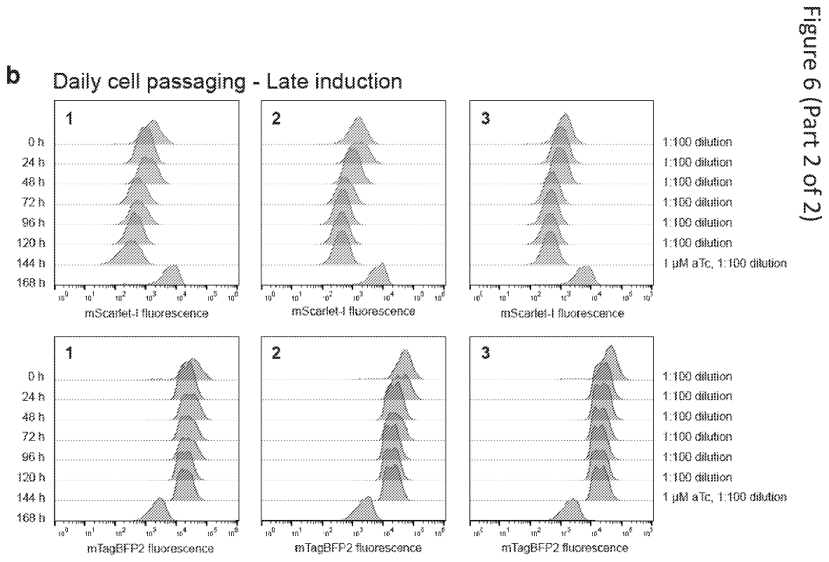

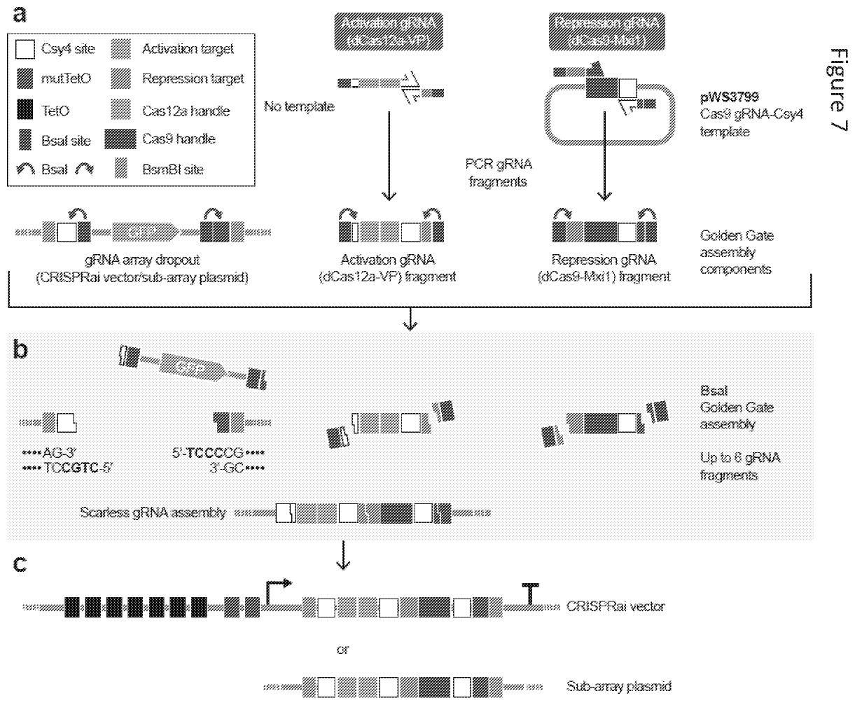

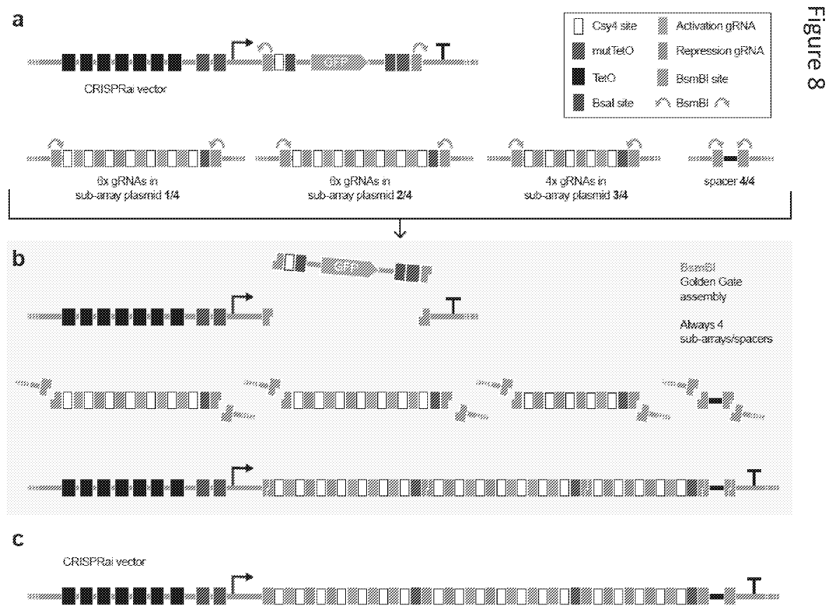

The new inducible gRNA array method removed almost all unwanted CRISPRi repression in the uninduced state, resulting in 96-98% of maximum reporter expression in the absence of aTc, demonstrating efficient silencing of the array from mutTetR-Mxi1 when interspersed between groups of gRNAs (FIG. 1d, Design 3). Strong silencing of the array is achievable with up to 6 gRNAs between mutTetO sites, with a small increase in basal CRISPRi activity above this number (FIG. 4a+b). Additionally, no significant difference was seen between the induced state and constitutive array expression, showing release of mutTetR-Mxi1 and the recruitment of rtTA-Gal4 to the promoter after addition with 1 μM aTc is highly efficient. Together, this resulted in up to 111-fold change in fluorescent protein expression after induction. Furthermore, the repression of the gRNA array in the uninduced state led to reduced growth defects after transformation compared to constitutive expression of the array, presumably due to lack of dCas9-Mxi1 targeting in the uninduced state (FIG. 4c).

### Example III—Design and Optimisation of the Inducible CRISPRai Platform

After developing the inducible gRNA array method with CRISPRi (gene repression), we next introduced a CRISPRa (gene activation) protein to complete the inducible CRISPRai platform. Building upon the previous work of Lian et al, who demonstrated the use of orthogonal Cas proteins to simultaneously up- 141 and down-regulate two target genes in yeast, we introduced the nuclease-deficient Cas12a from Lachnospiraceae bacterium, fused to the VP transcriptional activation domain, to play the role of activator (dCas12a-VP)¹¹. As CRISPR proteins are known to cause toxicity at high levels^{29,30}, we decided to explore the effect of protein expression on CRISPRai performance and cell fitness. We combinatorially varied the expression levels of dCas12a-VP, dCas9-Mxi1, and Csy4 using low and medium strength promoters from the Yeast MoClo Toolkit³¹ and assessed target gene regulation and cell growth (FIG. 2a-d).

To report on CRISPR gene activation and inhibition, we targeted dCas12a-VP and dCas9-Mxi1 to the RNR2 and TEF1 promoters driving the expression of mRuby2 and Venus using a constitutively expressed gRNA array (FIG. 2a). Varying the expression of the three CRISPR proteins had little effect on fluorescence reporter output, with Csy4 expression responsible for most of the minor differences (FIG. 2b). We also expressed the three CRISPR proteins in the absence of gRNAs and fluorescent proteins to determine the effect of protein expression on growth (FIG. 2c). As expected, increasing the strength of the promoters driving the expression of these proteins reduced the maximum growth rate (FIG. 2d). Based on these findings, we chose to build the inducible CRISPRai toolkit with the weak REV1, PSP2, and medium strength HTB2 promoters driving the expression of dCas12a-VP, dCas9-Mxi1, and Csy4, respectively, as higher expression did not incur a performance benefit but did lead to a fitness cost. As rtTA-Gal4 and mutTetR-Mxi1 were already under the control of the weak RAD27 and POP6 promoters, we kept these fixed.

The inducible CRISPRai platform consists of an all-in-one genomic integration vector containing the full set of proteins required for inducible CRISPRai and a gRNA array assembly method (FIG. 2e+f). The inducible CRISPRai vector has been designed to integrate at the HO locus, which is conserved between common lab strains, and is available with 6 auxotrophic and 4 antibiotic selectable markers (URA3, LEU2, HIS3, TRP1, LYS2, MET17, KanR, NatR, HygR, and ZeoR), and so should be appropriate for most yeast strains and applications. gRNA arrays are cloned into the vector using PCR generated 166 fragments that are assembled directly into the vector for up to 6 gRNAs in a single round of Golden Gate assembly, or up to 24 gRNAs via four intermediate sub-array plasmids in two rounds of Golden Gate assembly (FIGS. 7 and 8). gRNAs for gene activation (dCas12a-VP) and repression (dCas9-Mxi1) can be organized in any order, and Csy4 sites are positioned scarlessly either side of each guide to ensure processed RNA structures are equivalent. The limit of 6 gRNAs per vector or sub-array (24 gRNAs when sub-arrays are added together) is recommended to ensure a tight off state by keeping the distribution of mutTetO sites within the limits of mutTetR-Mxi1 silencing, and additionally simplifies validation of array identity by Sanger sequencing.

### Application of CRISPRai Toolkit for Metabolic Engineering

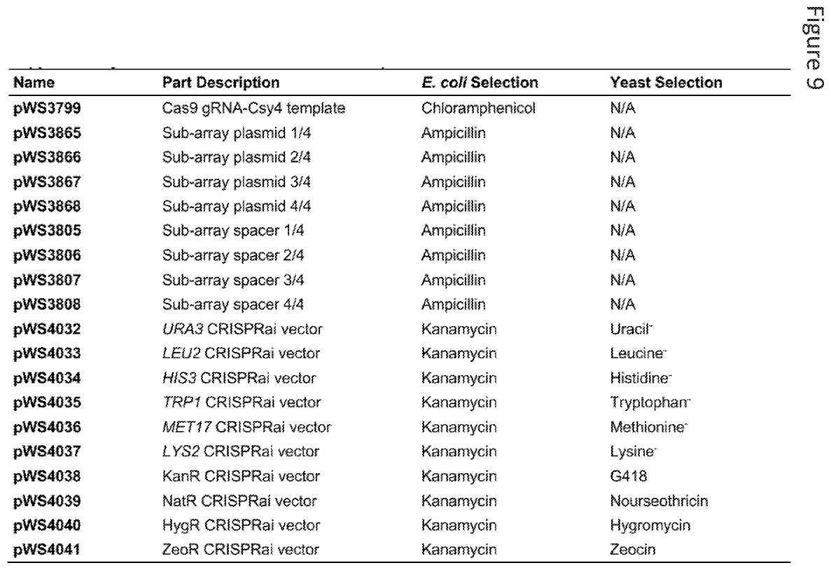

As we anticipate that metabolic engineering will be a major application of the inducible CRISPRai platform in yeast, we next sought to assess how the system would perform over time in batch culture, aiming to achieve stable activation and repression over time. We thus designed an experiment to repress and activate fluorescence reporter expression and measure the output at 24-hour intervals after a single induction at 0 h. We assembled a CRISPRai array consisting of 3 activation and 3 repression gRNAs targeting the RNR2 and TEF1 promoters driving the expression of mScarlet-I and mTagBFP2, respectively, and transformed this into the dual reporter strain (FIG. 3a). 1 day after induction, mScarlet-I expression increased by 800% and mTagBFP2 expression decreased by 90%. Repression and activation were maintained over at least five days (FIG. 2b and FIG. 5). Additionally, the array remained stable in the uninduced state over at least a week of daily cell passaging, thus avoiding possible phenotypic loss before the experiment has begun (FIG. 6).

To test whether the system can be practically used for increasing the production of metabolites, we 187 constructed an inducible array of 11 gRNAs targeting strategic nodes in central metabolism for repression and activation, based on past publications on succinic acid overproduction in yeast³²⁻³⁴ (FIG. 3c). The array contains 9 repression gRNAs targeting ADH1, ADH3, FUM1, IDP1, SDH1, SDH3, SER3, 190 SDH2, and SER33, and 2 activation gRNAs targeting SER33 and ADR1 (FIG. 3d, Targeted). gRNA targets were designed in Benchling, targeting activation gRNAs between −200 and −350 bp and repression gRNAs between −100 and +150 bp relative to the start codon location of the chosen genes. An additional control array was created using repression and activation gRNAs encoding a random spacer sequence that is not present within the genome, with this confirmed using the Benchling CRISPR tool off-target score and BLAST (Untargeted).

We transformed the arrays into wildtype (WT) BY4741 yeast, with the remaining auxotrophic markers introduced on a single-copy plasmid to create fully complemented strains for growth in minimal media³⁵. In the induced state, a 45-fold increase in succinic acid production was seen in the Targeted strain vs. WT strain after 2 days in batch culture (WT=9.37±3.8 mg/L, Targeted=426.9±13.3 mg/L), representing a 16-fold change in succinic acid when compared to the uninduced Targeted strain (FIG. 3f). No significant difference was seen between the induced and uninduced WT and Untargeted controls, validating that the increase in succinic acid was indeed due to the CRISPRai system. Finally, no major differences in succinic acid titres were measured between all 3 strains in the uninduced condition (WT=13.9±3.0 mg/L, Untargeted=19.2±0.6 mg/L, Targeted=26.4±0.5 mg/L), demonstrating that the inducibility of the CRISPRai system is highly controlled, as seen in our previous experiments with the regulation of fluorescent protein expression.

### REFERENCES

1. Gilbert, L. A. et al. Genome-Scale CRISPR-Mediated Control of Gene Repression and 394 Activation. Cell 159, 647-661 (2014).
2. Dominguez, A. A., Lim, W. A. & Qi, L. S. Beyond editing: repurposing CRISPR-Cas9 for precision genome regulation and interrogation. Nat. Rev. Mol. Cell Biol. 17, 5-15 (2016).
3. McCarty, N. S., Graham, A. E., Studená, L. & Ledesma-Amaro, R. Multiplexed CRISPR technologies for gene editing and transcriptional regulation. Nat. Commun. 11, 1281 (2020).
4. Kiattisewee, C. et al. Portable bacterial CRISPR transcriptional activation enables metabolic engineering in Pseudomonas putida. Metab. Eng. 66, 283-295 (2021).
5. Ho, H., Fang, J. R., Cheung, J. & Wang, H. H. Programmable CRISPR-Cas transcriptional activation in bacteria. Mol. Syst. Biol. 16, 1-12 (2020).
6. Dong, C. et al. A Single Cas9-VPR Nuclease for Simultaneous Gene Activation, Repression, and Editing in Saccharomyces cerevisiae. ACS Synth. Biol. 9, 2252-2257 (2020).
7. Deaner, M., Mejia, J. & Alper, H. S. Enabling Graded and Large-Scale Multiplex of Desired Genes Using a Dual-Mode dCas9 Activator in Saccharomyces cerevisiae. ACS Synth. Biol. 6, 1931-1943 (2017).
8. Schilling, C., Koffas, M. A. G., Sieber, V. & Schmid, J. Novel Prokaryotic CRISPR-Cas12a Based Tool for Programmable Transcriptional Activation and Repression. ACS Synth. Biol. 9, 410 3353-3363 (2020).
9. Dong, C., Fontana, J., Patel, A., Carothers, J. M. & Zalatan, J. G. Synthetic CRISPR-Cas gene activators for transcriptional reprogramming in bacteria. Nat. Commun. 9, 2489 (2018).
10. Gilbert, L. A. et al. CRISPR-Mediated Modular RNA-Guided Regulation of Transcription in Eukaryotes. Cell 154, 442-451 (2013).
11. Lian, J., HamediRad, M., Hu, S. & Zhao, H. Combinatorial metabolic engineering using an orthogonal tri-functional CRISPR system. Nat. Commun. 8, 1688 (2017).
12. Gao, Y. et al. Complex transcriptional modulation with orthogonal and inducible dCas9 regulators. Nat. Methods 13, 1043-1049 (2016).
13. Zalatan, J. G. et al. Engineering Complex Synthetic Transcriptional Programs with CRISPR RNA Scaffolds. Cell 160, 339-350 (2015),
14. Truong, V. A. et al. CRISPRai for simultaneous gene activation and inhibition to promote stem cell chondrogenesis and calvarial bone regeneration. Nucleic Acids Res. 47, e74-e74 (2019).
15. Martella, A. et al. Systematic Evaluation of CRISPRa and CRISPRi Modalities Enables Development of a Multiplexed, Orthogonal Gene Activation and Repression System. ACS 425 Synth. Biol. 8, 1998-2006 (2019).
16. Ye, L. et al. Programmable DNA repair with CRISPRa/i enhanced homology-directed repair efficiency with a single Cas9. Cell Discov. 4, 46 (2018).
17. Jensen, E. D. et al. Transcriptional reprogramming in yeast using dCas9 and combinatorial 429 gRNA strategies. Microb. Cell Fact. 16, 1-16 (2017).
18. Campa, C. C., Weisbach, N. R., Santinha, A. J., Incarnato, D. & Platt, R. J. Multiplexed genome engineering by Cas12a and CRISPR arrays encoded on single transcripts. Nat. Methods 16, 887-893 (2019).
19. McCarty, N. S., Shaw, W. M., Ellis, T. & Ledesma-Amaro, R. Rapid Assembly of gRNA Arrays via Modular Cloning in Yeast. ACS Synth. Biol. 8, (2019).
20. Zhang, Y. et al. A gRNA-tRNA array for CRISPR-Cas9 based rapid multiplexed genome editing in Saccharomyces cerevisiae. Nat. Commun. 10, 1053 (2019).
21. Chang, Y., Su, T., Qi, Q. & Liang, Q. Easy regulation of metabolic flux in Escherichia coli using an endogenous type I-E CRISPR-Cas system. Microb. Cell Fact. 15, 195 (2016).
22. Jack, B. R. et al. Predicting the Genetic Stability of Engineered DNA Sequences with the EFM Calculator. ACS Synth. Biol. 4, 939-943 (2015).
23. Zhang, R., Xu, W., Shao, S. & Wang, Q. Gene Silencing Through CRISPR Interference in 442 Bacteria: Current Advances and Future Prospects. Front. Microbiol. 12, 1-8 (2021).
24 Dai, X., Chen, X., Fang, Q., Li, J. & Bai, Z. Inducible CRISPR genome-editing tool: classifications and future trends. Crit. Rev. Biotechnol. 38, 573-586 (2018).
25. Santos-Moreno, J. & Schaerli, Y. CRISPR-based gene expression control for synthetic gene circuits. Biochem. Soc. Trans. 48, 1979-1993 (2020).
26. Gossen, M. & Bujard, H. Tight control of gene expression in mammalian cells by tetracycline responsive promoters. Proc. Natl. Acad. Sci. 89, 5547-5551 (1992).
27. Krueger, M., Scholz, O., Wisshak, S. & Hillen, W. Engineered Tet repressors with recognition 450 specificity for the tetO-4C5G operator variant. Gene, 93-100 (2007).
28. Chen, Y. et al. Genetic circuit design automation for yeast. Nat. Microbiol. 5, 1349-1360 (2020).
29. Ciurkot, K., Gorochowski, T. E., Roubos, J. A. & Verwaal, R. Efficient multiplexed gene regulation in Saccharomyces cerevisiae using dCas12a. Nucleic Acids Res. 49, 7775-7790 (2021).
30. Santos-Moreno, J., Tasiudi, E., Stelling, J. & Schaerli, Y. Multistable and dynamic CRISPRi based synthetic circuits. Nat. Commun. 11, 2746 (2020).
31. Lee, M. E., DeLoache, W. C., Cervantes, B. & Dueber, J. E. A Highly Characterized Yeast Toolkit for Modular, Multipart Assembly. ACS Synth. Biol. 4, 975-986 (2015). Raab, A. M., Gebhardt, G., Bolotina, N., Weuster-Botz, D. & Lang, C. Metabolic 32 engineering of Saccharomyces cerevisiae for the biotechnological production of succinic acid. Metab. Eng. 12, 518-525 (2010).
33. Ito, Y., Hirasawa, T. & Shimizu, H. Metabolic engineering of Saccharomyces cerevisiae to improve succinic acid production based on metabolic profiling. Biosci. Biotechnol. Biochem. 78, 151-159 (2014).
34. Franco-Duarte, R. et al. Genomic and transcriptomic analysis of Saccharomyces cerevisiae isolates with focus in succinic acid production. FEMS Yeast Res. 17, 1-12 (2017).
35. Mülleder, M., Campbell, K., Matsarskaia, O., Eckerstorfer, F. & Ralser, M. Saccharomyces cerevisiae single-copy plasmids for auxotrophy compensation, multiple marker selection, and for designing metabolically cooperating communities. F1000Research 5, 2351 (2016).
36. Gietz, R. D. & Schiestl, R. H. Microtiter plate transformation using the LiAc/SS carrier DNA/PEG method. Nat. Protoc. 2, 5-8 (2007).
37. Freundlieb, S., Schirra-Müller, C. & Bujard, H. A tetracycline controlled activation/repression system with increased potential for gene transfer into mammalian cells. J. Gene Med. 1, 4-12 (1999).

1. A nucleic acid construct comprising:
a) a promoter module wherein the promoter module comprises at least one promoter operator of a first sequence; and
b) a gene-regulating and/or gene-editing array module, that comprises at least one array sub-module, wherein each array sub-module comprises at least a first nucleic acid region that encodes a nucleic acid that is capable of directing RNA mediated gene regulation or RNA mediated gene editing,
wherein the gene-regulating and/or gene-editing array module is capable of being transcribed into a single polycistronic nucleic acid transcript from a single promoter,
and wherein between each nucleic acid region that encodes a nucleic acid that is capable of directing RNA mediated gene regulation or RNA mediated gene editing is a sequence that when in RNA form is an RNA cleavage site; and
wherein each array sub-module comprises at least one array operator of a second sequence
and wherein the gene-regulating and/or gene-editing array module is operably linked to promoter module.

2. The nucleic acid construct of claim 1, wherein the promotor module further comprises at least one promoter operator of a second sequence; optionally wherein:
a) the sequence of the at least one promoter operator of a first sequence and the at least one promoter operator of a second sequence are different; and/or
b) the sequence of the at least one promoter operator of a first sequence and the at least one array operator of a second sequence are the same.

3. The nucleic acid construct of claim 1, wherein the sequence of the at least one promoter operator of a first sequence and the at least one array operator of a second sequence are different.

4. The nucleic acid construct according to claim 1, wherein:
a) the promoter module comprises at least 2, at least 3, at least 4, at least 5, at least 6, at least 7, at least 8, at least 9, at least 10 or more promoter operators of a first sequence;
b) each array sub-module comprises at least 2, at least 3, at least 4, at least 5, at least 6, at least 7, at least 8, at least 9, or at least 10 array operators of a second sequence; and/or
c) where the promotor module further comprises at least one promoter operator of a second sequence, the promoter module comprises at least 2, at least 3, at least 4, at least 5, at least 6, at least 7, at least 8, at least 9, at least 10 or more promoter operators of a second sequence.

5. The nucleic acid construct of claim 1, wherein:
a) the gene-regulating and/or gene-editing array module comprises:
i) between 2 and 100 array sub-modules; optionally between 5 and 95, 10 and 90, 15 and 85, 20 and 80, 25 and 75, 30 and 70, 35 and 65, 40 and 60, 45 and 55 array sub-modules; and/or
ii) at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 77, at least 76, at least 77, at least 78, at least 79, at least 80, at least 81, at least 82, at least 83, at least 84, at least 88, at least 86, at least 87, at least 88, at least 89, at least 90, at least 91, at least 92, at least 93, at least 94, at least 99, at least 96, at least 97, at least 98, at least 99, or more array sub-modules; optionally comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 66, 66, 67, 68, 69, 70, 71, 72, 73, 74, 77, 76, 77, 78, 79, 80, 81, 82, 83, 84, 88, 86, 87, 88, 89, 90, 91, 92, 93, 94, 99, 96, 97, 98, 99, or 100 array sub-modules; and/or
b) wherein at least one array sub-module comprises:
i) between 2 and 100 nucleic acid regions that each encode a nucleic acid that is capable of directing RNA mediated gene regulation or RNA mediated gene editing; optionally between 5 and 95, 10 and 90, 15 and 85, 20 and 80, 25 and 75, 30 and 70, 35 and 65, 40 and 60, 45 and 55 nucleic acid regions that each encode a nucleic acid that is capable of directing RNA mediated gene regulation or RNA mediated gene editing; and/or
i) at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 77, at least 76, at least 77, at least 78, at least 79, at least 80, at least 81, at least 82, at least 83, at least 84, at least 88, at least 86, at least 87, at least 88, at least 89, at least 90, at least 91, at least 92, at least 93, at least 94, at least 99, at least 96, at least 97, at least 98, at least 99, or more nucleic acid regions that each encode a nucleic acid that is capable of directing RNA mediated gene regulation or RNA mediated gene editing; optionally comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 66, 66, 67, 68, 69, 70, 71, 72, 73, 74, 77, 76, 77, 78, 79, 80, 81, 82, 83, 84, 88, 86, 87, 88, 89, 90, 91, 92, 93, 94, 99, 96, 97, 98, 99, or 100 nucleic acid regions that each encode a nucleic acid that is capable of directing RNA mediated gene regulation or RNA mediated gene editing.

6. The nucleic acid construct according to claim 1, wherein each array sub-module comprises a single array operator of a second sequence; and/or
within each array sub-module:
a) the array operator is located upstream (5′) of the at least a first nucleic acid region that encodes a nucleic acid that is capable of directing RNA mediated gene regulation or RNA mediated gene editing; or
b) the array operator is located downstream (3′) of the at least a first nucleic acid region that encodes a nucleic acid that is capable of directing RNA mediated gene regulation or RNA mediated gene editing.

7. The nucleic acid construct according to claim 1, wherein:
a) at least one array sub-module comprises at least:
i) a first and a second nucleic acid region that encodes a nucleic acid that is capable of directing RNA mediated gene regulation or RNA mediated gene editing;
ii) a first, a second and a third nucleic acid region that encodes a nucleic acid that is capable of directing RNA mediated gene regulation or RNA mediated gene editing;
iii) a first, a second, a third and a fourth nucleic acid region that encodes a nucleic acid that is capable of directing RNA mediated gene regulation or RNA mediated gene editing;
iv) a first, a second, a third, a fourth and a fifth nucleic acid region that encodes a nucleic acid that is capable of directing RNA mediated gene regulation or RNA mediated gene editing;
v) a first, a second, a third, a fourth, a fifth and a sixth nucleic acid region that encodes a nucleic acid that is capable of directing RNA mediated gene regulation or RNA mediated gene editing;
vi) a first, a second, a third, a fourth, a fifth, a sixth and a seventh nucleic acid region that encodes a nucleic acid that is capable of directing RNA mediated gene regulation or RNA mediated gene editing;
vii) a first, a second, a third, a fourth, a fifth, a sixth, a seventh and an eighth nucleic acid region that encodes a nucleic acid that is capable of directing RNA mediated gene regulation or RNA mediated gene editing;
viii) a first, a second, a third, a fourth, a fifth, a sixth, a seventh, an eighth and a ninth nucleic acid region that encodes a nucleic acid that is capable of directing RNA mediated gene regulation or RNA mediated gene editing;
ix) a first, a second, a third, a fourth, a fifth, a sixth, a seventh, an eighth, a ninth and a tenth nucleic acid region that encodes a nucleic acid that is capable of directing RNA mediated gene regulation or RNA mediated gene editing;
and wherein the array operator is located:
upstream (5′) of the first nucleic acid region that encodes a nucleic acid that is capable of directing RNA mediated gene regulation or RNA mediated gene editing within the array sub-module; or
downstream (3′) of the last nucleic acid region that encodes a nucleic acid that is capable of directing RNA mediated gene regulation or RNA mediated gene editing within the array sub-module
so as to regulate transcription of sub-module;
and/or
b) the nucleic acid construct comprises at least a first array sub-module and a second array sub-module that each comprises at least:
i) a first and a second nucleic acid nucleic acid region that encodes a nucleic acid that is capable of directing RNA mediated gene regulation or RNA mediated gene editing;
ii) a first, a second and a third nucleic acid nucleic acid region that encodes a nucleic acid that is capable of directing RNA mediated gene regulation or RNA mediated gene editing;
iii) a first, a second, a third and a fourth nucleic acid region that encodes a nucleic acid that is capable of directing RNA mediated gene regulation or RNA mediated gene editing;
iv) a first, a second, a third, a fourth and a fifth nucleic acid region that encodes a nucleic acid that is capable of directing RNA mediated gene regulation or RNA mediated gene editing;
v) a first, a second, a third, a fourth, a fifth and a sixth nucleic acid region that encodes a nucleic acid that is capable of directing RNA mediated gene regulation or RNA mediated gene editing;
vi) a first, a second, a third, a fourth, a fifth, a sixth and a seventh nucleic acid region that encodes a nucleic acid that is capable of directing RNA mediated gene regulation or RNA mediated gene editing;
vii) a first, a second, a third, a fourth, a fifth, a sixth, a seventh and an eighth nucleic acid region that encodes a nucleic acid that is capable of directing RNA mediated gene regulation or RNA mediated gene editing;
viii) a first, a second, a third, a fourth, a fifth, a sixth, a seventh, an eighth and a ninth nucleic acid region that encodes a nucleic acid that is capable of directing RNA mediated gene regulation or RNA mediated gene editing; or
ix) a first, a second, a third, a fourth, a fifth, a sixth, a seventh, an eighth, a ninth and a tenth nucleic acid region that encodes a nucleic acid that is capable of directing RNA mediated gene regulation or RNA mediated gene editing;
and:
wherein the array operator is located upstream (5′) of the first nucleic acid sequence region that encodes a nucleic acid that is capable of directing RNA mediated gene regulation or RNA mediated gene editing within each array sub-module, so as to regulate transcription of each sub-module; and
wherein the first array sub-module is located upstream (5′) to the second array sub-module so that the array operator of the second array sub-module is positioned 3′ to the final nucleic acid region that encodes a nucleic acid that is capable of directing RNA mediated gene regulation or RNA mediated gene editing of the first array sub-module;
or
wherein the array operator is located upstream (3′) of the last nucleic acid region that encodes a nucleic acid that is capable of directing RNA mediated gene regulation or RNA mediated gene editing within each array sub-module, so as to regulate transcription of each sub-module; and
wherein the first array sub-module is located upstream (5′) to the second array sub-module so that the array operator of the first array sub-module is positioned 5′ to the first nucleic acid region that encodes a nucleic acid that is capable of directing RNA mediated gene regulation or RNA mediated gene editing of the second array sub-module.

8. The nucleic acid construct of claim 1, wherein:
a) the promoter operator of a first sequence:
i) is capable of binding to a first activator protein and/or a first repressor protein;
ii) is capable binding to a first activator protein in the presence of an inducing agent;
iii) is capable of binding to a first repressor protein in the absence of an inducing agent;
iv) is capable of binding to a first activator protein in the presence of an inducing agent and wherein said promoter operator of a first sequence is capable of binding to a first repressor protein in the absence of same said inducing agent;
v) the promoter operator of a first sequence is incapable of binding to a first repressor protein in the presence of an inducing agent;
and/or
b) the array operator of a second sequence:
i) is capable of binding to a second repressor protein;
ii) is capable of binding to a second repressor protein in the absence of an inducing agent;
iii) is incapable of binding to a protein in the presence of an inducing agent, optionally incapable of binding to the first activator protein, optionally is incapable of binding to the first activator protein in the presence of an inducing agent;
iv) is not capable of binding to an activator protein;
and/or
c) where the promotor module further comprises at least one promoter operator of a second sequence, the promoter operator of a second sequence:
i) is capable of binding to a second repressor protein;
ii) is capable of binding to a second repressor protein in the absence of an inducing agent; and/or
iii) is incapable of binding to a protein in the presence of an inducing agent, optionally incapable of binding to the first activator protein, optionally is incapable of binding to the first activator protein in the presence of an inducing agent;
optionally where the inducing agent is selected from the group comprising or consisting of: tetracycline (Tc); anhydrotetracycline (aTc); and Doxycycline; optionally wherein the inducer molecule is anhydrotetracycline (aTc).

9. The nucleic acid construct of claim 1 wherein:
a) the first repressor protein and the second repressor protein are the same repressor protein; or
b) the first repressor protein and the second repressor protein are different repressor proteins.

10. The nucleic acid construct of claim 1, wherein:
a) i) the promoter module is capable of initiating transcription of the gene-regulating and/or gene-editing array module in the presence of an inducing agent; and
ii) the promoter module is not capable of initiating transcription of the gene-regulating and/or gene-editing array module in the absence of said inducing agent;
and/or
b) i) the promoter module is capable of initiating transcription of the gene-regulating and/or gene-editing array module in the absence of the first repressor protein and/or the second repressor protein; and/or
ii) the promoter module is not capable of initiating transcription of the gene-regulating and/or gene-editing array module in the presence of the first repressor protein and/or the second repressor protein;
and/or
c) i) the promoter module is capable of initiating transcription of the gene-regulating and/or gene-editing array module when the first activator protein is present and the first repressor protein and/or the second repressor protein is absent; and/or
ii) the promoter module is not capable of initiating transcription of the gene-regulating and/or gene-editing array module when the first activator protein is absent and the first and/or second repressor protein is present.

11. The nucleic acid construct according to claim 1 wherein in the absence of an inducing agent:
a) the array operator(s) present in each array sub-module are occupied by a repressor protein; and/or
b) the promoter operator(s) present in the promoter module are occupied by a repressor protein.

12. The nucleic acid construct of claim 1, wherein:
a) the promoter operator of a first sequence is a TetO operator; optionally
wherein the promoter operator of a first sequence has a sequence that has at least 80%, or optionally at least 85%, at least 90%, at least 91%, at least 92%, at least 93%, at least 94%, at least 95%, at least 96%, at least 97%, at least 98%, at least 99%, or 100% sequence identity to SEQ ID NO: 1; or
wherein the first operator sequence has a sequence that is SEQ ID NO: 1;
and/or
b) the array operator of a second sequence is a mutTetO operator sequence;
optionally
wherein the array operator of a second sequence has is at least 80%, at least 85%, at least 90%, at least 91%, at least 92%, at least 93%, at least 94%, at least 95%, at least 96%, at least 97%, at least 98%, at least 99%, or more sequence identity to SEQ ID NO: 1; or
wherein the array operator of a second sequence has a sequence that is SEQ ID NO: 2; and/or
c) where the promotor module further comprises at least one promoter operator of a second sequence, the promoter operator of a second sequence is a mutTetO operator sequence; optionally
wherein the promoter operator of a second sequence has at least 80%, at least 85%, at least 90%, at least 91%, at least 92%, at least 93%, at least 94%, at least 95%, at least 96%, at least 97%, at least 98%, at least 99%, or more sequence identity to SEQ ID NO: 2; or
wherein the promoter operator of a second sequence has a sequence that is SEQ ID NO: 2.

13. The nucleic acid construct of claim 1, wherein:
a) the first activator protein is rtTA-VP or rtTA-Gal4;
b) the first repressor protein is TetR-Mxi1; and/or
c) the second repressor protein is mutTetR-Mxi1.

14. The nucleic acid construct of claim 1, wherein the at least one promoter of the promoter module is:
a) a Pol II promoter, optionally
wherein the promoter is an inducible promoter
wherein the Pol II promoter is classed as a strong promoter;
and/or
wherein the Pol II promoter is selected from the group consisting of a TDH3 promoter, a TEF1 promoter, a PGK1 promoter, a pCCW12 promoter, a pTEF2 promoter, a pHHF1 promoter, a pHHF2 promoter, a pALD6, promoter, a pGal1 promoter, a pPGK1 promoter, a pHTB2 promoter, a pCUP1 promoter, or a pTet promoter; or
b) a Pol III promoter, optionally
wherein the Pol III promoter is classed as a strong Pol III promoter;
wherein the Pol III promoter is an inducible promoter; and/or
wherein the Pol III promoter is selected from the group consisting of the tRNA Phe promoter with a 5′ HDV ribozyme, the U6 promoter or H1 promoter.

15. The nucleic acid construct of claim 1, wherein each nucleic acid that is capable of directing RNA mediated gene regulation or RNA mediated gene editing, when transcribed into RNA form, is independently capable of associating with a polypeptide, wherein said polypeptide is capable of regulating a gene,
optionally wherein said polypeptide is selected from the group comprising or consisting of:
Cas9 or Cas9-like polypeptide; dCas9 or dCas9-like polypeptide; Cas12a; dCas12a; Cas12b; dCas12b; Cas13a; dCas13a; Cas13b; dCas13b; LbCpf1; dLbCpf1; AsCpf1; dAsCpf1; or dFnCpf1; or FnCpf1; or a fusion protein thereof.

16. The nucleic acid construct of claim 1, wherein:
a) the nucleic acid region that encodes a nucleic acid that is capable of directing RNA mediated gene regulation or RNA mediated gene editing is complementary to a target nucleic acid region;
b) each nucleic acid region that encodes a nucleic acid that is capable of directing RNA mediated gene regulation or RNA mediated gene editing, is complementary to the same target nucleic acid region, optionally wherein the target nucleic acid region is a promoter of a target gene;
c) each nucleic acid region that encodes a nucleic acid that is capable of directing RNA mediated gene regulation or RNA mediated gene editing is complementary to the same target nucleic acid region, optionally wherein the target nucleic acid region is a promoter of a target gene,
but wherein the sequences of each nucleic acid region that encodes a nucleic acid that is capable of directing RNA mediated gene regulation or RNA mediated gene editing nucleic acid are different; and/or
d) within each array sub-module all of the nucleic acid regions that encode a nucleic acid that is capable of directing RNA mediated gene regulation or RNA mediated gene editing are each complementary to the same target nucleic acid region, optionally wherein the target nucleic acid region is a promoter of a target gene,
but wherein within each array sub-module the nucleic acid regions that encode a nucleic acid that is capable of directing RNA mediated gene regulation or RNA mediated gene editing are different.

17. The nucleic acid construct of claim 1 wherein:
each region that encodes a nucleic acid that is capable of directing RNA mediated gene regulation or RNA mediated gene editing, when transcribed into RNA form, are each independently capable of associating with a regulatory polypeptide, wherein said polypeptide is capable of regulating a gene,
optionally wherein said polypeptide is selected from the group comprising or consisting of:
Cas9 or Cas9-like polypeptide; dCas9 or dCas9-like polypeptide; Cas12a; dCas12a; Cas12b; dCas12b; Cas13a; dCas13a; Cas13b; dCas13b; LbCpf1; dLbCpf1; AsCpf1; dAsCpf1; or dFnCpf1; or FnCpf1; or a fusion protein thereof,
And wherein:
a) the region(s) that encode a nucleic acid that is capable of directing RNA mediated gene regulation or RNA mediated gene editing, when transcribed into RNA form, are each independently capable of associating with the same regulatory polypeptide; or
b) the region(s) that encode a nucleic acid that is capable of directing RNA mediated gene regulation or RNA mediated gene editing, when transcribed into RNA form, are each independently capable of associating with one of at least two different regulatory polypeptides.

18. The nucleic acid construct of claim 1, wherein the cleavage site is selected from:
i) a transcriptionally inert sequence;
ii) an endoribonuclease cleavage site, for example a site-specific RNA endonuclease site, for optionally a Csy4 cleavage sequence or an artificial site-specific RNA endonuclease
iii) a tRNA sequence
iv) a ribozyme sequence
v) an intron
vi) a target sequence for an RNA directed cleavage complex;
vii) a site cleavable by a Cas protein with RNA endonuclease activity, optionally cleavable by Cas12a; optionally
wherein the cleavage site is a Csy4 cleavage sequence, optionally
wherein the Csy4 cleavage site has a sequence that is at least 80%, at least 85%, at least 90%, at least 91%, at least 92%, at least 93%, at least 94%, at least 95%, at least 96%, at least 97%, at least 98%, at least 99%, or more sequence identity to SEQ ID NO: 18, SEQ ID NO: 19, SEQ ID NO: 20, and/or SEQ ID NO: 21. In some embodiments, the Csy4 cleavage site has a sequence that is SEQ ID NO: 18, SEQ ID NO: 19, SEQ ID NO: 20, or SEQ ID NO: 21.

19. The nucleic acid construct of claim 1, wherein the nucleic acid construct further comprises a regulatory protein module, wherein the regulatory protein module comprises:
a) a first nucleotide region encoding a first regulatory polypeptide; and/or
b) a second nucleotide region encoding a second regulatory polypeptide;
optionally wherein the first regulatory polypeptide and the second regulatory polypeptide are selected from the group comprising or consisting of:
Cas9 or Cas9-like polypeptide; dCas9 or dCas9-like polypeptide; Cas12a; dCas12a; Cas12b; dCas12b; Cas13a; dCas13a; Cas13b; dCas13b; LbCpf1; dLbCpf1; AsCpf1; dAsCpf1; or dFnCpf1; or FnCpf1; or a fusion protein thereof.

20. The nucleic acid construct of claim 19, wherein the regulatory polypeptide capable of regulating a gene, the first regulatory polypeptide and/or the second regulatory polypeptide is fused to an activator domain and/or a repressor domain; optionally wherein:
a) i) the activator domain is selected from the group comprising or consisting of: VP, VP16, VP64, GALA and B42; and/or
ii) wherein the repressor domain is selected from the group comprising or consisting of: KRAB-like effectors (optionally Mxi1), RD1152, RD11, RD5, and/or RD2;
and/or
b) i) the first regulatory polypeptide is selected from the group comprising or consisting of a Cas9-Mxi1 or Cas9-like-Mxi1 polypeptide; a dCas9-Mxi1 or dCas9-like-Mxi1 polypeptide; Cas12a-Mxi1; dCas12a-Mxi1; Cas12b-Mxi1; dCas12b-Mxi1; Cas13a-Mxi1; dCas13a-Mxi1; Cas13b-Mxi1; dCas13b-Mxi1; LbCpf1-Mxi1; dLbCpf1-Mxi1; AsCpf1-Mxi1; dAsCpf1-Mxi1; dFnCpf1-Mxi1; or FnCpf1-Mxi1, optionally is a dCas9-Mxi1 polypeptide; optionally
wherein the first regulatory polypeptide is encoded by a sequence that:
is at least 80%, at least 85%, at least 90%, at least 91%, at least 92%, at least 93%, at least 94%, at least 95%, at least 96%, at least 97%, at least 98%, at least 99%, or more identical to SEQ ID NO: 37; or
is 80%, 85%, 90%, 91%, 92%, 93%, 94%, 95%, 96%, 99%, 100% identical to SEQ ID NO: 37; or
is SEQ ID NO: 37;
and/or
ii) the second regulatory polypeptide is selected from the group comprising or consisting of a Cas9-VP or Cas9-like-VP polypeptide; a dCas9-VP or dCas9-like-VP polypeptide; Cas12a-VP; dCas12a-VP; Cas12b-VP; dCas12b-VP; Cas13a-VP; dCas13a-VP; Cas13b-VP; dCas13b-VP; LbCpf1-VP; dLbCpf1-VP; AsCpf1-VP; dAsCpf1-VP; dFnCpf1-VP; or FnCpf1-VP, optionally is a dCas12a-VP polypeptide; optionally
wherein the second regulatory polypeptide is encoded by a sequence that:
is at least 80%, at least 85%, at least 90%, at least 91%, at least 92%, at least 93%, at least 94%, at least 95%, at least 96%, at least 97%, at least 98%, at least 99%, or more identical to SEQ ID NO: 38; or
is 80%, 85%, 90%, 91%, 92%, 93%, 94%, 95%, 96%, 99%, 100% identical to SEQ ID NO: 38; or
is SEQ ID NO: 38.

21. The nucleic acid construct of claim 1, further comprising a nucleic acid region encoding a polypeptide that is capable of cleaving the cleavage site present in the array module when in RNA form; optionally
wherein the polypeptide that is capable of cleaving the cleavage site when in RNA form is:
i) an endoribonuclease, for example a site-specific RNA endonuclease, for example a Csy4 or an artificial site-specific RNA endonuclease;
ii) a polypeptide capable of cleaving a tRNA sequence
iii) a polypeptide capable of cleaving an intron sequence; or
v) polypeptide capable of cleaving a target sequence for an RNA directed cleavage complex; or
vi) a Cas protein with RNA endonuclease activity, optionally cleavable by Cas12a; optionally
wherein the polypeptide that is capable of cleaving the cleavage site when in RNA form is Csy4, optionally
wherein the polypeptide that is capable of cleaving the cleavage site when in RNA form is encoded by a sequence that is at least 80%, at least 85%, at least 90%, at least 91%, at least 92%, at least 93%, at least 94%, at least 95%, at least 96%, at least 97%, at least 98%, at least 99%, or more identical to SEQ ID NO: 39; or
is 80%, 85%, 90%, 91%, 92%, 93%, 94%, 95%, 96%, 99%, 100% identical to SEQ ID NO: 39; or
is SEQ ID NO: 39.

22. The nucleic acid construct of claim 19, wherein:
a) the first nucleotide region encoding a first regulatory polypeptide is operably linked to a promoter region;
b) the second nucleotide region encoding a second regulatory polypeptide is operably linked to a promoter region; and
c) the nucleic acid region encoding a polypeptide that is capable of cleaving the cleavage site present in the array module when in RNA form is operably linked to a promoter region;
optionally where the promoter region of (a), (b) and (c) are different promoters; and optionally
wherein the promoter region of (a), (b) and/or (c) is:
a weak promoter or a medium-strength promoter; optionally
wherein the promoter is selected from the group comprising or consisting of: REV1, PSP2, HTB2, RAD27, or POP6.

23. The nucleic acid construct of claim 1, further comprising:
i) a nucleic acid sequence encoding a first activator protein; optionally
wherein the first activator protein is selected from the group comprising or consisting of: rtTA-VP and rtTA-Gal4; optionally
wherein the first activator protein is rtTA-Gal4
ii) a nucleic acid sequence encoding a first repressor protein; optionally
wherein the first repressor protein is TetR-Mxi1;
and/or
iii) a nucleic acid sequence encoding a second repressor protein; optionally wherein the first repressor protein is mutTetR-Mxi1;
optionally wherein the nucleic acid sequence encoding the first activator protein;
the nucleic acid sequence encoding the first repressor protein; and/or the nucleic acid sequence encoding the second repressor protein are each independently operably linked to a promoter sequence; and optionally
wherein the promoter region of (i), (ii) and/or (iii) sequence is:
a weak promoter or a medium-strength promoter; optionally
wherein the promoter is selected from the group comprising or consisting of: REV1, PSP2, HTB2, RAD27, or POP6.

24. The nucleic acid construct of claim 1, wherein the first regulatory polypeptide and the second regulatory polypeptide are each separately capable of directing RNA mediated gene regulation are capable of:
a) activating a gene; and/or
b) repressing a gene.

25. The nucleic acid construct according to claim 1, wherein is a DNA construct, optionally wherein:
a) the nucleic acid construct is a circular nucleic acid construct or a linear nucleic acid construct; and/or
b) the nucleic acid construct comprises at least one, optionally two regions of homology to a target locus in a target genome, arranged so as to allow homologous recombination to occur between the regions of homology in the nucleic acid construct and the corresponding regions of homology in the target genome so as to result in incorporation of the nucleic acid construct into the target genome.

26. A vector comprising the nucleic acid construct of claim 1.

27. A single polycistronic nucleic acid transcript transcribed from the promoter module of the nucleic acid construct according to claim 1 or vector comprising the nucleic acid construct according to claim 1.

28. A cell comprising the nucleic acid construct according to claim 1; the vector comprising the nucleic acid construct according to claim 1; and/or the single polycistronic nucleic acid transcript transcribed from the promoter module of the nucleic acid construct according to claim 1.

29. The cell according to claim 28, wherein:
a) the cell is a eukaryotic cell, optionally selected from a fungal cell; a plant cell; and an animal cell, optionally wherein the animal cell is a mammalian cell; and/or
b) the cell is a fungal cell, optionally is a fungal cell belonging to a genus selected from the group comprising or consisting of: Candida, Hansenula, Komagatella, Pichia, Ashbya, Blastobotrys, Cryptococcus, Cutaneotrichosporon, Dekkera, Kluveromyces, Rhodosporidium, Rhodotorula, Lipomyces, Saccharomyces, and Yarrowia; optionally the cell is a Saccharomyces cell; optionally
wherein the cell is a Saccharomyces cerevisiae cell; or
c) the cell is a prokaryotic cell, optionally is a bacterial cell, optionally is a bacterial cell belonging to a genus selected from the group comprising or consisting of: Escherichia, Pseudomonas, Vibrio, Bacillus, Clostridium, Lactobacillus, Lactococcus, Streptomyces.

30. The cell according to claim 28, wherein the nucleic acid construct or the vector:
a) is integrated into one or more chromosomes of the cell; or
b) is maintained episomally.

31. The cell according to claim 28, wherein:
a) the cell comprises a target nucleic acid region and wherein the at least first nucleic acid region that encodes a nucleic acid that is capable of directing RNA mediated gene regulation or RNA mediated gene editing is complementary to the target nucleic acid region, optionally wherein the target nucleic acid region is a promoter of a target gene; and/or
b) the cell comprises:
i) between 2 and 100 target nucleic acid regions; optionally between 5 and 95, 10 and 90, 15 and 85, 20 and 80, 25 and 75, 30 and 70, 35 and 65, 40 and 60, 45 and 55 target nucleic acid regions; and/or
ii) at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 77, at least 76, at least 77, at least 78, at least 79, at least 80, at least 81, at least 82, at least 83, at least 84, at least 88, at least 86, at least 87, at least 88, at least 89, at least 90, at least 91, at least 92, at least 93, at least 94, at least 99, at least 96, at least 97, at least 98, at least 99, or more target nucleic acid regions; optionally comprises 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 66, 66, 67, 68, 69, 70, 71, 72, 73, 74, 77, 76, 77, 78, 79, 80, 81, 82, 83, 84, 88, 86, 87, 88, 89, 90, 91, 92, 93, 94, 99, 96, 97, 98, 99, or 100 target nucleic acid regions;
and wherein the nucleic acid construct or vector comprises a gene-regulating and/or gene-editing array module that comprises:
i) between 2 and 100 nucleic acid regions that each encode a nucleic acid that is capable of directing RNA mediated gene regulation or RNA mediated gene editing that is complementary to a target nucleic acid region in the cell; optionally between 5 and 95, 10 and 90, 15 and 85, 20 and 80, 25 and 75, 30 and 70, 35 and 65, 40 and 60, 45 and 55 nucleic acid regions that each encode a nucleic acid that is capable of directing RNA mediated gene regulation or RNA mediated gene editing that is complementary to a target nucleic acid region in the cell;
ii) at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 77, at least 76, at least 77, at least 78, at least 79, at least 80, at least 81, at least 82, at least 83, at least 84, at least 88, at least 86, at least 87, at least 88, at least 89, at least 90, at least 91, at least 92, at least 93, at least 94, at least 99, at least 96, at least 97, at least 98, at least 99, or more target nucleic acid regions; optionally comprises 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 66, 66, 67, 68, 69, 70, 71, 72, 73, 74, 77, 76, 77, 78, 79, 80, 81, 82, 83, 84, 88, 86, 87, 88, 89, 90, 91, 92, 93, 94, 99, 96, 97, 98, 99, or 100 nucleic acid regions that each encode a nucleic acid that is capable of directing RNA mediated gene regulation or RNA mediated gene editing that is complementary to a target nucleic acid region in the cell.

32. The cell of claim 28, wherein the cell constitutively expresses:
a) the first and/or second regulatory polypeptide; and/or
b) a polypeptide that is capable of cleaving the nucleic acid construct at the cleavage site when in RNA form.

33. A method of RNA mediated gene regulation of at least one target gene, the method comprising:
a) contacting the cell according to claim 28 with an inducer molecule; and
b) maintaining the cell in culture conditions suitable for the expression of the array module.

34. The method of claim 33, wherein:
a) the method comprises contacting the cell with a nuclease enzyme capable of cleaving the cleavage site when in RNA form, optionally
wherein said contacting is performed by expressing said nuclease enzyme within the cell; and/or
b) the inducer molecule is selected from the group comprising or consisting of: tetracycline (Tc); anhydrotetracycline (aTc); Doxycycline; optionally
wherein the inducer molecule is anhydrotetracycline (aTc).

35. The method according to claim 33 wherein the array module:
a) is not expressed in the absence of the inducer molecule;
b) is expressed only in the presence of the inducer molecule; and/or
c) has increased expression levels in the presence of the inducer molecule compared to the level of expression of the array module in the absence of the inducer molecule; optionally
wherein expression of the array module increases by at least 100%, at least 150%, at least 200%, at least 250%, at least 300%, at least 350%, at least 400%, at least 450%, at least 500%, at least 550%, at least 600%, at least 650%, at least 700%, at least 750%, at least 800%, at least 850%, at least 900%, at least 950%, at least 1000%, at least 1500%, at least 2000%, at least 2500%, at least 3000%, at least 3500%, at least 4000%, at least 4500%, at least 5000%, at least 5500%, at least 6000%, at least 6500%, at least 7000%, at least 7500%, at least 8000%, at least 8500%, at least 9000%, at least 10,000%, or more in the presence of the inducer molecule compared to the expression of the array module in the absence of the inducer molecule; optionally
wherein expression of the array module increases by 100%, 150%, 200%, 250%, 300%, 350%, 400%, 450%, 500%, 550%, 600%, 650%, 700%, 750%, 800%, 850%, 900%, 950%, 1000%, 1500%, 2000%, 2500%, 3000%, 3500%, 4000%, 4500%, 5000%, 5500%, 6000%, 6500%, 7000%, 7500%, 8000%, 8500%, 9000%, or 10,000% in the presence of the inducer molecule compared to the expression of the target gene in the absence of the inducer molecule.

36. Use of the method according to claim 33 in a process of producing at least one organic molecule.

37. A kit comprising:
(a) the nucleic acid construct according to claim 1;
(b) a vector comprising the nucleic acid construct according to claim 1;
(c) a single polycistronic nucleic acid transcript transcribed from the promoter module of the nucleic acid construct according to claim 1; or
(d) a cell comprising the nucleic acid construct according to claim 1; the vector comprising the nucleic acid construct according to claim 1; and/or the single polycistronic nucleic acid transcript transcribed from the promoter module of the nucleic acid construct according to claim 1.

38. A nucleic acid construct comprising:
a gene-regulating and/or gene-editing array module, that comprises at least one array sub-module, wherein each array sub-module comprises at least a first nucleic acid region that encodes a nucleic acid that is capable of directing RNA mediated gene regulation or RNA mediated gene editing,
wherein the gene-regulating and/or gene-editing array module is capable of being transcribed into a single polycistronic nucleic acid transcript from a single promoter,
and wherein between each nucleic acid region that encodes a nucleic acid that is capable of directing RNA mediated gene regulation or RNA mediated gene editing is a sequence that when in RNA form is an RNA cleavage site; and
wherein each array sub-module comprises at least one array operator of a second sequence
and wherein the gene-regulating and/or gene-editing array module is operably linked to promoter module.

39. A nucleic acid construct comprising:
a) a promoter module wherein the promoter module comprises at least one promoter operator of a first sequence; and
b) a gene-regulating and/or gene-editing array module, that comprises at least one array sub-module, wherein each array sub-module comprises at least a first nucleic acid region that encodes a nucleic acid that is capable of directing RNA mediated gene regulation or RNA mediated gene editing and a second nucleic acid region that encodes a nucleic acid that is capable of directing RNA mediated gene regulation or RNA mediated gene editing,
wherein the gene-regulating and/or gene-editing array module is capable of being transcribed into a single polycistronic nucleic acid transcript from a single promoter,
and wherein between each nucleic acid region that encodes a nucleic acid that is capable of directing RNA mediated gene regulation or RNA mediated gene editing is a sequence that when in RNA form is an RNA cleavage site; and
wherein each array sub-module comprises at least one array operator of a second sequence
and wherein the gene-regulating and/or gene-editing array module is operably linked to promoter module.

40. A nucleic acid construct comprising:
a) a promoter module wherein the promoter module comprises at least one promoter operator of a first sequence; and
b) a gene-regulating and/or gene-editing array module, that comprises at least two array sub-modules, wherein each array sub-module comprises at least a first nucleic acid region that encodes a nucleic acid that is capable of directing RNA mediated gene regulation or RNA mediated gene editing and a second nucleic acid region that encodes a nucleic acid that is capable of directing RNA mediated gene regulation or RNA mediated gene editing,
wherein the gene-regulating and/or gene-editing array module is capable of being transcribed into a single polycistronic nucleic acid transcript from a single promoter,
and wherein between each nucleic acid region that encodes a nucleic acid that is capable of directing RNA mediated gene regulation or RNA mediated gene editing is a sequence that when in RNA form is an RNA cleavage site; and
wherein each array sub-module comprises at least one array operator of a second sequence
and wherein the gene-regulating and/or gene-editing array module is operably linked to promoter module.
